SEQUENCE LISTING (3 sequences)

SEQ ID NO: 1 - protein, 5 aa, Homo sapiens
ARHCL

SEQ ID NO: 2 - protein, 4 aa, Homo sapiens
QNGS

SEQ ID NO: 3 - protein, 11 aa, Homo sapiens
PPFCVARCPSG

(19) United States
(12) Patent Application Publication
(10) Pub. No.: US 2024/0131180 A1
(43) Pub. Date: Apr. 25, 2024

(54) DRUG ANTIBODY CONJUGATES

(71) Applicant: PHARMA MAR, S.A., Madrid (ES)

(72) Inventors: Alfonso LATORRE LOZANO, Madrid (ES); Valentín MARTÍNEZ BARRASA, Madrid (ES); Andrés M. FRANCESCH SOLLOSO, Spain (ES); María Del Carmen CUEVAS MARCHANTE, Madrid (ES)

(21) Appl. No.: 17/920,000

(22) PCT Filed: Apr. 21, 2021
(86) PCT No.: PCT/EP2021/060352

§ 371 Date: Oct. 19, 2022

(30) Foreign Application Priority Data
Apr. 21, 2020 (EP) 20382320.8

(51) Int. Cl. A61K 47/68; A61K 47/54; A61P 35/00; C07D 515/22

(52) U.S. Cl. CPC A61K 47/6855; A61K 47/545; A61K 47/6803; A61K 47/6849; A61K 47/6889; A61P 35/00; C07D 515/22

(57) ABSTRACT

Drug conjugates having formula [D-(X)_{b}-(AA)_{w}-(T)_{g}-(L)-]ₙ-Ab wherein: D is a drug moiety having the following formula (I) or a pharmaceutically acceptable salt, ester, solvate, tautomer or stereoisomer thereof, (I) wherein D is covalently attached via a hydroxy or amine group to (X)_{b} if any, or (AA)_{w} if any, or to (T)_{g} if any, or (L); that are useful in the treatment of cancer.

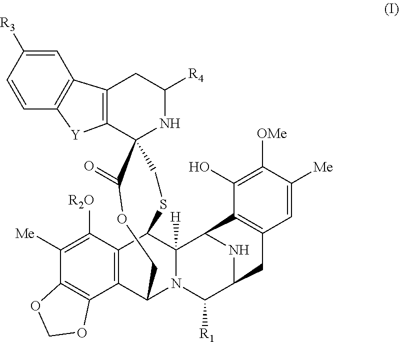

### FIELD OF THE INVENTION

The present invention relates to novel drug conjugates, drugs, drug-linker compounds, to methods for their preparation, pharmaceutical compositions containing said drug conjugates and their use as antitumoral agents.

### BACKGROUND TO THE INVENTION

The ecteinascidins are exceedingly potent antitumor agents isolated from the marine tunicate Ecteinascidia turbinata. One of these compounds, trabectedin, is been employed for the treatment of patients with advanced and metastatic soft tissue sarcoma (STS) after failure of anthracyclines and ifosfamide, or who are unsuited to receive such agents, and for the treatment of relapsed platinum-sensitive ovarian cancer in combination with pegylated liposomal doxorubicin.

U.S. Pat. No. 5,149,804 describes Ecteinascidin 722 (ET-722), isolated from the Caribbean tunicate Ecteinascidia turbinata, and its structure. ET-722 protects mice in vivo at very low concentrations against P388 lymphoma, B16 melanoma, and Lewis lung carcinoma.

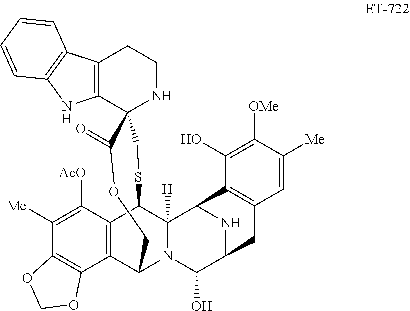 [SMILES: COc1c(C)cc2c(c1O)[C@H]1N[C@@H](C2)[C@H](O)N2[C@H]1[C@@H]1SC[C@]3(NCCc4c3[nH]c3ccccc43)C(=O)OC[C@H]2c2c3c(c(C)c(OC(C)=O)c21)OCO3]

WO03066638 describes several synthetic analogues of ET-722 and their cytotoxic activity against tumoral cells. In particular WO03066638 describes compounds 1 to 3 together with their cytotoxic activity against a panel of cancer cell lines.

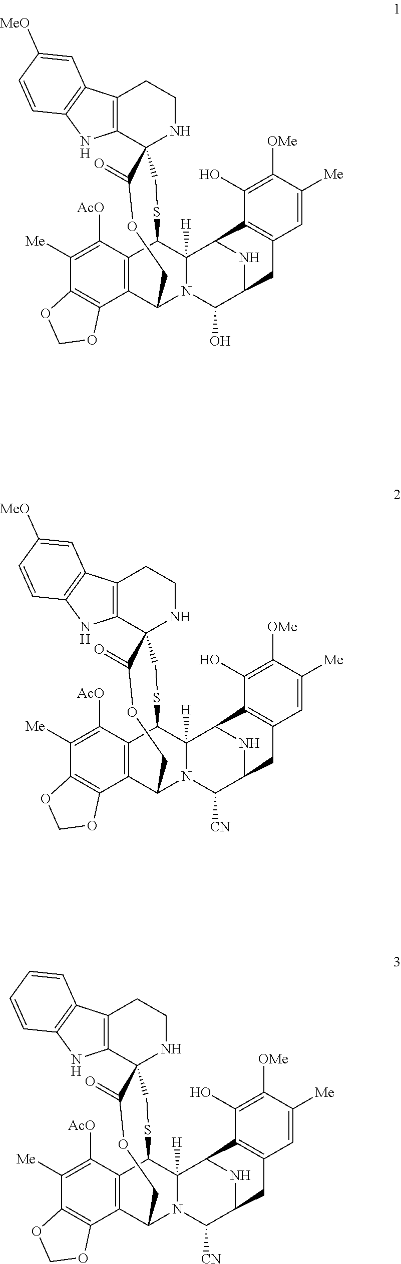 [SMILES: COc1ccc2[nH]c3c(c2c1)CCN[C@]31CS[C@@H]2c3c(OC(C)=O)c(C)c4c(c3[C@H](COC1=O)N1[C@@H]2[C@@H]2N[C@@H](Cc3cc(C)c(OC)c(O)c32)[C@@H]1O)OCO4 ; COc1ccc2[nH]c3c(c2c1)CCN[C@]31CS[C@@H]2c3c(OC(C)=O)c(C)c4c(c3[C@H](COC1=O)N1[C@@H]2[C@@H]2N[C@@H](Cc3cc(C)c(OC)c(O)c32)[C@@H]1C#N)OCO4 ; COc1c(C)cc2c(c1O)[C@H]1N[C@@H](C2)[C@H](C#N)N2[C@H]1[C@@H]1SC[C@]3(NCCc4c3[nH]c3ccccc43)C(=O)OC[C@H]2c2c3c(c(C)c(OC(C)=O)c21)OCO3]

Another compound described in WO 03/014127, lurbinectedin, is currently in clinical trials for the treatment of cancer. Lurbinectedin has the following chemical structure

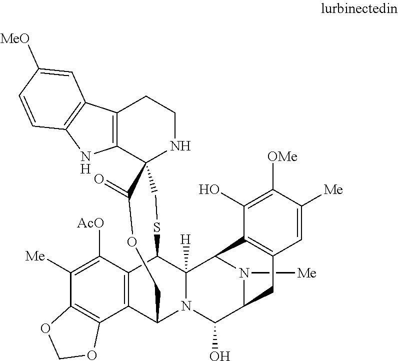 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

WO2018197663 is directed to novel ecteinascidin derivatives which demonstrate very promising anti-tumor activity. One of the compounds disclosed in such patent application is currently in Phase I clinical trials for the prevention and treatment of solid tumors.

The treatment of cancer has progressed significantly in recent years with the development of pharmaceutical entities that target and kill cancer cells more efficiently. Researchers have taken advantage of cell-surface receptors and antigens selectively expressed by target cells such as cancer cells to develop pharmaceutical entities based on antibodies that bind, in the example of tumors, the tumor-specific or tumor-associated antigens. In order to achieve this, cytotoxic molecules such as chemotherapeutic drugs, bacteria and plant toxins and radionuclides have been chemically linked to monoclonal antibodies that bind tumor-specific or tumor-associated cell surface antigens.

ADCs therefore represent a challenging area of development given the complex payload, linker and antibody structure but there remains a need for further ADCs to be developed.

### SUMMARY OF THE INVENTION

There is a need for novel active drug conjugates. The present invention addresses this need. It further provides novel drugs and drug-linker compounds for use in the preparation of drug conjugates of the present invention, processes for the preparation of the novel drug conjugates of the present invention, pharmaceutical compositions containing said drug conjugates and their use as antitumoral agents, as well as a kit comprising the drug conjugate of the present invention for use in the treatment of cancer.

In a first aspect of the present invention there is provided a drug conjugate comprising a drug moiety covalently attached to the rest of the drug conjugate, the drug conjugate having formula [D-(X)_{b}-(AA)_{w}-(T)_{g}-(L)-]ₙ-Ab wherein:
D is a drug moiety having the following formula (I) or a pharmaceutically acceptable salt, ester, solvate, tautomer or stereoisomer thereof,

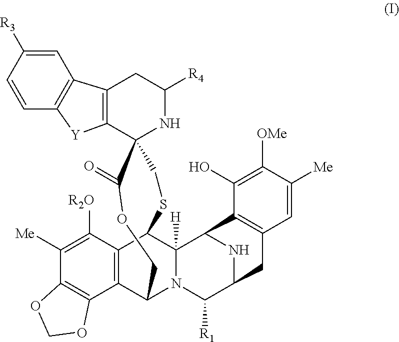

wherein:
D is covalently attached via a hydroxy or amine group to (X)_{b} if any, or (AA)_{w} if any, or to (T)_{g} if any, or (L);
Y is —NH— or —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂ alkenyl, and substituted or unsubstituted C₂-C₁₂ alkynyl;
R_{c} is selected from substituted or unsubstituted C₁-C₁₂ alkyl, substituted or unsubstituted C₂-C₁₂ alkenyl, and substituted or unsubstituted C₂-C₁₂ alkynyl; and
Prot^{NH} is a protecting group for amino;
X and T are extending groups that may be the same or different;
each AA is independently an amino acid unit;
L is a linker group;
w is an integer ranging from 0 to 12;
b is an integer of 0 or 1;
g is an integer of 0 or 1;
Ab is a moiety comprising at least one antigen binding site; and
n is the ratio of the group [D-(X)_{b}-(AA)_{w}-(T)_{g}-(L)-] to the moiety comprising at least one antigen binding site and is in the range from 1 to 20.

In a further aspect of the present invention there is provided a drug conjugate comprising a drug moiety covalently attached to the rest of the drug conjugate, the drug conjugate having formula [D-(X)_{b}-(AA)_{w}-(T)_{g}-(L)-]ₙ-Ab wherein:
D is a drug moiety having the following formula (I) or a pharmaceutically acceptable salt, ester, solvate, tautomer or stereoisomer thereof,

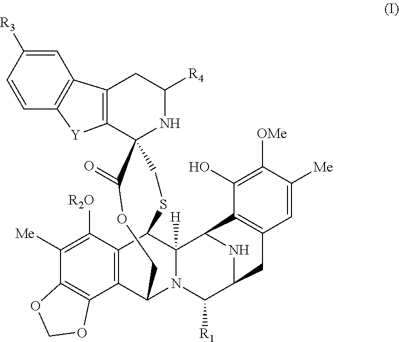

wherein:
D is covalently attached via a hydroxy or amine group to (X)_{b} if any, or (AA)_{w} if any, or to (T)_{g} if any, or (L);
Y is —NH— or —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂ alkenyl, and substituted or unsubstituted C₂-C₁₂ alkynyl;
R_{c} is selected from substituted or unsubstituted C₁-C₁₂ alkyl, substituted or unsubstituted C₂-C₁₂ alkenyl, and substituted or unsubstituted C₂-C₁₂ alkynyl; and
Prot^{NH} is a protecting group for amino;
with the proviso that when R₄ is hydrogen then Y is —O—;
X and T are extending groups that may be the same or different;
each AA is independently an amino acid unit;
L is a linker group;
w is an integer ranging from 0 to 12;
b is an integer of 0 or 1;
g is an integer of 0 or 1;
Ab is a moiety comprising at least one antigen binding site; and
n is the ratio of the group [D-(X)_{b}-(AA)_{w}-(T)_{g}-(L)-] to the moiety comprising at least one antigen binding site and is in the range from 1 to 20.

In a further aspect of the present invention there is provided a drug conjugate comprising a drug moiety covalently attached to the rest of the drug conjugate, the drug conjugate having formula [D-(X)_{b}-(AA)_{w}-(T)_{g}-(L)-]ₙ-Ab wherein:
D is a drug moiety having the following formula (IH) or a pharmaceutically acceptable salt, ester, solvate, tautomer or stereoisomer thereof,

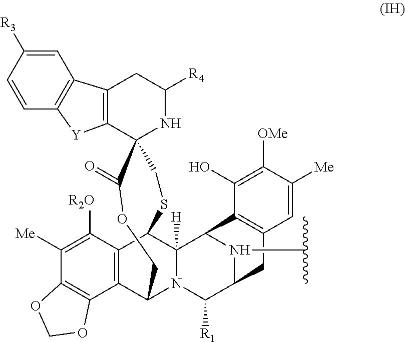

wherein:
the wavy line indicates the point of covalent attachment to (X)_{b} if any, or (AA)_{w} if any, or to (T)_{g} if any, or to (L);
Y is —NH— or —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂ alkenyl, and substituted or unsubstituted C₂-C₁₂ alkynyl;
R_{c} is selected from substituted or unsubstituted C₁-C₁₂ alkyl, substituted or unsubstituted C₂-C₁₂ alkenyl, and substituted or unsubstituted C₂-C₁₂ alkynyl; and
Prot^{NH} is a protecting group for amino;
X and T are extending groups that may be the same or different;
each AA is independently an amino acid unit;
L is a linker group;
w is an integer ranging from 0 to 12;
b is an integer of 0 or 1;
g is an integer of 0 or 1;
Ab is a moiety comprising at least one antigen binding site; and
n is the ratio of the group [D-(X)_{b}-(AA)_{w}-(T)_{g}-(L)-] to the moiety comprising at least one antigen binding site and is in the range from 1 to 20.

In a further aspect of the present invention there is provided a drug conjugate comprising a drug moiety covalently attached to the rest of the drug conjugate, the compound having formula [D-(X)_{b}-(AA)_{w}-(T)_{g}-(L)-]ₙ-Ab wherein:
D is a drug moiety having the following formula (I) or a pharmaceutically acceptable salt, ester, solvate, tautomer or stereoisomer thereof,

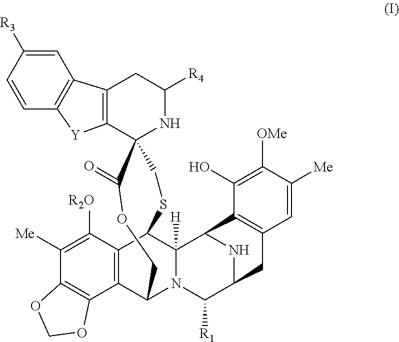

wherein:
D is covalently attached via a hydroxy or amine group to (X)_{b} if any, or (AA)_{w} if any, or to (T)_{g} if any, or (L);
Y is selected from —NH— and —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
R_{b} is selected from substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
R_{c} is selected from substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
Prot^{NH} is a protecting group for amino;
substituents Rₓ are selected from the group consisting of C₁-C₁₂ alkyl groups which may be optionally substituted with at least one group R_{y}, C₂-C₁₂ alkenyl groups which may be optionally substituted with at least one group R_{y}, C₂-C₁₂ alkynyl groups which may be optionally substituted with at least one group R_{y}, halogen atoms, oxo groups, thio groups, cyano groups, nitro groups, OR_{y}, OCOR_{y}, OCOOR_{y}, COR_{y}, COOR_{y}, OCONR_{y}R_{z}, CONR_{y}R_{z}, S(O)R_{y}, SO₂R_{y}, P(O)(R_{y})OR_{z}, NR_{y}R_{z}, NR_{y}COR_{z}, NR_{y}C(═O)NR_{y}R_{z}, NR_{y}C(═NR_{y})NR_{y}R_{z}, aryl groups having from 6 to 18 carbon atoms in one or more rings which may optionally be substituted with one or more substituents which may be the same or different selected from the group consisting of R_{y}, OR_{y}, OCOR_{y}, OCOOR_{y}, NR_{y}R_{z}, NR_{y}COR_{z}, and NR_{y}C(═NR_{y})NR_{y}R_{z}, aralkyl groups comprising an alkyl group having from 1 to 12 carbon atoms substituted with an optionally substituted aryl group as defined above, aralkyloxy groups comprising an alkoxy group having from 1 to 12 carbon atoms substituted with an optionally substituted aryl group as defined above, and a 5- to 14-membered saturated or unsaturated heterocyclic group having one or more rings and comprising at least one oxygen, nitrogen or sulphur atom in said ring(s), said heterocyclic group optionally being substituted with one or more substituents R_{y}, and where there is more than one optional substituents on any given group the optional substituents R_{y} may be the same or different;
each R_{y} and R_{z} is independently selected from the group consisting of hydrogen, C₁-C₁₂ alkyl groups, C₁-C₁₂ alkyl groups that are substituted with at least one halogen atom, aralkyl groups comprising a C₁-C₁₂ alkyl group that is substituted with an aryl group having from 6 to 18 carbon atoms in one or more rings and heterocycloalkyl groups comprising a C₁-C₁₂ alkyl group that is substituted with a 5- to 14-membered saturated or unsaturated heterocyclic group having one or more rings and comprising at least one oxygen, nitrogen or sulphur atom in said ring(s);
X and T are extending groups that may be the same or different;
each AA is independently an amino acid unit;
L is a linker group;
w is an integer ranging from 0 to 12;
b is an integer of 0 or 1;
g is an integer of 0 or 1;
where b+g+w is optionally not 0;
Ab is a moiety comprising at least one antigen binding site; and
n is the ratio of the group [D-(X)_{b}-(AA)_{w}-(T)_{g}-(L)-] to the moiety comprising at least one antigen binding site and is in the range from 1 to 20.

In a further aspect of the present invention there is provided a drug conjugate comprising a drug moiety covalently attached to the rest of the drug conjugate, the compound having formula [D-(X)_{b}-(AA)_{w}-(T)_{g}-(L)-]ₙ-Ab wherein:
D is a drug moiety having the following formula (IH) or a pharmaceutically acceptable salt, ester, solvate, tautomer or stereoisomer thereof,

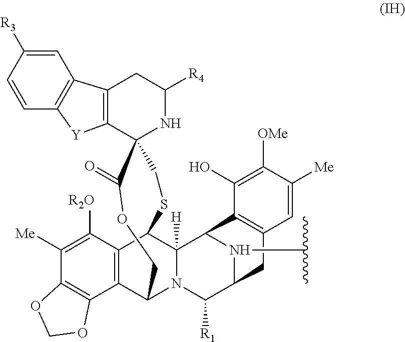

wherein:
the wavy line indicates the point of covalent attachment to (X)_{b} if any, or (AA)_{w} if any, or to (T)_{g} if any, or to (L);
Y is selected from —NH— and —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
R_{b} is selected from substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
R_{c} is selected from substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
Prot^{NH} is a protecting group for amino;
substituents Rₓ are selected from the group consisting of C₁-C₁₂ alkyl groups which may be optionally substituted with at least one group R_{y}, C₂-C₁₂ alkenyl groups which may be optionally substituted with at least one group R_{y}, C₂-C₁₂ alkynyl groups which may be optionally substituted with at least one group R_{y}, halogen atoms, oxo groups, thio groups, cyano groups, nitro groups, OR_{y}, OCOR_{y}, OCOOR_{y}, COR_{y}, COOR_{y}, OCONR_{y}R_{z}, CONR_{y}R_{z}, S(O)R_{y}, SO₂R_{y}, P(O)(R_{y})OR_{z}, NR_{y}R_{z}, NR_{y}COR_{z}, NR_{y}C(═O)NR_{y}R_{z}, NR_{y}C(═NR_{y})NR_{y}R_{z}, aryl groups having from 6 to 18 carbon atoms in one or more rings which may optionally be substituted with one or more substituents which may be the same or different selected from the group consisting of R_{y}, OR_{y}, OCOR_{y}, OCOOR_{y}, NR_{y}R_{z}, NR_{y}COR_{z}, and NR_{y}C(═NR_{y})NR_{y}R_{z}, aralkyl groups comprising an alkyl group having from 1 to 12 carbon atoms substituted with an optionally substituted aryl group as defined above, aralkyloxy groups comprising an alkoxy group having from 1 to 12 carbon atoms substituted with an optionally substituted aryl group as defined above, and a 5- to 14-membered saturated or unsaturated heterocyclic group having one or more rings and comprising at least one oxygen, nitrogen or sulphur atom in said ring(s), said heterocyclic group optionally being substituted with one or more substituents R_{y}, and where there is more than one optional substituents on any given group the optional substituents R_{y} may be the same or different;
each R_{y} and R_{z} is independently selected from the group consisting of hydrogen, C₁-C₁₂ alkyl groups, C₁-C₁₂ alkyl groups that are substituted with at least one halogen atom, aralkyl groups comprising a C₁-C₁₂ alkyl group that is substituted with an aryl group having from 6 to 18 carbon atoms in one or more rings and heterocycloalkyl groups comprising a C₁-C₁₂ alkyl group that is substituted with a 5- to 14-membered saturated or unsaturated heterocyclic group having one or more rings and comprising at least one oxygen, nitrogen or sulphur atom in said ring(s);
X and T are extending groups that may be the same or different;
each AA is independently an amino acid unit;
L is a linker group;
w is an integer ranging from 0 to 12;
b is an integer of 0 or 1;
g is an integer of 0 or 1;
where b+g+w is optionally not 0;
Ab is a moiety comprising at least one antigen binding site; and
n is the ratio of the group [D-(X)_{b}-(AA)_{w}-(T)_{g}-(L)-] to the moiety comprising at least one antigen binding site and is in the range from 1 to 20.

In a further aspect of the present invention there is provided a drug conjugate comprising a drug moiety covalently attached to the rest of the drug conjugate, the drug conjugate having formula [D-(X)_{b}-(AA)_{w}-(T)_{g}-(L)-]ₙ-Ab wherein:
D is a drug moiety having the following formula (I) or a pharmaceutically acceptable salt, ester, solvate, tautomer or stereoisomer thereof,

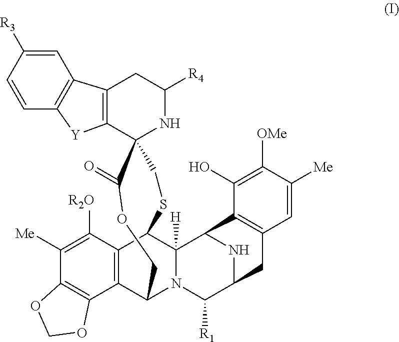

wherein:
D is covalently attached via a hydroxy or amine group to (X)_{b} if any, or (AA)_{w} if any, or to (T)_{g} if any, or (L);
Y is —NH— or —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂ alkenyl, and substituted or unsubstituted C₂-C₁₂ alkynyl;
R_{c} is selected from substituted or unsubstituted C₁-C₁₂ alkyl, substituted or unsubstituted C₂-C₁₂ alkenyl, and substituted or unsubstituted C₂-C₁₂ alkynyl; and
Prot^{NH} is a protecting group for amino;
X and T are extending groups that may be the same or different;
each AA is independently an amino acid unit;
L is a linker group;
w is an integer ranging from 0 to 12;
b is 1;
g is an integer of 0 or 1;
Ab is a moiety comprising at least one antigen binding site; and
n is the ratio of the group [D-(X)_{b}-(AA)_{w}-(T)_{g}-(L)-] to the moiety comprising at least one antigen binding site and is in the range from 1 to 20.

In a further aspect of the present invention there is provided a drug conjugate comprising a drug moiety covalently attached to the rest of the drug conjugate, the drug conjugate having formula [D-(X)_{b}-(AA)_{w}-(T)_{g}-(L)-]ₙ-Ab wherein:
D is a drug moiety having the following formula (I) or a pharmaceutically acceptable salt, ester, solvate, tautomer or stereoisomer thereof,

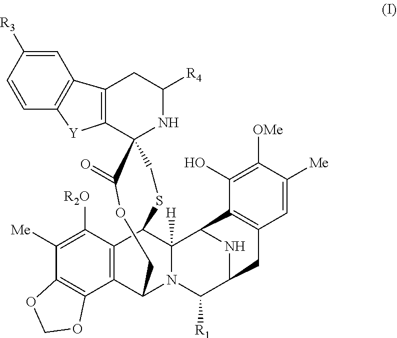

wherein:
D is covalently attached via a hydroxy or amine group to (X)_{b} if any, or (AA)_{w} if any, or to (T)_{g} if any, or (L);
Y is —NH— or —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂ alkenyl, and substituted or unsubstituted C₂-C₁₂ alkynyl;
R_{c} is selected from substituted or unsubstituted C₁-C₁₂ alkyl, substituted or unsubstituted C₂-C₁₂ alkenyl, and substituted or unsubstituted C₂-C₁₂ alkynyl; and
Prot^{NH} is a protecting group for amino;
X and T are extending groups that may be the same or different;
each AA is independently an amino acid unit;
L is a linker group;
w is 2;
b is 1;
g is an integer of 0 or 1;
Ab is a moiety comprising at least one antigen binding site; and
n is the ratio of the group [D-(X)_{b}-(AA)_{w}-(T)_{g}-(L)-] to the moiety comprising at least one antigen binding site and is in the range from 1 to 20.

As we shall explain and exemplify in greater detail below, the drug conjugates of formula [D-(X)_{b}-(AA)_{w}-(T)_{g}-(L)-]ₙ-Ab of the present invention represent a breakthrough in addressing the problems outlined above of requiring further drug conjugates in addition to those based on the three main families of cytotoxic drugs that have been used as payloads to date, that show excellent antitumor activity.

In a further aspect of the present invention, there is provided a compound of formula D-(X)_{b}-(AA)_{w}-(T)_{g}-L₁ or of formula D-(X)_{b}-(AA)_{w}-(T)_{g}-H, wherein:
L₁ is a linker selected from the group of formulas consisting of:

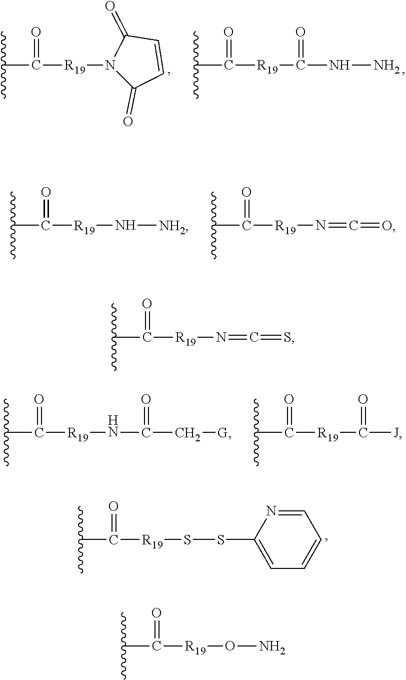

each of the the wavy lines indicates the point of covalent attachment to (T)_{g} if any, or (AA)_{w} if any, or to (X)_{b} if any, or to D;
G is selected from halo, —O-mesyl and —O-tosyl;
J is selected from halo, hydroxy, —N-succinimidoxy, —O-(4-nitrophenyl), —O— pentafluorophenyl, —O-tetrafluorophenyl and —O—C(O)—OR₂₀;
R₁₉ is selected from —C₁-C₁₂ alkylene-, —C₃-C₈ carbocyclo, —O—(C₁-C₁₂ alkylene), —C₆-C₁₈ arylene in one or more rings which may optionally be substituted with one or more substituents Rₓ, —C₁-C₁₂ alkylene-C₆-C₁₈ arylene- wherein the arylene group is in one or more rings which may optionally be substituted with one or more substituents Rₓ, —C₆-C₁₈ arylene-C₁-C₁₂ alkylene- wherein the arylene group is in one or more rings which may optionally be substituted with one or more substituents Rₓ, —C₁-C₁₂ alkylene-(C₃-C₈carbocyclo)-, —(C₃-C₈ carbocyclo)-C₁-C₁₂ alkylene-, —C₅-C₁₄ heterocyclo- wherein said heterocyclo group may be a saturated or unsaturated group having one or more rings and comprising at least one oxygen, nitrogen or sulphur atom in said ring(s), said group optionally being substituted with one or more substituents Rₓ, —C₁-C₁₂ alkylene-(C₅-C₁₄ heterocyclo)- wherein said heterocyclo group may be a saturated or unsaturated group having one or more rings and comprising at least one oxygen, nitrogen or sulphur atom in said ring(s), said group optionally being substituted with one or more substituents Rₓ, —(C₅-C₁₄ heterocyclo)-C₁-C₁₂ alkylene-, wherein said heterocyclo group may be a saturated or unsaturated group having one or more rings and comprising at least one oxygen, nitrogen or sulphur atom in said ring(s), said group optionally being substituted with one or more substituents Rₓ, —(OCH₂CH₂)ᵣ— and —CH₂—(OCH₂CH₂)ᵣ—, wherein each of the above alkylene substituents whether alone or attached to another moiety the carbon chain may optionally be substituted by one or more substituents Rₓ;
R₂₀ is a C₁-C₁₂ alkyl or an aryl group having from 6 to 18 carbon atoms in one or more aromatic rings, said aryl groups optionally being substituted with one or more substituents Rₓ;
r is an integer ranging from 1-10;
g is an integer of 0 or 1;
b is an integer of 0 or 1;
w is an integer ranging from 0 to 12; and
each of D, Rₓ, X, T, and AA is as defined in the first aspect of the invention.

In preferred embodiments of the present invention, b+g+w is not 0. In further embodiments, b+w is not 0. In yet further embodiments, when w is not 0, then b is 1. In a further embodiment, when w is 0 then b is 1.

In a further aspect of the present invention, there is provided a compound of formula D-(X)_{b}-(AA)_{w}-(T)_{g}-L₁ or of formula D-(X)_{b}-(AA)_{w}-(T)_{g}-H, or a pharmaceutically acceptable salt, ester, solvate, tautomer or stereoisomer thereof; wherein each of D, X, AA, T, L₁, b, g and w are as defined herein; but further wherein if the compound is a compound of formula D-(X)_{b}-(AA)_{w}-(T)_{g}-H then b+w+g≠0.

In a preferred embodiment according to aspects of the present invention, n is the ratio of the group [D-(X)_{b}-(AA)_{w}-(T)_{g}-(L)-] to the moiety comprising at least one antigen binding site and is in the range from 1 to 20. In further embodiments n is in the range from 1-12, 1-8, 3-8, 3-6, 3-5 or is 1, 2, 3, 4, 5 or 6 preferably, 3, 4 or 5 or 4.

In a further aspect of the present invention, there is provided a drug moiety D for use in an antibody drug conjugate. In a further aspect of the present invention, there is provided a drug moiety D for use as a payload in an antibody drug conjugate. In a further aspect of the present invention, there is provided the use of a drug moiety D as described herein, in the manufacture of an antibody drug conjugate.

In a further aspect of the present invention, there are provided drugs of formula (IA)

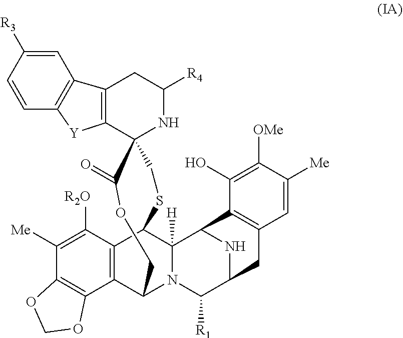

wherein:
Y is —NH— or —O—;
R₁ is —OH or —CN;
R₂ is a —C(═O)Rₐ group;
R₃ is hydrogen or a OR_{b} group;
R₄ is selected from hydrogen, —CH₂OH, —CH₂OC(═O)R_{c}, —CH₂NH₂ and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂ alkenyl, and substituted or unsubstituted C₂-C₁₂ alkynyl;
R_{c} is selected from substituted or unsubstituted C₁-C₁₂ alkyl, substituted or unsubstituted C₂-C₁₂ alkenyl, and substituted or unsubstituted C₂-C₁₂ alkynyl; and
Prot^{NH} is a protecting group for amino;
with the proviso that when R₄ is hydrogen, then Y is —O—.

In a further aspect of the present invention, there is provided a drug conjugate according to the present invention, for use as a medicament.

In a further aspect of the present invention, there is provided a pharmaceutical composition comprising a drug conjugate according to the present invention and a pharmaceutically acceptable carrier.

In a a further aspect of the present invention, there is provided a drug conjugate according to the present invention for use in the treatment of cancer.

In a further aspect of the present invention, there is provided a method for the prevention or treatment of cancer, comprising administering an effective amount of a drug conjugate according to the present invention to a patient in need thereof.

In a further aspect of the present invention, there is provided the use of a drug conjugate according to the present invention in the preparation of a medicament for the treatment of cancer.

In a further aspect of of the present invention, there is provided a kit comprising a therapeutically effective amount of a drug conjugate according to the present invention and a pharmaceutically acceptable carrier.

In the above aspects of the present invention, the cancer may be selected from lung cancer, including NSCLC, gastric cancer, colorectal cancer, breast cancer, pancreas carcinoma, endometrial cancer, bladder cancer, cervical cancer, esophageal cancer, gallbladder cancer, uterine cancer, salivary duct cancer, ovarian cancer, kidney cancer, leukaemia, multiple myeloma, and lymphoma. In a preferred embodiment, the cancer is a HER2 positive cancer. Preferred HER2 positive cancers include HER2 positive lung cancer including HER2 positive NSCLC, HER2 positive gastric cancer, HER2 positive colorectal cancer, HER2 positive breast cancer, HER2 positive pancreas carcinoma, HER2 positive endometrial cancer, HER2 positive bladder cancer, HER2 positive cervical cancer, HER2 positive esophageal cancer, HER2 positive gallbladder cancer, HER2 positive uterine cancer, HER2 positive salivary duct cancer and HER2 positive ovarian cancer. More preferred cancers are HER2 positive breast cancer, HER2 positive ovarian cancer and HER2 positive gastric cancer. Most preferred cancer is HER2 positive breast cancer.

In a further aspect of the present invention there is provided a process for the preparation of a drug conjugate according to the present invention comprising conjugating a moiety Ab comprising at least one antigen binding site and a drug D, Ab and D being as defined herein.

### DETAILED DESCRIPTION OF PREFERRED EMBODIMENTS

The following apply to all aspects of the present invention:

In the compounds of the present invention, the alkyl groups may be branched or unbranched, and preferably have from 1 to about 12 carbon atoms. One more preferred class of alkyl groups has from 1 to about 6 carbon atoms. Even more preferred are alkyl groups having 1, 2, 3 or 4 carbon atoms. Methyl, ethyl, n-propyl, isopropyl and butyl, including n-butyl, isobutyl, sec-butyl and tert-butyl are particularly preferred alkyl groups in the compounds of the present invention.

In the compounds of the present invention, the alkenyl groups may be branched or unbranched, have one or more double bonds and from 2 to about 12 carbon atoms. One more preferred class of alkenyl groups has from 2 to about 6 carbon atoms. Even more preferred are alkenyl groups having 2, 3 or 4 carbon atoms. Ethenyl, 1-propenyl, 2-propenyl, 1-methylethenyl, 1-butenyl, 2-butenyl, and 3-butenyl are particularly preferred alkenyl groups in the compounds of the present invention.

In the compounds of the present invention, the alkynyl groups may be branched or unbranched, have one or more triple bonds and from 2 to about 12 carbon atoms. One more preferred class of alkynyl groups has from 2 to about 6 carbon atoms. Even more preferred are alkynyl groups having 2, 3 or 4 carbon atoms.

Suitable aryl groups in the compounds of the present invention include single and multiple ring compounds, including multiple ring compounds that contain separate and/or fused aryl groups. Typical aryl groups contain from 1 to 3 separated and/or fused rings and from 6 to about 18 carbon ring atoms. Preferably aryl groups contain from 6 to about 10 carbon ring atoms. Specially preferred aryl groups included substituted or unsubstituted phenyl, substituted or unsubstituted naphthyl, substituted or unsubstituted biphenyl, substituted or unsubstituted phenanthryl and substituted or unsubstituted anthryl.

Suitable heterocyclic groups include heteroaromatic and heteroalicyclic groups containing from 1 to 3 separated and/or fused rings and from 5 to about 18 ring atoms. Preferably heteroaromatic and heteroalicyclic groups contain from 5 to about 10 ring atoms, most preferably 5, 6, or 7 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridyl, purinyl, oxadiazolyl, thiadiazolyl, furazanyl, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl and furopyridyl. Suitable heteroalicyclic groups in the compounds of the present invention contain one, two or three heteroatoms selected from N, O or S and include, e.g., pyrrolidinyl, tetrahydrofuranyl, tetrahydrothienyl, tetrahydrothiopyranyl, piperidyl, morpholinyl, thiomorpholinyl, thioxanyl, piperazinyl, azetidinyl, oxetanyl, thietanyl, homopiperidyl, oxepanyl, thiepanyl, oxazepinyl, diazepinyl, thiazepinyl, 1,2,3,6-tetrahydropyridyl, 2-pirrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

The groups above mentioned may be substituted at one or more available positions by one or more suitable groups such as OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, protected OH, protected amino, protected SH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, where each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. In addition, where there are more than one R′ groups on a substituent, each R′ may be the same or different.

In the compounds for the present invention, the halogen substituents include F, Cl, Br, and I.

More particularly, in the compounds of the present invention, the alkyl groups in the definitions of R₂₀, Rₐ, R_{b}, R_{c}, Rₓ, R_{y} and R_{z} may be straight chain or branched alkyl chain groups having from 1 to 12 carbon atoms, and they are preferably an alkyl group having from 1 to 6 carbon atoms, more preferably a methyl group, an ethyl group or an i-propyl group, and most preferably a methyl group. In the definitions of M and Q, they may be straight chain or branched alkyl chain groups having from 1 to 6 carbon atoms. Methyl, ethyl, n-propyl, isopropyl and butyl, including n-butyl, isobutyl, sec-butyl and tert-butyl are particularly preferred alkyl groups in the compounds of the present invention.

In the compounds of the present invention, the alkenyl groups in the definitions of Rₐ, R_{b}, R_{c} and Rₓ are branched or unbranched, and may have one or more double bonds and from 2 to 12 carbon atoms. Preferably, they have from 2 to 6 carbon atoms, and more preferably they are branched or unbranched alkenyl groups having 2, 3 or 4 carbon atoms. Ethenyl, 1-propenyl, 2-propenyl, 1-methylethenyl, 1-butenyl, 2-butenyl, and 3-butenyl are particularly preferred alkenyl groups in the compounds of the present invention.

In the compounds of the present invention, the alkynyl group in the definitions of Rₐ, R_{b}, R_{c} and Rₓ are branched or unbranched, and may have one or more triple bonds and from 2 to 12 carbon atoms. Preferably, they have from 2 to 6 carbon atoms, and more preferably they are branched or unbranched alkynyl groups having 2, 3 or 4 carbon atoms.

In the compounds of the present invention, the halogen substituents in the definitions of Rₓ, R_{y} and R_{z} include F, Cl, Br and I, preferably C₁.

In the compounds of the present invention, the 5- to 14-membered saturated or unsaturated heterocyclic group in the definitions of Rₓ is a heterocyclic group having one or more rings, comprising at least one oxygen, nitrogen or sulphur atom in said ring(s). The heterocyclic group is a group which may be a heteroaromatic group or a heteroalicyclic group, the latter of which may be partially unsaturated, both the aromatic and the alicyclic heterocyclic group containing from 1 to 3 separated or fused rings. Preferably the heteroaromatic and heteroalicyclic group contain from 5 to 10 ring atoms. Suitable heteroaromatic groups in the compounds of the present invention contain one, two or three heteroatoms selected from N, O and S atoms and include, for example, quinolyl including 8-quinolyl, isoquinolyl, coumarinyl including 8-coumarinyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl and furopyridyl. Suitable heteroalicyclic groups in the compounds of the present invention contain one, two or three heteroatoms selected from N, O and S atoms and include, for example, pyrrolidinyl, tetrahydrofuranyl, dihydrofuranyl, tetrahydrothienyl, tetrahydrothiopyranyl, piperidyl, morpholinyl, thiomorpholinyl, thioxanyl, piperazinyl, azetidinyl, oxetanyl, thietanyl, homopiperidyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

In the compounds of the present invention, the aryl group in the definition of Rₓ and R₂₀ is a single or multiple ring compound that contain separate and/or fused aryl groups and has from 6 to 18 ring atoms and is optionally substituted. Typical aryl groups contain from 1 to 3 separated or fused rings. Preferably aryl groups contain from 6 to 12 carbon ring atoms. Particularly preferred aryl groups include substituted or unsubstituted phenyl, substituted or unsubstituted naphthyl, substituted or unsubstituted biphenyl, substituted or unsubstituted phenanthryl and substituted or unsubstituted anthryl, and most preferred substituted or unsubstituted phenyl, wherein the substituents are as indicated above.

In the compounds of the present invention, the aralkyl groups in the definitions of Rₓ, R_{y} and R_{z} comprise an alkyl group as defined and exemplified above which is substituted with one or more aryl groups as defined and exemplified above. Preferred examples include optionally substituted benzyl, optionally substituted phenylethyl and optionally substituted naphthylmethyl.

In the compounds of the present invention, the aralkyloxy groups in the definitions of Rₓ comprise an alkoxy group having from 1 to 12 carbon atoms which is substituted with one or more aryl groups as defined and exemplified above. Preferably, the alkoxy moiety has from 1 to 6 carbon atoms and the aryl group contains from 6 to about 12 carbon ring atoms, and most preferably the aralkyloxy group is optionally substituted benzyloxy, optionally substituted phenylethoxy and optionally substituted naphthylmethoxy.

In the compounds of the present invention, the heterocycloalkyl groups in the definitions of R_{y} and R_{z} comprise an alkyl group as defined and exemplified above which is substituted with one or more heterocyclyl groups as defined and exemplified above. Preferably, the heterocycloalkyl groups comprise an alkyl group having from 1 to 6 carbon atoms which is substituted with a heterocyclyl group having from 5 to 10 ring atoms in 1 or 2 ring atoms and can be aromatic, partially saturated or fully saturated. More preferably, the heterocycloalkyl groups comprise a methyl or ethyl group which is substituted with a heterocyclyl group selected from the group consisting of pyrrolidinyl, imidazolidinyl, piperidinyl, piperazinyl, morpholinyl, tetrahydrofuranyl, oxanyl, thianyl, 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl and benzimidazole.

In the compounds of the present invention, the alkylene groups in the definition of R₁₉ are straight or branched alkylene groups having from 1 to 12 carbon atoms and the alkylene groups in the definitions of M, X, T, and R₃₀ are straight or branched alkylene groups having from 1 to 6 carbon atoms. Preferably, the alkylene groups in the definition of R₁₉ are straight or branched alkylene groups having from 1 to 8 carbon atoms, more preferably straight or branched alkylene groups having from 1 to 6 carbon atoms. For M, preferred are straight or branched alkylene groups having from 1 to 3 carbon atoms. In the definition of X, the alkylene groups in the definition of X are preferably straight or branched alkylene groups having from 2 to 4 carbon atoms. For T, preferred are straight or branched alkylene groups having from 2 to 4 carbon atoms. In the definition of R₃₀, preferred are straight or branched alkylene groups having from 2 to 4 carbon atoms, being most preferred a straight alkylene group having 3 carbon atoms. For the avoidance of doubt, the term “alkylene” is used to refer to alkanediyl groups.

In the compounds of the present invention, the carbocyclo groups in the definitions of R₁₉ and M are cycloalkyl groups having from 3 to 8 carbon atoms which have two covalent bonds at any position on the cycloalkyl ring connecting said cycloalkyl group to the remainder of the drug conjugate. Preferably, the carbocyclo groups in the definitions of R₁₉ and M are cycloalkyl groups having from 3 to 7 carbon atoms, and more preferably carbocyclo groups having from 5 to 7 carbon atoms.

In the compounds of the present invention, the arylene groups in the definition of R₁₉ are aryl groups having from 6 to 18 carbon atoms in one or more rings which have two covalent bonds at any position on the aromatic ring system connecting said arylene groups to the remainder of the drug conjugate. Preferably, the arylene groups in the definition of R₁₉ are aryl groups having from 6 to 12 carbon atoms in one or more rings which have two covalent bonds at any position on the aromatic ring system, and most preferably they are phenylene groups.

In the compounds of the present invention, the heterocyclo groups in the definition of R₁₉ are heterocyclyl groups containing from 1 to 3 separated or fused rings having from 5 to 14 ring atoms and comprising at least one oxygen, nitrogen or sulphur atom in said ring(s), wherein there are two covalent bonds at any position on the ring system of said heterocyclic groups. The heterocyclic groups are groups which may be heteroaromatic groups or heteroalicyclic groups (the latter may be partially unsaturated). Preferably, the heterocyclo groups in the definition of R₁₉ are heterocyclyl groups containing from 1 to 3 separated or fused rings having from 5 to 12 ring atoms and comprising at least one oxygen, nitrogen or sulphur atom in said ring(s), wherein there are two covalent bonds at any position on the ring system of said heterocyclic groups.

Where there are more than one optional substituents Rₓ, R_{y} or R_{z} on a substituent, each substituent Rₓ may be the same or different, each substituent R_{y} may be the same or different and each R_{z} may be the same or different.

In an embodiment, D may be a drug moiety of formula (I) or a pharmaceutically acceptable salt or ester thereof:

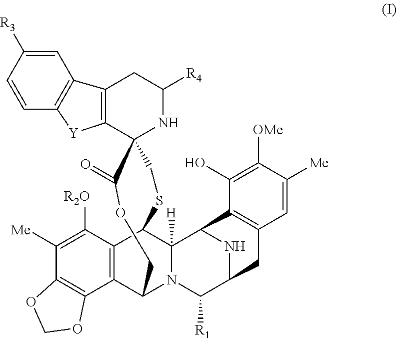

wherein:
D is covalently attached via a hydroxy or amine group to (X)_{b} if any, or (AA)_{w} if any, or to (T)_{g} if any, or (L);
Y is —NH— or —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂ alkenyl, and substituted or unsubstituted C₂-C₁₂ alkynyl;
R_{c} is selected from substituted or unsubstituted C₁-C₁₂ alkyl, substituted or unsubstituted C₂-C₁₂ alkenyl, and substituted or unsubstituted C₂-C₁₂ alkynyl; and
Prot^{NH} is a protecting group for amino.

In embodiments according to all aspects of the present invention, substituted groups are substituted with one or more substituents Rₓ that are independently selected from the group consisting of C₁-C₁₂ alkyl groups which may be optionally substituted with at least one group R_{y}, C₂-C₁₂ alkenyl groups which may be optionally substituted with at least one group R_{y}, C₂-C₁₂ alkynyl groups which may be optionally substituted with at least one group R_{y}, halogen atoms, oxo groups, thio groups, cyano groups, nitro groups, OR_{y}, OCOR_{y}, OCOOR_{y}, COR_{y}, COOR_{y}, OCONR_{y}R_{z}, CONR_{y}R_{z}, S(O)R_{y}, SO₂R_{y}, P(O)(R_{y})OR_{z}, NR_{y}R_{z}, NR_{y}COR_{z}, NR_{y}C(═O)NR_{y}R_{z}, NR_{y}C(═NR_{y})NR_{y}R_{z}, aryl groups having from 6 to 18 carbon atoms in one or more rings which may optionally be substituted with one or more substituents which may be the same or different selected from the group consisting of R_{y}, OR_{y}, OCOR_{y}, OCOOR_{y}, NR_{y}R_{z}, NR_{y}COR_{z}, and NR_{y}C(═NR_{y})NR_{y}R_{z}, aralkyl groups comprising an alkyl group having from 1 to 12 carbon atoms substituted with an optionally substituted aryl group as defined above, aralkyloxy groups comprising an alkoxy group having from 1 to 12 carbon atoms substituted with an optionally substituted aryl group as defined above, and a 5- to 14-membered saturated or unsaturated heterocyclic group having one or more rings and comprising at least one oxygen, nitrogen or sulphur atom in said ring(s), said heterocyclic group optionally being substituted with one or more substituents R_{y}, and where there is more than one optional substituents on any given group the optional substituents R_{y} may be the same or different;
each R_{y} and R_{z} is independently selected from the group consisting of hydrogen, C₁-C₁₂ alkyl groups, C₁-C₁₂ alkyl groups that are substituted with at least one halogen atom, aralkyl groups comprising a C₁-C₁₂ alkyl group that is substituted with an aryl group having from 6 to 18 carbon atoms in one or more rings and heterocycloalkyl groups comprising a C₁-C₁₂ alkyl group that is substituted with a 5- to 14-membered saturated or unsaturated heterocyclic group having one or more rings and comprising at least one oxygen, nitrogen or sulphur atom in said ring(s).

In another embodiment, D may be a drug moiety of formula (IH) or a pharmaceutically acceptable salt or ester thereof:

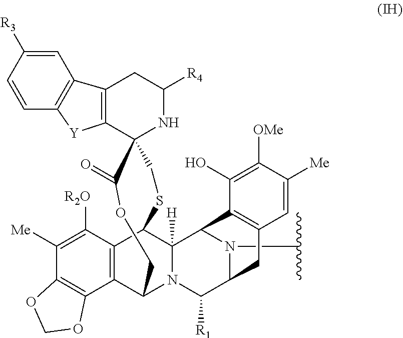

wherein the wavy line indicates the point of covalent attachment to (X)_{b} if any, or (AA)_{w} if any, or to (T)_{g} if any, or to (L);
Y is selected from —NH— and —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
R_{b} is selected from substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
R_{c} is selected from substituted or unsubstituted C₁-C₁₂ alkyl, substituted or unsubstituted C₂-C₁₂ alkenyl, and substituted or unsubstituted C₂-C₁₂ alkynyl, wherein the optional substituents are one or more substituents Rₓ;
Prot^{NH} is a protecting group for amino;
wherein substituted groups are substituted with one or more substituents Rₓ that are independently selected from the group consisting of C₁-C₁₂ alkyl groups which may be optionally substituted with at least one group R_{y}, C₂-C₁₂ alkenyl groups which may be optionally substituted with at least one group R_{y}, C₂-C₁₂ alkynyl groups which may be optionally substituted with at least one group R_{y}, halogen atoms, oxo groups, thio groups, cyano groups, nitro groups, OR_{y}, OCOR_{y}, OCOOR_{y}, COR_{y}, COOR_{y}, OCONR_{y}R_{z}, CONR_{y}R_{z}, S(O)R_{y}, SO₂R_{y}, P(O)(R_{y})OR_{z}, NR_{y}R_{z}, NR_{y}COR_{z}, NR_{y}C(═O)NR_{y}R_{z}, NR_{y}C(═NR_{y})NR_{y}R_{z}, aryl groups having from 6 to 18 carbon atoms in one or more rings which may optionally be substituted with one or more substituents which may be the same or different selected from the group consisting of R_{y}, OR_{y}, OCOR_{y}, OCOOR_{y}, NR_{y}R_{z}, NR_{y}COR_{z}, and NR_{y}C(═NR_{y})NR_{y}R_{z}, aralkyl groups comprising an alkyl group having from 1 to 12 carbon atoms substituted with an optionally substituted aryl group as defined above, aralkyloxy groups comprising an alkoxy group having from 1 to 12 carbon atoms substituted with an optionally substituted aryl group as defined above, and a 5- to 14-membered saturated or unsaturated heterocyclic group having one or more rings and comprising at least one oxygen, nitrogen or sulphur atom in said ring(s), said heterocyclic group optionally being substituted with one or more substituents R_{y}, and where there is more than one optional substituents on any given group the optional substituents R_{y} may be the same or different;
each R_{y} and R_{z} is independently selected from the group consisting of hydrogen, C₁-C₁₂ alkyl groups, C₁-C₁₂ alkyl groups that are substituted with at least one halogen atom, aralkyl groups comprising a C₁-C₁₂ alkyl group that is substituted with an aryl group having from 6 to 18 carbon atoms in one or more rings and heterocycloalkyl groups comprising a C₁-C₁₂ alkyl group that is substituted with a 5- to 14-membered saturated or unsaturated heterocyclic group having one or more rings and comprising at least one oxygen, nitrogen or sulphur atom in said ring(s).

Preferred compounds of the compounds of general formula (I) or (IH) and drugs of general formula (IA), are those having general formula a or b, or a pharmaceutically acceptable salt, ester, solvate, tautomer or stereoisomer thereof:

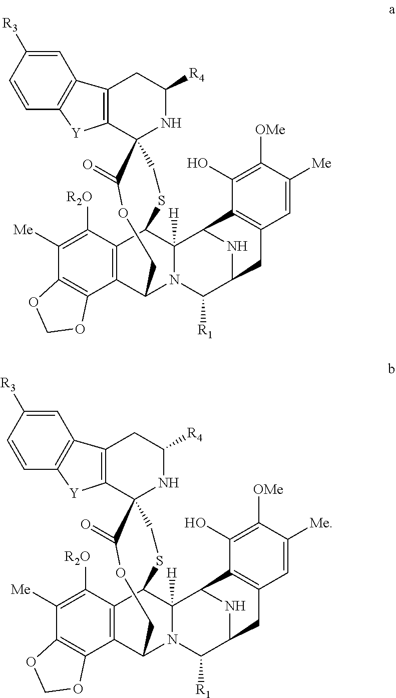

Note where the compounds have general formula a or b, R₄ may not be hydrogen.

Preferred drug moieties and drugs include moieties of general formula (I) or (IH) and drugs of general formula (IA), wherein:
Y is —NH—;
and R₁; R₂; R₃; R₄; Rₐ; R_{b}; R_{c}; and Prot^{NH} are as defined as above.

Preferred drug moieties and drugs include moieties of general formula (I) or (IH) and drugs of general formula (IA), wherein:
Y is —O—;
and R₁; R₂; R₃; R₄; Rₐ; R_{b}; R_{c}; and Prot^{NH} are as defined as above.

Further preferred drug moieties and drugs include moieties of general formula (I) or (IH) and drugs of general formula (IA), wherein:
R₁ is —OH;
and Y; R₂; R₃; R₄; Rₐ; R_{b}; R_{c}; and Prot^{NH} are as defined as above.

Further preferred drug moieties and drugs include moieties of general formula (I) or (IH) and drugs of general formula (IA), wherein:
R₁ is —CN;
and Y; R₂; R₃; R₄; Rₐ; R_{b}; R_{c}; and Prot^{NH} are as defined as above.

Further preferred drug moieties and drugs include moieties of general formula (I) or (IH) and drugs of general formula (IA), wherein:
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
and Y; R₁; R₃; R₄; R_{b}; R_{c}; and Prot^{NH} are as defined as above.

Further preferred drug moieties and drugs include moieties of general formula (I) or (IH) and drugs of general formula (IA), wherein:
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
and Y; R₁; R₂; R₄; Rₐ; R_{c}; and Prot^{NH} are as defined as above.

Further preferred drug moieties and drugs include moieties of general formula (I) or (IH) and drugs of general formula (IA), wherein:
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and Y; R₁; R₂; R₃; Rₐ; and R_{b} are as defined as above.

Further preferred drug moieties and drugs include moieties of general formula (I) or (IH) and drugs of general formula (IA), wherein:
Y is —NH—;
R₁ is —OH;
and R₂; R₃; R₄; Rₐ; R_{b}; R_{c}; and Prot^{NH} are as defined as above.

Further preferred drug moieties and drugs include moieties of general formula (I) or (IH) and drugs of general formula (IA), wherein:
Y is —NH—;
R₂ is a —C(═O)Rₐ; where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
and R₁; R₃; R₄; R_{b}; R_{c}; and Prot^{NH} are as defined as above.

Further preferred drug moieties and drugs include moieties of general formula (I) or (IH) and drugs of general formula (IA), wherein:
Y is —NH—;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
and R₁; R₂; R₄; Rₐ; R_{c}; and Prot^{NH} are as defined as above.

Further preferred drug moieties include moieties of general formula (I) or (IH), wherein:
Y is —NH—;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, or substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₁; R₂; R₃; Rₐ; and R_{b} are as defined as above.

Further preferred drugs of general formula (IA), wherein:
Y is —NH—;
R₄ is selected from —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, or substituted or unsubstituted tert-butyl. Most preferred R_{c} is methyl. More preferred R₄ is —CH₂OH or —CH₂NH₂. Most preferred R₄ is —CH₂OH;
and R₁; R₂; R₃; Rₐ; and R_{b} are as defined as above.

Further preferred drug moieties and drugs include moieties of general formula (I) or (IH) and drugs of general formula (IA), wherein:
Y is —NH—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl; and R₃; R₄; R_{b}; R_{c}; and Prot^{NH} are as defined as above.

Further preferred drug moeities and drugs include moieties of general formula (I) or (IH) and drugs of general formula (IA), wherein:
Y is —NH—;
R₁ is —OH;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group; and R₂; R₄; Rₐ; R_{c}; and Prot^{NH} are as defined as above.

Further preferred drug moieties include moieties of general formula (I) or (IH), wherein:
Y is —NH—;
R₁ is —OH;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₂; R₃; Rₐ; and R_{b} are as defined as above.

Further preferred drugs of general formula (IA), wherein:
Y is —NH—;
R₁ is —OH;
R₄ is selected from —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from —CH₂OH and —CH₂NH₂. Most preferred R₄ is —CH₂OH;
and R₂; R₃; Rₐ; and R_{b} are as defined as above.

Further preferred drug moieties and drugs include moieties of general formula (I) or (IH) and drugs of general formula (IA), wherein:
Y is —NH—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group; and R₁; R₄; R_{c}; and Prot^{NH} are as defined as above.

Further preferred drug moieties include moieties of general formula (I) or (IH), wherein:
Y is —NH—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₁; R₃; and R_{b} are as defined as above.

Further preferred drugs include drugs of general formula (IA), wherein:
Y is —NH—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₄ is selected from —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c}, is methyl. More preferred R₄ is selected from —CH₂OH and —CH₂NH₂. Most preferred R₄ is —CH₂OH;
and R₁; R₃; and R_{b} are as defined as above.

Further preferred drug moieties include moieties of general formula (I) or (IH), wherein:
Y is —NH—;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₁; R₂; and Rₐ; are as defined as above.

Further preferred drugs include drugs of general formula (IA), wherein:
Y is —NH—;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from —CH₂OH and —CH₂NH₂. Most preferred R₄ is —CH₂OH;
and R₁; R₂; and Rₐ; are as defined as above.

Further preferred drug moieties and drugs include moieties of general formula (I) or (IH) and drugs of general formula (IA), wherein:
Y is —NH—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group; and R₄; R_{c}; and Prot^{NH} are as defined as above.

Further preferred drug moieties include moieties of general formula (I) or (IH), wherein:
Y is —NH—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₃; and R_{b} are as defined as above.

Further preferred drugs include drugs of general formula (IA), wherein:
Y is —NH—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₄ is selected from —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from —CH₂OH and —CH₂NH₂. Most preferred R₄ is —CH₂OH;
and R₃; and R_{b} are as defined as above.

Further preferred drug moieties include moieties of general formula (I) or (IH), wherein:
Y is —NH—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₁ is as defined as above.

Further preferred drugs include drugs of general formula (IA), wherein:
Y is —NH—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from —CH₂OH and —CH₂NH₂. Most preferred R₄ is —CH₂OH;
and R₁ is as defined as above.

Further preferred drug moieties include moieties of general formula (I) or (IH), wherein:
Y is —NH—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen, —CH₂OH. Most preferred R₄ is hydrogen.

Further preferred drugs include drugs of general formula (IA), wherein:
Y is —NH—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from —CH₂OH and —CH₂NH₂. Most preferred R₄ is —CH₂OH.

Further preferred drug moieties and drugs include moieties of general formula (I) or (IH) and drugs of general formula (IA), wherein:
Y is —O—;
R₁ is —OH;
and R₂; R₃; R₄; Rₐ; R_{b}; R_{c}; and Prot^{NH} are as defined as above.

Further preferred drug moieties and drugs include moieties of general formula (I) or (IH) and drugs of general formula (IA), wherein:
Y is —O—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
and R₁; R₃; R₄; R_{b}; R_{c}; and Prot^{NH} are as defined as above.

Further preferred drug moieties and drugs include moieties of general formula (I) or (IH) and drugs of general formula (IA), wherein:
Y is —O—;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ is hydrogen and methoxy, being methoxy the most preferred R₃ group;
and R₁; R₂; R₄; Rₐ; R_{c}; and Prot^{NH} are as defined as above.

Further preferred drug moieties and drugs include moieties of general formula (I) or (IH) and drugs of general formula (IA), wherein:
Y is —O—;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₁; R₂; R₃; Rₐ; and R_{b} are as defined as above.

Further preferred drug moieties and drugs include moieties of general formula (I) or (IH) and drugs of general formula (IA), wherein:
Y is —O—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
and R₃; R₄; R_{b}; R_{c}; and Prot^{NH} are as defined as above.

Further preferred drug moieties and drugs include moieties of general formula (I) or (IH) and drugs of formula (IA), wherein:
Y is —O—;
R₁ is —OH;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
and R₂; R₄; Rₐ; R_{c}; and Prot^{NH} are as defined as above.

Further preferred drug moieties and drugs include moieties of general formula (I) or (IH) and drugs of general formula (IA), wherein:
Y is —O—;
R₁ is —OH;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₂; R₃; Rₐ; and R_{b} are as defined as above.

Further preferred drug moieties and drugs include moieties of general formula (I) or (IH) and drugs of general formula (IA), wherein:
Y is —O—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group; and R₁; R₄; R_{c}; and Prot^{NH} are as defined as above.

Further preferred drug moieties and drugs include moieties of general formula (I) or (IH) and drugs of general formula (IA), wherein:
Y is —O—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R_{c} is acetyl;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₁; R₃; and R_{b} are as defined as above.

Further preferred drug moieties and drugs include moieties of general formula (I) or (IH) and drugs of general formula (IA), wherein:
Y is —O—;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₁; R₂; and Rₐ are as defined as above.

Further preferred drug moieties and drugs include moieties of general formula (I) or (IH) and drugs of general formula (IA), wherein:
Y is —O—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
and R₄; R_{c}; and Prot^{NH} are as defined as above.

Further preferred drug moieties and drugs include moieties of general formula (I) or (IH) and drugs of general formula (IA), wherein:
Y is —O—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₃; and R_{b} are as defined as above.

Further preferred drug moieties and drugs include moieties of general formula (I) or (IH) and drugs of general formula (IA), wherein:
Y is —O—;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen;
and R₁ is as defined as above.

Further preferred drug moieties and drugs include moieties of general formula (I) or (IH) and drugs of general formula (IA), wherein:
Y is —O—;
R₁ is —OH;
R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl;
R₃ is hydrogen or a —OR_{b} group; where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group;
R₄ is selected from hydrogen, —CH₂OH, —CH₂OC(═O)R_{c} and —CH₂NH₂; where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. More preferably, R₄ may be hydrogen or —CH₂OH. Most preferred R₄ is hydrogen.

The following preferred substituents (where allowed by possible substituent groups) apply to drug moieties of formula (I) or (IH) and to drugs of formula (IA):

Particularly preferred R₁ is —OH.

Particularly preferred R₂ is a —C(═O)Rₐ group where Rₐ is a substituted or unsubstituted C₁-C₆ alkyl. Particularly preferred Rₐ is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. Most preferred R₂ is acetyl.

Particularly preferred R₃ is hydrogen or a —OR_{b} group where R_{b} is a substituted or unsubstituted C₁-C₆ alkyl. Particularly preferred R_{b} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl and substituted or unsubstituted tert-butyl. More preferred R₃ are hydrogen and methoxy, being methoxy the most preferred R₃ group.

Particularly preferred R₄ is selected from hydrogen, —CH₂OH, —CH₂OC(═O)R_{c} and —CH₂NH₂ where R_{c} is a substituted or unsubstituted C₁-C₆ alkyl. Particularly preferred R_{c} is selected from substituted or unsubstituted methyl, substituted or unsubstituted ethyl, substituted or unsubstituted n-propyl, substituted or unsubstituted isopropyl, substituted or unsubstituted n-butyl, substituted or unsubstituted isobutyl, substituted or unsubstituted sec-butyl, and substituted or unsubstituted tert-butyl. Most preferred R_{c} is methyl. More preferred R₄ is selected from hydrogen, —CH₂OH and —CH₂NH₂. Even more preferred R₄ is hydrogen or —CH₂OH and most preferred R₄ is hydrogen.

Particularly preferred drug moieties and drugs according to the present invention include:
Moieties of formula (I) or (IH) wherein
   Y is —NH—; and
   R₄ is selected from hydrogen, —CH₂OH, and —CH₂NH₂.
Drugs of formula (IA) wherein
   Y is —NH—; and
   R₄ is selected from —CH₂OH, and —CH₂NH₂.
Moieties of formula (I) or (IH) or drugs of formula (IA) wherein
   Y is —O—;
   R₄ is selected from hydrogen, —CH₂OH and —CH₂NH₂.
Moieties of formula (I) or (IH) or drugs of formula (IA) wherein
   R₂ is a —C(═O)Rₐ group;
   R₃ is hydrogen or a —OR_{b} group;
   R₄ is selected from hydrogen, —CH₂OH, and —CH₂NH₂;
   Rₐ is selected from hydrogen, and substituted or unsubstituted C₁-C₆ alkyl; and
   R_{b} is substituted or unsubstituted C₁-C₆ alkyl.

More preferred drug moieties according to the present invention include
Moieties of formula (I) or (IH) wherein
   Y is —NH—;
   R₂ is a —C(═O)Rₐ group;
   R₃ is hydrogen or a —OR_{b} group;
   R₄ is hydrogen or —CH₂OH;
   Rₐ is selected from hydrogen and substituted or unsubstituted C₁-C₆ alkyl; and
   R_{b} is substituted or unsubstituted C₁-C₆ alkyl.
Drugs of formula (IA) wherein
   Y is —NH—;
   R₂ is a —C(═O)Rₐ group;
   R₃ is hydrogen or a —OR_{b} group;
   R₄ is —CH₂OH;
   Rₐ is selected from hydrogen and substituted or unsubstituted C₁-C₆ alkyl; and
   R_{b} is substituted or unsubstituted C₁-C₆ alkyl.
Moieties of formula (I) or (IH) or drugs of formula (IA) wherein
   Y is —O—;
   R₂ is a —C(═O)Rₐ group;
   R₃ is hydrogen or a —OR_{b} group;
   R₄ is hydrogen or —CH₂OH;
   Rₐ is selected from hydrogen and substituted or unsubstituted C₁-C₆ alkyl; and
   R_{b} is substituted or unsubstituted C₁-C₆ alkyl.
Moieties of formula (I) or (IH) or drugs of formula (IA) wherein
   R₂ is a —C(═O)Rₐ group;
   R₃ is hydrogen or a —OR_{b} group;
   R₄ is hydrogen or —CH₂OH;
   Rₐ is substituted or unsubstituted C₁-C₆ alkyl; and
   R_{b} is substituted or unsubstituted C₁-C₆ alkyl. Particularly more preferred drug moieties according to the present invention include:
Moieties of formula (I) or (IH) wherein
   Y is —NH—;
   R₂ is a —C(═O)Rₐ group;
   R₃ is hydrogen or methoxy;
   R₄ is hydrogen or —CH₂OH; and
   Rₐ is substituted or unsubstituted C₁-C₆ alkyl.
Drugs of formula (IA) wherein
   Y is —NH—;
   R₂ is a —C(═O)Rₐ group;
   R₃ is hydrogen or methoxy;
   R₄ is —CH₂OH; and
   Rₐ is substituted or unsubstituted C₁-C₆ alkyl.
Moieties of formula (I) or (IH) or drugs of formula (IA) wherein
   Y is —O—;
   R₂ is a —C(═O)Rₐ group;
   R₃ is hydrogen or methoxy;
   R₄ is hydrogen or —CH₂OH; and
   Rₐ is substituted or unsubstituted C₁-C₆ alkyl.
Moieties of formula (I) or (IH) or drugs of formula (IA) wherein
   R₂ is a —C(═O)Rₐ group;
   R₃ is hydrogen or methoxy;
   R₄ is hydrogen or —CH₂OH; and
   Rₐ is selected from methyl, ethyl, n-propyl, isopropyl and butyl, including n-butyl, sec-butyl, isobutyl and tert-butyl.

Even more preferred drug moieties according to the present invention include:
Moieties of formula (I) or (IH) wherein
   Y is —NH—;
   R₂ is acetyl;
   R₃ is methoxy; and
   R₄ is hydrogen.
Drugs of formula (IA) wherein
   Y is —NH—;
   R₂ is acetyl;
   R₃ is methoxy; and
   R₄ is —CH₂OH.
Moieties of formula (I) or (IH) or drugs of formula (IA) wherein
   Y is —O—;
   R₂ is acetyl;
   R₃ is methoxy; and
   R₄ is hydrogen.
Moieties of formula (I) or (IH) or drugs of formula (IA) wherein
   R₂ is acetyl;
   R₃ is methoxy; and
   R₄ is hydrogen.
Moieties of formula (I) or (IH) or drugs of formula (IA) wherein
   R₁ is —OH;
   R₂ is acetyl;
   R₃ is methoxy; and
   R₄ is hydrogen.
A moiety according to the present invention of formula:

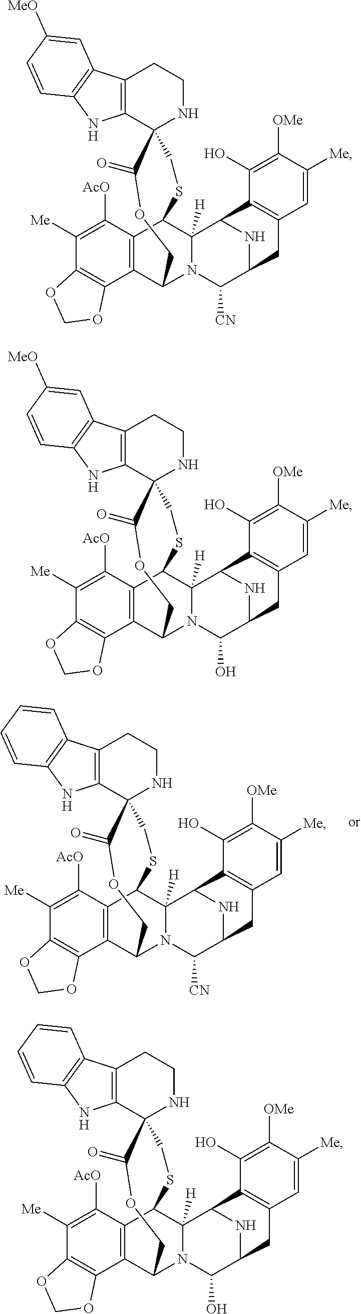

or a pharmaceutically acceptable salt or ester thereof; wherein D is covalently attached via a hydroxy or amine group to (X)_{b} if any, or (AA)_{w} if any, or to (T)_{g} if any, or (L).

Being particularly preferred moieties of formula:

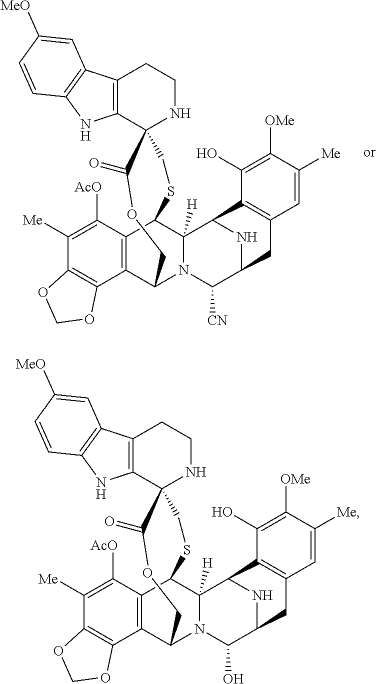

or a pharmaceutically acceptable salt or ester thereof; wherein D is covalently attached via a hydroxy or amine group to (X)_{b} if any, or (AA)_{w} if any, or to (T)_{g} if any, or (L).
A drug of formula:

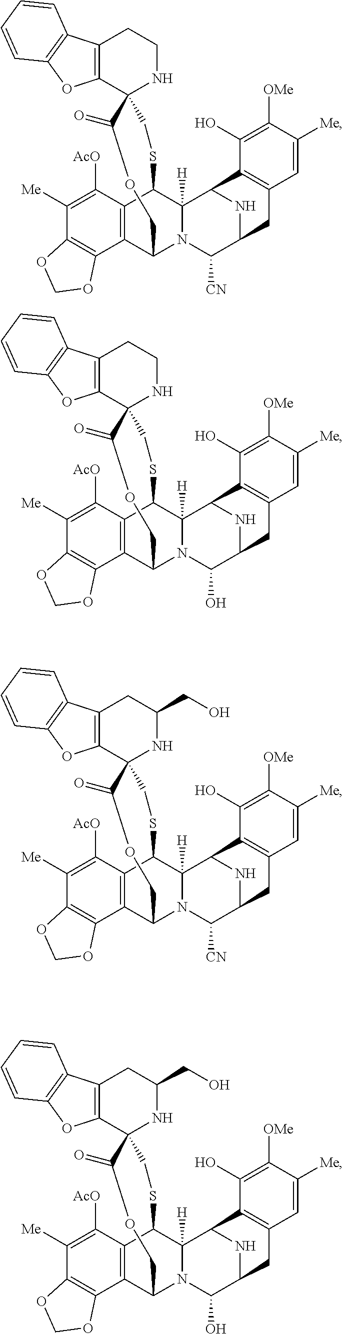

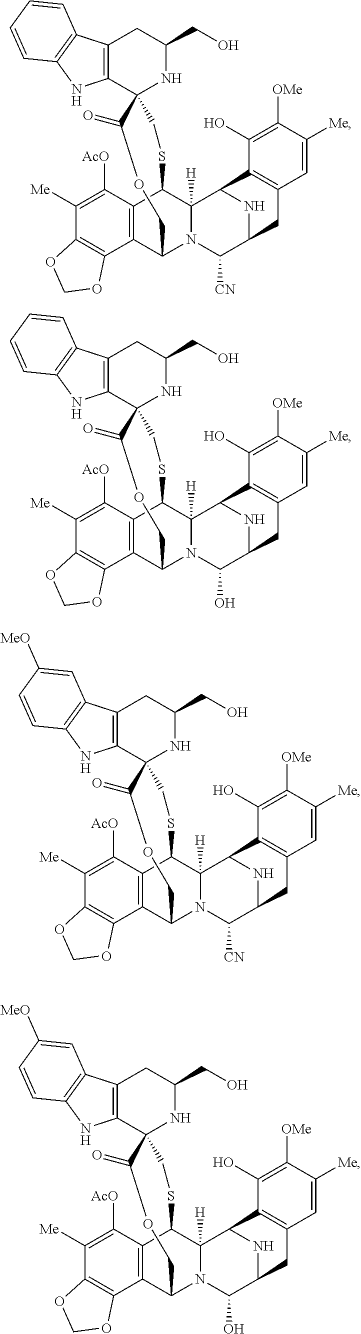

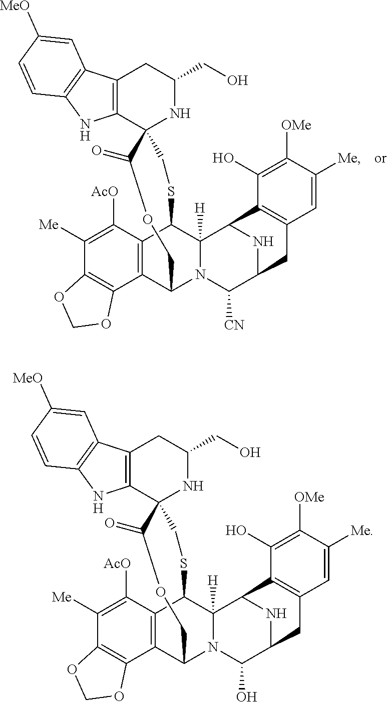 [SMILES: COc1ccc2[nH]c3c(c2c1)C[C@H](CO)N[C@]31CS[C@@H]2c3c(OC(C)=O)c(C)c4c(c3[C@H](COC1=O)N1[C@@H]2[C@@H]2N[C@@H](Cc3cc(C)c(OC)c(O)c32)[C@@H]1C#N)OCO4 ; COc1ccc2[nH]c3c(c2c1)C[C@H](CO)N[C@]31CS[C@@H]2c3c(OC(C)=O)c(C)c4c(c3[C@H](COC1=O)N1[C@@H]2[C@@H]2N[C@@H](Cc3cc(C)c(OC)c(O)c32)[C@@H]1O)OCO4]

In additional preferred embodiments, the preferences described above for the different substituents are combined. The present invention is also directed to such combinations of preferred substitutions (where allowed by possible substituent groups) in drug moieties of formula (I) or (IH) and in drugs of formula (IA) according to the present invention.

For the avoidance of doubt, the compounds above may be the drug moiety D and are covalently attached via a hydroxy or amine group to (X)_{b} if any, or (AA)_{w} if any, or to (T)_{g} if any, or (L). Thus, when conjugated, a covalent bond replaces a proton on a hydroxy or amine group on the compound.

Preferred drug conjugates according to the the present invention are given below. The preferred definitions of (X)_{b}, (AA)_{w}, (T)_{g}, and (L) as set out below are applicable to all of the drug moiety D compounds described above. Preferred drug conjugates according to the present invention include:
a drug conjugate of formula [D-(X)_{b}-(AA)_{w}-(T)_{g}-(L)-]ₙ-Ab according to the present invention wherein L is a linker group selected from the group consisting of:

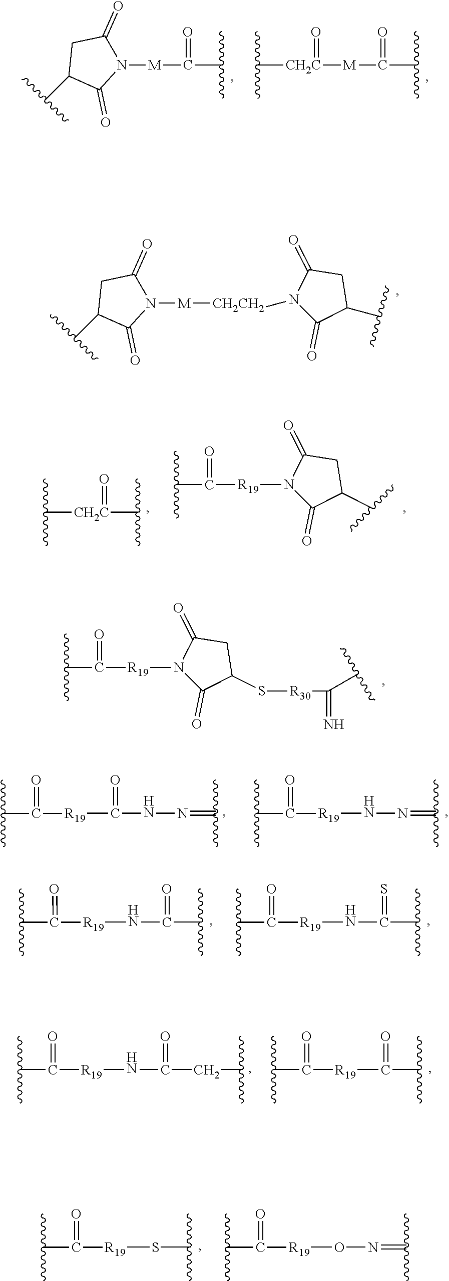

wherein
the wavy lines indicate the point of covalent attachments to an Ab (the wavy line to the right) and to (T)_{g} if any, or (AA)_{w} if any, or to (X)_{b} if any, or to D (the wavy line to the left);
R₁₉ is selected from —C₁-C₁₂ alkylene-, —C₃-C₈ carbocyclo, —O—(C₁-C₁₂ alkylene), —C₆-C₁₈ arylene in one or more rings which may optionally be substituted with one or more substituents Rₓ, —C₁-C₁₂ alkylene-C₆-C₁₈ arylene- wherein the arylene group is in one or more rings which may optionally be substituted with one or more substituents Rₓ, —C₆-C₁₈ arylene-C₁-C₁₂ alkylene- wherein the arylene group is in one or more rings which may optionally be substituted with one or more substituents Rₓ, —C₁-C₁₂ alkylene-(C₃-C₈ carbocyclo)-, —(C₃-C₈ carbocyclo)-C₁-C₁₂ alkylene-, —C₅-C₁₄ heterocyclo- wherein said heterocyclo group may be a saturated or unsaturated group having one or more rings and comprising at least one oxygen, nitrogen or sulphur atom in said ring(s), said group optionally being substituted with one or more substituents Rₓ, —C₁-C₁₂ alkylene-(C₅-C₁₄ heterocyclo)- wherein said heterocyclo group may be a saturated or unsaturated group having one or more rings and comprising at least one oxygen, nitrogen or sulphur atom in said ring(s), said group optionally being substituted with one or more substituents Rₓ, —(C₅-C₁₄ heterocyclo)-C₁-C₁₂ alkylene- wherein said heterocyclo group may be a saturated or unsaturated group having one or more rings and comprising at least one oxygen, nitrogen or sulphur atom in said ring(s), said group optionally being substituted with one or more substituents Rₓ, —(OCH₂CH₂)ᵣ—, and —CH₂—(OCH₂CH₂)ᵣ—, wherein each of the above alkylene substituents whether alone or attached to another moiety the carbon chain may optionally be substituted by one or more substituents Rₓ;
R₃₀ is a —C₁-C₆ alkylene- group;
M is selected from the group consisting of —C₁-C₆ alkylene-, —C₁-C₆ alkylene-(C₃-C₈ carbocyclo)-, —(CH₂CH₂O)ₛ—, —C₁-C₆ alkylene-(C₃-C₈ carbocyclo)-CON(H or C₁-C₆ alkyl)-C₁-C₆ alkylene-, phenylene which may optionally be substituted with one or more substituents Rₓ, phenylene-C₁-C₆ alkylene- wherein the phenylene moiety may optionally be substituted with one or more substituents Rₓ and —C₁-C₆ alkylene-CON(H or C₁-C₆ alkyl)C₁-C₆ alkylene-;
Q is selected from the group consisting of —N(H or C₁-C₆ alkyl)phenylene- and —N(H or C₁-C₆ alkyl)-(CH₂)ₛ;
r is an integer ranging from 1 to 10; and
s is an integer ranging from 1 to 10.
a drug conjugate of formula [D-(X)_{b}-(AA)_{w}-(T)_{g}-(L)-]ₙ-Ab according to the present invention wherein L is selected from the group consisting of:

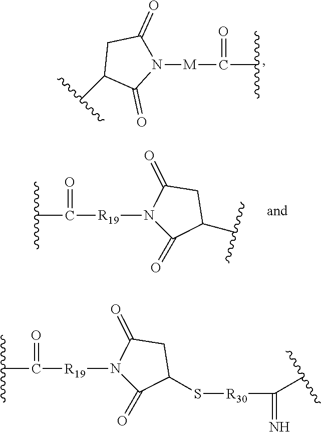

wherein:
the wavy lines indicate the point of covalent attachments to an Ab (the wavy line to the right) and to (T)_{g} if any, or (AA)_{w} if any, or to (X)_{b} if any, or to D (the wavy line to the left);
R₁₉ is selected from —C₁-C₁₂ alkylene-, —O—(C₁-C₁₂ alkylene), —C₆-C₁₂ arylene in one or more rings which may optionally be substituted with one or more substituents Rₓ, —C₁-C₁₂ alkylene-C₆-C₁₂ arylene- wherein the arylene group is in one or more rings which may optionally be substituted with one or more substituents Rₓ, —C₆-C₁₂ arylene-C₁-C₁₂ alkylene- wherein the arylene group is in one or more rings which may optionally be substituted with one or more substituents Rₓ, —C₅-C₁₂ heterocyclo- wherein said heterocyclo group may be a saturated or unsaturated group having one or more rings and comprising at least one oxygen, nitrogen or sulphur atom in said ring(s), said group optionally being substituted with one or more substituents Rₓ, —C₁-C₁₂ alkylene-(C₅-C₁₂ heterocyclo)- wherein said heterocyclo group may be a saturated or unsaturated group having one or more rings and comprising at least one oxygen, nitrogen or sulphur atom in said ring(s), said group optionally being substituted with one or more substituents Rₓ, —(C₅-C₁₂ heterocyclo)-C₁-C₁₂ alkylene- wherein said heterocyclo group may be a saturated or unsaturated group having one or more rings and comprising at least one oxygen, nitrogen or sulphur atom in said ring(s), said group optionally being substituted with one or more substituents Rₓ, —(OCH₂CH₂)ᵣ—, and —CH₂—(OCH₂CH₂)ᵣ— wherein each of the above alkylene substituents whether alone or attached to another moiety the carbon chain may optionally be substituted by one or more substituents Rₓ;
R₃₀ is a —C₁-C₆ alkylene- group;
M is selected from the group consisting of —C₁-C₆ alkylene-, —C₁-C₆ alkylene-(C₃-C₈ carbocyclo)- and phenylene which may optionally be substituted with one or more substituents Rₓ; and
r is an integer ranging from 1-6.
a drug conjugate of formula [D-(X)_{b}-(AA)_{w}-(T)_{g}-(L)-]ₙ-Ab according to the present invention selected from formulas (IV), (V) and (VI):

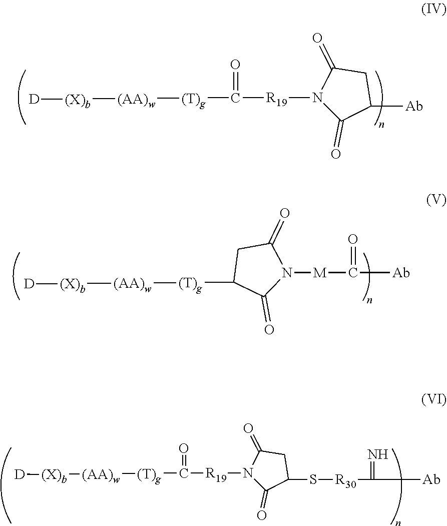

wherein:
X and T are extending groups as defined herein;
each AA is independently an amino acid unit as defined herein;
w is an integer ranging from 0 to 12;
b is an integer of 0 or 1;
g is an integer of 0 or 1;
where b+g+w is optionally not 0;
D is a drug moiety;
Ab is a moiety comprising at least one antigen binding site;
n is the ratio of the group [D-(X)_{b}-(AA)_{w}-(T)_{g}-(L)-] wherein L is as defined in formula (IV), (V) or (VI) to the moiety comprising at least one antigen binding site and is in the range from 1 to 20;
R₁₉ is selected from —C₁-C₈ alkylene-, —O—(C₁-C₈ alkylene), —C₁-C₈ alkylene-C₆-C₁₂ arylene- wherein the arylene group is in one or more rings which may optionally be substituted with one or more substituents Rₓ, and —C₆-C₁₂ arylene-C₁-C₈ alkylene- wherein the arylene group is in one or more rings which may optionally be substituted with one or more substituents Rₓ, wherein each of the above alkylene substituents whether alone or attached to another moiety the carbon chain may optionally be substituted by one or more substituents Rₓ;
R₃₀ is a —C₂-C₄ alkylene- group; and
M is selected from the group consisting of —C₁-C₃ alkylene- and —C₁-C₃ alkylene-(C₅-C₇ carbocyclo)-.
a drug conjugate of formula [D-(X)_{b}-(AA)_{w}-(T)_{g}-(L)-]ₙ-Ab according to the present invention, selected from formulas (IV), (V) and (VI):

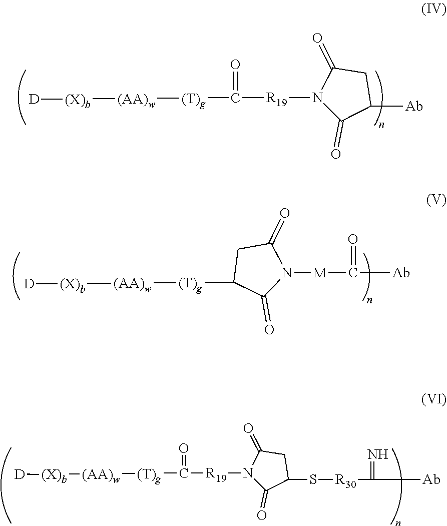

wherein:
X and T are extending groups that may be the same or different;
each AA is independently an amino acid unit;
w is an integer ranging from 0 to 12;
b is an integer of 0 or 1;
g is an integer of 0 or 1;
where b+g+w is optionally not 0;
D is a drug moiety;
Ab is a moiety comprising at least one antigen binding site;
n is the ratio of the group [D-(X)_{b}-(AA)_{w}-(T)_{g}-(L)-] wherein L is as defined in formulas (IV), (V) or (VI) to the moiety comprising at least one antigen binding site and is in the range from 1 to 20;
R₁₉ is selected from —C₁-C₆ alkylene-, phenylene-C₁-C₆ alkylene- wherein the phenylene group may optionally be substituted with one or more substituents Rₓ selected from the group consisting of alkyl groups having from 1 to 6 carbon atoms, alkoxy groups having from 1 to 6 carbon atoms, halogen atoms, nitro groups and cyano groups, wherein each of the above alkylene substituents whether alone or attached to another moiety in the carbon chain may optionally be substituted by one or more substituents Rₓ selected from the group consisting of alkyl groups having from 1 to 6 carbon atoms, alkoxy groups having from 1 to 6 carbon atoms, aryl groups having from 6 to 12 carbon atoms, halogen atoms, nitro groups and cyano groups, and preferably R₁₉ is a —C₁-C₆ alkylene group;
R₃₀ is a —C₂-C₄ alkylene- group; and
M is —C₁-C₃ alkylene-(C₅-C₇ carbocyclo)-.
It is preferred that in the definition of the drug conjugate of formula [D-(X)_{b}-(AA)_{w}-(T)_{g}-(L)-]ₙ-Ab, L is as defined in the preferred definitions for said group above and (AA)_{w} is of formula (II):

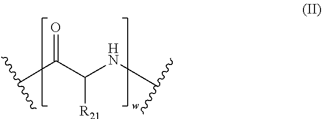

wherein the wavy lines indicate the point of covalent attachments to (X)_{b} if any, or to the drug moiety (the wavy line to the left) and to (T)_{g} if any, or to the linker (the wavy line to the right); and
R₂₁ is, at each occurrence, selected from the group consisting of hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

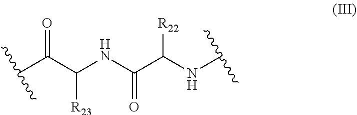

and w is an integer ranging from 0 to 12.
a drug conjugate of formula [D-(X)_{b}-(AA)_{w}-(T)_{g}-(L)-]ₙ-Ab according to the first aspect of the present invention, wherein L is as defined in the preferred definitions for said group above and (AA)_{w} is of formula (II) wherein:
   R₂₁ is selected, at each occurrence, from the group consisting of hydrogen, methyl, isopropyl, sec-butyl, benzyl, indolylmethyl, —(CH₂)₃NHCONH₂, —(CH₂)₄NH₂, —(CH₂)₃NHC(═NH)NH₂ and —(CH₂)₄NHC(═NH)NH₂; and w is an integer ranging from 0 to 6.
a drug conjugate of formula [D-(X)_{b}-(AA)_{w}-(T)_{g}-(L)-]ₙ-Ab according to the first aspect of the present invention, wherein L is as defined in the preferred definitions for said group above, wherein w is 0 or 2, and when w is 2, then (AA)_{w} is of formula (III) wherein:

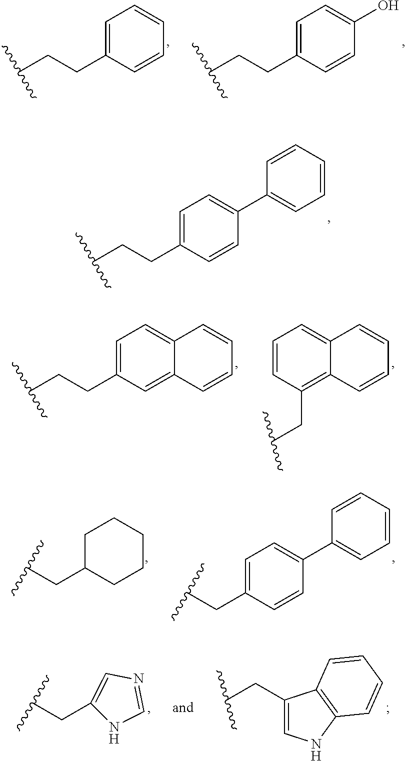

the wavy lines indicate the point of covalent attachments to (X)_{b} if any, or to the drug moiety (the wavy line to the left) and to (T)_{g} if any, or to the linker (the wavy line to the right);
R₂₂ is selected from methyl, benzyl, isopropyl, sec-butyl and indolylmethyl; and
R₂₃ is selected from methyl, —(CH₂)₄NH₂, —(CH₂)₃NHCONH₂ and —(CH₂)₃NHC(═NH)NH₂.
In embodiments of the present invention b+g+w is not 0. In further embodiments, b+w is not 0. In yet further embodiments, when w is not 0, then b is 1. Further, it is preferred that in the definition of the drug conjugate of formula [D-(X)_{b}-(AA)_{w}-(T)_{g}-(L)-]ₙ-Ab, L and (AA), are as defined in the preferred definitions for said groups above and X is an extending group selected from:
   where D is conjugated via an amine group:
      —COO—(C₁-C₆ alkylene)NH—;
      —COO—CH₂-(phenylene which may optionally be substituted with one or more substituents Rₓ)—NH—;
      —COO—(C₁-C₆ alkylene)NH—COO—CH₂-(phenylene which may optionally be substituted with one or more substituents Rₓ)—NH—;
      —COCH₂NH—COCH₂—NH—;
      —COCH₂NH—;
      —COO—(C₁-C₆ alkylene)S—;
      —COO—(C₁-C₆ alkylene)NHCO(C₁-C₆ alkylene)S—; or
   where D is conjugated via an hydroxy group:
      —CONH—(C₁-C₆ alkylene)NH—;
      —COO—CH₂-(phenylene which may optionally be substituted with one or more substituents Rₓ)—NH—;
      —CONH—(C₁-C₆ alkylene)NH—COO—CH₂-(phenylene which may optionally be substituted with one or more substituents Rₓ)—NH—;
      —COCH₂NH—COCH₂—NH—;
      —COCH₂NH—;
      —CONH—(C₁-C₆ alkylene)S—;
      —CONH—(C₁-C₆ alkylene)NHCO(C₁-C₆ alkylene)S—; and
   b is 0 or 1, preferably 1.
a drug conjugate of formula [D-(X)_{b}-(AA)_{w}-(T)_{g}-(L)-]ₙ-Ab according to the present invention, wherein L and (AA)_{w} are as defined in the preferred definitions for said groups above and X is an extending group selected from the group consisting of:
   where D is conjugated via an amine group:
      —COO—(C₂-C₄ alkylene)NH—;
      —COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups;
      —COO—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—;
      —COCH₂NH—COCH₂—NH—;
      —COO—(C₂-C₄ alkylene)S—;
      —COO—(C₂-C₄ alkylene)NHCO(C₁-C₃ alkylene)S—; or
   where D is conjugated via an hydroxy group:
      —CONH—(C₂-C₄ alkylene)NH—;
      —COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups;
      —CONH—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—;
      —COCH₂NH—COCH₂—NH—;
      —CONH—(C₂-C₄ alkylene)S—;
      —CONH—(C₂-C₄ alkylene)NHCO(C₁-C₃ alkylene)S—; and
   b is 0 or 1, preferably 1.
a drug conjugate of formula [D-(X)_{b}-(AA)_{w}-(T)_{g}-(L)-]ₙ-Ab according to the present invention, wherein L and (AA)_{w} are as defined in the preferred definitions for said groups above and X is an extending group selected from the group consisting of:
   where D is conjugated via an amine group:
      —COO—CH₂-phenylene-NH—
      —COO(CH₂)₃NHCOOCH₂-phenylene-NH—;
      —COO(CH₂)₃NH—;
      —COO(CH₂)₃—S—;
      —COO(CH₂)₃NHCO(CH₂)₂S—; or
      where D is conjugated via an hydroxy group:
      —COO—CH₃-phenylene-NH—
      —CONH(CH₂)₃NHCOOCH₂-phenylene-NH—;
      —CONH(CH₂)₃NH—;
      —CONH(CH₂)₃—S—;
      —CONH(CH₂)₃NHCO(CH₂)₂S—; and
   b is 0 or 1, preferably 1.
a drug conjugate of formula [D-(X)_{b}-(AA)_{w}-(T)_{g}-(L)-]ₙ-Ab according to the present invention, wherein L, (AA)_{w}, and (X)_{b} are as defined in the preferred definitions for said groups above and T is an extending group selected from the group consisting of:
   —CO—(C₁-C₆ alkylene)-NH—;
   —CO—(C₁-C₆ alkylene)-[O—(C₂-C₆ alkylene)]ⱼ—NH—;
   —COO—(C₁-C₆ alkylene)-[O—(C₂-C₆ alkylene)]ⱼ—NH—;
   where j is an integer from 1 to 25, and
   g is 0 or 1.
A drug conjugate of formula [D-(X)_{b}-(AA)_{w}-(T)_{g}-(L)-]ₙ-Ab according to the present invention, wherein L, (AA)_{w}, and (X)_{b} are as defined in the preferred definitions for said groups above and T is an extending group selected from the group consisting of:
   —CO—(C₁-C₄ alkylene)NH—
   —CO—(C₁-C₄ alkylene)-[O—(C₂-C₄ alkylene)]ⱼ—NH—;
   —COO—(C₁-C₄ alkylene)-[O—(C₂-C₄ alkylene)]ⱼ—NH—;
   where j is an integer from 1 to 10; and
   g is 0 or 1.
A drug conjugate of formula [D-(X)_{b}-(AA)_{w}-(T)_{g}-(L)-]ₙ-Ab according to the present invention, wherein L, (AA)_{w}, and (X)_{b} are as defined in the preferred definitions for said groups above and T is an extending group selected from the group consisting of:
   —CO—(C₁-C₄ alkylene)NH—
   —CO—(C₁-C₄ alkylene)-[O—(C₂-C₄ alkylene)]ⱼ—NH—;
   —COO—(C₁-C₄ alkylene)-[O—(C₂-C₄ alkylene)]ⱼ—NH—;
   where j is an integer from 1 to 5; and
   g is 0 or 1.
A preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is a compound of formula (I) or (IH), or a pharmaceutically acceptable salt, ester, solvate, tautomer or stereoisomer thereof, wherein R₁ is CN or OH, and more preferably R₁ is CN.
Another preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is a compound of formula (I) or (IH), or a pharmaceutically acceptable salt, ester, solvate, tautomer or stereoisomer thereof, wherein R₂ is C(═O)Rₐ, wherein Rₐ is selected from hydrogen and substituted or unsubstituted C₁-C₆ alkyl, wherein the optional substituents are one or more substituents Rₓ, and more preferably R₂ is acetyl.
Another preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is a compound of formula (I) or (IH), or a pharmaceutically acceptable salt, ester, solvate, tautomer or stereoisomer thereof, wherein R₃ is hydrogen or a —OR_{b} group, wherein R_{b} is a substituted or unsubstituted C₁-C₆ alkyl group, wherein the optional substituents are one or more substituents Rₓ, and more preferably R₃ is hydrogen or methoxy. Most preferably R₃ is methoxy.
Another preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is a compound of formula (I) or (IH), or a pharmaceutically acceptable salt, ester, solvate, tautomer or stereoisomer thereof, wherein R₄ is selected from hydrogen, —CH₂OH and —CH₂NH₂, and more preferably R₄ is hydrogen or —CH₂OH. Most preferably R₄ is hydrogen.
Another preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is a compound of formula (I) or (IH), or a pharmaceutically acceptable salt, ester, solvate, tautomer or stereoisomer thereof, wherein Y is —NH— or —O—.
A further preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is a compound of formula (I) or (IH), or a pharmaceutically acceptable salt, ester, solvate, tautomer or stereoisomer thereof, wherein:
   R₁ is —CN or —OH;
   R₂ is —C(═O)Rₐ, wherein Rₐ is selected from hydrogen and substituted or unsubstituted C₁-C₆ alkyl, wherein the optional substituents are one or more substituents Rₓ;
   R₃ is hydrogen or a —OR_{b} group wherein R_{b} is a substituted or unsubstituted C₁-C₆ alkyl group, wherein the optional substituents are one or more substituents Rₓ;
   R₄ is hydrogen, —CH₂OH or —CH₂NH₂; and
   Y is —NH— or —O.
A further preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is a compound of formula (I) or (IH), or a pharmaceutically acceptable salt, ester, solvate, tautomer or stereoisomer thereof, wherein:
   R₁ is —CN or —OH;
   R₂ is acetyl;
   R₃ is hydrogen or methoxy, more preferably methoxy;
   R₄ is hydrogen or —CH₂OH; and
   Y is —NH— or —O—.
A further preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is a compound of formula (I) or (IH), or a pharmaceutically acceptable salt, ester, solvate, tautomer or stereoisomer thereof, wherein:
   R₁ is —CN;
   R₂ is acetyl:
   R₃ is methoxy;
   R₄ is hydrogen and
   Y is —NH— or —O—.
A further preferred drug conjugate of formula [D-(X)_{b}-(AA)_{w}-(T)_{g}-(L)-]ₙ-Ab according to the present invention is one wherein L, (AA)_{w}, (X)_{b}, and (T)_{g} are as defined above and wherein D is selected from:

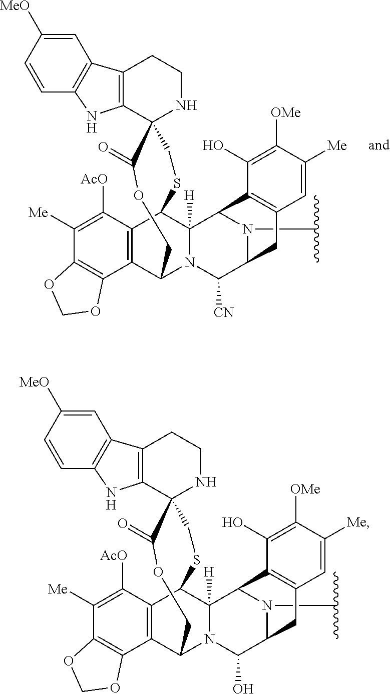 [SMILES: *N1[C@@H]2c3c(cc(C)c(OC)c3O)C[C@H]1[C@H](C#N)N1[C@H]2[C@@H]2SC[C@]3(NCCc4c3[nH]c3ccc(OC)cc43)C(=O)OC[C@H]1c1c3c(c(C)c(OC(C)=O)c12)OCO3 ; *N1[C@@H]2c3c(cc(C)c(OC)c3O)C[C@H]1[C@H](O)N1[C@H]2[C@@H]2SC[C@]3(NCCc4c3[nH]c3ccc(OC)cc43)C(=O)OC[C@H]1c1c3c(c(C)c(OC(C)=O)c12)OCO3]

or a pharmaceutically acceptable salt, ester, solvate, tautomer or stereoisomer thereof; wherein the wavy lines indicate the point of covalent attachment to (X)_{b} if any, or (AA)_{w} if any, or to (T)_{g} if any or to (L).
A further preferred drug conjugate of formula [D-(X)_{b}-(AA)_{w}-(T)_{g}-(L)-]ₙ-Ab according to the present invention is one wherein L, (AA)_{w}, (X)_{b}, (T)_{g} and D are as defined above and wherein the moiety Ab comprising at least one antigen binding site is an antigen-binding peptide.
A further preferred drug conjugate of formula [D-(X)_{b}-(AA)_{w}-(T)_{g}-(L)-]ₙ-Ab according to the present invention is one wherein L, (AA)_{w}, (X)_{b}, (T)_{g} and D are as defined above and the moiety Ab comprising at least one antigen binding site is an antibody, a single domain antibody or an antigen-binding fragment thereof.
A further preferred drug conjugate of formula [D-(X)_{b}-(AA)_{w}-(T)_{g}-(L)-]ₙ-Ab according to the present invention is one wherein L, (AA)_{w}, (X)_{b}, (T)_{g} and D are as defined above and the moiety Ab comprising at least one antigen binding site is a monoclonal, polyclonal antibody or bispecific antibody and wherein the antibody or antigen-binding fragment thereof is derived from any species, preferably a human, mouse or rabbit.
A further preferred drug conjugate of formula [D-(X)_{b}-(AA)_{w}-(T)_{g}-(L)-]ₙ-Ab according to the present invention is one wherein L, (AA)_{w}, (X)_{b}, (T)_{g} and D are as defined above and the moiety Ab comprising at least one antigen binding site is an antibody or antigen-binding fragment thereof which is selected from the group consisting of a human antibody, an antigen-binding fragment of a human antibody, a humanized antibody, an antigen-binding fragment of a humanized antibody, a chimeric antibody, an antigen-binding fragment of a chimeric antibody, a glycosylated antibody and a glycosylated antigen binding fragment.
A further preferred drug conjugate of formula [D-(X)_{b}-(AA)_{w}-(T)_{g}-(L)-]ₙ-Ab according to the present invention is one wherein L, (AA)_{w}, (X)_{b}, (T)_{g} and D are as defined above and the moiety Ab comprising at least one antigen binding site is an antibody or antigen-binding fragment thereof, wherein the antibody or antigen-binding fragment thereof is an antigen-binding fragment selected from the group consisting of an Fab fragment, an Fab′ fragment, an F(ab′)₂ fragment and an Fv fragment.
A further preferred drug conjugate of formula [D-(X)_{b}-(AA)_{w}-(T)_{g}-(L)-]ₙ-Ab according to the present invention is one wherein L, (AA)_{w}, (X)_{b}, (T)_{g} and D are as defined above and the moiety Ab comprising at least one antigen binding site is an antibody or antigen-binding fragment thereof, wherein the antibody or antigen-binding fragment thereof is a monoclonal antibody which immunospecifically binds to cancer cell antigens, viral antigens, antigens of cells that produce autoimmune antibodies associated with autoimmune disease, microbial antigens, and preferably a monoclonal antibody which immunospecifically binds to cancer cell antigens.
A further preferred drug conjugate of formula [D-(X)_{b}-(AA)_{w}-(T)_{g}-(L)-]ₙ-Ab according to the the present invention is one wherein L, (AA)_{w}, (X)_{b}, (T)_{g} and D are as defined herein and the moiety Ab comprising at least one antigen binding site is an antibody selected from the group consisting of Alemtuzumab, Anetumab, Atezolizumab, Avelumab, Bevacizumab, Blinatomumab, Brentuximab, Catumaxomab, Cetuximab, Coltuximab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Lifastuzumab, Lorvotuzumab, Milatuzumab, Mirvetuximab, Naratuximab, Necitumumab, Nimotuzumab, Nivolumab, Obinutuzumab, Ofatumumab, Olaratumab, Panitumumab, Pembrolizumab, Pertuzumab, Pinatuzumab, Polatuzumab, Ramucirumab, Rovalpituzumab, Sacituzumab, Siltuximab, Sirtratumab, Sofituzumab, Vadastuximab, Vorsetuzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof, wherein preferably the antibody is selected from Alemtuzumab, Anetumab, Atezolizumab, Avelumab, Bevacizumab, Blinatomumab, Brentuximab, Catumaxomab, Cetuximab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Mirvetuximab, Naratuximab, Necitumumab, Nimotuzumab, Nivolumab, Obinutuzumab, Ofatumumab, Olaratumab, Panitumumab, Pembrolizumab, Pertuzumab, Polatuzumab, Ramucirumab, Rovalpituzumab, Sacituzumab, Siltuximab, Sirtratumab, Vadastuximab, Vorsetuzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof, and yet more preferably Alemtuzumab, Atezolizumab, Avelumab, Bevacizumab, Blinatomumab, Brentuximab, Catumaxomab, Cetuximab, Daratumumab, Denosumab, Dinutuximab, Durvalumab, Elotuzumab, Gemtuzumab, Ibritumomab, Inotuzumab, Ipilimumab, Labetuzumab, Necitumumab, Nimotuzumab, Nivolumab, Obinutuzumab, Ofatumumab, Olaratumab, Panitumumab, Pembrolizumab, Pertuzumab, Ramucirumab, Rovalpituzumab, Siltuximab, an anti-HER2 antibody such as Trastuzumab, an anti-CD4 antibody, an anti-CD5 antibody, an anti-CD13 antibody and an anti-CD30 antibody, or an antigen-binding fragment or an immunologically active portion thereof. Of these, particularly preferred are Brentuximab, Gemtuzumab, Inozutumab, Rovalpituzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof; or the antibody is selected from an anti-HER2 antibody such as Trastuzumab and anti-CD13 antibody or an antigen-binding fragment or an immunologically active portion thereof, particularly Trastuzumab or an antigen-binding fragment or an immunologicallly active portion thereof.
Particularly preferred drug conjugates of formula [D-(X)_{b}-(AA)_{w}-(T)_{g}-(L)-]ₙ-Ab according to the present invention include the following:
(a) a drug conjugate according to the present invention wherein:
L is selected from the group consisting of:

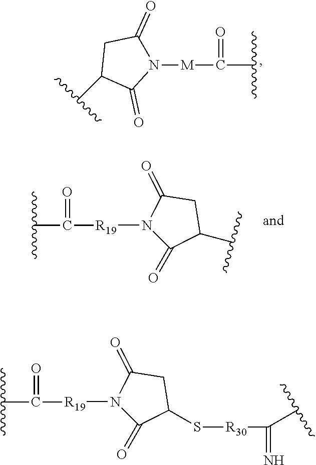

wherein:
the wavy lines indicate the point of covalent attachments to an Ab (the wavy line to the right) and to (T)_{g} if any, or (AA)_{w} if any, or to (X)_{b} if any, or to (D) (the wavy line to the left);
R₁₉ is selected from —C₁-C₁₂ alkylene-, —O—(C₁-C₁₂ alkylene), —C₆-C₁₂ arylene in one or more rings which may optionally be substituted with one or more substituents Rₓ, —C₁-C₁₂ alkylene-C₆-C₁₂ arylene- wherein the arylene group is in one or more rings which may optionally be substituted with one or more substituents Rₓ, —C₆-C₁₂ arylene-C₁-C₁₂ alkylene- wherein the arylene group is in one or more rings which may optionally be substituted with one or more substituents Rₓ, —C₅-C₁₂ heterocyclo- wherein said heterocyclo group may be a saturated or unsaturated group having one or more rings and comprising at least one oxygen, nitrogen or sulphur atom in said ring(s), said group optionally being substituted with one or more substituents Rₓ, —C₁-C₁₂ alkylene-(C₅-C₁₂ heterocyclo)- wherein said heterocyclo group may be a saturated or unsaturated group having one or more rings and comprising at least one oxygen, nitrogen or sulphur atom in said ring(s), said group optionally being substituted with one or more substituents Rₓ, —(C₅-C₁₂ heterocyclo)-C₁-C₁₂ alkylene- wherein said heterocyclo group may be a saturated or unsaturated group having one or more rings and comprising at least one oxygen, nitrogen or sulphur atom in said ring(s), said group optionally being substituted with one or more substituents Rₓ, —(OCH₂CH₂)ᵣ— and —CH₂—(OCH₂CH₂)ᵣ—, wherein each of the above alkylene substituents whether alone or attached to another moiety the carbon chain may optionally be substituted by one or more substituents Rₓ;
R₃₀ is a —C₁-C₆ alkylene- group;
M is selected from the group consisting of —C₁-C₆ alkylene-, —C₁-C₆ alkylene-(C₃-C₈ carbocyclo)- and phenylene which may optionally be substituted with one or more substituents Rₓ;
r is an integer ranging from 1-6;
(AA)_{w} is of formula (II):

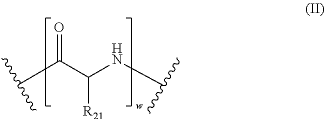

wherein the wavy lines indicate the point of covalent attachments to (X)_{b} if any, or to the drug moiety (the wavy line to the left) and to (T)_{g} if any, or to the linker (the wavy line to the right);
R₂₁ is, at each occurrence, selected from the group consisting of hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

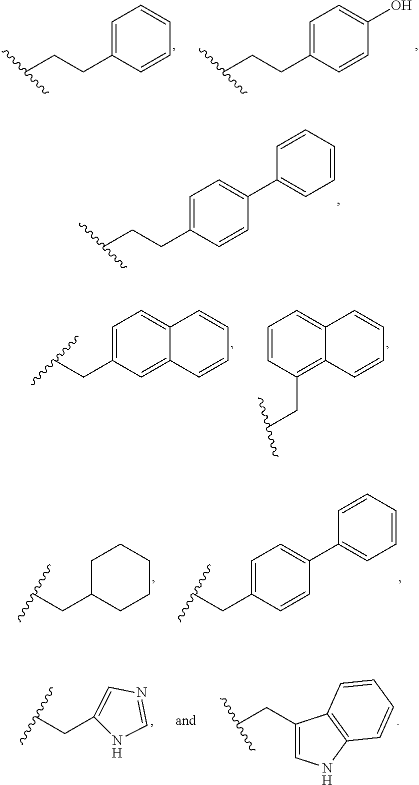

w is an integer ranging from 0 to 12;
wherein X is an extending group selected from
where D is conjugated via an amine group: —COO—(C₁-C₆ alkylene)NH—, —COO—CH₂-(phenylene which may optionally be substituted with one or more substituents Rₓ)—NH—, —COO—(C₁-C₆ alkylene)NH—COO—CH₂-(phenylene which may optionally be substituted with one or more substituents Rₓ)—NH—, —COCH₂NH—COCH₂—NH—, —COCH₂—NH—, —COO—(C₁-C₆ alkylene)S—, —COO—(C₁-C₆ alkylene)NHCO(C₁-C₆ alkylene)S—; or
where D is conjugated via an hydroxy group: —CONH—(C₁-C₆ alkylene)NH—, —COO—CH₂-(phenylene which may optionally be substituted with one or more substituents Rₓ)—NH—, —CONH—(C₁-C₆ alkylene)NH—COO—CH₂-(phenylene which may optionally be substituted with one or more substituents Rₓ)—NH—, —COCH₂NH—COCH₂—NH—, —COCH₂NH—, —CONH—(C₁-C₆ alkylene)S—, and —CONH—(C₁-C₆ alkylene)NHCO(C₁-C₆ alkylene)S—;
b is 0 or 1, preferably 1;
wherein T is an extending group selected from —CO—(C₁-C₆ alkylene)-NH—, —CO—(C₁-C₆ alkylene)-[O—(C₂-C₆ alkylene)]-NH—, and —COO—(C₁-C₆ alkylene)-[O—(C₂-C₆ alkylene)]ⱼ—NH—, where j is an integer from 1 to 25;
g is 0 or 1;
D is a drug moiety of formula (I) or (IH), or a pharmaceutically acceptable salt, ester, solvate, tautomer or stereoisomer thereof wherein:
R₂ is C(═O)Rₐ, wherein Rₐ is selected from hydrogen and substituted or unsubstituted C₁-C₆ alkyl, wherein the optional substituents are one or more substituents Rₓ;
R₃ is hydrogen or a —OR_{b} group, wherein R_{b} is a substituted or unsubstituted C₁-C₆ alkyl group, wherein the optional substituents are one or more substituents Rₓ;
R₄ is selected from hydrogen, —CH₂OH and —CH₂NH₂;
the moiety Ab comprising at least one antigen binding site is an antibody or an antigen-binding fragment thereof and it is selected from the group consisting of a human antibody, an antigen-binding fragment of a human antibody, a humanized antibody, an antigen-binding fragment of a humanized antibody, a chimeric antibody, an antigen-binding fragment of a chimeric antibody, a glycosylated antibody and a glycosylated antigen binding fragment; and
n is the ratio of the group [D-(X)_{b}-(AA)_{w}-(T)_{g}-(L)-] to the moiety Ab comprising at least one antigen binding site and is in the range from 1 to 12.
(b) a drug conjugate according to the present invention selected from the formulas (IV), (V) and (VI):

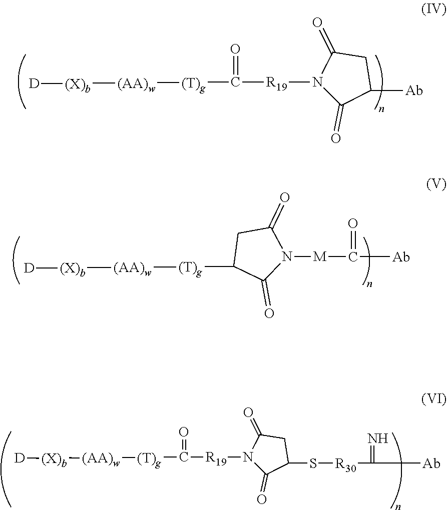

wherein:
R₁₉ is selected from —C₁-C₈ alkylene-, —O—(C₁-C₈ alkylene), —C₁-C₈ alkylene-C₆-C₁₂ arylene- wherein the arylene group is in one or more rings which may optionally be substituted with one or more substituents Rₓ and —C₆-C₁₂ arylene-C₁-C₈ alkylene- wherein the arylene group is in one or more rings which may optionally be substituted with one or more substituents Rₓ, wherein each of the above alkylene substituents whether alone or attached to another moiety the carbon chain may optionally be substituted by one or more substituents Rₓ;
R₃₀ is a —C₂-C₄ alkylene- group;
M is selected from the group consisting of —C₁-C₃ alkylene- and —C₁-C₃ alkylene-(C₅-C₇ carbocyclo)-;
(AA)_{w} is of formula (II)

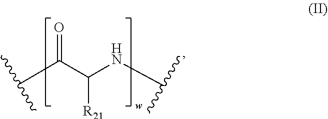

wherein:
the wavy lines indicate the point of covalent attachments to (X)_{b} if any, or to the drug moiety (the wavy line to the left) and to (T)_{g} if any, or to the linker (the wavy line to the right);
R₂₁ is, at each occurrence, selected from the group consisting of hydrogen, methyl, isopropyl, sec-butyl, benzyl, indolylmethyl, —(CH₂)₃NHCONH₂, —(CH₂)₄NH₂,
—(CH₂)₃NHC(═NH)NH₂ and —(CH₂)₄NHC(═NH)NH₂;
w is an integer from 0 to 6;
X is an extending group selected from the group consisting of
where D is conjugated via an amine group: —COO—(C₂-C₄ alkylene)NH—, —COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups, —COO—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—, —COCH₂NH—COCH₂—NH—, —COO—(C₂-C₄ alkylene)S—, and —COO—(C₂-C₄ alkylene)NHCO(C₁-C₃ alkylene)S—; or
where D is conjugated via an hydroxy group: —CONH—(C₂-C₄ alkylene)NH—, —COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups, —CONH—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—, —COCH₂NH—COCH₂—NH—, —CONH—(C₂-C₄ alkylene)S—, and —CONH—(C₂-C₄ alkylene)NHCO(C₁-C₃ alkylene)S—;
b is 0 or 1, preferably 1;
wherein T is an extending group selected from —CO—(C₁-C₄ alkylene)-NH—, —CO—(C₁-C₄ alkylene)-[O—(C₂-C₄ alkylene)]ⱼ—NH—, and —COO—(C₁-C₄ alkylene)-[O—(C₂-C₄ alkylene)]ⱼ—NH—, where j is an integer from 1 to 10;
g is 0 or 1;
D is a drug moiety of formula (I) or (IH), or a pharmaceutically acceptable salt, ester, solvate, tautomer or stereoisomer thereof wherein:
R₂ is acetyl;
R₃ is hydrogen or methoxy, preferably R₃ is methoxy;
R₄ is hydrogen or —CH₂OH, preferably R₄ is hydrogen;
the moiety Ab comprising at least one antigen binding site is an antibody or an antigen-binding fragment thereof, wherein the antibody or antigen-binding fragment is a monoclonal antibody which immunospecifically binds to cancer cell antigens, viral antigens, antigens of cells that produce autoimmune antibodies associated with autoimmune disease, microbial antigens, and preferably a monoclonal antibody which immunospecifically binds to cancer cell antigens; and
n is the ratio of the group [D-(X)_{b}-(AA)_{w}-(T)_{g}-(L)-] wherein L is as defined in formulas (IV), (V) or (VI) to the moiety Ab comprising at least one antigen binding site and is in the range from 3 to 8.
(c) a drug conjugate according to the present invention selected from the formulas (IV), (V) and (VI):

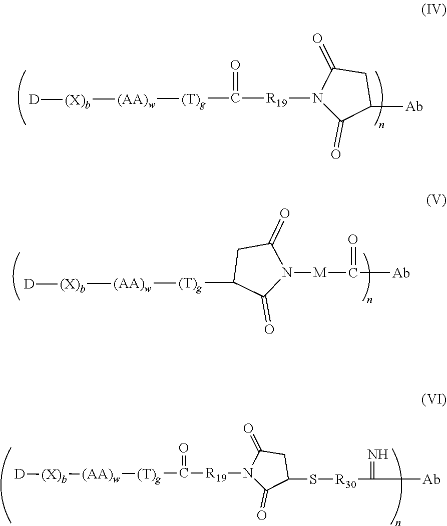

wherein:
R₁₉ is selected from —C₁-C₆ alkylene-, -phenylene-C₁-C₆ alkylene- wherein the phenylene group may optionally be substituted with one or more substituents Rₓ selected from the group consisting of alkyl groups having from 1 to 6 carbon atoms, alkoxy groups having from 1 to 6 carbon atoms, halogen atoms, nitro groups and cyano groups, wherein each of the above alkylene substituents whether alone or attached to another moiety in the carbon chain may optionally be substituted by one or more substituents Rₓ selected from the group consisting of alkyl groups having from 1 to 6 carbon atoms, alkoxy groups having from 1 to 6 carbon atoms, aryl groups having from 6 to 12 carbon atoms, halogen atoms, nitro groups and cyano groups, and preferably R₁₉ is a C₁-C₆ alkylene group;
R₃₀ is a —C₂-C₄ alkylene- group;
M is —C₁-C₃ alkylene-(C₅-C₇ carbocyclo)-;
w is 0 or 2, and where w is 2, then (AA)_{w} is of formula (III):

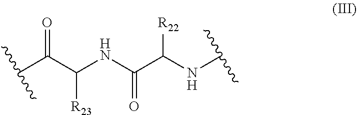

wherein the wavy lines indicate the point of covalent attachments to (X)_{b} if any, or to the drug moiety (the wavy line to the left) and to (T)_{g} if any, or to the linker (the wavy line to the right);
R₂₂ is selected from methyl, benzyl, isopropyl, sec-butyl and indolylmethyl;
R₂₃ is selected from methyl, —(CH₂)₄NH₂, —(CH₂)₃NHCONH₂ and —(CH₂)₃NHC(═NH)NH₂;
X is an extending group selected from the group consisting of —COO—(C₂-C₄ alkylene)NH—, —COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups, —COO—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups or cyano groups)-NH—, —COCH₂NH—COCH₂—NH—, —COO—(C₂-C₄ alkylene)S—, and —COO—(C₂-C₄ alkylene)NHCO(C₁-C₃ alkylene)S—;
b is 0 or 1, preferably 1;
wherein T is an extending group selected from —CO—(C₁-C₄ alkylene)-NH—, —CO—(C₁-C₄ alkylene)-[O—(C₂-C₄ alkylene)]ⱼ—NH—, and —COO—(C₁-C₄ alkylene)-[O—(C₂-C₄ alkylene)]ⱼ—NH—, where j is an integer from 1 to 5;
g is 0 or 1;
D is a drug moiety of formula (I) or (IH), or a pharmaceutically acceptable salt, ester, solvate, tautomer or stereoisomer thereof wherein:
R₁ is CN;
R₂ is acetyl:
R₃ is methoxy;
R₄ is hydrogen;
Y is —NH— or —O—;
the moiety Ab comprising at least one antigen binding site is a monoclonal antibody selected from the group consisting of Alemtuzumab, Anetumab, Atezolizumab, Avelumab, Bevacizumab, Blinatomumab, Brentuximab, Catumaxomab, Cetuximab, Coltuximab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Lifastuzumab, Lorvotuzumab, Milatuzumab, Mirvetuximab, Naratuximab, Necitumumab, Nimotuzumab, Nivolumab, Obinutuzumab, Ofatumumab, Olaratumab, Panitumumab, Pembrolizumab, Pertuzumab, Pinatuzumab, Polatuzumab, Ramucirumab, Rovalpituzumab, Sacituzumab, Siltuximab, Sirtratumab, Sofituzumab, Vadastuximab, Vorsetuzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof, wherein preferably the antibody is selected from Alemtuzumab, Anetumab, Atezolizumab, Avelumab, Bevacizumab, Blinatomumab, Brentuximab, Catumaxomab, Cetuximab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Mirvetuximab, Naratuximab, Necitumumab, Nimotuzumab, Nivolumab, Obinutuzumab, Ofatumumab, Olaratumab, Panitumumab, Pembrolizumab, Pertuzumab, Polatuzumab, Ramucirumab, Rovalpituzumab, Sacituzumab, Siltuximab, Sirtratumab, Vadastuximab, Vorsetuzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof, and yet more preferably Alemtuzumab, Atezolizumab, Avelumab, Bevacizumab, Blinatomumab, Brentuximab, Catumaxomab, Cetuximab, Daratumumab, Denosumab, Dinutuximab, Durvalumab, Elotuzumab, Gemtuzumab, Ibritumomab, Inotuzumab, Ipilimumab, Labetuzumab, Necitumumab, Nimotuzumab, Nivolumab, Obinutuzumab, Ofatumumab, Olaratumab, Panitumumab, Pembrolizumab, Pertuzumab, Ramucirumab, Rovalpituzumab, Siltuximab, an anti-HER2 antibody such as Trastuzumab, an anti-CD4 antibody, an anti-CD5 antibody, an anti-CD13 antibody and an anti-CD30 antibody, or an antigen-binding fragment or an immunologically active portion thereof. Of these, particularly preferred are Brentuximab, Gemtuzumab, Inozutumab, Rovalpituzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof; or the antibody is selected from an anti-HER2 antibody such as Trastuzumab and anti-CD13 antibody or an antigen-binding fragment or an immunologically active portion thereof, particularly Trastuzumab or an antigen-binding fragment or an immunologicallly active portion thereof; and
n is the ratio of the group [D-(X)_{b}-(AA)_{w}-(T)_{g}-(L)-] wherein L is as defined in formulas (IV), (V) or (VI) to the moiety Ab comprising at least one antigen binding site and is in the range from 3 to 5.
(d) A drug conjugate according to the present invention selected from the formulas (IV), (V) and (VI):

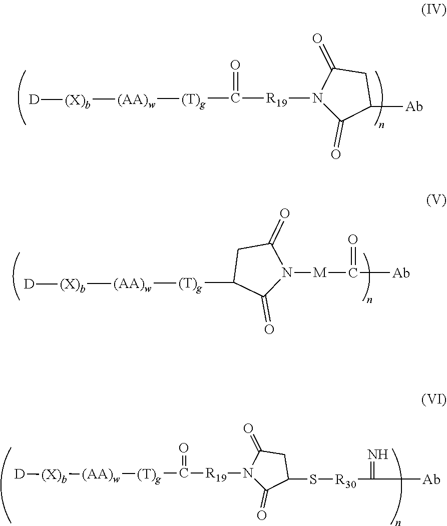

wherein:
R₁₉ is —C₂-C₆ alkylene-;
R₃₀ is a —C₂-C₄ alkylene-;
M is —C₁-C₃ alkylene-(C₅-C₇ carbocyclo)-;
w is 0 or 2, and where w is 2, then (AA)_{w} is of formula (III):

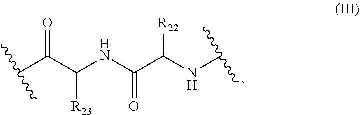

wherein R₂₂ is isopropyl, R₂₃ is selected from methyl and —(CH₂)₃NHCONH₂, wherein the wavy lines indicate the point of covalent attachments to (X)_{b} if any, or to the drug moiety (the wavy line to the left) and to (T)_{g} if any, or to the linker (the wavy line to the right);
X is an extending group selected from the group consisting of —COO—(C₂-C₄ alkylene)NH—, —COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups, —COO—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—, —COCH₂NH—COCH₂—NH—, —COO—(C₂-C₄ alkylene)S—, and —COO—(C₂-C₄ alkylene)NHCO(C₁-C₃ alkylene)S—;
b is 0 or 1, preferably 1; wherein T is an extending group selected from —CO—(C₁-C₄ alkylene)-NH—, —CO—(C₁-C₄ alkylene)-[O—(C₂-C₄ alkylene)]ⱼ—NH—, and —COO—(C₁-C₄ alkylene)-[O—(C₂-C₄ alkylene)]ⱼ—NH—, where j is an integer from 1 to 5;
g is 0 or 1;
D is a drug moiety selected from:

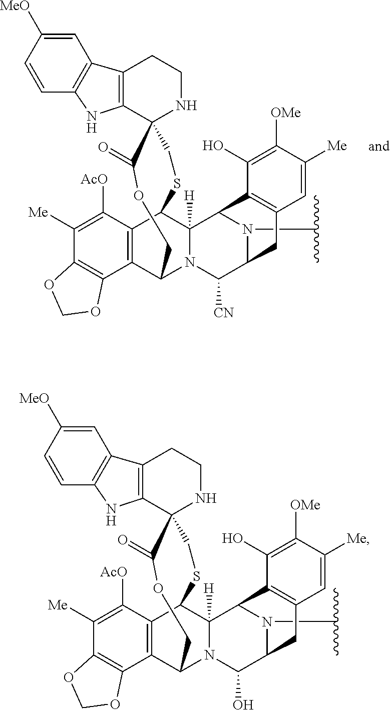 [SMILES: *N1[C@@H]2c3c(cc(C)c(OC)c3O)C[C@H]1[C@H](C#N)N1[C@H]2[C@@H]2SC[C@]3(NCCc4c3[nH]c3ccc(OC)cc43)C(=O)OC[C@H]1c1c3c(c(C)c(OC(C)=O)c12)OCO3 ; "and" ; *N1[C@@H]2c3c(cc(C)c(OC)c3O)C[C@H]1[C@H](O)N1[C@H]2[C@@H]2SC[C@]3(NCCc4c3[nH]c3ccc(OC)cc43)C(=O)OC[C@H]1c1c3c(c(C)c(OC(C)=O)c12)OCO3]

or a pharmaceutically acceptable salt, ester, solvate, tautomer or stereoisomer thereof; wherein the wavy line indicates the point of covalent attachment to (X)_{b} if any, or (AA)_{w} if any, or to (T)_{g} if any, or to (L);
the moiety Ab comprising at least one antigen binding site is selected from Brentuximab, Gemtuzumab, Inozutumab, Rovalpituzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof, and more preferably its is selected from an anti-HER2 antibody such as Trastuzumab and anti-CD13 antibody or an antigen-binding fragment or an immunologically active portion thereof, particularly Trastuzumab or an antigen-binding fragment or an immunologicallly active portion thereof; and
n is the ratio of the group [D-(X)_{b}-(AA)_{w}-(T)_{g}-(L)-] wherein L is as defined in formulas (IV), (V) or (VI) to the moiety Ab comprising at least one antigen binding site and is in the range from 3 to 5.
(e) A drug conjugate according to the present invention selected from the formulas (IV), (V), and (VI):

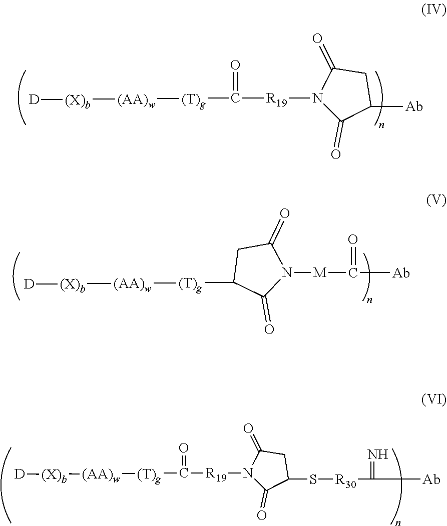

wherein:
R₁₉ is —C₂-C₆ alkylene-;
R₃₀ is —C₂-C₄ alkylene-;
M is —C₁-C₃ alkylene-(C₅-C₇ carbocyclo)-;
w is 0 or 2, and where w is 2, then (AA)_{w} is of formula (III):

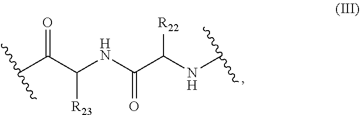

wherein R₂₂ is isopropyl, R₂₃ is selected from methyl and —(CH₂)₃NHCONH₂, and the wavy lines indicate the point of covalent attachments to (X)_{b} if any, or to the drug moiety (the wavy line to the left) and to (T)_{g} if any, or to the linker (the wavy line to the right);
X is an extending group selected from the group consisting of —COO—(C₂-C₄ alkylene)NH—, —COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups, —COO—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—, —COCH₂NH—COCH₂—NH—, —COO—(C₂-C₄ alkylene)S—, and —COO—(C₂-C₄ alkylene)NHCO(C₁-C₃ alkylene)S—;
b is 0 or 1, preferably 1;
wherein T is an extending group selected from —CO—(C₁-C₄ alkylene)-NH—, —CO—(C₁-C₄ alkylene)-[O—(C₂-C₄ alkylene)]ⱼ—NH—, and —COO—(C₁-C₄ alkylene)-[O—(C₂-C₄ alkylene)]ⱼ—NH—, where j is an integer from 1 to 5;
g is 0 or 1;
D is a drug moiety selected from:

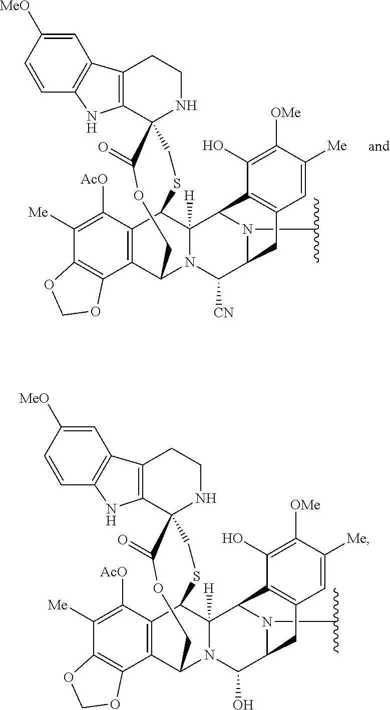 [SMILES: *N1[C@@H]2c3c(cc(C)c(OC)c3O)C[C@H]1[C@H](C#N)N1[C@H]2[C@@H]2SC[C@]3(NCCc4c3[nH]c3ccc(OC)cc43)C(=O)OC[C@H]1c1c3c(c(C)c(OC(C)=O)c12)OCO3 ; "and" ; *N1[C@@H]2c3c(cc(C)c(OC)c3O)C[C@H]1[C@H](O)N1[C@H]2[C@@H]2SC[C@]3(NCCc4c3[nH]c3ccc(OC)cc43)C(=O)OC[C@H]1c1c3c(c(C)c(OC(C)=O)c12)OCO3]

or a pharmaceutically acceptable salt, ester, solvate, tautomer or stereoisomer thereof; wherein the wavy line indicates the point of covalent attachment to (X)_{b} if any, or (AA)_{w} if any, or to (T)_{g} if any, or to (L);
the moiety Ab comprising at least one antigen binding site is selected from Brentuximab, Gemtuzumab, Inozutumab, Rovalpituzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof, and more preferably its is selected from an anti-HER2 antibody such as Trastuzumab and anti-CD13 antibody or an antigen-binding fragment or an immunologically active portion thereof, particularly Trastuzumab or an antigen-binding fragment or an immunologicallly active portion thereof; and
n is the ratio of the group [D-(X)_{b}-(AA)_{w}-(T)_{g}-(L)-] wherein L is as defined in formulas (IV), (V) or (VI) to the moiety comprising at least one antigen binding site and is in the range from 3 to 5.
(f) A drug conjugate according to the present invention of formula (IV):

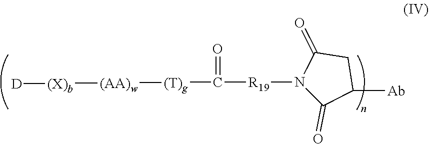

wherein:
R₁₉ is C₂-C₈ alkylene-;
w is 0 or 2, and where w is 2, then (AA)_{w} is of formula (III):

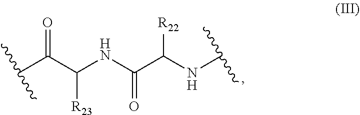

wherein R₂₂ is isopropyl, R₂₃ is selected from methyl and —(CH₂)₃NHCONH₂, and the wavy lines indicate the point of covalent attachments to (X)_{b} (the wavy line to the left) and to (T)_{g} if any, or to the linker (the wavy line to the right); and
X is a —COOCH₂-phenylene-NH group;
b is 1;
T is an extending group of formula —CO—(C₁-C₄ alkylene)-[O—(C₂-C₄ alkylene)]₄—NH—;
g is 0 or 1;
or of formula (V)

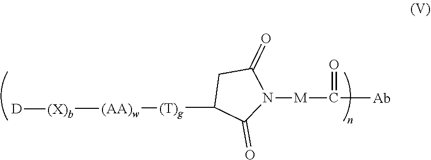

wherein M is -methyl-cyclohexylene-;
b is 1;
w is 0;
X is an extending group selected from —(CH₂)₃S— and —(CH₂)₃NHCO(CH₂)₂S—
g is 0;
or of formula (VI)

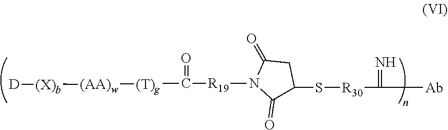

wherein R₁₉ is —C₂-C₅ alkylene-;
R₃₀ is —C₃ alkylene-;
w is 0 or 2, and where w is 2, then (AA)_{w} is of formula (III):

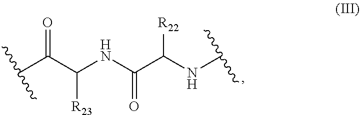

wherein R₂₂ is isopropyl, R₂₃ is selected from methyl and —(CH₂)₃NHCONH₂, and the wavy lines indicate the point of covalent attachments to (X)_{b} (the wavy line to the left) and to (T)_{g} if any, or to the linker (the wavy line to the right); and
X is a —COOCH₂-phenylene-NH group;
b is 1;
T is an extending group of formula —CO—(C₁-C₄ alkylene)-[O—(C₂-C₄ alkylene)]₄—NH—;
g is 0 or 1;
D is a drug moiety selected from:

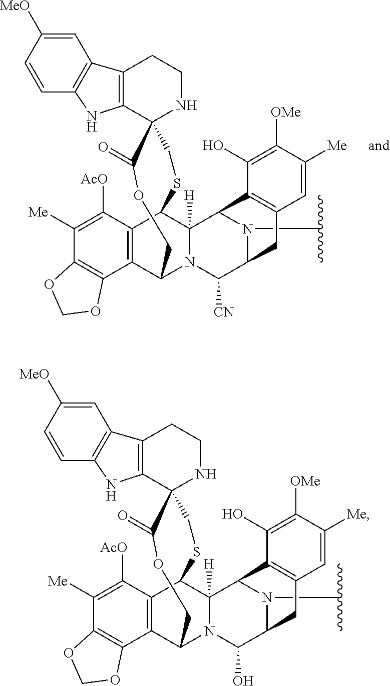 [SMILES: *N1[C@@H]2c3c(cc(C)c(OC)c3O)C[C@H]1[C@H](C#N)N1[C@H]2[C@@H]2SC[C@]3(NCCc4c3[nH]c3ccc(OC)cc43)C(=O)OC[C@H]1c1c3c(c(C)c(OC(C)=O)c12)OCO3 ; "and" ; *N1[C@@H]2c3c(cc(C)c(OC)c3O)C[C@H]1[C@H](O)N1[C@H]2[C@@H]2SC[C@]3(NCCc4c3[nH]c3ccc(OC)cc43)C(=O)OC[C@H]1c1c3c(c(C)c(OC(C)=O)c12)OCO3]

or a pharmaceutically acceptable salt, ester, solvate, tautomer or stereoisomer thereof; wherein the wavy line indicates the point of covalent attachment to (X)_{b};
the moiety Ab comprising at least one antigen binding site is Brentuximab, Gemtuzumab, Inozutumab, Rovalpituzumab, an anti-HER2 antibody such as Trastuzumab, an anti-CD4 antibody, an anti-CD5 antibody, an anti-CD13 antibody and an anti-CD30 antibody, or an antigen-binding fragment or an immunologicallly active portion thereof, and more preferably its is selected from an anti-HER2 antibody such as Trastuzumab and anti-CD13 antibody or an antigen-binding fragment or an immunologically active portion thereof, particularly Trastuzumab or an antigen-binding fragment or an immunologicallly active portion thereof; and
n is the ratio of the group [D-(X)_{b}-(AA)_{w}-(T)_{g}-(L)-] wherein L is as defined in formula (IV) to the moiety Ab comprising at least one antigen binding site and is in the range from 3 to 5, and preferably 4.
g) an antibody drug conjugate according according to the present invention, selected from the group consisting of:

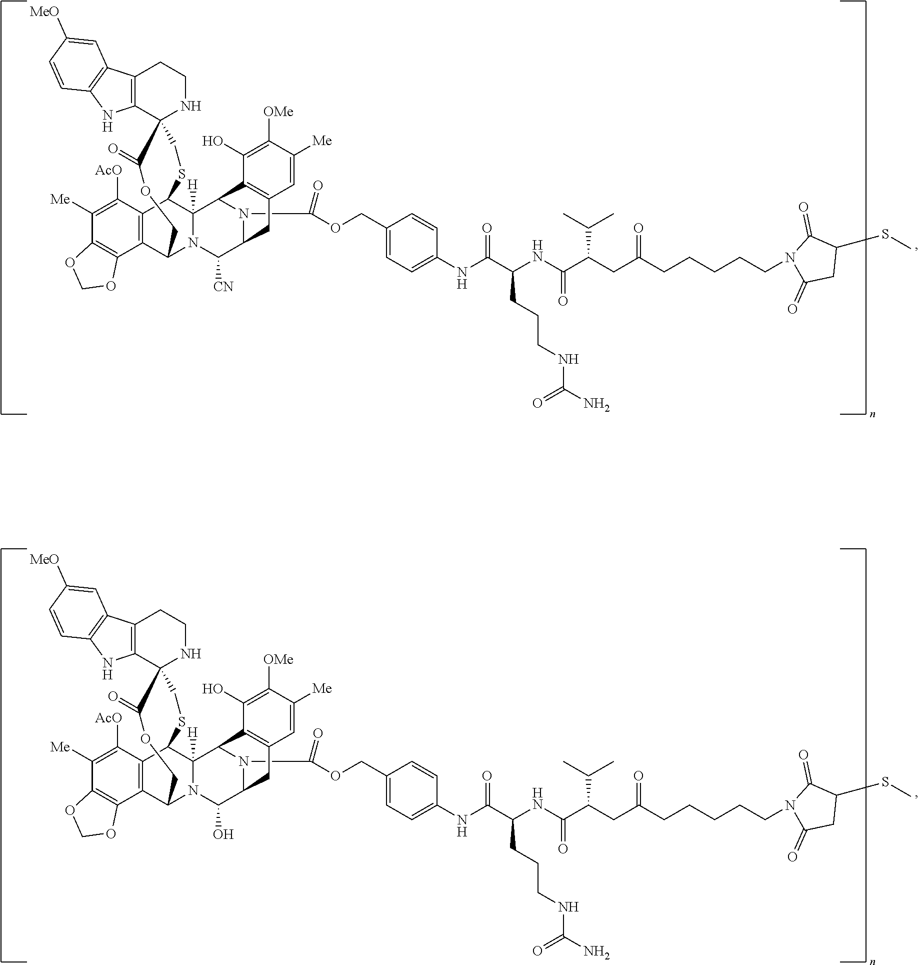

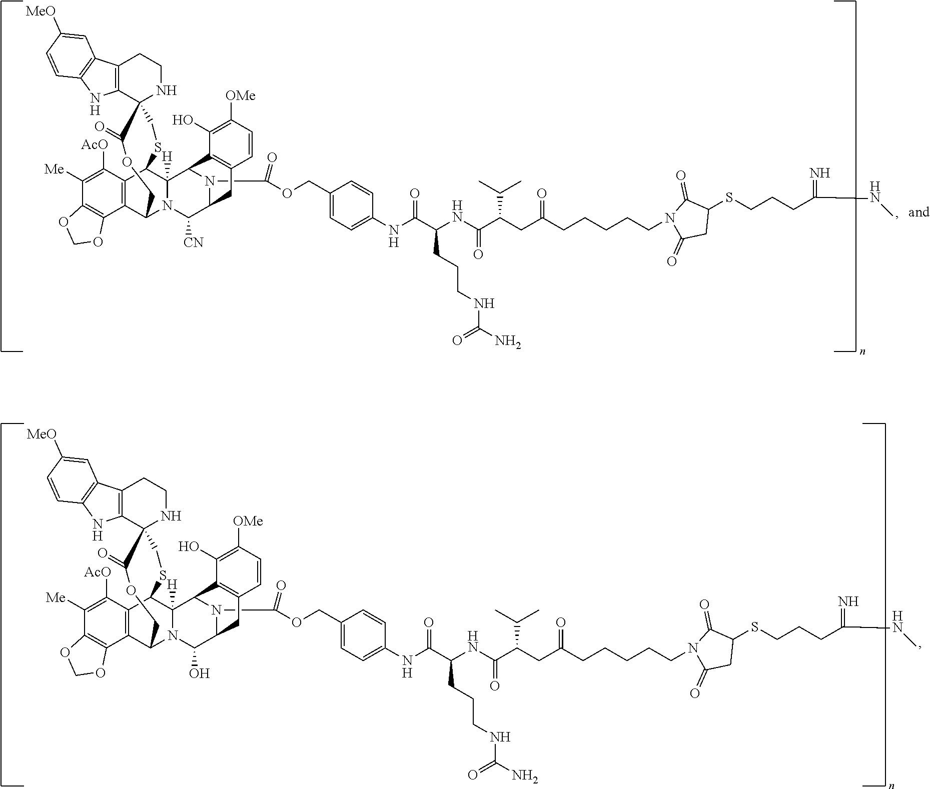

wherein n is from 2 to 6, more preferably 3, 4, or 5 and each [symbol] and [symbol] is independently selected from Brentuximab, Gemtuzumab, Inozutumab, Rovalpituzumab, an anti-HER2 antibody such as Trastuzumab, an anti-CD4 antibody, an anti-CD5 antibody, an anti-CD13 antibody and an anti-CD30 antibody, or an antigen-binding fragment or an immunologically active portion thereof, and more preferably its is selected from an anti-HER2 antibody such as Trastuzumab and anti-CD13 antibody or an antigen-binding fragment or an immunologically active portion thereof, particularly Trastuzumab or an antigen-binding fragment or an immunologically active portion thereof.

More preferably the antibody drug conjugate is selected from the group consisting of:

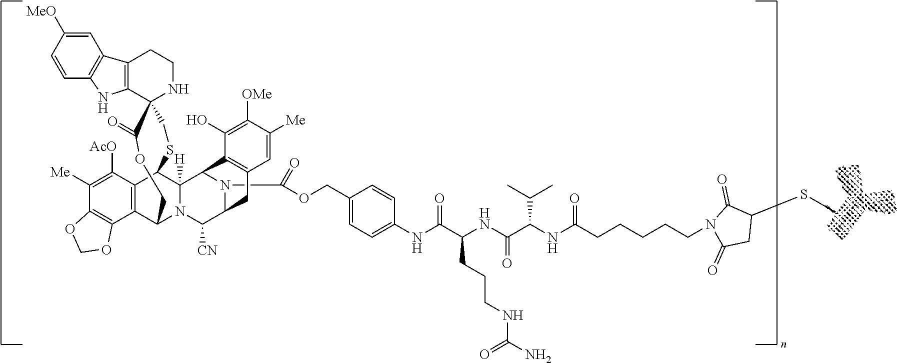

wherein n is from 2 to 6, more preferably 3, 4, or 5 and [symbol] is selected from an anti-HER2 antibody such as Trastuzumab and an anti-CD13 antibody or an antigen-binding fragment or an immunologically active portion thereof, more preferably is Trastuzumab or an antigen binding fragment or an immunologically active portion thereof,

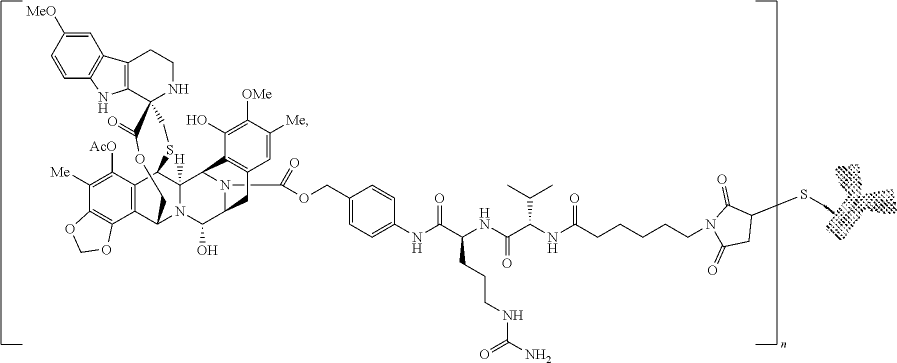

wherein n is from 2 to 6, more preferably 3, 4, or 5 and [symbol] is selected from an anti-HER2 antibody such as Trastuzumab and an anti-CD13 antibody or an antigen-binding fragment or an immunologically active portion thereof, more preferably is Trastuzumab or an antigen-binding fragment or an immunologically active portion thereof,

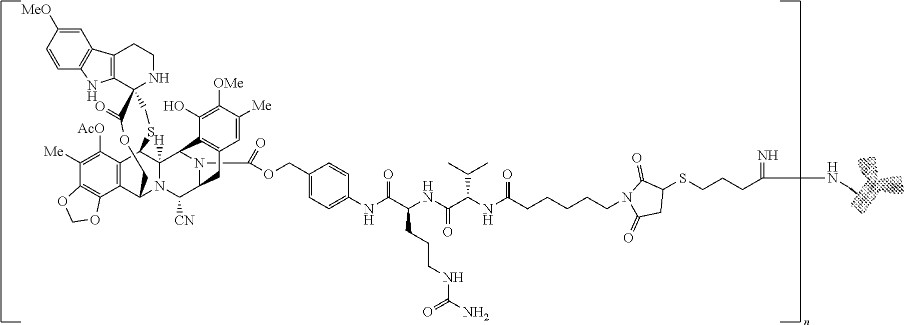

wherein n is from 2 to 6, more preferably 3, 4, or 5 and [symbol] is selected from an anti-HER2 antibody such as Trastuzumab and an anti-CD13 antibody or an antigen-binding fragment or an immunologically active portion thereof, more preferably is Trastuzumab or an antigen-binding fragment or an immunologically active portion thereof,

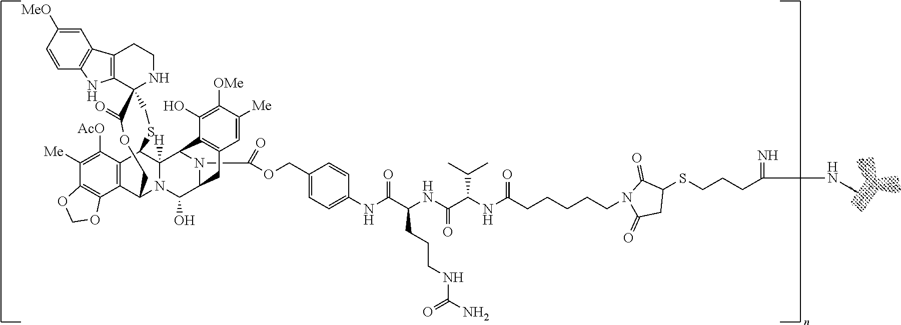

wherein n is from 2 to 6, more preferably 3, 4, or 5 and [symbol] is selected from an anti-HER2 antibody such as Trastuzumab and an anti-CD13 antibody or an antigen-binding fragment or an immunologically active portion thereof, more preferably is Trastuzumab or an antigen-binding fragment or an immunologically active portion thereof.

Particularly preferably, the antibody drug conjugates according to the present invention should be in isolated or purified form.

Preferred compounds of formula D-(X)_{b}-(AA)_{w}-(T)_{g}-L₁ or of formula D-(X)_{b}-(AA)_{w}-(T)_{g}-H according to the present invention include:
a compound of formula D-(X)_{b}-(AA)_{w}-(T)_{g}-L₁ or of formula D-(X)_{b}-(AA)_{w}-(T)_{g}-H wherein each of D, X, AA, T, L₁, b, g and w are as defined herein in the present invention; but further wherein if the compound is a compound of formula D-(X)_{b}-(AA)_{w}(T)_{g}-H then b+w+g≠0.
a compound of formula D-(X)_{b}-(AA)_{w}-(T)_{g}-L₁ or of formula D-(X)_{b}-(AA)_{w}-(T)_{g}-H according to the present invention wherein:
L₁ is a linker of formula:

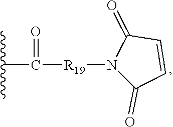

wherein:
the wavy line indicates the point of covalent attachment to (T)_{g} if any, or (AA)_{w} if any, or to (X)_{b} if any, or to D;
R₁₉ is selected from —C₁-C₁₂ alkylene-, —O—(C₁-C₁₂ alkylene), —C₆-C₁₂ arylene in one or more rings which may optionally be substituted with one or more substituents Rₓ, —C₁-C₁₂ alkylene-C₆-C₁₂ arylene- wherein the arylene group is in one or more rings which may optionally be substituted with one or more substituents Rₓ, —C₆-C₁₂ arylene-C₁-C₁₂ alkylene- wherein the arylene group is in one or more rings which may optionally be substituted with one or more substituents Rₓ, —C₅-C₁₂ heterocyclo- wherein said heterocyclo group may be a saturated or unsaturated group having one or more rings and comprising at least one oxygen, nitrogen or sulphur atom in said ring(s), said group optionally being substituted with one or more substituents Rₓ, —C₁-C₁₂ alkylene-(C₅-C₁₂ heterocyclo)- wherein said heterocyclo group may be a saturated or unsaturated group having one or more rings and comprising at least one oxygen, nitrogen or sulphur atom in said ring(s), said group optionally being substituted with one or more substituents Rₓ, —(C₅-C₁₂ heterocyclo)-C₁-C₁₂ alkylene- wherein said heterocyclo group may be a saturated or unsaturated group having one or more rings and comprising at least one oxygen, nitrogen or sulphur atom in said ring(s), said group optionally being substituted with one or more substituents Rₓ, —(OCH₂CH₂)ᵣ— and —CH₂—(OCH₂CH₂)ᵣ—, wherein each of the above alkylene substituents whether alone or attached to another moiety the carbon chain may optionally be substituted by one or more substituents Rₓ;
r is an integer ranging from 1-6; and
each of D, Rₓ, X, AA, T, b, g and w is as defined in the present invention; but wherein if the compound is a compound of formula D-(X)_{b}-(AA)_{w}-(T)_{g}-H then b+w+g≠0.
   a compound of formula D-(X)_{b}-(AA)_{w}-(T)_{g}-L₁ or of formula D-(X)_{b}-(AA)_{w}-(T)_{g}-H according to the present invention wherein:
L₁ is linker of formula:

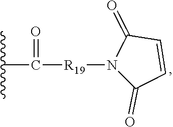

wherein:
the wavy line indicates the point of covalent attachment to (T)_{g} if any, or (AA)_{w} if any, or to (X)_{b} if any, or to D;
R₁₉ is selected from —C₁-C₈ alkylene-, —O—(C₁-C₈ alkylene), —C₁-C₈ alkylene-C₆-C₁₂ arylene- wherein the arylene group is in one or more rings which may optionally be substituted with one or more substituents Rₓ, and —C₆-C₁₂ arylene-C₁-C₈ alkylene- wherein the arylene group is in one or more rings which may optionally be substituted with one or more substituents Rₓ, wherein each of the above alkylene substituents whether alone or attached to another moiety the carbon chain may optionally be substituted by one or more substituents Rₓ;
(AA)_{w} is of formula (II):

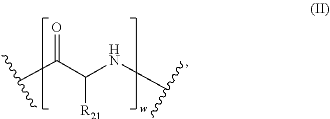

wherein the wavy lines indicate the point of covalent attachments to (X)_{b}, if any, or to D (the wavy line to the left) and to (T)_{g} if any, or L₁ or to a hydrogen atom (the wavy line to the right);
wherein R₂₁ is selected, at each occurrence, from the group consisting of hydrogen, methyl, isopropyl, sec-butyl, benzyl, indolylmethyl, —(CH₂)₃NHCONH₂, —(CH₂)₄NH₂, —(CH₂)₃NHC(═NH)NH₂ and —(CH₂)₄NHC—(═NH)NH₂, and w is an integer from 0 to 6;
X is an extending group selected from the group consisting of
where D is conjugated via an amine group: —COO—(C₂-C₄ alkylene)NH—, —COO—CH₂-phenylene-NH, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups, —COO—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—, —COCH₂NH—COCH₂—NH—, —COO—(C₂-C₄ alkylene)S—, and —COO—(C₂-C₄ alkylene)-NHCO(C₁-C₃ alkylene)S— or
where D is conjugated via an hydroxy group: —CONH—(C₂-C₄ alkylene)NH—, —COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups, —CONH—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—, —COCH₂NH—COCH₂—NH—, —CONH—(C₂-C₄ alkylene)S—, and —CONH—(C₂-C₄ alkylene)NHCO(C₁-C₃ alkylene)S—;
T is an extending group selected from —CO—(C₁-C₄ alkylene)-NH—; —CO—(C₁-C₄ alkylene)-[O—(C₂-C₄ alkylene)]ⱼ—NH— and —COO—(C₁-C₄ alkylene)-[O—(C₂-C₄ alkylene)]ⱼ—NH—, where j is an integer from 1 to 10;
b is 0 or 1;
g is 0 or 1;
wherein if the compound is a compound of formula D-(X)_{b}-(AA)_{w}-(T)_{g}-H then b+w+g≠0; and
D is a drug moiety of formula (I); and is covalently attached via a hydroxy or amine group; or
is a drug moiety of formula (IH), or a pharmaceutically acceptable salt, ester, solvate, tautomer or stereoisomer thereof wherein:

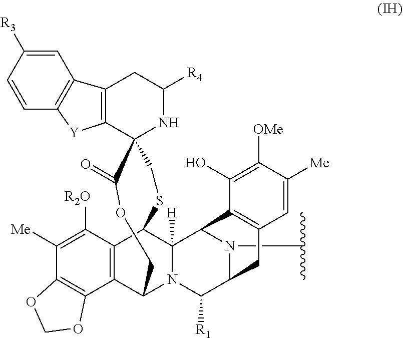

wherein the wavy line of (IH) indicate the point of covalent attachment to (X)_{b} if any, or (AA)_{w} if any, or to (T)_{g} if any, or to L₁;
R₁ is —OH or —CN;
R₂ is a —C(═O)Rₐ group, wherein Rₐ is selected from hydrogen and substituted or unsubstituted C₁-C₆ alkyl, wherein the optional substituents are one or more substituents Rₓ;
R₃ is hydrogen or a —OR_{b} group wherein R_{b} is a substituted or unsubstituted C₁-C₆ alkyl group, wherein the optional substituents are one or more substituents Rₓ;
R₄ is selected from hydrogen, —CH₂OH and —CH₂NH₂; and
Y is —NH— or —O—.
   a compound of formula D-(X)_{b}-(AA)_{w}-(T)_{g}-L₁ or of formula D-(X)_{b}-(AA)_{w}-(T)_{g}-H according to the present invention wherein:
L₁ is a group of formula:

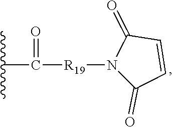

wherein:
the wavy line indicates the point of covalent attachment to (T)_{g} if any, or (AA)_{w} if any, or to (X)_{b} if any, or to D;
R₁₉ is selected from —C₁-C₆ alkylene-, phenylene-C₁-C₆ alkylene- wherein the phenylene group may optionally be substituted with one or more substituents Rₓ selected from the group consisting of alkyl groups having from 1 to 6 carbon atoms, alkoxy groups having from 1 to 6 carbon atoms, halogen atoms, nitro groups and cyano groups, wherein each of the above alkylene substituents whether alone or attached to another moiety in the carbon chain may optionally be substituted by one or more substituents Rₓ selected from the group consisting of alkyl groups having from 1 to 6 carbon atoms, alkoxy groups having from 1 to 6 carbon atoms, aryl groups having from 6 to 12 carbon atoms, halogen atoms, nitro groups and cyano groups, and preferably R₁₉ is a C₁-C₆ alkylene group;
w is 0 or 2, and where w is 2, then (AA)_{w} is of formula (III):

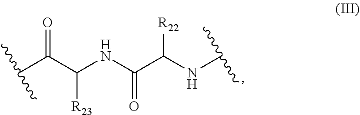

wherein the wavy lines indicate the point of covalent attachments to X (the wavy line to the left) and to (T)_{g} if any, or L₁ or to a hydrogen atom (the wavy line to the right);
R₂₂ is selected from methyl, benzyl, isopropyl, sec-butyl and indolylmethyl;
R₂₃ is selected from methyl, —(CH₂)₄NH₂, —(CH₂)₃NHCONH₂ and —(CH₂)₃NHC(═NH)NH₂;
X is an extending group selected from
where D is conjugated via an amine group: —COO—CH₂-phenylene-NH—, —COO(CH₂)₃NHCOO—CH₂-phenylene-NH, —COO—(CH₂)₃)NH—, —COO(CH₂)₃—S—, and —COO—(CH₂)₃NHCO—(CH₂)₂S—, or
where D is conjugated via an hydroxy group: —COO—CH₂-phenylene-NH—, —CONH(CH₂)₃NHCOOCH₂-phenylene-NH—, —CONH(CH₂)₃NH—, —CONH(CH₂)₃—S—, and —CONH(CH₂)₃NHCO(CH₂)₂S—.
wherein T is an extending group selected from —CO—(C₁-C₄ alkylene)-NH—, —CO—(C₁-C₄ alkylene)-[O—(C₂-C₄ alkylene)]ⱼ—NH—, and —COO—(C₁-C₄ alkylene)-[O—(C₂-C₄ alkylene)]ⱼ—NH—, where j is an integer from 1 to 5;
b is 0 or 1;
g is 0 or 1;
wherein if the compound is a compound of formula D-(X)_{b}-(AA)_{w}-(T)_{g}-H then b+w+g≠0; and
D is a drug moiety of formula (I); and is covalently attached via a hydroxy or amine group; or
is a drug moiety of formula (IH), or a pharmaceutically acceptable salt, ester, solvate, tautomer or stereoisomer thereof:

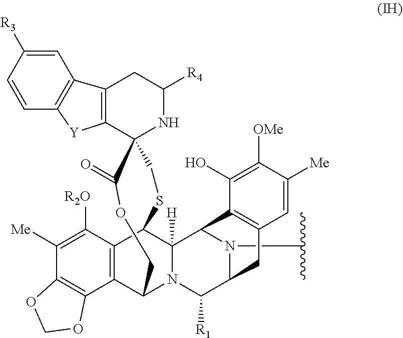

wherein the wavy line of (IH) indicates the point of covalent attachment;
R₁ is —CN or —OH;
R₂ is acetyl;
R₃ is hydrogen or methoxy, preferably methoxy;
R₄ is hydrogen or —CH₂OH, preferably hydrogen;
Y is —NH— or —O—.
   a compound of formula D-(X)_{b}-(AA)_{w}-(T)_{g}-L₁ or of formula D-(X)_{b}-(AA)_{w}-(T)_{g}-H according to the present invention wherein:
L₁ is a linker of formula:

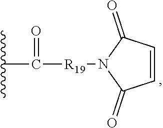

wherein:
the wavy line indicates the point of covalent attachment to (T)_{g} if any, or (AA)_{w} if any, or to (X)_{b} if any, or to D;
R₁₉ is —C₂-C₆ alkylene-;
w is 0 or 2, and where w is 2, then (AA)_{w} is of formula (III):

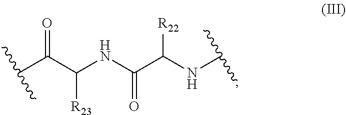

R₂₂ is isopropyl, R₂₃ is selected from methyl and —(CH₂)₃NHCONH₂, wherein the wavy lines indicate the point of covalent attachments to X (the wavy line to the left) and to (T)_{g} if any, or L₁ or to a hydrogen atom (the wavy line to the right);
X is an extending group selected from —COO—CH₂-phenylene-NH—, —COO(CH₂)₃NHCOO—CH₂-phenylene-NH, —COO—(CH₂)₃)NH—, —COO(CH₂)₃—S—, and —COO—(CH₂)₃NHCO—(CH₂)₂S—;
wherein T is an extending group selected from —CO—(C₁-C₄ alkylene)-NH—, —CO—(C₁-C₄ alkylene)-[O—(C₂-C₄ alkylene)]ⱼ—NH—, and —COO—(C₁-C₄ alkylene)-[O—(C₂-C₄ alkylene)]ⱼ—NH—, where j is an integer from 1 to 5;
b is 0 or 1;
g is 0 or 1;
wherein if the compound is a compound of formula D-(X)_{b}-(AA)_{w}-(T)_{g}-H then b+w+g≠0; and
D is a drug moiety selected from:

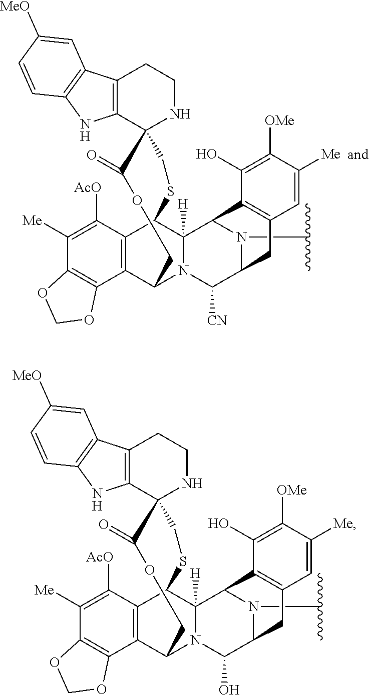 [SMILES: *CN1[C@@H]2c3c(cc(C)c(OC)c3O)C[C@H]1[C@H](C#N)N1[C@H]2[C@@H]2SC[C@]3(NCCc4c3[nH]c3ccc(OC)cc43)C(=O)OCC[C@H]1c1c3c(c(C)c(OC(C)=O)c12)OCO3 ; *CN1[C@@H]2c3c(cc(C)c(OC)c3O)C[C@H]1[C@H](O)N1[C@H]2[C@@H]2SC[C@]3(NCCc4c3[nH]c3ccc(OC)cc43)C(=O)OCC[C@H]1c1c3c(c(C)c(OC(C)=O)c12)OCO3]

or a pharmaceutically acceptable salt, ester, solvate, tautomer or stereoisomer thereof; wherein the wavy line indicates the point of covalent attachment.
   a compound of formula D-(X)_{b}-(AA)_{w}-(T)_{g}-L₁ or of formula D-(X)_{b}-(AA)_{w}-(T)_{g}-H according to the present invention wherein:
L₁ is a group of formula:

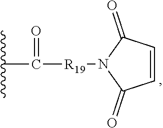

wherein:
the wavy line indicates the point of covalent attachment to (T)_{g} if any, or (AA)_{w} if any, or to (X)_{b}, if any or to D;
R₁₉ is a —C₂-C₅ alkylene-;
w is 0 or 2, and where w is 2, then (AA) is of formula (III):

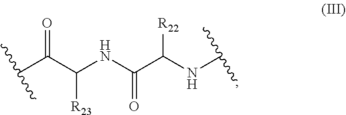

wherein R₂₂ is isopropyl, R₂₃ is selected from methyl and —(CH₂)₃NHCONH₂, wherein the wavy lines indicate the point of covalent attachments to X (the wavy line to the left) and to (T)_{g} if any, or L₁ or to a hydrogen atom (the wavy line to the right);
X is a —COO—CH₂-phenylene-NH— group;
T is a —CO—(CH₂)₂—[O—(CH₂)₂]₄—NH— group;
b is 0 or 1;
g is 0 or 1;
wherein if the compound is a compound of formula D-(X)_{b}-(AA)_{w}-(T)_{g}-H then b+w+g≠0; and
D is a drug moiety selected from:

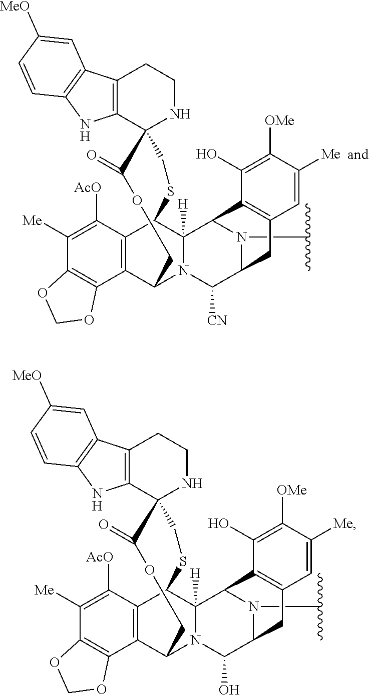 [SMILES: *CN1[C@@H]2c3c(cc(C)c(OC)c3O)C[C@H]1[C@H](C#N)N1[C@H]2[C@@H]2SC[C@]3(NCCc4c3[nH]c3ccc(OC)cc43)C(=O)OCC[C@H]1c1c3c(c(C)c(OC(C)=O)c12)OCO3 ; *CN1[C@@H]2c3c(cc(C)c(OC)c3O)C[C@H]1[C@H](O)N1[C@H]2[C@@H]2SC[C@]3(NCCc4c3[nH]c3ccc(OC)cc43)C(=O)OCC[C@H]1c1c3c(c(C)c(OC(C)=O)c12)OCO3]

or a pharmaceutically acceptable salt, ester, solvate, tautomer or stereoisomer thereof; wherein the wavy line indicates the point of covalent attachment.
   a compound of formula D-X-(AA)_{w}-(T)_{g}-L₁ selected from:

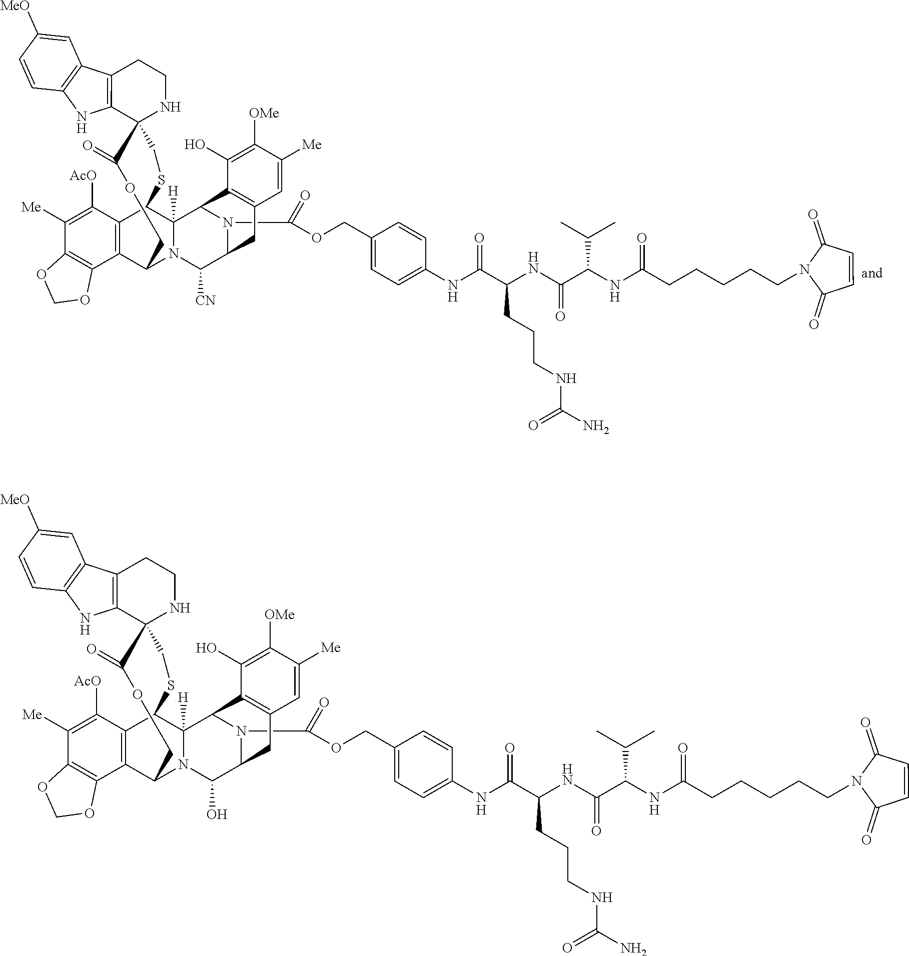 [SMILES: COc1ccc2[nH]c3c(c2c1)CCN[C@]31CS[C@@H]2c3c(OC(C)=O)c(C)c4c(c3[C@H](CCOC1=O)N1[C@@H]2[C@H]2c3c(cc(C)c(OC)c3O)C[C@@H]([C@@H]1C#N)N2CC(=O)OCc1ccc(NC(=O)[C@H](CCCNC(N)=O)NC(=O)[C@@H](NC(=O)CCCCCN2C(=O)C=CC2=O)C(C)C)cc1)OCO4 ; COc1ccc2[nH]c3c(c2c1)CCN[C@]31CS[C@@H]2c3c(OC(C)=O)c(C)c4c(c3[C@H](CCOC1=O)N1[C@@H]2[C@H]2c3c(cc(C)c(OC)c3O)C[C@@H]([C@@H]1O)N2CC(=O)OCc1ccc(NC(=O)[C@H](CCCNC(N)=O)NC(=O)[C@@H](NC(=O)CCCCCN2C(=O)C=CC2=O)C(C)C)cc1)OCO4]

The term “pharmaceutically acceptable salts, esters, solvates, tautomers or stereoisomers” in the drug conjugates of the present invention refers to any pharmaceutically acceptable salt, ester, solvate, hydrate or stereoisomeric form or any other compound which, upon administration to the patient is capable of providing a compound as described herein, whether directly or indirectly. However, it will be appreciated that non-pharmaceutically acceptable salts also fall within the scope of the invention since those may be useful in the preparation of pharmaceutically acceptable salts. The preparation of salts, prodrugs and derivatives can be carried out by methods known in the art.

For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

The drug conjugates of the present invention may be in crystalline form either as free compounds or as solvates (e.g. hydrates) and it is intended that both forms are within the scope of the present invention. Methods of solvation are generally known within the art.

Any compound that is a prodrug of the drug conjugate of the present invention is within the scope and spirit of the invention. The term “prodrug” is used in its broadest sense and encompasses those derivatives that are converted in vivo to the compounds of the invention. Such derivatives would readily occur to those skilled in the art, and include, for example, compounds where a free hydroxy group is converted into an ester derivative. Many suitable prodrugs are well-known to the person in the art and can be found, for example, in Burger “Medicinal Chemistry and Drug Discovery 6^{th} ed. (Donald J. Abraham ed., 2001, Wiley) and “Design and Applications of Prodrugs” (H. Bundgaard ed., 1985, Harwood Academic Publishers), the contents of which are incorporated herein by reference.

In relations to the compounds of the present invention, the pharmacologically acceptable esters are not particularly restricted, and can be selected by a person with an ordinary skill in the art. In the case of said esters, it is preferable that such esters can be cleaved by a biological process such as hydrolysis in vivo. The group constituting the said esters (the group shown as R when the esters thereof are expressed as —COOR) can be, for example, a C₁-C₄ alkoxy C₁-C₄ alkyl group such as methoxyethyl, 1-ethoxyethyl, 1-methyl-1-methoxyethyl, 1-(isopropoxy)ethyl, 2-methoxyethyl, 2-ethoxyethyl, 1,1-dimethyl-1-methoxymethyl, ethoxymethyl, propoxymethyl, isopropoxymethyl, butoxymethyl or t-butoxymethyl; a C₁-C₄ alkoxylated C₁-C₄ alkoxy C₁-C₄ alkyl group such as 2-methoxyethoxymethyl; a C₆-C₁₀ aryloxy C₁-C₄ alkyl group such as phenoxymethyl; a halogenated C₁-C₄ alkoxy C₁-C₄ alkyl group such as 2,2,2-trichloroethoxymethyl or bis(2-chloroethoxy)methyl; a C₁-C₄ alkoxycarbonyl C₁-C₄ alkyl group such as methoxycarbonylmethyl; a cyano C₁-C₄ alkyl group such as cyanomethyl or 2-cyanoethyl; a C₁-C₄ alkylthiomethyl group such as methylthiomethyl or ethylthiomethyl; a C₆-C₁₀ arylthiomethyl group such as phenylthiomethyl or naphthylthiomethyl; a C₁-C₄ alkylsulfonyl C₁-C₄ lower alkyl group, which may be optionally substituted with a halogen atom(s) such as 2-methanesulfonylethyl or 2-trifluoromethanesulfonylethyl; a C₆-C₁₀ arylsulfonyl C₁-C₄ alkyl group such as 2-benzenesulfonylethyl or 2-toluenesulfonylethyl; a C₁-C₇ aliphatic acyloxy C₁-C₄ alkyl group such as formyloxymethyl, acetoxymethyl, propionyloxymethyl, butyryloxymethyl, pivaloyloxymethyl, valeryloxymethyl, isovaleryloxymethyl, hexanoyloxymethyl, 1-formyloxyethyl, 1-acetoxyethyl, 1-propionyloxyethyl, 1-butyryloxyethyl, 1-pivaloyloxyethyl, 1-valeryloxyethyl, 1-isovaleryloxyethyl, 1-hexanoyloxyethyl, 2-formyloxyethyl, 2-acetoxyethyl, 2-propionyloxyethyl, 2-butyryloxyethyl, 2-pivaloyloxyethyl, 2-valeryloxyethyl, 2-isovaleryloxyethyl, 2-hexanoyloxyethyl, 1-formyloxypropyl, 1-acetoxypropyl, 1-propionyloxypropyl, 1-butyryloxypropyl, 1-pivaloyloxypropyl, 1-valeryloxypropyl, 1-isovaleryloxypropyl, 1-hexanoyloxypropyl, 1-acetoxybutyl, 1-propionyloxybutyl, 1-butyryloxybutyl, 1-pivaloyloxybutyl, 1-acetoxypentyl, 1-propionyloxypentyl, 1-butyryloxypentyl, 1-pivaloyloxypentyl or 1-pivaloyloxyhexyl; a C₅-C₆ cycloalkylcarbonyloxy C₁-C₄ alkyl group such as cyclopentylcarbonyloxymethyl, cyclohexylcarbonyloxymethyl, 1-cyclopentylcarbonyloxyethyl, 1-cyclohexylcarbonyloxyethyl, 1-cyclopentylcarbonyloxypropyl, 1-cyclohexylcarbonyloxypropyl, 1-cyclopentylcarbonyloxybutyl or 1-cyclohexylcarbonyloxybutyl; a C₆-C₁₀ arylcarbonyloxy C₁-C₄ alkyl group such as benzoyloxymethyl; a C₁-C₆ alkoxycarbonyloxy C₁-C₄ alkyl group such as methoxycarbonyloxymethyl, 1-(methoxycarbonyloxy)ethyl, 1-(methoxycarbonyloxy)propyl, 1-(methoxycarbonyloxy)butyl, 1-(methoxycarbonyloxy)pentyl, 1-(methoxycarbonyloxy)hexyl, ethoxycarbonyloxymethyl, 1-(ethoxycarbonyloxy)ethyl, 1-(ethoxycarbonyloxy)propyl, 1-(ethoxycarbonyloxy)butyl, 1-(ethoxycarbonyloxy)pentyl, 1-(ethoxycarbonyloxy)hexyl, propoxycarbonyloxymethyl, 1-(propoxycarbonyloxy)ethyl, 1-(propoxycarbonyloxy)propyl, 1-(propoxycarbonyloxy)butyl, isopropoxycarbonyloxymethyl, 1-(isopropoxycarbonyloxy)ethyl, 1-(isopropoxycarbonyloxy)butyl, butoxycarbonyloxymethyl, 1-(butoxycarbonyloxy)ethyl, 1-(butoxycarbonyloxy)propyl, 1-(butoxycarbonyloxy)butyl, isobutoxycarbonyloxymethyl, 1-(isobutoxycarbonyloxy)ethyl, 1-(isobutoxycarbonyloxy)propyl, 1-(isobutoxycarbonyloxy)butyl, t-butoxycarbonyloxymethyl, 1-(t-butoxycarbonyloxy)ethyl, pentyloxycarbonyloxymethyl, 1-(pentyloxycarbonyloxy)ethyl, 1-(pentyloxycarbonyloxy)propyl, hexyloxycarbonyloxymethyl, 1-(hexyloxycarbonyloxy)ethyl or 1-(hexyloxycarbonyloxy)propyl; a C₅-C₆ cycloalkyloxycarbonyloxy C₁-C₄ alkyl group such as cyclopentyloxycarbonyloxymethyl, 1-(cyclopentyloxycarbonyloxy)ethyl, 1-(cyclopentyloxycarbonyloxy)propyl, 1-(cyclopentyloxycarbonyloxy)butyl, cyclohexyloxycarbonyloxymethyl, 1-(cyclohexyloxycarbonyloxy)ethyl, 1-(cyclohexyloxycarbonyloxy)propyl or 1-(cyclohexyloxycarbonyloxy)butyl; a [5-(C₁-C₄ alkyl)-2-oxo-1,3-dioxolen-4-yl]methyl group 50 such as (5-methyl-2-oxo-1,3-dioxolen-4-yl)methyl, (5-ethyl-2-oxo-1,3-dioxolen-4-yl)methyl, (5-propyl-2-oxo-1,3-dioxolen-4-yl)methyl, (5-isopropyl-2-oxo-1,3-dioxolen-4-yl)methyl or (5-butyl-2-oxo-1,3-dioxolen-4-yl)methy; a [5-(phenyl, which may be optionally substituted with a C₁-C₄ alkyl, C₁-C₄ alkoxy or halogen atom(s))-2-oxo-1,3-dioxolen-4-yl]methyl group such as (5-phenyl-2-oxo-1,3-dioxolen-4-yl)methyl, [5-(4-methylphenyl)-2-oxo-1,3-dioxolen-4-yl]methyl, [5-(4-methoxyphenyl)-2-oxo-1,3-dioxolen-4-yl]methyl, [5-(4-fluorophenyl)-2-oxo-1,3-dioxolen-4-yl]methyl or [5-(4-chlorophenyl)-2-oxo-1,3-dioxolen-4-yl]methyl; or a phthalidyl group, which may be optionally substituted with a C₁-C₄ alkyl or C₁-C₄ alkoxy group(s), such as phthalidyl, dimethylphthalidyl or dimethoxyphthalidyl, and is preferably a pivaloyloxymethyl group, phthalidyl group or (5-methyl-2-oxo-1,3-dioxolen-4-yl)methyl group, and more preferably a (5-methyl-2-oxo-1,3-dioxolen-4-yl)methyl group.

Any compound referred to herein is intended to represent such specific compound as well as certain variations or forms. In particular, compounds referred to herein may have asymmetric centres and therefore exist in different enantiomeric forms. All optical isomers and stereoisomers of the compounds referred to herein, and mixtures thereof, are considered within the scope of the present invention. Thus any given compound referred to herein is intended to represent any one of a racemate, one or more enantiomeric forms, one or more diastereomeric forms, one or more atropisomeric forms, and mixtures thereof. Particularly, the drug conjugates of formula [D-(X)_{b}-(AA)_{w}-(T)_{g}-(L)]ₙ-Ab and compounds of formula D-X-(AA)_{w}-(T)_{g}-L₁ or D-X-(AA)_{w}-(T)_{g}-H may include enantiomers depending on their asymmetry or diastereoisomers. Stereoisomerism about the double bond is also possible, therefore in some cases the molecule could exist as (E)-isomer or (Z)-isomer. If the molecule contains several double bonds, each double bond will have its own stereoisomerism, that could be the same or different than the stereoisomerism of the other double bonds of the molecule. The single isomers and mixtures of isomers fall within the scope of the present invention.

Furthermore, compounds referred to herein may exist as geometric isomers (i.e., cis and trans isomers), as tautomers, or as atropisomers. Specifically, the term tautomer refers to one of two or more structural isomers of a compound that exist in equilibrium and are readily converted from one isomeric form to another. Common tautomeric pairs are amine-imine, amide-imide, keto-enol, lactam-lactim, etc. Additionally, any compound referred to herein is intended to represent hydrates, solvates, and polymorphs, and mixtures thereof when such forms exist in the medium. In addition, compounds referred to herein may exist in isotopically-labelled forms. All geometric isomers, tautomers, atropisomers, hydrates, solvates, polymorphs, and isotopically labelled forms of the compounds referred to herein, and mixtures thereof, are considered within the scope of the present invention.

Protected forms of the compounds disclosed herein are considered within the scope of the present invention. Suitable protecting groups are well known for the skilled person in the art. A general review of protecting groups in organic chemistry is provided by Wuts, PGM and Greene TW in Protecting Groups in Organic Synthesis, 4^{th} Ed. Wiley-Interscience, and by Kocienski PJ in Protecting Groups, 3^{rd} Ed. Georg Thieme Verlag. These references provide sections on protecting groups for OH, amino and SH groups. All these references are incorporated by reference in their entirety.

Within the scope of the present invention an OH protecting group is defined to be the O-bonded moiety resulting from the protection of the OH through the formation of a suitable protected OH group. Examples of such protected OH groups include ethers, silyl ethers, esters, sulfonates, sulfenates and sulfinates, carbonates, and carbamates. In the case of ethers the protecting group for the OH can be selected from methyl, methoxymethyl, methylthiomethyl, (phenyldimethylsilyl)methoxymethyl, benzyloxymethyl, p-methoxybenzyloxymethyl, [(3,4-dimethoxybenzyl)oxy]methyl, p-nitrobenzyloxymethyl, o-nitrobenzyloxymethyl, [(R)-1-(2-nitrophenyl)ethoxy]methyl, (4-methoxyphenoxy)methyl, guaiacolmethyl, [(p-phenylphenyl)oxy]methyl, t-butoxymethyl, 4-pentenyloxymethyl, siloxymethyl, 2-methoxyethoxymethyl, 2-cyanoethoxymethyl, bis(2-chloroethoxy)methyl, 2,2,2-trichloroethoxymethyl, 2-(trimethylsilyl)ethoxymethyl, menthoxymethyl, O-bis(2-acetoxy-ethoxy)methyl, tetrahydropyranyl, fluorous tetrahydropyranyl, 3-bromotetrahydropyranyl, tetrahydrothiopyranyl, 1-methoxycyclohexyl, 4-methoxytetrahydropyranyl, 4-methoxy-tetrahydrothiopyranyl, 4-methoxytetrahydrothiopyranyl S,S-dioxide, 1-[(2-chloro-4-methyl)-phenyl]-4-methoxypiperidin-4-yl, 1-(2-fluorophenyl)-4-methoxypiperidin-4-yl, 1-(4-chlorophenyl)-4-methoxypiperidin-4-yl, 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 2-hydroxyethyl, 2-bromoethyl, 1-[2-(trimethylsilyl)ethoxy]ethyl, 1-methyl-1-methoxyethyl, 1-methyl-1-benzyloxyethyl, 1-methyl-1-benzyloxy-2-fluoroethyl, 1-methyl-1-phenoxyethyl, 2,2,2-trichloroethyl, 1,1-dianisyl-2,2,2-trichloroethyl, 1,1,1,3,3,3-hexafluoro-2-phenylisopropyl, 1-(2-cyanoethoxy)ethyl, 2-trimethylsilylethyl, 2-(benzylthio)ethyl, 2-(phenylselenyl)ethyl, t-butyl, cyclohexyl, 1-methyl-1′-cyclopropylmethyl, allyl, prenyl, cinnamyl, 2-phenallyl, propargyl, p-chlorophenyl, p-methoxyphenyl, p-nitrophenyl, 2,4-dinitrophenyl, 2,3,5,6-tetrafluoro-4-(trifluoromethyl)phenyl, benzyl, p-methoxybenzyl, 3,4-dimethoxybenzyl, 2,6-dimethoxybenzyl, o-nitrobenzyl, p-nitrobenzyl, pentadienylnitrobenzyl, pentadienylnitropiperonyl, halobenzyl, 2,6-dichlorobenzyl, 2,4-dichlorobenzyl, 2,6-difluorobenzyl, p-cyanobenzyl, fluorous benzyl, 4-fluorousalkoxybenzyl, trimethylsilylxylyl, p-phenylbenzyl, 2-phenyl-2-propyl, p-acylaminobenzyl, p-azidobenzyl, 4-azido-3-chlorobenzyl, 2-trifluoromethylbenzyl, 4-trifluoromethylbenzyl, p-(methylsulfinyl)benzyl, p-siletanylbenzyl, 4-acetoxybenzyl, 4-(2-trimethylsilyl)ethoxymethoxybenzyl, 2-naphthylmethyl, 2-picolyl, 4-picolyl, 3-methyl-2-picolyl N-oxide, 2-quinolinylmethyl, 6-methoxy-2-(4-methylphenyl)-4-quinolinemethyl, 1-pyrenylmethyl, diphenylmethyl, 4-methoxydiphenylmethyl, 4-phenyldiphenylmethyl, p,p′-dinitrobenzhydryl, 5-dibenzosuberyl, triphenylmethyl, tris(4-t-butylphenyl)methyl, α-naphthyldiphenylmethyl, p-methoxyphenyldiphenylmethyl, di(p-methoxyphenyl)phenyl-methyl, tri(p-methoxyphenyl)methyl, 4-(4′-bromophenacyloxy)phenyldiphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-dimethoxy-3″-[N-(imidazolylmethyl)]trityl, 4,4′-dimethoxy-3″-[N-(imidazolylethyl)carbamoyl]trityl, bis(4-methoxyphenyl)-1′-pyrenylmethyl, 4-(17-tetrabenzo[a,c,g,i]fluorenylmethyl)-4,4″-dimethoxytrityl, 9-anthryl, 9-(9-phenyl)xanthenyl, 9-phenylthioxanthyl, 9-(9-phenyl-10-oxo)anthryl, 1,3-benzodithiolan-2-yl, 4,5-bis(ethoxycarbonyl)-[1,3]-dioxolan-2-yl, benzisothiazolyl S,S-dioxide. In the case of silyl ethers the protecting group for the OH can be selected from trimethylsilyl, triethylsilyl, triisopropylsilyl, dimethylisopropylsilyl, diethylisopropylsilyl, dimethylhexylsilyl, 2-norbornyldimethylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, bis(t-butyl)-1-pyrenylmethoxysilyl, tris(trimethylsilyl)silyl, (2-hydroxystyryl)dimethylsilyl, (2-hydroxystyryl)diisopropylsilyl, t-butylmethoxyphenylsilyl, t-butoxydiphenylsilyl, 1,1,3,3-tetraisopropyl-3-[2-(triphenylmethoxy) ethoxy]disiloxane-1-yl, and fluorous silyl. In the case of esters the protecting group for the OH together with the oxygen atom of the unprotected OH to which it is attached form an ester that can be selected from formate, benzoylformate, acetate, chloroacetate, dichloroacetate, trichloroacetate, trichloroacetamidate, trifluoroacetate, methoxyacetate, triphenylmethoxyacetate, phenoxyacetate, p-chlorophenoxyacetate, phenylacetate, diphenylacetate, 3-phenylpropionate, bisfluorous chain type propanoyl, 4-pentenoate, 4-oxopentanoate, 4,4-(ethylenedithio)pentanoate, 5[3-bis(4-methoxyphenyl)hydro-xymethylphenoxy]levulinate, pivaloate, 1-adamantoate, crotonate, 4-methoxycrotonate, benzoate, p-phenylbenzoate, 2,4,6-trimethylbenzoate, 4-bromobenzoate, 2,5-difluorobenzoate, p-nitrobenzoate, picolinate, nicotinate, 2-(azidomethyl)benzoate, 4-azido-butyrate, (2-azidomethyl)phenylacetate, 2-{[(tritylthio)oxy]methyl}benzoate, 2-{[(4-methoxytritylthio)oxy]methyl}benzoate, 2-{[methyl(tritylthio)amino]methyl}benzoate, 2-{{[(4-methoxytrityl)thio]methylamino}methyl}benzoate, 2-(allyloxy)phenylacetate, 2-(prenyloxymethyl)benzoate, 6-(levulinyloxymethyl)-3-methoxy-2-nitrobenzoate, 6-50 (levulinyloxymethyl)-3-methoxy-4-nitrobenzoate, 4-benzyloxybutyrate, 4-trialkylsilyloxy-butyrate, 4-acetoxy-2,2-dimethylbutyrate, 2,2-dimethyl-4-pentenoate, 2-iodobenzoate, 4-nitro-4-methylpentanoate, o-(dibromomethyl)benzoate, 2-formylbenzenesulfonate, 4-(methylthio-methoxy)butyrate, 2-(methylthiomethoxymethyl)benzoate, 2-(chloroacetoxymethyl)benzoate, 2-[(2-chloroacetoxy)ethyl]benzoate, 2-[2-55 (benzyloxy)ethyl]benzoate, 2-[2-(4-methoxybenzyl-oxy)ethyl]benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenyl-acetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, and 2-chlorobenzoate. In the case of sulfonates, sulfenates and sulfinates the protecting group for the OH together with the oxygen atom of the unprotected OH to which it is attached form a sulfonate, sulfenate or sulfinates that can be selected from sulfate, allylsulfonate, methanesulfonate, benzylsulfonate, tosylate, 2-[(4-nitrophenyl)ethyl]sulfonate, 2-trifluoromethylbenzenesulfonate, 4-monomethoxytritylsulfenate, alkyl 2,4-dinitrophenylsulfenate, 2,2,5,5-tetramethylpyrrolidin-3-one-1-sulfinate, and dimethylphosphinothioyl. In the case of carbonates the protecting group for the OH together with the oxygen atom of the unprotected OH to which it is attached form a carbonate that can be selected from methyl carbonate, methoxymethyl carbonate, 9-fluorenylmethyl carbonate, ethyl carbonate, bromoethyl carbonate, 2-(methylthiomethoxy)ethyl carbonate, 2,2,2-trichloroethyl carbonate, 1,1-dimethyl-2,2,2-trichloroethyl carbonate, 2-(trimethylsilyl)ethyl carbonate, 2-[dimethyl(2-naphthylmethyl)silyl]ethyl carbonate, 2-(phenylsulfonyl)ethyl carbonate, 2-(triphenylphosphonio)ethyl carbonate, cis-[4-[[(methoxytrityl)sulfenyl]oxy]tetrahydrofuran-3-yl]oxy carbonate, isobutyl carbonate, t-butyl carbonate, vinyl carbonate, allyl carbonate, cinnamyl carbonate, propargyl carbonate, p-chlorophenyl carbonate, p-nitrophenyl carbonate, 4-ethoxy-1-naphthyl carbonate, 6-bromo-7-hydroxycoumarin-4-ylmethyl carbonate, benzyl carbonate, o-nitrobenzyl carbonate, p-nitrobenzyl carbonate, p-methoxybenzyl carbonate, 3,4-dimethoxybenzyl carbonate, anthraquinon-2-ylmethyl carbonate, 2-dansylethyl carbonate, 2-(4-nitrophenyl)ethyl carbonate, 2-(2,4-dinitrophenyl)ethyl carbonate, 2-(2-nitrophenyl)propyl carbonate, 2-(3,4-methylenedioxy-6-nitrophenyl)propyl carbonate, 2-cyano-1-phenylethyl carbonate, 2-(2-pyridyl)amino-1-phenylethyl carbonate, 2-[N-methyl-N-(2-pyridyl)]amino-1-phenylethyl carbonate, phenacyl carbonate, 3′,5′-dimethoxybenzoin carbonate, methyl dithiocarbonate, and S-benzyl thiocarbonate. And in the case of carbamates the protecting group for OH together with the oxygen atom of the unprotected OH to which it is attached forms a carbamate that can be selected from dimethyl thiocarbamate, N-phenyl carbamate, and N-methyl-N-(o-nitrophenyl) carbamate.

Within the scope of the present invention an amino protecting group is defined to be the N-bonded moiety resulting from the protection of the amino group through the formation of a suitable protected amino group. Examples of protected amino groups include carbamates, ureas, amides, heterocyclic systems, N-alkyl amines, N-alkenyl amines, N-alkynyl amines, N-aryl amines, imines, enamines, N-metal derivatives, N—N derivatives, N—P derivatives, N—Si derivatives, and N—S derivatives. In the case of carbamates the protecting group for the amino group together with the amino group to which it is attached form a carbamate that can be selected from methyl carbamate, ethyl carbamate, 9-fluorenylmethyl carbamate, 2,6-di-t-butyl-9-fluorenylmethyl carbamate, 2,7-bis(trimethylsilyl)fluorenylmethyl carbamate, 9-(2-sulfo)fluorenylmethyl carbamate, 9-(2,7-dibromo)fluorenylmethyl carbamate, 17-tetrabenzo[a,c,g,i]fluorenylmethyl carbamate, 2-chloro-3-indenylmethyl carbamate, benz]inden-3-ylmethyl carbamate, 1,1-dioxobenzo[b]-thiophene-2-ylmethyl carbamate, 2-methylsulfonyl-3-phenyl-1-prop-2-enyl carbamate, 2,7-di-t-butyl-[9,(10,10-dioxo-10,10,10,10-tetrahydrothioxanthyl)]methyl carbamate, 2,2,2-trichloroethyl carbamate, 2-trimethylsilylethyl carbamate, (2-phenyl-2-trimethylsilyl)ethyl carbamate, 2-phenylethyl carbamate, 2-chloroethyl carbamate, 1,1-dimethyl-2-haloethyl carbamate, 1,1-dimethyl-2,2-dibromoethyl carbamate, 1,1-dimethyl-2,2,2-trichloroethyl carbamate, 2-(2′-pyridyl)ethyl carbamate, 2-(4′-pyridyl)ethyl carbamate, 2,2-bis(4′-nitrophenyl)ethyl carbamate, 2-[(2-nitrophenyl)dithio]-1-phenylethyl carbamate, 2-(N,N-dicyclohexylcarboxamido)ethyl 50 carbamate, t-butyl carbamate, fluorous BOC carbamate, 1-adamantyl carbamate, 2-adamantyl carbamate, 1-(1-adamantyl)-1-methylethyl carbamate, 1-methyl-1-(4-byphenylyl)ethyl carbamate, 1-(3,5-di-t-butylphenyl)-1-methylethyl carbamate, triisopropylsilyloxy carbamate, vinyl carbamate, allyl carbamate, prenyl carbamate, 1-isopropylallyl carbamate, cinnamyl carbamate, 4-nitrocinnamyl carbamate, 3-(3′-pyridyl)prop-2-enyl carbamate, hexadienyl carbamate, propargyl carbamate, 1,4-but-2-ynyl biscarbamate, 8-quinolyl carbamate, N-hydroxypiperidinyl carbamate, alkyl dithiocarbamate, benzyl carbamate, 3,5-di-t-butylbenzyl carbamate, p-methoxybenzyl carbamate, p-nitrobenzyl carbamate, p-bromobenzyl carbamate, p-chlorobenzyl carbamate, 2,4-dichlorobenzyl carbamate, 4-methylsulfinylbenzyl carbamate, 4-trifluoromethylbenzyl carbamate, fluorous benzyl carbamate, 2-naphthylmethyl carbamate, 9-anthrylmethyl carbamate, diphenylmethyl carbamate, 4-phenylacetoxybenzyl carbamate, 4-azidobenzyl carbamate, 4-azido-methoxybenzyl carbamate, m-chloro-p-acyloxybenzyl carbamate, p-(dihydroxyboryl)-benzyl carbamate, 5-benzisoxazolylmethyl carbamate, 2-(trifluoromethyl)-6-chromonylmethyl carbamate, 2-methylthioethyl carbamate, 2-methylsulfonylethyl carbamate, 2-(p-toluenesulfonyl)ethyl carbamate, 2-(4-nitrophenylsulfonyl)ethyl carbamate, 2-(2,4-dinitrophenylsulfonyl)ethyl carbamate, 2-(4-trifluoromethylphenylsulfonyl)ethyl carbamate, [2-(1,3-dithianyl)]methyl carbamate, 2-phosphonioethyl carbamate, 2-[phenyl(methyl)sulfonio]ethyl carbamate, 1-methyl-1-(triphenylphosphonio)ethyl carbamate, 1,1-dimethyl-2-cyanoethyl carbamate, 2-dansylethyl carbamate, 2-(4-nitrophenyl)ethyl carbamate, 4-methylthiophenyl carbamate, 2,4-dimethylthiophenyl carbamate, m-nitrophenyl carbamate, 3,5-dimethoxybenzyl carbamate, 1-methyl-1-(3,5-dimethoxyphenyl)ethyl carbamate, α-methylnitropiperonyl carbamate, o-nitrobenzyl carbamate, 3,4-dimethoxy-6-nitrobenzyl carbamate, phenyl(o-nitrophenyl)methyl carbamate, 2-nitrophenylethyl carbamate, 6-nitroveratryl carbamate, 4-methoxyphenacyl carbamate, 3′,5′-dimethoxybenzoin carbamate, 9-xanthenylmethyl carbamate, N-methyl-N-(o-nitrophenyl) carbamate, t-amyl carbamate, 1-methylcyclobutyl carbamate, 1-methylcyclohexyl carbamate, 1-methyl-1-cyclopropylmethyl carbamate, cyclobutyl carbamate, cyclopentyl carbamate, cyclohexyl carbamate, isobutyl carbamate, isobornyl carbamate, cyclopropylmethyl carbamate, p-decyloxybenzyl carbamate, diisopropylmethyl carbamate, 2,2-dimethoxy-carbonylvinyl carbamate, o-(N,N-dimethylcarboxamido)benzyl carbamate, 1,1-dimethyl-3-(N,N-dimethyl-carboxamido)propyl carbamate, butynyl carbamate, 1,1-dimethylpropynyl carbamate, 2-iodoethyl carbamate, 1-methyl-1-(4′-pyridyl)ethyl carbamate, 1-methyl-1-(p-phenylazophenyl)ethyl carbamate, p-(p′-methoxyphenylazo)benzyl carbamate, p-(phenylazo)benzyl carbamate, 2,4,6-trimethylbenzyl carbamate, isonicotinyl carbamate, 4-(trimethyl-ammonium)benzyl carbamate, p-cyanobenzyl carbamate, di(2-pyridyl)methyl carbamate, 2-furanylmethyl carbamate, phenyl carbamate, 2,4,6-tri-t-butylphenyl carbamate, 1-methyl-1-phenylethyl carbamate, and S-benzyl thiocarbamate. In the case of ureas the protecting groups for the amino group can be selected from phenothiazinyl-(10)-carbonyl, N′p-toluenesulfonylaminocarbonyl, N′-phenylaminothiocarbonyl, 4-hydroxyphenylaminocarbonyl, 3-hydroxytryptaminocarbonyl, and N′-phenylaminothiocarbonyl. In the case of amides the protecting group for the amino together with the amino group to which it is attached form an amide that can be selected from formamide, acetamide, chloroacetamide, trichloroacetamide, trifluoroacetamide, phenylacetamide, 3-phenylpropanamide, pent-4-enamide, picolinamide, 3-pyridylcarboxamide, N-benzoylphenylalanyl amide, benzamide, p-phenylbenzamide, o-nitrophenylacetamide, 2,2-dimethyl-2-(o-nitrophenyl)acetamide, o-nitrophenoxyacetamide, 3-(o-nitrophenyl)propanamide, 2-methyl-2-(o-nitrophenoxy)propanamide, 3-methyl-3-nitrobutanamide, o-nitrocinnamide, o-nitrobenzamide, 3-(4-t-butyl-2,6-dinitrophenyl)-2,2-dimethylpropanamide, o-(benzoyloxyme-thyl)benzamide, 2-(acetoxymethyl)benzamide, 2-[(t-butyldiphenylsiloxy)methyl]benzamide, 3-(3′,6′-dioxo-2′,4′,5′-trimethylcyclohexa-1′,4′-diene)-3,3-dimethylpropionamide, o-hydroxy-trans-cinnamide, 2-methyl-2-(o-phenylazophenoxy)propanamide, 4-chlorobutanamide, aceto-acetamide, 3-(p-hydroxyphenyl)propanamide, (N′-dithiobenzyloxycarbonylamino)acetamide, and N-acetylmethionine amide. In the case of heterocyclic systems the protecting group for the 50 amino group together with the amino group to which it is attached form a heterocyclic system that can be selected from 4,5-diphenyl-3-oxazolin-2-one, N-phthalimide, N-dichlorophthalimide, N-tetrachlorophthalimide, N-4-nitrophthalimide, N-thiodiglycoloyl, N-dithiasuccinimide, N-2,3-diphenylmaleimide, N-2,3-dimethylmaleimide, N-2,5-dimethylpyrrole, N-2,5-bis(triisopropylsiloxy)pyrrole, N-1,1,4,4-55 tetramethyldisilylazacyclopentane adduct, N-1,1,3,3-tetramethyl-1,3-disilaisoindoline, N-diphenylsilyldiethylene, N-5-substituted-1,3-dimethyl-1,3,5-triazacyclohexan-2-one, N-5-substituted-1,3-benzyl-1,3,5-triazacyclohexan-2-one, 1-substituted 3,5-dinitro-4-pyridone, and 1,3,5-dioxazine. In the case of N-alkyl, N-alkenyl, N-alkynyl or N-aryl amines the protecting group for the amino group can be selected from N-methyl, N-t-butyl, N-allyl, N-prenyl, N-cinnamyl, N-phenylallyl, N-propargyl, N-methoxymethyl, N-[2-(trimethylsilyl)ethoxy]methyl, N-3-acetoxypropyl, N-cyanomethyl, N-2-azanorbornenes, N-benzyl, N-4-methoxybenzyl, N-2,4-dimethoxybenzyl, N-2-hydroxybenzyl, N-ferrocenylmethyl, N-2,4-dinitrophenyl, o-methoxyphenyl, p-methoxyphenyl, N-9-phenylfluorenyl, N-fluorenyl, N-2-picolylamine N′-oxide, N-7-methoxycoumar-4-ylmethyl, N-diphenylmethyl, N-bis(4-methoxyphenyl)methyl, N-5-dibenzosuberyl, N-triphenylmethyl, N-(4-methylphenyl)diphenylmethyl, and N-(4-methoxyphenyl)diphenylmethyl. In the case of imines the protecting group for the amino group can be selected from N-1,1-dimethylthiomethylene, N-benzylidene, N-p-methoxybenzylidene, N-diphenylmethylene, N-[2-pyridyl)mesityl]methylene, N—(N,N-dimethylaminomethylene), N—(N′,N′-dibenzylaminomethylene), N—(N-t-butylaminome-thylene), N,N-isopropylidene, N-p-nitrobenzylidene, N-salicylidene, N-5-chlorosalicylidene, N-(5-chloro-2-hydroxyphenyl)phenylmethylene, N-cyclohexylidene, and N-t-butylidene. In the case of enamines the protecting group for the amino group can be selected from N-(5,5-dimethyl-3-oxo-1-cyclohexenyl), N-2,7-dichloro-9-fluorenylmethylene, N-1-(4,4-dimethyl-2,6-dioxocyclohexylidene)ethyl, N-(1,3-dimethyl-2,4,6-(1H,3H,5H)-trioxopyrimidine-5-ylidene)-methyl, N-4,4,4-trifluoro-3-oxo-1-butenyl, and N-(1-isopropyl-4-nitro-2-oxo-3-pyrrolin-3-yl).

In the case of N-metal derivatives the protecting group for the amino group can be selected from N-borane, N-diphenylborinic ester, N-diethylborinic ester, N-9-borabicyclononane, N-difluoroborinic ester, and 3,5-bis(trifluoromethyl)phenylboronic acid; and also including N-phenyl(pentacarbonylchromium)carbenyl, N-phenyl(pentacarbonyl-tungsten)carbenyl, N-methyl(pentacarbonylchromium)carbenyl, N-methyl(pentacarbonyltungsten)carbenyl, N-copper chelate, N-zinc chelate, and a 18-crown-6-derivative. In the case of N—N derivatives the protecting group for the amino group together with the amino group to which it is attached form a N—N derivative that can be selected from N-nitroamino, N-nitrosoamino, amine N-oxide, azide, triazene derivative, and N-trimethylsilylmethyl-N-benzylhydrazine. In the case of N—P derivatives the protected group for the amino group together with the amino group to which it is attached form a N—P derivative that can be selected from diphenylphosphinamide, dimethylthiophosphinamide, diphenylthiophosphinamide, dialkyl phosphoramidate, dibenzyl phosphoramidate, diphenyl phosphoramidate, and iminotriphenylphosphorane. In the case of N—Si derivatives the protecting group for the NH₂ can be selected from t-butyldiphenylsilyl and triphenylsilyl. In the case of N—S derivatives the protected amino group can be selected from N-sulfenyl or N-sulfonyl derivatives. The N-sulfenyl derivatives can be selected from benzenesulfenamide, 2-nitrobenzenesulfenamide, 2,4-dinitrobenzenesulfenamide, pentachlorobenzenesulfenamide, 2-nitro-4-methoxybenzenesulfenamide, triphenylmethylsulfe-namide, 1-(2,2,2-trifluoro-1,1-diphenyl)ethylsulfenamide, and N-3-nitro-2-pyridinesulfenamide. The N-sulfonyl derivatives can be selected from methanesulfonamide, trifluoromethanesulfonamide, t-butylsulfonamide, benzylsulfonamide, 2-(trimethylsilyl) ethanesulfonamide, p-toluenesulfonamide, benzenesulfonamide, o-anisylsulfonamide, 2-nitrobenzenesulfonamide, 4-nitrobenzenesulfonamide, 2,4-dinitrobenzenesulfonamide, 2-naphthalenesulfonamide, 4-(4′,8′-dimethoxynaphthylmethyl)benzenesulfonamide, 2-(4-methylphenyl)-6-methoxy-4-methylsulfonamide, 9-anthracenesulfonamide, pyridine-2-sulfonamide, benzothiazole-2-sulfonamide, phenacylsulfonamide, 2,3,6-trimethyl-4-methoxybenzenesulfonamide, 2,4,6-trimethoxybenzenesulfonamide, 2,6-dimethyl-4-methoxy-benzenesulfonamide, pentamethylbenzenesulfonamide, 2,3,5,6-tetramethyl-4-methoxyben-zenesulfonamide, 4-methoxybenzenesulfonamide, 2,4,6-trimethylbenzenesulfonamide, 2,6-dimethoxy-4-methylbenzenesulfonamide, 3-methoxy-4-t-butylbenzenesulfonamide, and 2,2,5,7,8-pentamethylchroman-6-sulfonamide.

Within the scope of the present invention a protecting group for SH is defined to be the S-bonded moiety resulting from the protection of the SH group through the formation of a suitable a protected SH group. Examples of such protected SH groups include thioethers, disulfides, silyl thioethers, thioesters, thiocarbonates, and thiocarbamates. In the case of thioethers the protecting group for the SH can be selected from S-alkyl, S-benzyl, S-p-methoxybenzyl, S-o-hydroxybenzyl, S-p-hydroxybenzyl, S-o-acetoxybenzyl, S-p-acetoxybenzyl, S-p-nitrobenzyl, S-o-nitrobenzyl, S-2,4,6-trimethylbenzyl, S-2,4,6-trimethoxybenzyl, S-4-picolyl, S-2-picolyl-N-oxide, S-2-quinolinylmethyl, S-9-anthrylmethyl, S-9-fluorenylmethyl, S-xanthenyl, S-ferrocenylmethyl, S-diphenylmethyl, S-bis(4-methoxyphenyl)methyl, S-5-dibenzosuberyl, S-triphenylmethyl, 4-methoxytrityl, S-diphenyl-4-pyridylmethyl, S-phenyl, S-2,4-dinitrophenyl, S-2-quinolyl, S-t-butyl, S-1-adamantyl, S-methoxymethyl, S-isobutoxymethyl, S-benzyloxymethyl, S-1-ethoxyethyl, S-2-tetrahydropyranyl, S-benzylthiomethyl, S-phenylthiomethyl, S-acetamidomethyl (Acm), S-trimethylacetamidomethyl, S-benzamidomethyl, S-allyloxycarbonylaminomethyl, S—N-[2,3,5,6-tetrafluoro-4-(N′-piperidino)-phenyl-N-allyloxycarbonylaminomethyl, S-phthalimidomethyl, S-phenylacetamidomethyl, S-acetylmethyl, S-carboxymethyl, S-cyanomethyl, S-(2-nitro-1-phenyl)ethyl, S-2-(2,4-dinitrophenyl)ethyl, S-2-(4′-pyridyl)ethyl, S-2-cyanoethyl, S-2-(trimethylsilyl)ethyl, S-2,2-bis(carboethoxy)ethyl, S-(1-m-nitrophenyl-2-benzoyl)ethyl, S-2-phenylsulfonylethyl, S-1-(4-methylphenylsulfonyl)-2-methylprop-2-yl, and S-p-hydroxyphenacyl. In the case of disulfides the protected SH group can be selected from S-ethyl disulfide, S-t-butyl disulfide, S-2-nitrophenyl disulfide, S-2,4-dinitrophenyl disulfide, S-2-phenylazophenyl disulfide, S-2-carboxyphenyl disulfide, and S-3-nitro-2-pyridyl disulfide. In the case of silyl thioethers the protecting group for the SH can be selected from the list of groups that was listed above for the protection of OH with silyl ethers. In the case of thioesters the protecting group for the SH can be selected from S-acetyl, S-benzoyl, S-2-methoxyisobutyryl, S-trifluoroacetyl, S—N-[[p-biphenylyl)-isopropyloxy]carbonyl]-N-methyl-γ-aminothiobutyrate, and S—N-(t-butoxycarbonyl)-N-methyl-γ-aminothiobutyrate. In the case of thiocarbonate protecting group for the SH can be selected from S-2,2,2-trichloroethoxycarbonyl, S-t-butoxycarbonyl, S-benzyloxycarbonyl, S-p-methoxybenzyloxycarbonyl, and S-fluorenylmethylcarbonyl. In the case of thiocarbamate the protected SH group can be selected from S—(N-ethylcarbamate) and S—(N-methoxymethylcarbamate).

The mention of these groups should not be interpreted as a limitation of the scope of the invention, since they have been mentioned as a mere illustration of protecting groups for OH, amino and SH groups, but further groups having said function may be known by the skilled person in the art, and they are to be understood to be also encompassed by the present invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

“Antibody-drug-conjugates (ADCs)” represent a targeted strategy to deliver a cytotoxic molecule to a cancer cell (see, for example, International Patent Applications WO-A-2004/010957, WO-A-2006/060533 and WO-A-2007/024536). Such compounds are typically referred to as drug, toxin and radionuclide “conjugates”. Tumor cell killing occurs upon binding of the drug conjugate to a tumor cell and release and/or activation of the cytotoxic activity of the drug moiety. The selectivity afforded by drug conjugates minimizes toxicity to normal cells, thereby enhancing tolerability of the drug in the patient. Three examples of drug antibody conjugates of this type that have received marketing approval are: Gemtuzumab ozogamicin for acute myelogenous leukemia, Brentuximab vedotin for relapsed and refractory Hodgkin lymphoma and anaplastic large cell lymphoma, and ado-Trastuzumab emtansine for breast cancer, especially HER2+.

The effectiveness of drugs for cancer chemotherapy generally relies on differences in growth rates, biochemical pathways, and physiological characteristics between cancer and normal tissues. Consequently, most standard chemotherapeutics are relatively nonspecific and exhibit dose-limiting toxicities that contribute to suboptimal therapeutic effects. One approach to selectively target malignant cells and not healthy tissues is to use specific monoclonal antibodies (mAbs) that recognize tumor-associated antigens expressed on the surface of tumor cells [Meyer, D. L. & Senter, P. D. (2003) Recent advances in antibody drug conjugates for cancer therapy. Annu. Rep. Med. Chem., 38, 229-237; Chari, R. V. (2008) Targeted cancer therapy: conferring specificity to cytotoxic drugs. Acc. Chem. Res. 41, 98-107]. More than 30 G-type immunoglobulins (IgG) and related agents have been approved over the past 25 years mainly for cancers and inflammatory diseases.

An alternative strategy is to look to chemically conjugate small anti-neoplastic molecules to mAbs, used both as carriers (increased half-life) and as targeting agents (selectivity). Considerable effort has been directed toward the use of monoclonal antibodies (mAbs) for targeted drug delivery due to their high selectivities for tumor-associated antigens, favorable pharmacokinetics, and relatively low intrinsic toxicities. The mAb-drug conjugates (ADCs) are formed by covalently linking anticancer drugs to mAbs, usually through a conditionally stable linker system. Upon binding to cell surface antigens, mAbs used for most ADCs are actively transported to lysosomes or other intracellular compartments, where enzymes, low pH, or reducing agents facilitate drug release. There are, however, currently limited ADCs in development.

Antigens must have high tumor cell selectivity to limit toxicity and off-target effects. A plethora of tumor-associated antigens have been investigated in pre-clinical models and in clinical trials including antigens over-expressed in B-cells (e.g., CD20, CD22, CD40, CD79), T-cells (CD25, CD30), carcinoma cells (HER2, EGFR, EpCAM, EphB2, PSMA), endothelial (endoglin), or stroma cells (fibroblast activated protein), to name a few [Teicher BA. Antibody-drug conjugate targets. Curr Cancer Drug Targets 9(8):982-1004, 2009]. An important property for ADC targets is their ability to be internalized; this can be an intrinsic feature of the antigen by itself, or it can be induced by the binding of the antibody to its antigen. Indeed, ADC internalization is crucial to reduce toxicity associated with an extracellular delivery of the drug payload.

Regarding the conjugated small molecules and in contrast to the vast variety of putative antigen targets, a limited number of families of cytotoxic drugs used as payloads in ADCs are currently actively investigated in clinical trials: calicheamycin (Pfizer), duocarmycins (Synthon), pyrrolobenzodiazepines (Spirogen), irinotecan (Immunomedics), maytansinoids (DM1 and DM4; ImmunoGen+Genentech/Roche, Sanofi-Aventis, Biogen Idec, Centocor/Johnson & Johnson, Millennium/Takeda), and auristatins (MMAE and MMAF; Seattle Genetics+Genentech/Roche, MedImmune/AstraZeneca, Bayer-Schering, Celldex, Progenics, Genmab). Calicheamycin, duocarmycins and pyrrolobenzodiazepines are DNA minor groove binders, irinotecan is a topoisomerase I inhibitor, whereas maytansinoids and auristatins are tubulin depolymerization agents.

Interestingly, a representative of three of these cytotoxic-derived ADCs has reached late stage clinical trials. Trastuzumab emtansine (T-DM1), trastuzumab linked to a maytansinoid hemi-synthetic drug by a stable linker (FDA approval on Feb. 22, 2013 for advanced HER2 positive breast cancer); Inotuzumab ozogamicin (CMC-544), a humanized anti-CD22 mAb (G5/44, IgG4) conjugated to calicheamycin with an acid labile linker (acetylphenoxy-butanoic) (B-cell non-Hodgkin's lymphoma); Brentuximab vedotin, a humanized anti-CD30 mAb linked to monomethyl auristatin E (MMAE), via a maleimidecaproyl-valyl-citrullinyl-p-aminobenzylcarbamate linker (FDA approval on Aug. 19, 2011 for anaplastic large cell lymphoma and Hodking's lymphoma).

Linkers represent the key component of ADC structures. Several classes of second generation linkers have been investigated, including acid-labile hydrazone linkers (lysosomes) (e.g. gemtuzumab and inotuzumab ozogamicin); disulfide-based linkers (reductive intracellular environment); non-cleavable thioether linkers (catabolic degradation in lysosomes) (e.g., trastuzumab emtansine); peptide linkers (e.g. citruline-valine) (lysosomal proteases like cathepsin-B) (e.g. brentuximab vedotin): see, for example, WO-A-2004/010957, WO-A-2006/060533 and WO-A-2007/024536. Purification of antibody-drug conjugates by size exclusion chromatography (SEC) has also been described [see, e.g., Liu et al., Proc. Natl. Acad. Sci. USA, 93: 8618-8623 (1996), and Chari et al., Cancer Research, 52: 127-131 (1992)].

Trastuzumab (Herceptin) is a monoclonal antibody that interferes with the HER2/neu receptor. Its main use is to treat certain breast cancers. The HER receptors are proteins that are embedded in the cell membrane and communicate molecular signals from outside the cell (molecules called EGFs) to inside the cell, and turn genes on and off. The HER proteins stimulate cell proliferation. In some cancers, notably certain types of breast cancer, HER2 is over-expressed, and causes cancer cells to reproduce uncontrollably.

The HER2 gene is amplified in 20-30% of early-stage breast cancers, which makes it overexpress epidermal growth factor (EGF) receptors in the cell membrane. In some types of cancer, HER2 may send signals without growth factors arriving and binding to the receptor, making its effect in the cell constitutive; however, trastuzumab is not effective in this case.

The HER2 pathway promotes cell growth and division when it is functioning normally; however when it is overexpressed, cell growth accelerates beyond its normal limits. In some types of cancer the pathway is exploited to promote rapid cell growth and proliferation and hence tumor formation. In cancer cells the HER2 protein can be expressed up to 100 times more than in normal cells (2 million versus 20,000 per cell). This overexpression leads to strong and constant proliferative signaling and hence tumor formation. Overexpression of HER2 also causes deactivation of checkpoints, allowing for even greater increases in proliferation.

In the compounds of the present invention, Ab is a moiety comprising at least one antigen binding site. In an alternative embodiment, Ab can be any suitable agent that is capable of binding to a target cell, preferably an animal cell and more preferably, a human cell. Examples of such agents include lymphokines, hormones, growth factors and nutrient-transport molecules (e.g. transferrin). In another example, Ab may be an aptamer, and may include a nucleic acid or a peptide aptamer.

Where Ab is a moiety comprising at least one antigen binding site, the moiety is preferably an antigen-binding peptide or polypeptide. In a preferred embodiment, the moiety is an antibody or an antigen-binding fragment thereof.

The term ‘antibody’ in the drug conjugates of the present invention refers to any immunolglobulin, preferably a full-length immunoglobulin. Preferably, the term covers monoclonal antibodies, polyclonal antibodies, multispecific antibodies, such as bispecific antibodies, and antibody fragments thereof, so long as they exhibit the desired biological activity. Antibodies may be derived from any species, but preferably are of rodent, for examples rat or mouse, human or rabbit origin. Alternatively, the antibodies, preferably monoclonal antibodies, may be humanised, chimeric or antibody fragments thereof. The term ‘chimeric antibodies’ may also include “primatised” antibodies comprising variable domain antigen-binding sequences derived from a non-human primate (e.g., Old World Monkey, Ape etc) and human constant region sequences. The immunoglobulins can also be of any type (e.g. IgG, IgE, IgM, IgD, and IgA), class (e.g., IgG1, IgG2, IgG3, IgG4, IgA1 and IgA2) or subclass of immunoglobulin molecule.

The term ‘monoclonal antibody’ refers to a substantially homogenous population of antibody molecules (i.e. the individual antibodies comprising the population are identical except for possible naturally occurring mutations that may be present in minor amounts), produced by a single clone of B lineage cells, often a hybridoma. Importantly, each monoclonal has the same antigenic specificity—i.e. it is directed against a single determinant on the antigen.

The production of monoclonal antibodies can be carried out by methods known in the art. However, as an example, the monoclonal antibodies can be made by the hybridoma method (Kohler et al (1975) Nature 256:495), the human B cell hybridoma technique (Kozbor et al., 1983, Immunology Today 4: 72), or the EBV-hybridoma technique (Cole et al., 1985, Monoclonal Antibodies and Cancer Therapy, Alan R. Liss, Inc., pp. 77-96). Alternatively, the monoclonal antibody can be produced using recombinant DNA methods (see, U.S. Pat. No. 4,816,567) or isolated from phage antibody libraries using the techniques described in Clackson et al (1991) Nature, 352:624-628; Marks et al (1991) J. Mol. Biol., 222:581-597.

Polyclonal antibodies are antibodies directed against different determinants (epitopes). This heterogenous population of antibody can be derived from the sera of immunised animals using various procedures well known in the art.

The term ‘bispecific antibody’ refers to an artificial antibody composed of two different monoclonal antibodies. They can be designed to bind either to two adjacent epitopes on a single antigen, thereby increasing both avidity and specificity, or bind two different antigens for numerous applications, but particularly for recruitment of cytotoxic T- and natural killer (NK) cells or retargeting of toxins, radionuclides or cytotoxic drugs for cancer treatment (Holliger & Hudson, Nature Biotechnology, 2005, 23(9), 1126-1136). The bispecific antibody may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation (WO 94/04690; Suresh et al., Methods in Enzymology, 1986, 121:210; Rodrigues et al., 1993, J. of Immunology 151:6954-6961; Carter et al., 1992, Bio/Technology 10:163-167; Carter et al., 1995, J. of Hematotherapy 4:463-470; Merchant et al., 1998, Nature Biotechnology 16:677-681.

Methods to prepare hybrid or bispecific antibodies are known in the art. In one method, bispecific antibodies can be produced by fusion of two hybridomas into a single ‘quadroma’ by chemical cross-linking or genetic fusion of two different Fab or scFv modules (Holliger & Hudson, Nature Biotechnology, 2005, 23(9), 1126-1136).

The term ‘chimeric’ antibody refers to an antibody in which different portions are derived from different animal species. For example, a chimeric antibody may derive the variable region from a mouse and the constant region from a human. In contrast, a ‘humanised antibody’ comes predominantly from a human, even though it contains non-human portions. Specifically, humanised antibodies are human immunoglobulins (recipient antibody) in which residues from a hypervariable region of the recipient are replaced by residues from hypervariable regions of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanised antibodies may comprise residues that are not found in the recipient antibody or in the donor antibody. These modifications are made to further refine antibody performance. In general, the humanised antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanised antibody optionally also will comprise at least a portion of an immunoglobulin constant region (Fc), typically that of a human immunoglobulin.

Recombinant antibodies such as chimeric and humanised monoclonal antibodies can be produced by recombinant DNA techniques known in the art. Completely human antibodies can be produced using transgenic mice that are incapable of expressing endogenous immunoglobulin heavy and light chains genes, but which can express human heavy and light chain genes. The transgenic mice are immunized in the normal fashion with a selected antigen. Monoclonal antibodies directed against the antigen can be obtained using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, for example, U.S. Pat. Nos. 5,625,126; 5,633,425; 5,569,825; 5,661,016; 5,545,806; each of which is incorporated herein by reference in its entirety. Other human antibodies can be obtained commercially from, for example, Abgenix, Inc. (Freemont, CA) and Genpharm (San Jose, CA).

The term ‘antigen-binding fragment’ in the drug conjugates of the present invention refers to a portion of a full length antibody where such antigen-binding fragments of antibodies retain the antigen-binding function of a corresponding full-length antibody. The antigen-binding fragment may comprise a portion of a variable region of an antibody, said portion comprising at least one, two, preferably three CDRs selected from CDR1, CDR2 and CDR3. The antigen-binding fragment may also comprise a portion of an immunoglobulin light and heavy chain. Examples of antibody fragments include Fab, Fab′, F(ab′)₂, scFv, di-scFv, sdAb, and BiTE (Bi-specific T-cell engagers), Fv fragments including nanobodies, diabodies, diabody-Fc fusions, triabodies and, tetrabodies; minibodies; linear antibodies; fragments produced by a Fab expression library, anti-idiotypic (anti-Id) antibodies, CDR (complementary determining region), and epitope-binding fragments of any of the above that immunospecifically bind to a target antigen such as a cancer cell antigens, viral antigens or microbial antigens, single-chain or single-domain antibody molecules including heavy chain only antibodies, for example, camelid VHH domains and shark V-NAR; and multispecific antibodies formed from antibody fragments. For comparison, a full length antibody, termed ‘antibody’ is one comprising a VL and VH domains, as well as complete light and heavy chain constant domains.

The antibody may also have one or more effector functions, which refer to the biological activities attributable to the Fc region (a native sequence Fc region or amino acid sequence variant Fc region engineered according to methods in the art to alter receptor binding) of an antibody. Examples of antibody effector functions include CIq binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

The antibody can also be a functionally active fragment (also referred to herein as an immunologically active portion), derivative or analog of an antibody that immunospecifically binds to a target antigen such as a cancer cell antigen, viral antigen, or microbial antigen or other antibodies bound to tumour cells. In this regard, functionally active means that the fragment, derivative or analog is able to elicit anti-idiotype antibodies that recognise the same antigen that the antibody from which the fragment, derivative or analog is derived recognised. Specifically, in an exemplary embodiment the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIA core assay), see, for example, Kabat et al., 1991, Sequences of Proteins of Immunological Interest, Fifth Edition, National Institute of Health, Bethesda, Md; Kabat E et al., 1980, J. of Immunology 125(3):961-969).

The term ‘antibody’ may also include a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody. The antibody or fragment thereof may be covalently linked to the other protein at the N-terminus of the constant domain.

Furthermore, the antibody or antigen-binding fragments of the present invention may include analogs and derivatives of antibodies or antigen-binding fragments thereof that are either modified, such as by the covalent attachment of any type of molecule as long as such covalent attachment permits the antibody to retain its antigen binding immunospecificity. Examples of modifications include glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies or antigen-binding fragments of the present invention may also have modifications (e.g., substitutions, deletions or additions) in the Fc domain of the antibody. Specifically, the modifications may be in the Fc-hinge region and result in an increased binding for the FcRn receptor (WO 97/34631).

In one embodiment, the antibody in the drug conjugate of the present invention may be any antibody or antigen-binding fragment thereof, preferably a monoclonal antibody that is useful in the treatment of a disease, preferably cancer and more preferably a cancer selected from lung cancer, including NSCLC, gastric cancer, colorectal cancer, breast cancer, pancreas carcinoma, endometrial cancer, bladder cancer, cervical cancer, esophageal cancer, gallbladder cancer, uterine cancer, salivary duct cancer, ovarian cancer, kidney cancer, leukaemia, multiple myeloma, and lymphoma, wherein the cancer is preferably a HER2 positive cancer, wherein the HER2 positive cancers include HER2 positive lung cancer including HER2 positive NSCLC, HER2 positive gastric cancer, HER2 positive colorectal cancer, HER2 positive breast cancer, HER2 positive pancreas carcinoma, HER2 positive endometrial cancer, HER2 positive bladder cancer, HER2 positive cervical cancer, HER2 positive esophageal cancer, HER2 positive gallbladder cancer, HER2 positive uterine cancer, HER2 positive salivary duct cancer and HER2 positive ovarian cancer, more preferably HER2 positive breast cancer, HER2 positive ovarian cancer and HER2 positive gastric cancer, most preferably HER2 positive breast cancer.

Antibodies that may be useful in the treatment of cancer include, but are not limited to, antibodies against the following antigens: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas) for example EGF receptor 2 protein (breast cancer), MAGE-I (carcinomas), MAGE-2 (carcinomas), MAGE-3 (carcinomas), MAGE-4 (carcinomas), anti-transferrin receptor (carcinomas), p97 (melanoma), MUCl-KLH (breast cancer), CEA (colorectal), gplOO (melanoma), MARTI (melanoma), PSA (prostate), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies include, but are not limited to, BR96 mAb (Trail, P. A., et al Science (1993) 261, 212-215), BR64 (Trail, P A, et al Cancer Research (1997) 57, 100-105, mAbs against the CD40 antigen, such as S2C6 mAb (Francisco, J. A., et al Cancer Res. (2000) 60:3225-3231), mAbs against the CD70 antigen, such as 1F6 mAb, and mAbs against the CD30 antigen, such as AClO (Bowen, M. A., et al (1993) J. Immunol., 151:5896-5906; Wahl et al., 2002 Cancer Res. 62(13):3736-3742). Many other internalizing antibodies that bind to tumor associated antigens can be used and have been reviewed (Franke, A. E., et al Cancer Biother Radiopharm. (2000) 15:459-476; Murray, J. L., (2000) Semin Oncol, 27:64-70; Breitling, F., and Dubel, S., Recombinant Antibodies, John Wiley, and Sons, New York, 1998).

Other tumour-associated antigens include, but are not limited to, BMPR1B, E16, STEAP1, STEAP2, 0772P. MPF, Napi3b, Sema5b, PSCA hlg, ETBR, MSG783, TrpM4, CRIPTO, CD21, CD79b, FcRH2, HER2, NCA, MDP, IL20Rα, Brevican, EphB2R, ASLG659, PSCA, GEDA, BAFF-R, CD79A, CXCR5, HLA-DOB, P2X5, CD72, LY64, FCRH1, IRTA2 and TENB2.

In a further embodiment, the antibody or antigen-binding fragment binds to an epitope that is present on a cell, such as a tumour cell. Preferably, where the cell is a tumour cell, the tumour cell epitope is not present on non-tumour cells, or is present at a lower concentration or in a different steric configuration than in tumour cells.

In one embodiment, the antibody or antigen-binding fragment binds to an epitope present in the context of one of the following antigens: CA125, CA15-3, CA19-9 L6, Lewis Y, Lewis X, alpha fetoprotein, CA 242, placental alkaline phosphatase, prostate specific antigen, prostatic acid phosphatase, epidermal growth factor for example EGF receptor 2 protein, MAGE-I, MAGE-2, MAGE-3, MAGE-4, anti-transferrin receptor, p97, MUCl-KLH, CEA, gplOO, MART1, PSA, IL-2 receptor, CD20, CD52, CD33, CD22, human chorionic gonadotropin, CD38, CD40, mucin, P21, MPG, Neu oncogene product, BMPR1B, E16, STEAP1, STEAP2, 0772P. MPF, Napi3b, Sema5b, PSCA hlg, ETBR, MSG783, TrpM4, CRIPTO, CD21, CD79b, FcRH2, HER2, NCA, MDP, IL20Rα, Brevican, EphB2R, ASLG659, PSCA, GEDA, BAFF-R, CD79A, CXCR5, HLA-DOB, P2X5, CD72, LY64, FCRH1, IRTA2, TENB2.

In one embodiment, where the antigen is ErBB2 (also known as ERBB2, CD340 or HER2; such terms may be used interchangeably), the antibody or antigen-binding fragment may bind to one or more of the following epitopes: ARHC L (SEQ ID NO: 1), QNGS (SEQ ID NO: 2) and PPFCVARC PSG (SEQ ID NO: 3). These epitopes correspond to positions 557-561, 570-573 and 593-603 respectively of the human HER2 polypeptide sequence (Accession: NM_004448, Version: NM_004448.3).

An antibody that binds a molecular target or an antigen of interest, e.g., ErbB2 antigen, is one capable of binding that antigen with sufficient affinity such that the antibody is useful in targeting a cell expressing the antigen. Where the antibody is one which binds ErbB2, it will usually preferentially bind ErbB2 as opposed to other ErbB receptors, and may be one which does not significantly cross-react with other proteins such as EGFR, ErbB 3 or ErbB4. In such embodiments, the extent of binding of the antibody to these non-ErbB2 proteins (e.g., cell surface binding to endogenous receptor) will be less than 10% as determined by fluorescence activated cell sorting (FACS) analysis or radioimmunoprecipitation (RIA). Sometimes, the anti-ErbB2 antibody will not significantly cross-react with the rat neu protein, e.g., as described in Schecter et al., Nature 312:513-516 (1984) and Drebin et al., Nature 312:545-548 (1984).

In another embodiment, the antibody of the drug conjugate or target of the present invention may be selected from an antibody or target in the below table. Such antibodies are immunospecific for a target antigen and can be obtained commercially or produced by any method known in the art such as, e.g., recombinant expression techniques.

TABLE 1
Therapeutic monoclonal antibodies

| Name | Trade name | Target |
|---|---|---|
| 3F8 | | GD2 ganglioside |
| 8H9 | | B7-H3 |
| Abagovomab | | CA-125 (imitation) |
| Abituzumab | | CD51 |
| Adecatumumab | | EpCAM |
| Alemtuzumab | Campath, | CD52 |
| | Lemtrada | |
| Altumomab | Hybri-ceaker | CEA |
| Amatuximab | | Mesothelin |
| Andecaliximab | | gelatinase B |
| Anetumab | | MSLN |
| Aprutumab | | FGFR2 |
| Ascrinvacumab | | Activin receptor-like kinase 1 |
| Atezolizumab | Tecentriq | PD-L1 |
| Atinumab | | RTN4 |
| Avelumab | Bavencio | PD-L1 |
| Azintuxizumab | | CD319 |
| Bavituximab | | phosphatidylserine |
| BCD-100 | | PD-1 |
| Belantamab | | BCMA |
| Bemarituzumab | | FGFR2 |
| Bermekimab | Xilonix | IL1A |
| Bersanlimab | | ICAM-1 |
| Besilesomab | Scintimun | CEA-related antigen |
| Bevacizumab | Avastin | VEGF-A |
| Bivatuzumab | | CD44 v6 |
| Blontuvetmab | Blontress | CD20 |
| Brentuximab | Adcentris | CD30 (TNFRSF8) |
| Brontictuzumab | | Notch 1 |
| Cabiralizumab | | CSF1R |
| Camidanlumab | | CD25 |
| Camrelizumab | | Programmed cell death 1 |
| Cantuzumab | | MUC-1 |
| Capromab | Prostascint | prostatic carcinoma cells |
| Carlumab | | MCP-1 |
| Carotuximab | | endoglin |
| Catumaxomab | Removab | EpCAM, CD3 |
| cBR96 | | Lewis-Y antigen |
| Cemiplimab | | PCDC1 |
| Cergutuzumab | | IL2 |
| Cetrelimab | | Programmed cell death 1 |
| Cetuximab | Erbitux | EGFR |
| Cibisatamab | | CEACAM5 |
| Cirmtuzumab | | ROR1 |
| Cixutumumab | | IGF-1 receptor (CD221) |
| Clivatuzumab | hPAM4-Cide | MUC1 |
| Codrituzumab | | glypican 3 |
| Cofetuzumab | | PTK7 |
| Coltuximab | | CD19 |
| Conatumumab | | TRAIL-R2 |
| Cusatuzumab | | CD70 |
| Dacetuzumab | | CD40 |
| Dalotuzumab | | IGF-1 receptor (CD221) |
| Dapirolizumab | | CD154 (CD40L) |
| pegol | | |
| Daratumumab | Darzalex | CD38 |
| Dectrekumab | | IL-13 |
| Demcizumab | | DLL4 |
| Denintuzumab | | CD19 |
| Denosumab | Prolia | RANKL |
| Depatuxizumab | | EGFR |
| Derlotuximab | | Histone complex |
| Detumomab | | B-lymphoma cell |
| Dinutuximab | Unituxin | GD2 ganglioside |
| Dostarlimab | | PCDP1 |
| Drozitumab | | DR5 |
| DS-8201 | | HER2 |
| Duligotuzumab | | ERBB3 (HER3) |
| Durvalumab | Imfinzi | PD-L1 |
| Dusigitumab | | ILGF2 |
| Ecromeximab | | GD3 ganglioside |
| Edrecolomab | Panorex | EpCAM |
| Elgemtumab | | ERBB3 (HER3) |
| Elotuzumab | Empliciti | SLAMF7 |
| Elsilimomab | | IL-6 |
| Emactuzumab | | CSF1R |
| Emibetuzumab | | HHGFR |
| Enapotamab | | AXL |
| Enavatuzumab | | TWEAK receptor |
| Enfortumab | | nectin-4 |
| Enlimomab pegol | | ICAM-1 (CD54) |
| Enoblituzumab | | CD276 |
| Ensituximab | | 5AC |
| Epitumomab | | episialin |
| Epratuzumab | | CD22 |
| Ertumaxomab | Rexomun | HER2/neu, CD3 |
| Etaracizumab | Abegrin | integrin αᵥβ₃ |
| Etigilimab | | TIGIT |
| Faricimab | | VEGF-A and Ang-2 |
| Farletuzumab | | folate receptor 1 |
| FBTA05 | Lymphomun | CD20 |
| Fibatuzumab | | Ephrin receptor A3 |
| Ficlatuzumab | | HGF |
| Figitumumab | | IGF-1 receptor (CD221) |
| Flanvotumab | | TYRP1 (glycoprotein 75) |
| Futuximab | | EGFR |
| Galiximab | | CD80 |
| Ganitumab | | 1 receptor (CD221) |
| Gatipotuzumab | | MUC1 |
| Gedivumab | | Hemagglutinin HA |
| Gemtuzumab | Mylotarg | CD33 |
| Gilvetmab | | PCDC1 |
| Girentuximab | Rencarex | carbonic anhydrase 9 (CA-IX) |
| Glembatumumab | | GPNMB |
| IBI308 | | PD1 |
| Ibritumomab | Zevalin | CD20 |
| Icrucumab | | VEGFR-1 |
| Ifabotuzumab | | EPHA3 |
| Iladatuzumab | | CD97B |
| IMAB362 | | CLDN18.2 |
| Imalumab | | MIF |
| Imaprelimab | | MCAM |
| Imgatuzumab | | EGFR |
| Indatuximab | | SDC1 |
| indusatumab | | GUCY2C |
| inebilizumab | | CD19 |
| Inotuzumab | Besponsa | CD22 |
| Intetumumab | | CD51 |
| Ipilimumab | Yervoy | CD152 |
| Iratumumab | | CD30 (TNFRSF8) |
| Isatuximab | | CD38 |
| Iscalimab | | CD40 |
| Istiratumab | | IGF1R, CD221 |
| Labetuzumab | CEA-Cide | CEA |
| Lacnotuzumab | | CSF1, MCSF |
| Ladiratuzumab | | LIV-1 |
| Laprituximab | | EGFR |
| Lendalizumab | | C5 |
| Lenzilumab | | CSF2 |
| Leronlimab | | CCR5 |
| Lesofavumab | | Hemagglutinin HA |
| Lexatumumab | | TRAIL-R2 |
| Lifastuzumab | | Phosphate-sodium co-transporter |
| Lilotomab | | CD37 |
| Lintuzumab | | CD33 |
| Lirilumab | | KIR2D |
| Loncastuximab | | CD19 |
| Losatuxizumab | | EGFR, ERBB1 HER1 |
| Lorvotuzumab | | CD56 |
| Lucatumumab | | CD40 |
| Lumiliximab | | CD23 (IgE receptor) |
| Lumretuzumab | | ERBB3 (HER3) |
| Lupartumab | | LYPD3 |
| Lutikizumab | | Interleukin 1 alpha |
| Mapatumumab | | TRAIL-R1 |
| Margetuximab | | HER2 |
| Matuzumab | | EGFR |
| Milatuzumab | | CD74 |
| Minretumomab | | TAG-72 |
| Mirvetuximab | | Folate receptor alpha |
| Mitumomab | | GD3 ganglioside |
| Modotuximab | | EGFR extracellular domain III |
| Mogamulizumab | Poteligeo | CCR4 |
| Monalizumab | | NKG2A |
| Morolimumab | | Rhesus factor |
| Mosunetuzumab | | CD3E, MS4A1, CD20 |
| Moxetumomab | | CD22 |
| Namilumab | | CSF2 |
| Naratuximab | | CD37 |
| Narnatumab | | RON |
| Navicixizumab | | DLL4 |
| Naxitamab | | C-Met |
| Necitumumab | Portrazza | EGFR |
| Nerelimomab | | TNF-α |
| Nesvacumab | | angiopoietin 2 |
| Nimotuzumab | Theracim, | EGFR |
| | Theraloc | |
| Nivolumab | Opdivo | PD-1 |
| Obinutuzumab | Gazyva | CD20 |
| Ocaratuzumab | | CD20 |
| Ofatumumab | Arzerra | CD20 |
| Olaratumab | Lartruvo | PDGF-R α |
| Oleclumab | | 5′-nucleotidase |
| Omburtamab | | CD276 |
| Onartuzumab | | human scatter factor receptor |
| | | kinase |
| Ontuxizumab | | TEM1 |
| Onvatilimab | | VSIR |
| Oregovomab | OvaRex | CA-125 |
| Otelixizumab | | CD3 |
| Otlertuzumab | | CD37 |
| Pamrevlumab | | CTGF |
| Panitumumab | Vectibix | EGFR |
| Pankomab | | Tumor specific glycosylation |
| | | of MUC1 |
| Parsatuzumab | | EGFL7 |
| Pasotuxizumab | | Folate hydrolase |
| Patritumab | | ERBB3 (HER3) |
| PDR001 | | PD-1 |
| Pembrolizumab | Keytruda | PD1 |
| Pemtumomab | Theragyn | MUC1 |
| Pertuzumab | Omnitarg | HER2/neu |
| Pidilizumab | | PD-1 |
| Pinatuzumab | | CD22 |
| Pintumomab | | adenocarcinoma antigen |
| Pogalizumab | | TNFR superfamily member 4 |
| Polatuzumab | | CD79B |
| Prezalizumab | | ICOSL |
| Pritumumab | | vimentin |
| Racotumomab | Vaxira | NGNA ganglioside |
| Radretumab | | fibronectin extra domain-B |
| Ramucirumab | Cyramza | VEGFR2 |
| Relatlimab | | LAG3 |
| Remtolumab | | Interleukin 17 alpha, TNF |
| Rilotumumab | | HGF |
| Rituximab | MabThera, Rituxan | CD20 |
| Robatumumab | | IGF-1 receptor (CD221) |
| Romilkimab | | Interleukin 13 |
| Rosmantuzumab | | Root plate-specific spondin 3 |
| Rovalpituzumab | | DLL3 |
| Sacituzumab | | TROP-2 |
| Samalizumab | | CD200 |
| Samrotamab | | LRRC15 |
| Satumomab | | TAG-72 |
| Selicrelumab | | CD40 |
| Seribantumab | | ERBB3 (HER3) |
| Setrusumab | | SOST |
| Sibrotuzumab | | FAP |
| SGN-CD19A | | CD19 |
| Siltuximab | Sylvant | IL-6 |
| Sintilimab | | PD-1 |
| Sirtratumab | | SLITRK6 |
| Sofituzumab | | CA-125 |
| Sontuzumab | | episialin |
| Spartalizumab | | PDCD1, CD279 |
| Tabalumab | | BAFF |
| Tacatuzumab | AFP-Cide | alpha-fetoprotein |
| Talacotuzumab | | CD123 |
| Tamtuvetmab | Tactress | CD52 |
| Taplitumomab | | CD19 |
| Tarextumab | | Notch receptor |
| Tavolimab | | CD134 |
| Telisotuzumab | | HGFR |
| Tenatumomab | | tenascin C |
| Tepoditamab | | Dendritic cell-associated lectin 2 |
| Tesidolumab | | C5 |
| Tetulomab | | CD37 |
| Tigatuzumab | | TRAIL-R2 |
| Timigutuzumab | | HER2 |
| Timolumab | | AOC3 |
| Tiragolumab | | TIGIT |
| Tislelizumab | | PCDC1, CD279 |
| Tisotumab | | Coagulation factor III |
| Tomuzotuximab | | EGFR, HER1 |
| Tositumomab | Bexxar | CD20 |
| Tovetumab | | CD140a |
| Trastuzumab | Herceptin | HER2/neu |
| TRBS07 | Ektomab | GD2 ganglioside |
| Tregalizumab | | CD4 |
| Tremelimumab | | CTLA-4 |
| Tucotuzumab | | EpCAM |
| Ublituximab | | MS4A1 |
| Ulocuplumab | | CXCR4 (CD184) |
| Urelumab | | 4-1BB (CD137) |
| Utomilumab | | 4-1BB (CD137) |
| Vadastuximab | | CD33 |
| Vanalimab | | CD40 |
| Vandortuzumab | | STEAP1 |
| Vantictumab | | Frizzled receptor |
| Vanucizumab | | angiopoietin 2 |
| Vapaliximab | | AOC3 (VAP-1) |
| Varisacumab | | VEGF-A |
| Varlilumab | | CD27 |
| Vatelizumab | | ITGA2 (CD49b) |
| Veltuzumab | | CD20 |
| Vesencumab | | NRP1 |
| Volociximab | | integrin α₅β₁ |
| Vonlerolizumab | | CD134 |
| Vopratelimab | | ICOS |
| Vorsetuzumab | | CD70 |
| Votumumab | HumaSPECT | tumor antigen CTAA16.88 |
| Vunakizumab | | Interleukin 17 alpha |
| Xentuzumab | | IGF1, IGF2 |
| XMAB-5574 | | CD19 |
| Zalutumumab | HuMax-EGFr | EGFR |
| Zanolimumab | HuMax-CD4 | CD4 |
| Zatuximab | | HER1 |
| Zenocutuzumab | | ERBB3, HER3 |
| Ziralimumab | | CD147 (basigin) |
| Zolbetuximab | | CLDN18 |

In addition to the above, the antibody of the drug antibody conjugate of the present invention may be Vitaxin which is a humanised antibody for the treatment of sarcoma; Smart IDlO which is a humanised anti-HLA-DR antibody for the treatment of non-Hodgkin's lymphoma; Oncolym which is a radiolabeled murine anti-HLA-DrlO antibody for the treatment of non-Hodgkin's lymphoma; and Allomune which is a humanised anti-CD2 mAb for the treatment of Hodgkin's Disease or non-Hodgkin's lymphoma.

The antibody of the drug conjugate of the present invention may also be any antibody-fragment known for the treatment of any disease, preferably cancer. Again, such antibody fragments are immunospecific for a target antigen and can be obtained commercially or produced by any method known in the art such as, e.g., recombinant expression techniques. Examples of such antibodies available include any from the below table.

TABLE 2
Therapeutic monoclonal antibody fragments

| Fragment | | | |
|---|---|---|---|
| type/format | Name | Trade name | Target |
| F(ab′)₂/humanised | Alacizumab | | VEGFR2 |
| | pegol | | |
| Fab/mouse | Anatumomab | | TAG-72 |
| Fab/ovine | | CroFab | Snake venom |
| Fab/ovine | | DigiFab | Digoxin |
| Fab/ovine | | Digibind | Digoxin |
| Fab′/mouse | arcitumomab | CEA-scan | CEA |
| Fab′/mouse | bectumomab | LymphoScan | CD22 |
| BiTE/mouse | Blinatumomab | Blincyto | CD19 |
| Fab/humanised | citatuzumab | | EpCAM |
| scFv/chimeric | duvortuxizumab | | CD19, CD3E |
| humanised | | | |
| scFv/human | gancotamab | | unknown |
| F(ab′)₂/mouse | igovomab | Indimacis-125 | CA-125 |
| Fab/mouse | nacolomab | | C242 antigen |
| Fab/mouse | naptumomab | | 5T4 |
| Fab/mouse | nofetumomab | | unknown |
| scFv/humanised | oportuzumab | Vicinium | EpCAM |
| BiTE/mouse | Solitomab | | EpCAM |
| Fab/humanised | | Thromboview | D-dimer |
| Fab/PEGylated | CDP791 | | VEGF |
| humanised | | | |
| Fab/bispecific | MDX-H210 | | Her2/Neu & |
| humanised | | | CD64 (γFcR1) |
| (ScFv)₄ fused to | CC49 | | TAG-72 |
| streptavidin mouse | | | Pancarcinoma |
| | | | antigen |
| ScFv fused to β- | SGN-17 | | P97 antigen |
| lactamase | | | |
| human | | | |
| ScFv fused to PEG | F5 scFv-PEG | | Her2 |
| human | Immunoliposome | | |
| Diabody | C6.5K-A | | Her2/Neu |
| (V_{H}-V_{L})₂ | | | |
| human | | | |
| Diabody | L19 | | EDB domain of |
| (V_{H}-V_{L})₂ | L19-γIFN | | fibronectin |
| human | | | |
| Diabody | T84.66 | | CEA |
| (V_{L}-V_{H})₂ | | | |
| human | | | |
| Minibody | T84.66 | | CEA |
| (scFᵥ-C_{H}3)₂ | | | |
| murine-human | | | |
| chimera | | | |
| (minibody) | | | |
| Minibody | 10H8 | | Her2 |
| murine-human | | | |
| chimera | | | |
| (minibody) | | | |
| S_{c}Fᵥ dimer Fc | T84.66 | | CEA |
| (S_{c}Fᵥ)₂-Fc | | | |
| murine-human | | | |
| chimera | | | |
| (minibody) | | | |
| Bispecific scFv | r28M | | CD28 and |
| (V_{L}-V_{H}-V_{H}-V_{L}) | | | MAP |
| mouse | | | |
| Bispecific scFv | BiTE MT103 | | CD19 and CD3 |
| (V_{L}-V_{H}-V_{H}-V_{L}) | | | |
| origin unknown | | | |
| Bispecific scFv | BiTE | | Ep-CAM and |
| (V_{L}-V_{H}-V_{H}-V_{L}) | | | CD3 |
| origin unknown | | | |
| Bispecific tandem | Tandab | | CD19 & CD3 |
| diabody | | | |
| (VH-VL- VH -VL) | | | |
| (mouse) | | | |
| VhH-β-lactamase | Nanobody | | CEA |
| fusion | | | |
| camelid | | | |
| Dab/human | Anti-TNFα dAb | | TNFα |
| VhH/camelid | Nanobody | | TNFα |
| VhH/camelid | Nanobody | | Von |
| | | | Willebrand |
| | | | factor |

Fa fragment, antigen-binding (one arm)
F(ab′)₂fragment, antigen-binding, including hinge region (both arms)
Fab′ fragment, antigen-binding, including hinge region (one arm)
scFv single-chain variable fragment
di-scFv dimeric, single-chain variable fragment
(Holliger & Hudson, Nature Biotechnology, 2005, 23(9), 1126-1136).

In a preferred embodiment, the antibody in the drug conjugates of the present invention targets a cell surface antigen.

In preferred embodiments, the antibody in the drug conjugates of the present invention may bind to a receptor encoded by the ErbB gene. The antibody may bind specifically to an ErbB receptor selected from EGFR, HER2, HER3 and HER4. Preferably, the antibody in the drug conjugate may specifically bind to the extracellular domain of the HER2 receptor and inhibit the growth of tumour cells which overexpress the HER2 receptor. The antibody of the drug conjugate may be a monoclonal antibody, e.g. a murine monoclonal antibody, a chimeric antibody, or a humanised antibody. Preferably, the humanised antibody may be huMAb4D5-1, huMAb4D5-2, huMAb4D5-3, huMAb4D5-4, huMAb4D5-5, huMAb4D5-6, huMAb4D5-7 or huMAb4D5-8 (Trastuzumab), particularly preferably Trastuzumab. The antibody may also be an antibody fragment, e.g. a Fab fragment.

Other preferred antibodies include:
(i) anti-CD4 antibodies. The antibody of the drug conjugate may be a monoclonal antibody, e.g. a murine monoclonal antibody, a chimeric antibody, or a humanised antibody;
(ii) anti-CD5 antibodies. The antibody of the drug conjugate may be a monoclonal antibody, e.g. a murine monoclonal antibody, a chimeric antibody, or a humanised antibody;
(iii) anti-CD13 antibodies. The antibody of the drug conjugate may be a monoclonal antibody, e.g. a murine monoclonal antibody, a chimeric antibody, or a humanised antibody;
(iv) anti-CD20 antibodies. The antibody of the drug conjugate may be a monoclonal antibody, e.g. a murine monoclonal antibody, a chimeric antibody, or a humanised antibody. Preferably, the humanised antibody is Rituximab or an antibody fragment thereof, e.g. a Fab fragment; and
(v)anti-CD30 antibodies. The antibody of the drug conjugate may be a monoclonal antibody, e.g. a murine monoclonal antibody, a chimeric antibody, or a humanised antibody. Preferably the humanised antibody is Brentuximab vedotin or an antibody fragment thereof.

In one embodiment of the invention, the drug antibody conjugate may demonstrate one or more of the following: (i) increased cytotoxicity (or a decrease in cell survival), (ii) increased cytostatic activity (cytostasis), (iii) increased binding affinity to the target antigen or epitope, (iv) increased internalisation of the conjugate, (v) reduction of patient side effects, and/or (vi) improved toxicity profile. Such increase may be relative to a known drug antibody conjugate in the art that binds the same or a different epitope or antigen.

### Processes for the Preparation of the Drug Antibody Conjugates

The drug antibody conjugates of the present invention can be prepared according to techniques that are well known in the art. Processes for conjugating moieties comprising at least one antigen binding site antibodies such as antibodies to a number of different drugs using different processes have been described and exemplified previously in, for example, WO-A-2004/010957, WO-A-2006/060533 and WO-A-2007/024536, the contents of which are incorporated herein by reference thereto. These involve use of a linker group that derivatises the drug, toxin or radionuclide in such a way that it can then be attached to the moiety such as an antibody. Attachment to the moiety such as an antibody is typically by one of three routes: via free thiol groups in cysteines after partial reduction of disulfide groups in the antibody; via free amino groups in lysines in the antibody; and via free hydroxyl groups in serines and/or threonines in the antibody. The attachment method varies depending upon the site of attachment on the moiety such as an antibody. Purification of antibody-drug conjugates by size exclusion chromatography (SEC) has also been described [see, e.g., Liu et al., Proc. Natl. Acad. Set (USA), 93: 8618-8623 (1996), and Chari et al., Cancer Research, 52: 127-131 (1992)].

As noted earlier, there is provided a process for the preparation of a drug conjugate according to the present invention comprising conjugating a moiety Ab comprising at least one antigen binding site and a drug D of formula (I) or (IH), Ab and D being as defined herein.

One example of a process for the preparation of a drug conjugate of the present invention involves the preparation of drug antibody conjugates of formula (G) or (G′) of the present invention as follows:

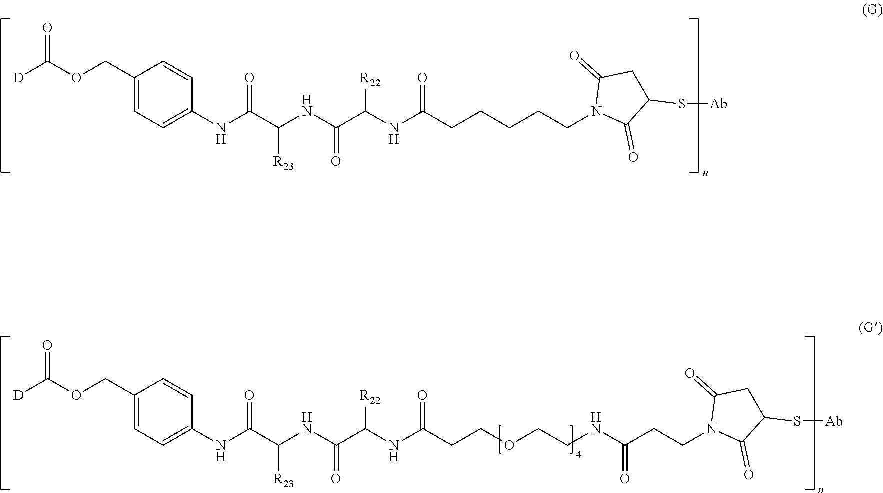

said process comprising the following steps:
(i) reacting a drug (D-H) of formula (IH)-H:

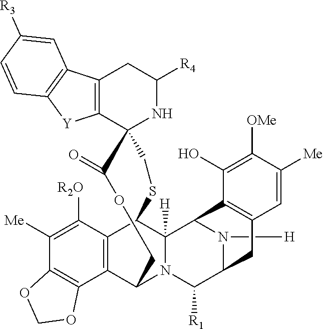

wherein the substituents in the definitions of (IH)-H are as defined above for formula (IH), with a compound of formula (D′) or (E):

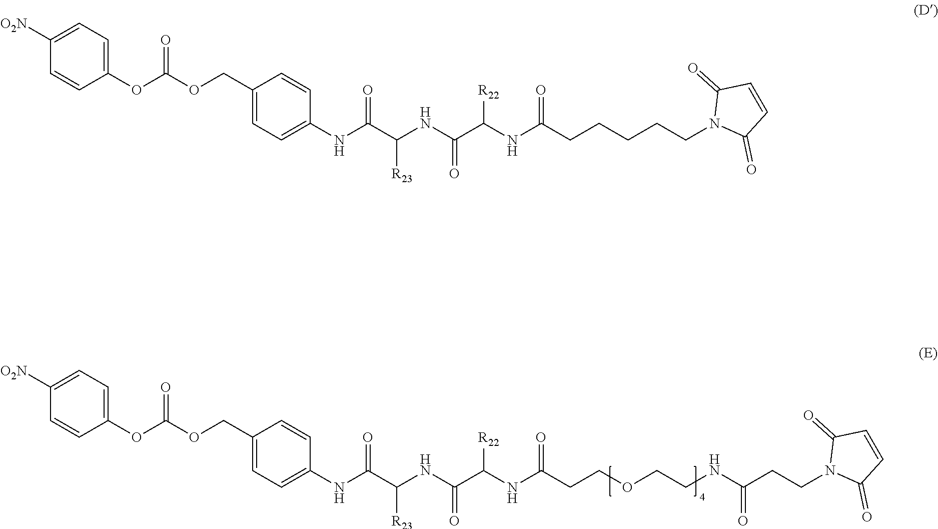

to give a compound of formula (F) or (F′), respectively:

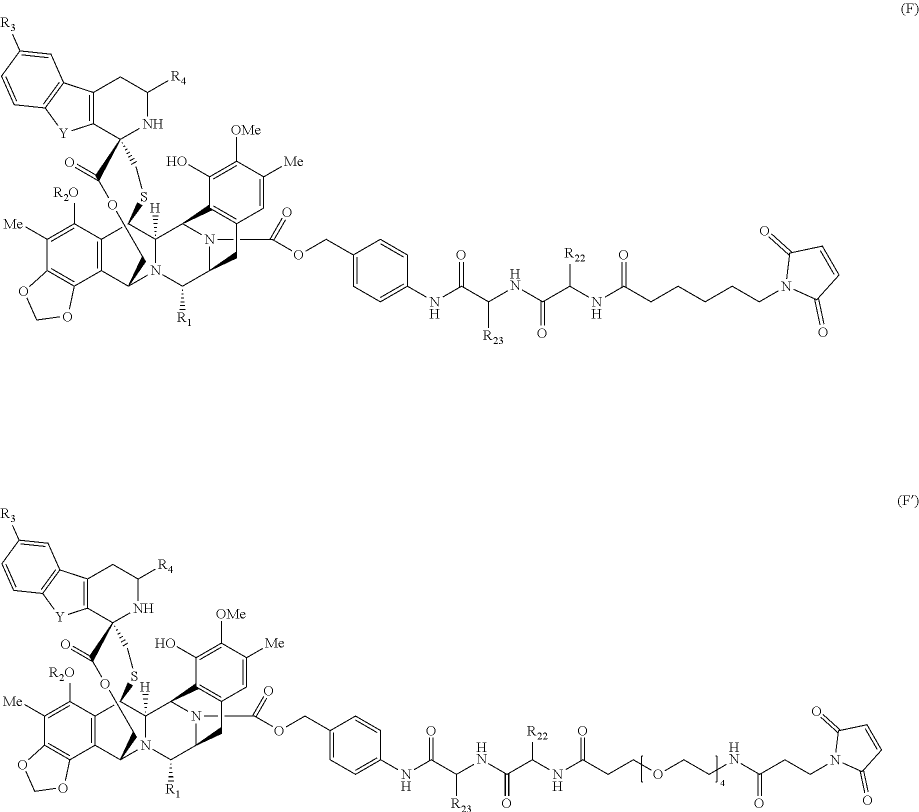

(ii) partial reduction of one or more disulfide bonds in the antibody to be conjugated to give a reduced antibody Ab-SH having free thiol groups:

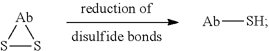

and
(iii) reaction of the partially reduced antibody Ab-SH having free thiol groups with the compound of formula (F) or (F′) produced in step (i) to give the desired drug antibody conjugate of formula (G) or (G′) respectively:

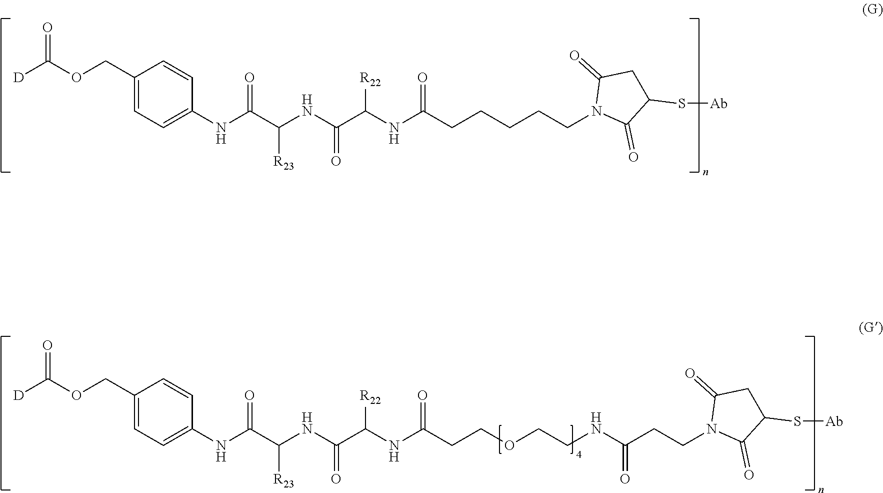

In another preferred embodiment of this process, the antibody is selected from Brentuximab, Gemtuzumab, Inozutumab, Rovalpituzumab, an anti-HER2 antibody such as Trastuzumab, an anti-CD4 antibody, an anti-CD5 antibody, an anti-CD13 antibody and an anti-CD30 antibody, or an antigen-binding fragment or an immunologically active portion thereof, or it is selected from an anti-HER2 antibody such as Trastuzumab and anti-CD13 antibody or an antigen-binding fragment or an immunologically active portion thereof, and most preferably it is Trastuzumab or an antigen-binding fragment or an immunologically active portion thereof. Furthermore, the partial reduction of this monoclonal antibody is performed using tris[2-carboxyethyl]phosphine hydrochloride (TCEP).

Another example of a process for the preparation of a drug conjugate of the present invention involves the preparation of drug antibody conjugates of formula (W) or (W′) of the present invention as follows:

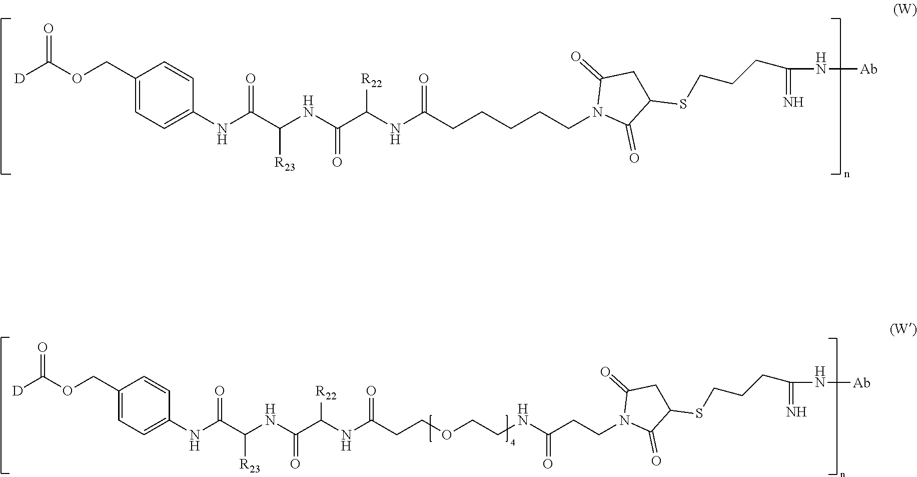

said process comprising the following steps:
(i) reacting the antibody with 2-iminothiolane hydrochloride (Traut's reagent) to give a thiol-activated antibody:

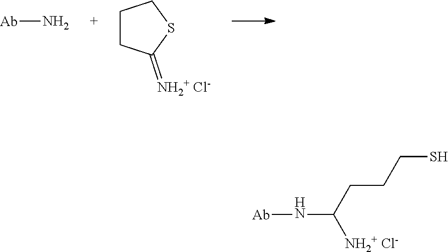

(ii) reacting the thiol-activated antibody with the compound of formula (F) or (F′), to give the desired drug antibody conjugate of formula (W) or (W′), respectively.

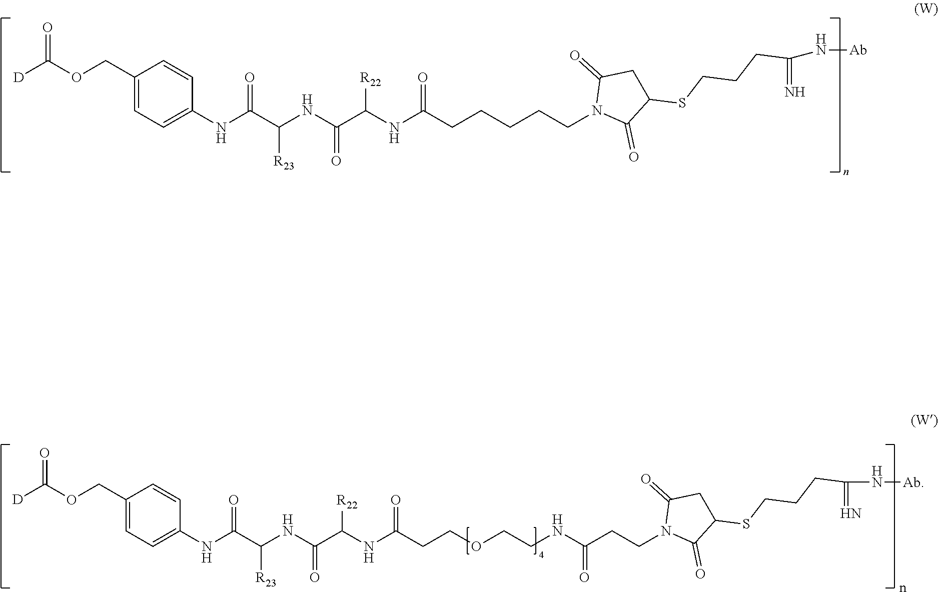

In another preferred embodiment of this process, the antibody is selected from Brentuximab, Gemtuzumab, Inozutumab, Rovalpituzumab, an anti-HER2 antibody such as Trastuzumab, an anti-CD4 antibody, an anti-CD5 antibody, an anti-CD13 antibody and an anti-CD30 antibody, or an antigen-binding fragment or an immunologically active portion thereof, or it is selected from an anti-HER2 antibody such as Trastuzumab and anti-CD13 antibody or an antigen-binding fragment or an immunologically active portion thereof, and most preferably it is Trastuzumab or an antigen-binding fragment or an immunologically active portion thereof.

Another example of a process for the preparation of a drug antibody conjugate of the present invention, involves the preparation of drug antibody conjugates of formula (0) or (P) as follows:

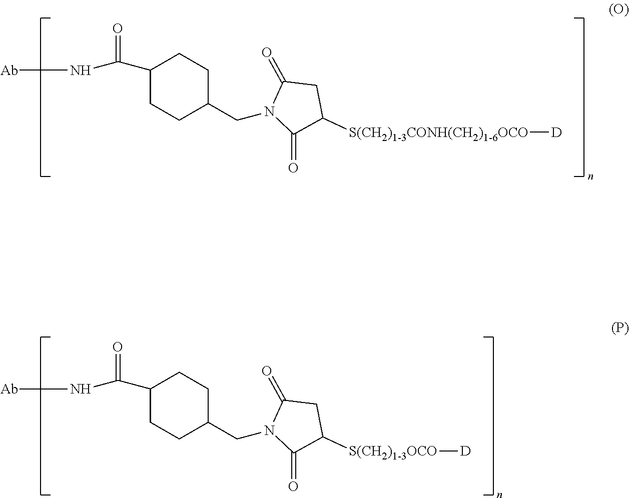

said process comprising the following steps:
(i) either:
(a) reacting a drug (D-H) of formula (IH)-H:

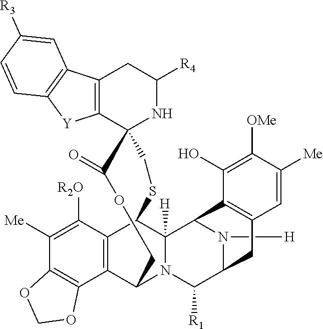

wherein the substituents in the definitions of (IH)-H are as defined above, with a compound of formula X₂—C(O)—X₁ wherein X₁ and X₂ are leaving groups to give a compound of formula (B):

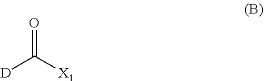

and the point of attachment of the —(C═O)X₁ moiety is the free —NH₂ group of the compound of formula D-H, or
(b) reacting said drug (D-H) of formula (IH)-H as defined above with 4-nitro-phenylchloroformate to give a compound of formula (J):

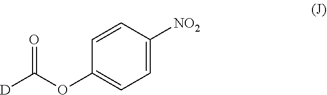

and the point of attachment of the (4-nitrophenyl)-O—CO— group is the same as that for the X₁(CO) moiety in (a) above;
(ii) either:
   (c) reacting the compound of formula (B) produced in step (i) with a hydroxy compound of formula HO—(CH₂)₁₋₆NHProt^{NH} and removing the Prot^{NH} group from the coupled compound to give a compound of formula (C):

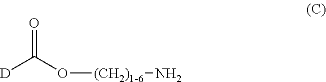

and then reacting the resulting compound of formula (C) with a compound of formula Me-S—S—(CH₂)₁₋₃—CO₂H to give a compound of formula (K):

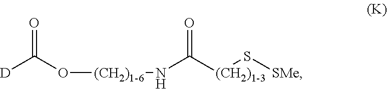 [SMILES: [2H]C(=O)OCNC(=O)CSSC]

or
(d) reacting the compound (J) produced in step (i) with a compound of formula HO—(CH₂)₁₋₃SProt^{SH} and removing the Prot^{SH} group from the coupled compound to give a compound of formula (L):

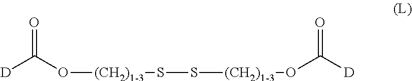 [SMILES: [2H]C(=O)OCSSCOC([2H])=O]

(iii) reacting (K) or (L) produced in step (ii) with dithiothreitol under disulfide reducing conditions to give compounds of formula (M) and (N) respectively:

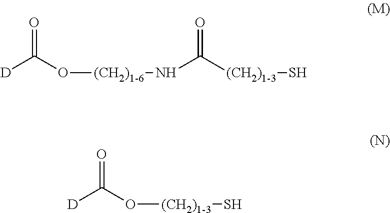 [SMILES: [2H]C(=O)OCNC(=O)CS ; [2H]C(=O)OCS]

(iv) reacting the antibody to be conjugated with succininimidyl-4-(N-maleimidomethyl)cyclohexane-1-carboxylate to derivatise said antibody at one or more lysine groups with a succininimidyl-4-(N-maleimidomethyl)cyclohexane-1-carbonyl group:

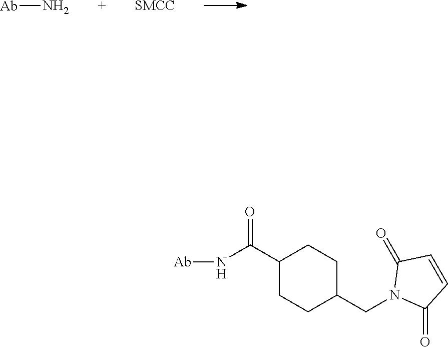

(v) reacting the derivatised antibody produced in step (iv) with either (M) or (N) produced in step (iii) to give the desired drug antibody conjugate of formula (O) or (P):

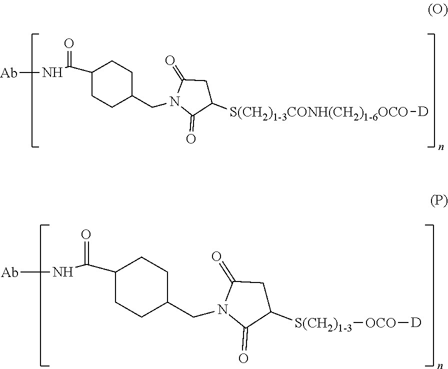

The compound of formula X₂—C(O)—X₁ is preferably 1,1′-carbonyldiimidazole. Similarly, the hydroxy compound reacted with the compound of formula (B) is preferably HO—(CH₂)₂₋₄-NHProt^{NH}, and more preferably HO—(CH₂)₃-NHProt^{NH}.

In one preferred embodiment of this invention, the compound reacted with the compound of formula (C) to give the compound of formula (K) is 3-(methyldisulfanyl)propanoic acid.

In another preferred embodiment, the compound HO—(CH₂)₁₋₃SProt^{SH} that is reacted with a compound of formula (J) to give a compound of formula (L) is HO—(CH₂)₃SProt^{SH}.

Where attachment to the drug-linker moiety is via free thiol groups in cysteines after partial reduction of disulfide groups in the moiety comprising at least one antigen binding site such as a monoclonal antibody, the partial reduction is typically conducted by first diluting to a suitable concentration and buffering the solution before partial reduction of the disulfide bonds by means of the addition of a suitable reducing agent such as tris[2-carboxyethyl]phosphine hydrochloride (TCEP) or dithiothreitol (DTT). By choosing appropriate ratios of the moiety to be reduced such as a monoclonal antibody and the reducing agent, the reaction conditions and the time of the reduction it is possible to obtain a desired free thiol to moiety ratio, e.g. four free thiol groups per monoclonal antibody.

The partially reduced moiety such as the partially reduced monoclonal antibody having the free thiol groups, prepared as described above, is then reacted with drug-linker compounds of the invention of formula D-(X)_{b}-(AA)_{w}-(T)_{g}-L₁ (wherein the group L₁ in such compound is a maleimide group which is free to react with the thiol groups). The resulting drug antibody conjugates are purified by any suitable means known in the art, e.g. by size exclusion chromatography (SEC) [see, e.g., Liu et al., Proc. Natl. Acad. Sci. USA, 93: 8618-8623 (1996), and Chari et al., Cancer Research, 52: 127-131 (1992)].

In one preferred embodiment of this invention, the partially reduced monoclonal antibody is an anti-HER2 antibody such as Trastuzumab or an anti-CD13 antibody or an antigen-binding fragment or an immunologically active portion thereof, preferably Trastuzumab or an antigen-binding fragment or an immunologically active portion thereof; or preferably an anti-CD13 antibody or an antigen-binding fragment or an immunologically active portion thereof.

In an alternative embodiment of the invention, lysines in the moiety comprising at least one antigen binding site such as a monoclonal antibody can first be reacted with succinimidyl-4-(N-maleimidomethyl)cyclohexane-1-carboxylate. A free amine group on an antibody can react with the N-hydroxysuccinimide ester to give a maleimide-activated antibody:

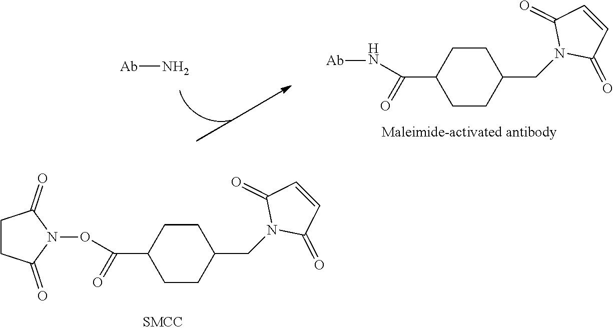

The maleimide-activated antibody can then be reacted with a compound of formula D-(X)_{b}-(AA)_{w}-(T)_{g}-H having a reactive thiol moiety.

In an alternative embodiment of the invention, lysines in the moiety comprising at least one antigen binding site such as a monoclonal antibody can first be reacted with 2-iminothiolane hydrochloride (Traut's reagent). A free amine group on an antibody can react with the imidic thiolactone to give a thiol-activated antibody.

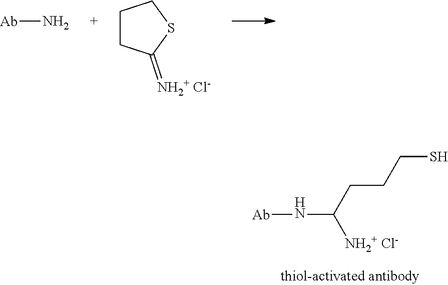

One specific example of processes for the preparation of drug antibody conjugates of formula [D-(X)_{b}-(AA)_{w}-(T)_{g}-(L)-]ₙ-Ab of the present invention by conjugation via free thiol groups in cysteines after partial reduction of disulfide groups in the antibody is shown in FIG. 1.

Another specific example of processes for the preparation of drug antibody conjugates of formula [D-(X)_{b}-(AA)_{w}-(T)_{g}-(L)-]ₙ-Ab of the present invention by conjugation with free amino groups in lysines after reaction of the antibody with Traut's reagent is shown in FIG. 2.

### Compositions Comprising the Drug Antibody Conjugate of the Invention and Uses Thereof

There is also provided a pharmaceutical composition comprising a drug conjugate according to the present invention and a pharmaceutically acceptable carrier. Examples of the administration form of a drug conjugate having the general formula [D-(X)_{b}-(AA)_{w}-(T)_{g}-(L)-]ⱼ-Ab of the present invention include without limitation oral, topical, parenteral, sublingual, rectal, vaginal, ocular, and intranasal. Parenteral administration includes subcutaneous injections, intravenous, intramuscular, intrasternal injection or infusion techniques. Preferably, the compositions are administered parenterally. Pharmaceutical compositions of the invention can be formulated so as to allow a drug conjugate of the present invention to be bioavailable upon administration of the composition to an animal, preferably human. Compositions can take the form of one or more dosage units, where for example, a tablet can be a single dosage unit, and a container of a drug antibody conjugate of the present invention in aerosol form can hold a plurality of dosage units.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, for example, inhalatory administration. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the drug antibody conjugates of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When the composition is in the form of a capsule (e.g. a gelatin capsule), it can contain, in addition to materials of the above type, a liquid carrier such as polyethylene glycol, cyclodextrin or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, the present drug antibody conjugates of the present invention are administered intravenously.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides, polyethylene glycols, glycerin, or other solvents; antibacterial agents such as benzyl alcohol or methyl paraben; and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant.

The amount of the drug conjugate of the present invention that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of a drug conjugate of the present invention such that a suitable dosage will be obtained. The correct dosage of the compounds will vary according to the particular formulation, the mode of application, and its particular site, host and the disease being treated, e.g. cancer and, if so, what type of tumor. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

The drug conjugate of the present invention or compositions can be administered by any convenient route, for example by infusion or bolus injection, by absorption through epithelial or mucocutaneous linings.

In specific embodiments, it can be desirable to administer one or more drug conjugates of the present invention or compositions locally to the area in need of treatment. In one embodiment, administration can be by direct injection at the site (or former site) of a cancer, tumor or neoplastic or pre-neoplastic tissue. In another embodiment, administration can be by direct injection at the site (or former site) of a manifestation of an autoimmune disease.

Pulmonary administration can also be employed, e.g. by use of an inhaler or nebulizer, and formulation with an aerosolizing agent, or via perfusion in a fluorocarbon or synthetic pulmonary surfactant. In certain embodiments, the drug antibody conjugate of the present invention or compositions can be formulated as a suppository, with traditional binders and carriers such as triglycerides.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Other examples of suitable pharmaceutical carriers are described in “Remington's Pharmaceutical Sciences” by E. W. Martin.

The pharmaceutical compositions can be prepared using methodology well known in the pharmaceutical art. For example, a composition intended to be administered by injection can be prepared by combining a drug conjugate of the present invention with water so as to form a solution. A surfactant can be added to facilitate the formation of a homogeneous solution or suspension.

We have found that the drug conjugates and compositions of the present invention are particularly effective in the treatment of cancer.

Thus, as described earlier, the present invention provides a method of treating a patient in need thereof, notably a human, affected by cancer which comprises administering to the affected individual a therapeutically effective amount of a drug conjugate or a composition of the present invention. The present invention provides a drug conjugate according to the present invention for use in the treatment of cancer, and more preferably a cancer selected from lung cancer including NSCLC, gastric cancer, colorectal cancer, breast cancer, pancreas carcinoma, endometrial cancer, bladder cancer, cervical cancer, esophageal cancer, gallbladder cancer, uterine cancer, salivary duct cancer, ovarian cancer, kidney cancer, leukaemia, multiple myeloma, and lymphoma. Most preferred cancer is breast cancer. The cancer is preferably a HER2 positive cancer, wherein the HER2 positive cancers include HER2 positive lung cancer including HER2 positive NSCLC, HER2 positive gastric cancer, HER2 positive colorectal cancer, HER2 positive breast cancer, HER2 positive pancreas carcinoma, HER2 positive endometrial cancer, HER2 positive bladder cancer, HER2 positive cervical cancer, HER2 positive esophageal cancer, HER2 positive gallbladder cancer, HER2 positive uterine cancer, HER2 positive salivary duct cancer and HER2 positive ovarian cancer, more preferably HER2 positive breast cancer, HER2 positive ovarian cancer and HER2 positive gastric cancer, most preferably HER2 positive breast cancer.

The drug conjugates and compositions of the present invention are useful for inhibiting the multiplication of a tumor cell or cancer cell, or for treating cancer in an animal. The drug conjugates and compositions of the present invention can be used accordingly in a variety of settings for the treatment of animal cancers. The conjugates of the invention comprising Drug-Linker-Moiety comprising at least one antigen binding site can be used to deliver a Drug or Drug unit to a tumor cell or cancer cell. Without being bound by theory, in one embodiment, the Moiety comprising at least one antigen binding site of a drug conjugate of the present invention binds to or associates with a cancer-cell or a tumor-cell-associated antigen, and the drug conjugate of the present invention can be taken up inside a tumor cell or cancer cell through receptor-mediated endocytosis. The antigen can be attached to a tumor cell or cancer cell or can be an extracellular matrix protein associated with the tumor cell or cancer cell. Once inside the cell, one or more specific sequences within the Linker unit are hydrolytically cleaved by one or more tumor-cell or cancer-cell-associated proteases or hydrolases, resulting in release of a Drug or a Drug-Linker Compound. The released Drug or Drug-Linker Compound is then free to migrate in the cell and induce cytotoxic activities. In an alternative embodiment, the Drug or Drug unit is cleaved from the drug conjugate of the present invention outside the tumor cell or cancer cell, and the Drug or Drug-Linker Compound subsequently penetrates the cell.

In one embodiment, the Moiety comprising at least one antigen binding site binds to the tumor cell or cancer cell. In another embodiment, the Moiety comprising at least one antigen binding site binds to a tumor cell or cancer cell antigen which is on the surface of the tumor cell or cancer cell. In yet another embodiment, the Moiety comprising at least one antigen binding site binds to a tumor cell or cancer cell antigen which is an extracellular matrix protein associated with the tumor cell or cancer cell.

The specificity of the Moiety comprising at least one antigen binding site for a particular tumor cell or cancer cell can be important for determining those tumors or cancers that are most effectively treated. For example, drug conjugates of the present invention having a Trastuzumab unit can be useful for treating antigen positive carcinomas including leukaemias, lung cancer, colon cancer, lymphomas (e.g. Hodgkin's disease, non-Hodgkin's Lymphoma), solid tumors such as, sarcoma and carcinomas, Multiple myeloma, kidney cancer and melanoma. The cancer may preferably be lung cancer, colorectal cancer, breast cancer, pancreas carcinoma, kidney cancer, leukaemia, multiple myeloma, lymphoma or ovarian cancer. For example, drug conjugates of the present invention having a Rituximab unit can be useful for treating CD-20 expressing tumors such as haematological cancers including leukemias and lymphomas. For example, drug conjugates of the present invention having an anti-CD4 antibody unit can be useful for treating CD-4 expressing tumors such as haematological cancers including lymphomas. For example, drug conjugates of the present invention having an anti-CD5 antibody unit can be useful for treating CD-5 expressing tumors such as haematological cancers including leukemias and lymphomas. For example, drug conjugates of the present invention having an anti-CD13 antibody unit can be useful for treating CD-13 expressing tumors such as haematological cancers including leukemias and lymphomas.

Other particular types of cancers that can be treated with drug conjugates of the present invention include, but are not limited to: blood-borne cancers including all forms of leukemia; lymphomas, such as Hodgkin's disease, non-Hodgkin's Lymphoma and Multiple myeloma.

In particular, the drug conjugates and compositions of the present invention show excellent activity in the treatment of breast cancer.

Drug conjugates and compositions of the present invention provide conjugation specific tumor or cancer targeting, thus reducing general toxicity of these conjugates. The Linker units stabilize the drug antibody conjugates in blood, yet are cleavable by tumor-specific proteases and hydrolases within the cell, liberating a Drug.

The drug conjugates and compositions of the present invention can be administered to an animal that has also undergone surgery as treatment for the cancer. In one embodiment of the present invention, the additional method of treatment is radiation therapy.

In a specific embodiment of the present invention, the drug conjugate or composition of the present invention may be administered with radiotherapy. Radiotherapy may be administered at the same time, prior to or after treatment with the drug conjugate or composition of the present invention. In an embodiment, the drug conjugate or composition of the present invention is administered concurrently with radiation therapy. In another specific embodiment, the radiation therapy is administered prior or subsequent to administration of a drug conjugate or composition of the present invention, preferably at least an hour, five hours, 12 hours, a day, a week, a month, more preferably several months (e.g. up to three months), prior or subsequent to administration of a drug antibody conjugate or composition of the present invention.

With respect to radiation, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

In the present invention, there is provided a kit comprising a therapeutically effective amount of a drug conjugate according to the present invention and a pharmaceutically acceptable carrier. In an embodiment, there is provided a kit comprising a composition according to the present invention and, optionally, instructions for use in the treatment of cancer, and more preferably a cancer selected from lung cancer including NSCLC, gastric cancer, colorectal cancer, breast cancer, pancreas carcinoma, endometrial cancer, bladder cancer, cervical cancer, esophageal cancer, gallbladder cancer, uterine cancer, salivary duct cancer, ovarian cancer, kidney cancer, leukaemia, multiple myeloma, and lymphoma.

In one embodiment, the kit according to this aspect is for use in the treatment of cancer, and more preferably a cancer selected from lung cancer including NSCLC, gastric cancer, colorectal cancer, breast cancer, pancreas carcinoma, endometrial cancer, bladder cancer, cervical cancer, esophageal cancer, gallbladder cancer, uterine cancer, salivary duct cancer, ovarian cancer, kidney cancer, leukaemia, multiple myeloma, and lymphoma. Most preferred kit is for use in the treatment of breast cancer.

### BRIEF DESCRIPTION OF THE DRAWINGS

The invention is diagrammatically illustrated, by way of example, in the accompanying drawings in which:

FIG. 1 is a schematic illustration of one process according to the present invention wherein conjugation to the antibody is via free thiol groups;

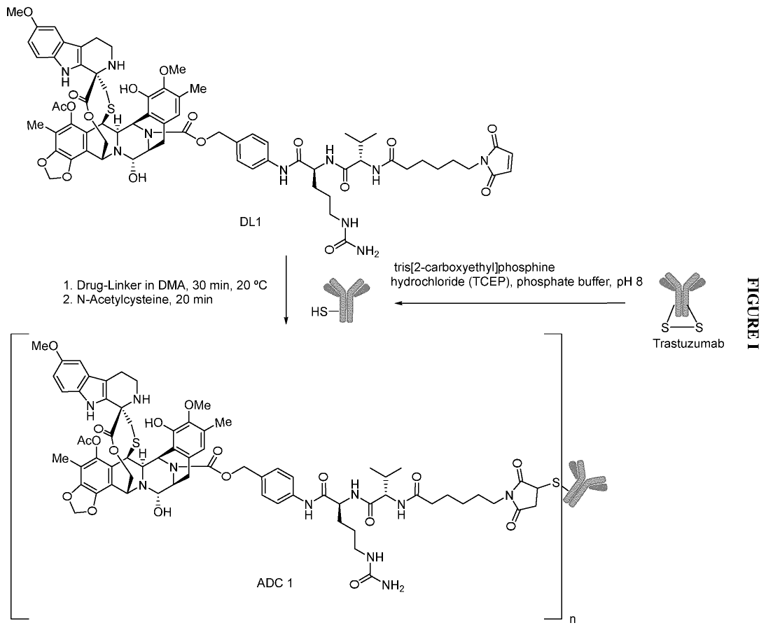

FIG. 2 is a schematic illustration of one process according to the present invention wherein conjugation to the antibody is via free amino groups.

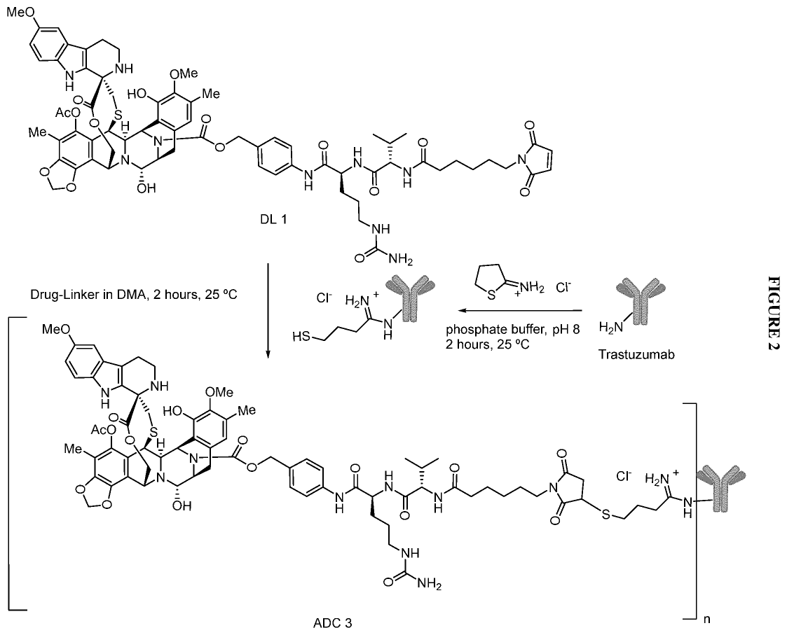

### EXAMPLES

The present invention is further illustrated by way of the following, non-limiting examples. In the examples, the following abbreviations are used:
CDI, 1,1′-Carbonyldiimidazole
DIPEA, N,N-Diisopropylethylamine
Hex, Hexane
EtOAc, Ethyl acetate
DCM, Dichloromethane
NMP, N-Methyl-2-pyrrolidone
DMF, Dimethylformamide
EDC, N-(3-Dimethylaminopropyl)-N′-ethylcarbodiimide hydrochloride
EDTA, Ethylenediaminetetraacetic acid
MeOH, Methanol
DTT, Dithiothreitol
Py, Pyridine
THF, Tetrahydrofuran
TCEP, Tris[2-carboxyethyl]phosphine hydrochloride
MC, 6-Maleimidocaproyl
Fmoc, 9-Fluorenylmethoxycarbonyl
Cit, Citrulline
Val, Valine
DMSO, Dimethylsulfoxide
Trt, Triphenylmethyl
HOBt, 1-Hydroxybenzotriazole
DIPCDI, N,N′-Diisopropylcarbodiimide
TFA, Trifluoroacetic acid
PABOH, 4-Aminobenzyl alcohol
bis-PNP, bis(4-Nitrophenyl) carbonate
NAC, N-Acetylcysteine
SEC, Size-Exclusion Chromatography
HPLC, High Performance Liquid Chromatography
ADC, Antibody Drug Conjugate
ATCC, American Type Culture Collection
DMEM, Dulbecco's Modified Eagle's Medium
RPMI, Rosmell Park Memorial Institute Medium
ITS, Insulin-transferrin-sodium selenite media supplement
FCS, Fetal Calf Serum
SRB, Sulforhodamine B
PBS, Phosphate Buffered Saline
DR, Dose-Response
UV, Ultraviolet
SMCC, Succinimidyl-4-(N-maleimidomethyl)cyclohexane-1-carboxylate
LAR, Linker to Antibody Ratio

### Example 1. Synthesis of Drugs

Compounds 1 and 2 were obtained following the procedures described in WO2003066638 (Examples 69 and 65, respectively, at pages 112-116).

Compound 4 was obtained following the procedure described in WO2003066638 (Example 12, at pages 61-62).

Compounds 8-S and 8-R were obtained following the procedure described in WO2018197663 (Example 8, at pages 97-98).

Compound 16-S was obtained following the procedure described in WO2018197663 (Example 19, at page 117).

### Example 1-1

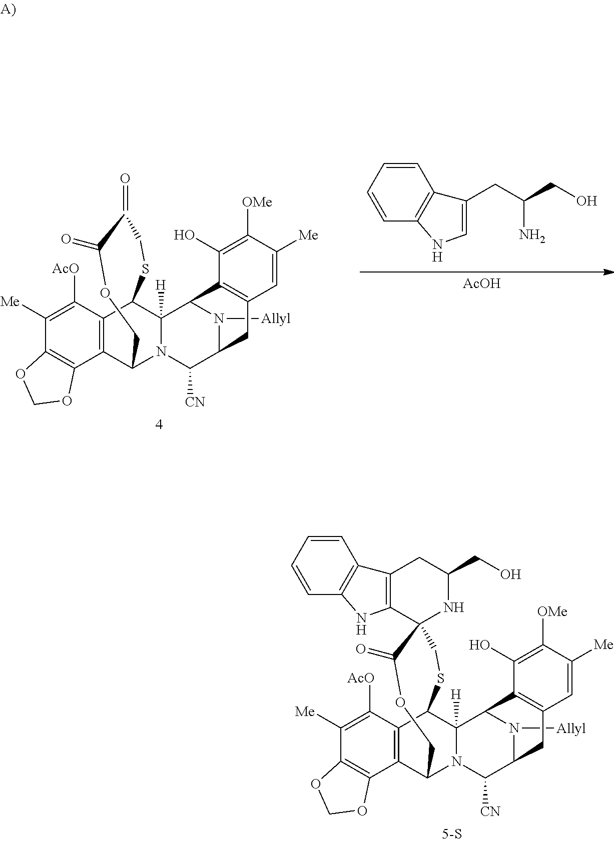

To a solution of 4 (35 mg, 0.054 mmol) in acetic acid (0.7 mL, 0.08 M) was added L-Tryptophanol (36 mg, 0.189 mmol, Sigma-Aldrich). The reaction mixture was stirred a 50° C. for 3 h and then acetic acid was evaporated. An aqueous saturated solution of NaHCO₃ was added and the mixture was extracted with CH₂Cl₂. The combined organic layers were dried over Na₂SO₄. Flash chromatography (Hexane:EtOAc, 1:1) gives pure compound 5-S (28 mg, 63%).

R_{f}=0.25 (Hexane:EtOAc, 1:1).

¹H NMR (400 MHz, CDCl₃): δ 7.72 (s, 1H), 7.35 (d, J=7.9 Hz, 1H), 7.26 (d, J=7.9 Hz, 1H), 7.12 (t, J=7.9 Hz, 1H), 7.02 (t, J=8.0 Hz, 1H), 6.62 (s, 1H), 6.25 (s, 1H), 6.03 (s, 1H), 5.91-5.80 (m, 1H), 5.75 (s, 1H), 5.17-5.04 (m, 3H), 4.60 (s, 1H), 4.41 (s, 1H), 4.36 (d, J=11.5 Hz, 1H), 4.29 (dd, J=11.7, 2.1 Hz, 1H), 4.22 (d, J=2.7 Hz, 1H), 3.81 (s, 3H), 3.59-3.44 (m, 3H), 3.35 (dd, J=11.1, 9.0 Hz, 1H), 2.97-2.64 (m, 5H), 2.61 (dd, J=15.3, 4.6 Hz, 1H), 2.43-2.29 (m, 1H), 2.37 (s, 3H), 2.28 (s, 3H), 2.05 (s, 3H).

ESI-MS m/z: Calcd. for C₄₄H₄₅N₅O₉S: 819.3. Found: 820.3 (M+1)⁺.

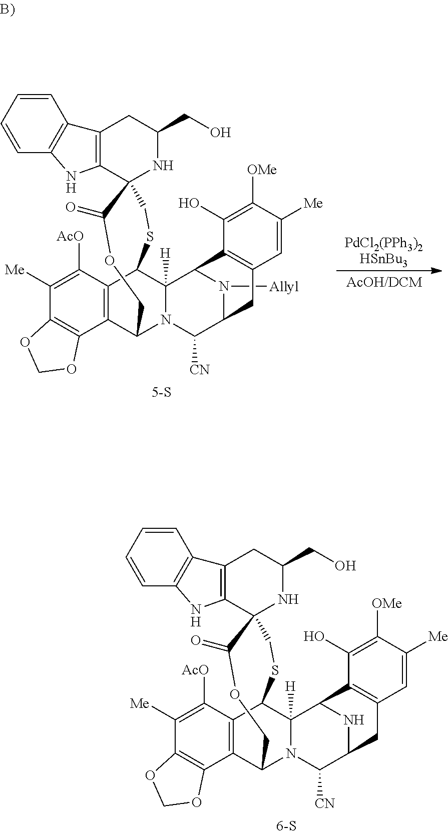

To a solution of 5-S (26 mg, 0.032 mmol) in CH₂Cl₂ was added PdCl₂(PPh₃)₂ (13 mg, 0.02 mmol), acetic acid (0.069 mL, 1.2 mmol) and HSnBu₃ (0.17 mL, 0.64 mmol). The reaction mixture was stirred a 23° C. for 2 h. The crude was concentrated under vacuum. Flash chromatography (Hexane:EtOAc, from 1:9 to 9:1) gives pure compound 6-S (17 mg, 68%).

R_{f}=0.15 (Hexane:EtOAc, 1:1).

¹H NMR (400 MHz, CDCl₃): δ 7.74 (s, 1H), 7.38 (d, J=7.9 Hz, 1H), 7.26 (d, J=7.9 Hz, 1H), 7.12 (t, J=8.0 Hz, 1H), 7.02 (t, J=7.9 Hz, 1H), 6.63 (s, 1H), 6.26 (s 1H), 6.03 (s, 1H), 5.82 (s, 1H), 5.15 (d, J=11.6 Hz, 1H), 4.59 (s, 1H), 4.50 (d, J=5.1 Hz, 1H), 4.41 (s, 1H), 4.29 (dd, J=11.7, 2.1 Hz, 1H), 4.22 (d, J=2.7 Hz, 1H), 3.90-3.81 (m, 1H), 3.80 (s, 3H), 3.67-3.49 (m, 1H), 3.49 (d, J=5.2 Hz, 1H), 3.36 (dd, J=11.0, 9.1 Hz, 1H), 3.11-2.85 (m, 3H), 2.60 (dd, J=15.3, 4.5 Hz, 1H), 2.42 (d, J=15.3 Hz, 1H), 2.38-2.22 (m, 2H), 2.35 (s, 3H), 2.25 (s, 3H), 2.06 (s, 3H).

ESI-MS m/z: Calcd. for C₄₁H₄₁N₅O₉S: 779.3. Found: 780.2 (M+1)⁺.

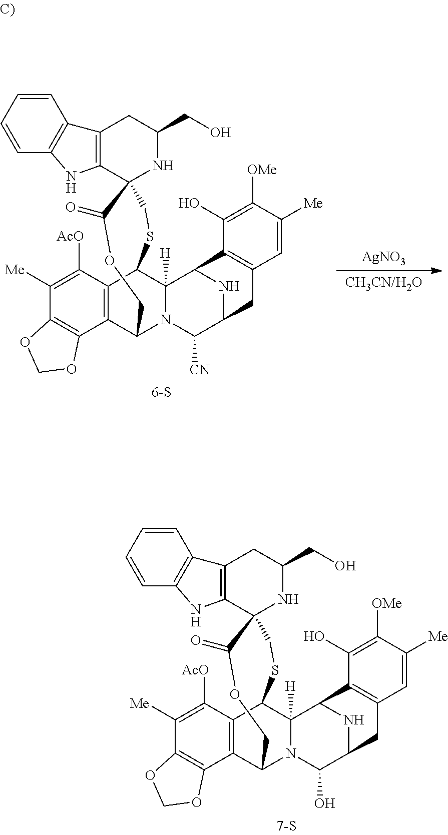

To a solution of 6-S (14 mg, 0.018 mmol) in CH₃CN:H₂O (1.39:1, 1.3 mL, 0.015 M) was added AgNO₃ (61 mg, 0.36 mmol). After 17 h at 23° C., the reaction was quenched with a mixture 1:1 of saturated aqueous solutions of brine and NaHCO₃, stirred for 15 min, diluted with CH₂Cl₂, stirred for 5 min, and extracted with CH₂Cl₂. The combined organic layers were dried over anhydrous Na₂SO₄, filtered, and concentrated under vacuum. The residue obtained was purified by flash chromatography (CH₂Cl₂:CH₃OH, from 99:1 to 85:15) to give pure 7-S (3 mg, 22%).

R_{f}=0.15 (CH₂Cl₂:CH₃OH, 9:1).

¹H NMR (500 MHz, CD₃OD): δ 7.70 (s, 1H), 7.35 (d, J=7.9 Hz, 1H), 7.26 (d, J=7.9 Hz, 1H), 7.11 (t, J=8.2 Hz, 1H), 7.02 (t, J=8.2 Hz, 1H), 6.63 (s, 1H), 6.23 (s, 1H), 6.01 (s, 1H), 5.76 (s, 1H), 5.26 (d, J=11.5 Hz, 1H), 4.92 (s, 1H), 4.54 (s, 1H), 4.48 (s, 2H), 4.37 (d, J=5.3 Hz, 1H), 4.21 (d, J=10.2 Hz, 1H), 3.80 (s, 3H), 3.67-3.50 (m, 4H), 3.36 (t, J=10.2 Hz, 1H), 3.04-2.82 (m, 3H), 2.61 (dd, J=15.2, 5.8 Hz, 1H), 2.42-2.28 (m, 2H), 2.36 (s, 3H), 2.27 (s, 3H), 2.02 (s, 3H).

ESI-MS m/z: Calcd. for C₄₀H₄₂N₄O₁₀S: 770.3. Found: 753.2 (M−H₂O+1)⁺.

### Example 1-2

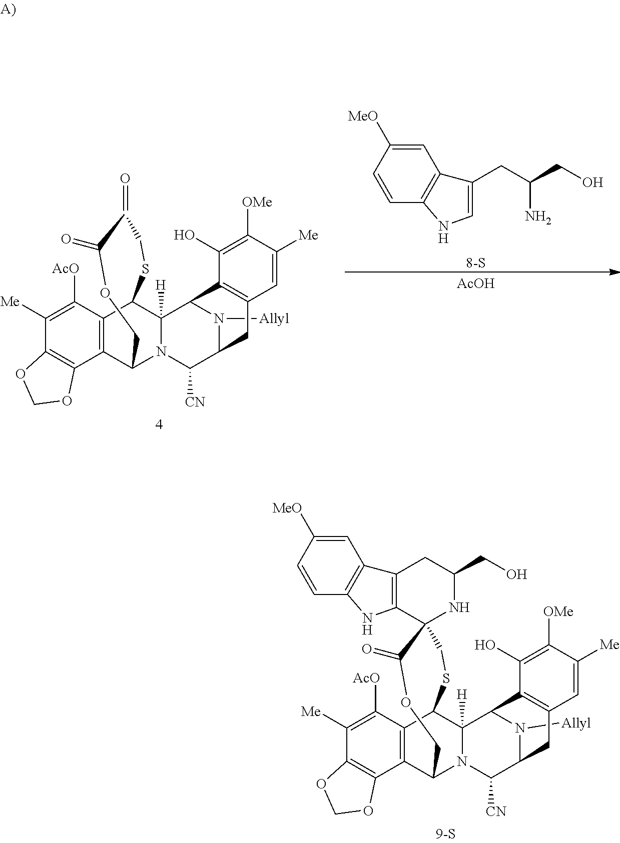

To a solution of 4 (400 mg, 0.62 mmol) in acetic acid (8 mL, 0.08 M) was added 8-S (468 mg, 2.13 mmol). The reaction mixture was stirred a 52° C. for 17 h and then acetic acid was evaporated. An aqueous saturated solution of NaHCO₃ was added and the mixture was extracted with CH₂Cl₂. The combined organic layers were dried over Na₂SO₄, filtered, and concentrated under vacuum. Flash chromatography (Hexane:EtOAc, 1:1) gives pure compound 9-S (325 mg, 62%).

R_{f}=0.30 (Hexane:EtOAc, 1:1).

¹H NMR (400 MHz, CDCl₃): δ 7.65 (s, 1H), 7.16 (d, J=8.6 Hz, 1H), 6.78 (s, 1H), 6.77 (m, 1H), 6.62 (s, 2H), 6.23 (d, J=1.3 Hz, 2H), 6.02 (d, J=1.3 Hz, 2H), 5.85 (dddd, J=17.1, 10.2, 6.8, 5.8 Hz, 1H), 5.75 (s, 1H), 5.15-5.00 (m, 3H), 4.59 (s, 1H), 4.43-4.22 (m, 4H), 3.80 (s, 3H), 3.78 (s, 3H), 3.53 (d, J=12.9 Hz, 2H), 3.46 (d, J=5.0 Hz, 1H), 3.38 (s, 1H), 2.93 (s, 1H), 2.86 (d, J=4.4 Hz, 1H), 2.85-2.70 (m, 2H), 2.58 (dd, J=15.2, 4.6 Hz, 1H), 2.42-2.30 (m, 2H), 2.37 (s, 3H), 2.26 (s, 3H), 2.04 (s, 3H).

ESI-MS m/z: Calcd. for C₄₅H₄₇N₅O₁OS: 849.9. Found: 850.3 (M+1)⁺.

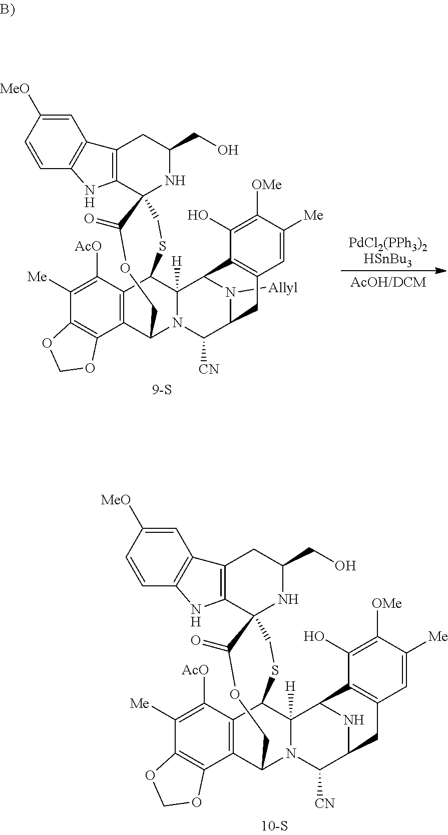

To a solution of 9-S (325 mg, 0.38 mmol) in CH₂Cl₂ was added PdCl₂(PPh₃)₂ (160 mg, 0.23 mmol), acetic acid (0.82 mL, 14.2 mmol) and HSnBu₃ (1.7 mL, 6.27 mmol). The reaction mixture was stirred a 23° C. for 1.5 h. The crude was concentrated under vacuum. Flash chromatography (Hexane:EtOAc, from 1:9 to 9:1) gives pure compound 10-S (180 mg, 59%).

R_{f}=0.15 (Hexane:EtOAc, 1:1).

¹H NMR (400 MHz, CDCl₃): δ 7.19 (s, 1H), 6.79 (m, 2H), 6.65 (s, 1H), 6.26 (s, 1H), 6.03 (d, J=1.4 Hz, 2H), 5.77 (d, J=11.5 Hz, 1H), 5.10 (s, 1H), 4.59 (s, 1H), 4.48 (d, J=4.9 Hz, 1H), 4.39-4.29 (m, 3H), 3.79 (s, 3H), 3.79 (s, 3H), 3.64-3.33 (m, 4H), 3.03-2.90 (m, 4H), 2.59 (d, J=14.6 Hz, 2H), 2.44-2.32 (m, 2H), 2.37 (s, 3H), 2.26 (s, 3H), 2.04 (s, 3H).

ESI-MS m/z: Calcd. for C₄₂H₄₃N₅O₁OS: 809.3. Found: 810.3 (M+1)⁺.

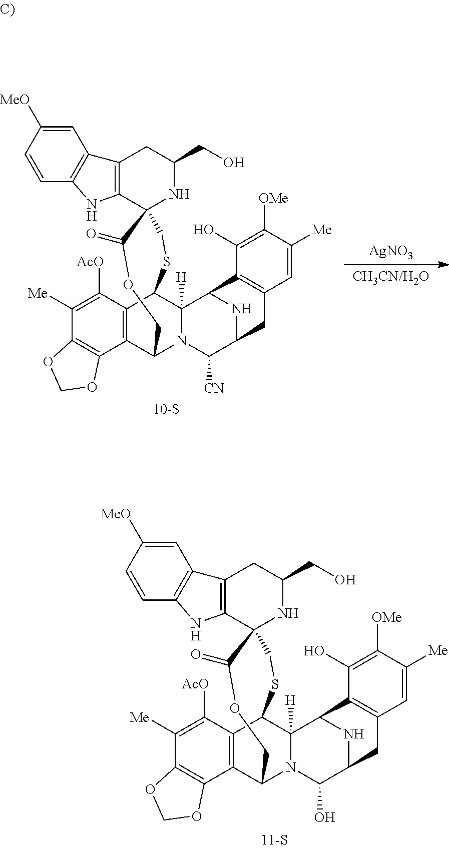

To a solution of 10-S (180 mg, 0.22 mmol) in CH₃CN:H₂O (1.39:1, 16 mL, 0.015 M) was added AgNO₃ (756 mg, 4.40 mmol). After 18 h at 23° C., the reaction was quenched with a mixture 1:1 of saturated aqueous solutions of brine and NaHCO₃, stirred for 15 min, diluted with CH₂C₂, stirred for 5 min, and extracted with CH₂C₂. The combined organic layers were dried over anhydrous Na₂SO₄, filtered, and concentrated under vacuum. The residue obtained was purified by flash chromatography (CH₂Cl₂:CH₃OH, from 99:1 to 85:15) to give pure 11-S (100 mg, 56%).

R_{f}=0.35 (CH₂Cl₂:CH₃OH, 9:1).

¹H NMR (500 MHz, CD₃OD): δ 7.15 (dd, J=8.8, 0.6 Hz, 1H), 6.82 (dd, J=2.5, 0.6 Hz, 1H), 6.68 (dd, J=8.9, 2.5 Hz, 1H), 6.56 (s, 1H), 6.27 (d, J=1.3 Hz, 1H), 6.08 (d, J=1.3 Hz, 1H), 5.31 (d, J=11.5 Hz, 1H), 4.62-4.55 (m, 1H), 4.44 (ddtd, J=4.9, 1.5, 1.0, 0.5 Hz, 2H) 4.38-4.27 (m, 1H), 4.25-4.18 (m, 1H), 3.75 (s, 3H), 3.74 (s, 3H), 3.64 (d, J=4.8 Hz, 1H), 3.61-3.42 (m, 3H), 3.13-2.95 (m, 3H), 2.80 (dd, J=10.4, 5.4 Hz, 2H), 2.68 (dd, J=15.1, 4.2 Hz, 2H), 2.55 (d, J=15.4 Hz, 1H), 2.51-2.36 (m, 3H), 2.34 (s, 3H), 2.29 (s, 3H), 2.00 (s, 3H).

¹³C NMR (126 MHz, CD₃OD): δ 172.6, 169.2, 155.1, 148.0, 147.2, 144.7, 142.4, 142.1, 133.1, 132.6, 132.2, 131.1, 128.2, 125.5, 122.2, 122.0, 116.3, 112.9, 112.8, 111.4, 109.0, 103.5, 100.9, 91.0, 66.6, 65.0, 61.8, 60.3, 59.2, 57.1, 56.1, 51.7, 47.2, 45.5, 43.8, 39.0, 28.2, 25.4, 20.6, 16.3, 9.5.

ESI-MS m/z: Calcd. for C₄₁H₄₄N₄O₁₁S: 800.3. Found: 783.4 (M−H₂O+1)⁺.

(+)-HR-ESI-TOF-MS m z 800.2796 [M+H]⁺ (calcd. for C₄₁H₄₄N₄O₁₁S: 800.2727).

### Example 1-3

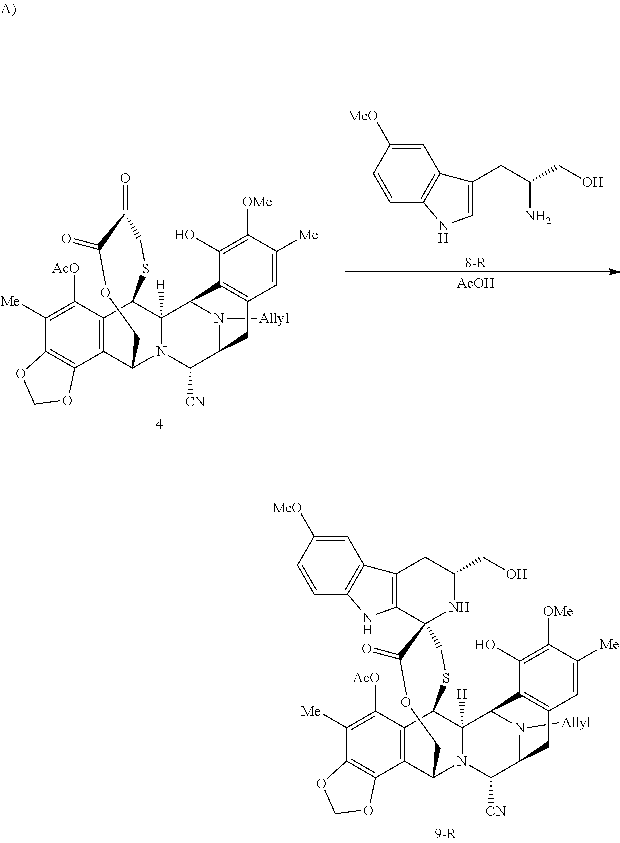

To a solution of 4 (400 mg, 0.62 mmol) in acetic acid (8 mL, 0.08 M) was added 8-R (468 mg, 2.13 mmol). The reaction mixture was stirred at 52° C. for 17 h and then acetic acid was evaporated. An aqueous saturated solution of NaHCO₃ was added and the mixture was extracted with CH₂C₂. The combined organic layers were dried over Na₂SO₄, filtered, and concentrated under vacuum. Flash chromatography (Hexane:EtOAc, 1:1) gives pure compound 9-R (390 mg, 77%).

R_{f}=0.30 (Hexane:EtOAc, 1:1).

¹H NMR (400 MHz, CDCl₃): δ 7.64 (s, 1H), 7.14 (d, J=8.8 Hz, 1H), 6.81 (d, J=2.6 Hz, 1H), 6.74 (dd, J=8.8, 2.4 Hz, 1H), 6.59 (s, 1H), 6.18 (d, J=1.4 Hz, 1H), 5.97 (d, J=1.4 Hz, 1H), 5.91-5.80 (m, 1H), 5.79 (s, 1H), 5.15-4.92 (m, 3H), 4.62 (s, 1H), 4.42-4.23 (m, 2H), 4.23-4.03 (m, 3H), 3.79 (s, 3H), 3.78 (s, 3H), 3.68-3.48 (m, 2H), 3.43 (d, J=5.1 Hz, 2H), 3.01-2.68 (m, 3H), 2.57-2.41 (m, 3H), 2.39 (s, 3H), 2.25 (s, 3H), 2.22-2.20 (m, 1H), 2.07 (s, 3H).

ESI-MS m/z: Calcd. for C₄₅H₄₇N₅O₁OS: 849.9. Found: 850.4 (M+1)⁺.

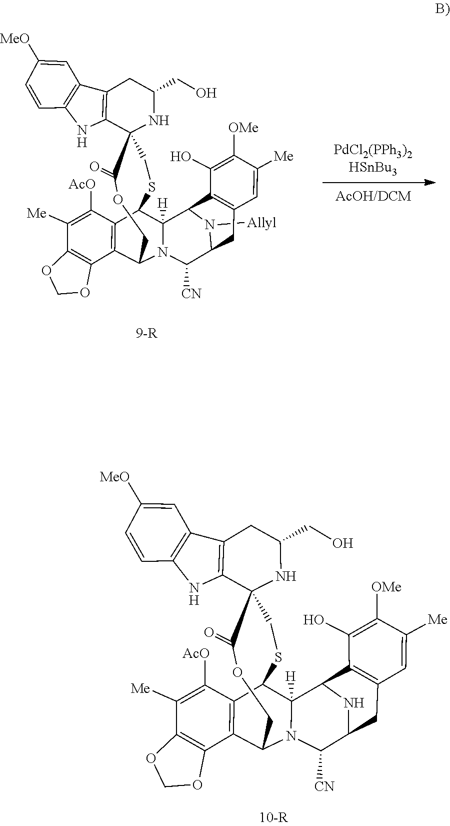

To a solution of 9-R (390 mg, 0.46 mmol) in CH₂Cl₂ was added PdCl₂(PPh₃)₂ (193 mg, 0.28 mmol), acetic acid (1.0 mL, 17.2 mmol) and HSnBu₃ (2.04 mL, 7.60 mmol). The reaction mixture was stirred a 23° C. for 1.5 h. The crude was concentrated under vacuum. Flash chromatography (Hexane:EtOAc, from 1:9 to 9:1) gave pure compound 10-R (210 mg, 57%).

R_{f}=0.15 (Hexane:EtOAc, 1:1).

¹H NMR (400 MHz, CDCl₃): δ 7.16 (d, J=8.8 Hz, 1H), 6.82 (d, J=2.4 Hz, 1H), 6.76 (dd, J=8.8, 2.5 Hz, 1H), 6.62 (s, 1H), 6.22 (d, J=1.5 Hz, 1H), 6.00 (d, J=1.5 Hz, 1H), 5.03 (d, J=11.5 Hz, 1H), 4.62 (s, 1H), 4.51 (d, J=5.0 Hz, 1H), 4.36 (s, 1H), 4.23-4.11 (m, 3H), 3.85 (dd, J=8.1, 4.1 Hz, 1H), 3.80 (s, 3H), 3.78 (s, 3H), 3.72-3.57 (m, 1H), 3.45 (d, J=5.2 Hz, 2H), 3.12 (d, J=17.6 Hz, 1H), 2.99 (dd, J=17.9, 9.6 Hz, 1H), 2.54 (s, 2H), 2.46 (d, J=14.7 Hz, 2H), 2.38 (s, 3H), 2.33-2.19 (m, 1H), 2.26 (s, 3H), 2.08 (s, 3H).

ESI-MS m/z: Calcd. for C₄₂H₄₃N₅O₁OS: 809.3. Found: 810.5 (M+1)⁺.

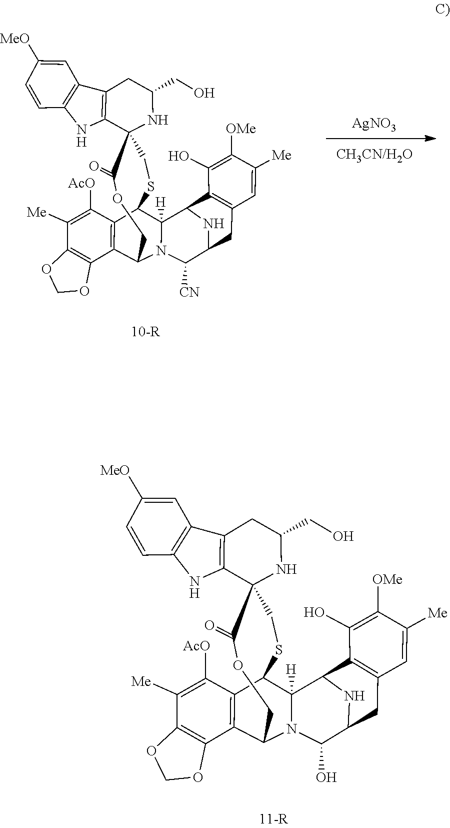 [SMILES: COc1ccc2[nH]c3c(c2c1)C[C@H](CO)N[C@]31CS[C@@H]2c3c(OC(C)=O)c(C)c4c(c3[C@H](COC1=O)N1[C@@H]2[C@@H]2N[C@@H](Cc3cc(C)c(OC)c(O)c32)[C@@H]1C#N)OCO4 ; "AgNO3" ; COc1ccc2[nH]c3c(c2c1)C[C@H](CO)N[C@]31CS[C@@H]2c3c(OC(C)=O)c(C)c4c(c3[C@H](COC1=O)N1[C@@H]2[C@@H]2N[C@@H](Cc3cc(C)c(OC)c(O)c32)[C@@H]1O)OCO4]

To a solution of 10-R (210 mg, 0.26 mmol) in CH₃CN:H₂O (1.39:1, 18 mL, 0.015 M) was added AgNO₃ (883 mg, 5.20 mmol). After 18 h at 23° C., the reaction was quenched with a mixture 1:1 of saturated aqueous solutions of brine and NaHCO₃, stirred for 15 min, diluted with CH₂Cl₂, stirred for 5 min, and extracted with CH₂Cl₂. The combined organic layers were dried over anhydrous Na₂SO₄, filtered, and concentrated under vacuum. The residue obtained was purified by flash chromatography (CH₂Cl₂:CH₃OH, from 99:1 to 85:15) to give pure 11-R (140 mg, 66%).

R_{f}=0.30 (CH₂Cl₂:CH₃OH, 9:1).

¹H NMR (500 MHz, CD₃OD): δ 7.13 (dd, J=8.8, 0.6 Hz, 1H), 6.82 (d, J=8.8 Hz, 1H), 6.67 (dd, J=8.8, 2.5 Hz, 1H), 6.61 (s, 1H), 6.25 (d, J=1.3 Hz, 1H), 6.07 (d, J=1.4 Hz, 1H), 5.21 (d, J=11.3 Hz, 1H), 4.80-4.72 (m, 2H), 4.58 (s, 1H), 4.45 (d, J=5.4 Hz, 1H), 4.21 (d, J=2.7 Hz, 1H), 4.16-4.06 (m, 1H), 3.75 (s, 3H), 3.72 (s, 3H), 3.64 (d, J=7.7 Hz, 2H), 3.55-3.48 (m, 3H), 3.16 (d, J=17.6 Hz, 1H), 3.03 (dd, J=17.7, 9.8 Hz, 1H), 2.76-2.63 (m, 2H), 2.32 (s, 3H), 2.29 (s, 3H), 2.25-2.11 (m, 2H), 2.04 (s, 3H).

¹³C NMR (126 MHz, CD₃OD): δ 171.6, 153.6, 146.6, 145.9, 143.4, 141.3, 140.9, 132.1, 131.1, 130.8, 129.7, 126.4, 121.2, 120.7, 114.8, 112.1, 111.5, 110.0, 108.8, 107.6, 107.6, 102.1, 99.5, 89.6, 65.4, 63.1, 60.1, 59.0, 57.8, 55.9, 54.7, 52.7, 45.9, 26.6, 25.1, 24.2, 19.4, 19.1, 14.8, 13.0, 8.2.

ESI-MS m/z: Calcd. for C₄₁H₄₄N₄O₁₁S: 800.3. Found: 783.3 (M−H₂O+1)⁺.

(+)-HR-ESI-TOF-MS m z 800.2781 [M+H]⁺ (calcd. for C₄₁H₄₄N₄O₁₁S: 800.2727).

### Example 1-4

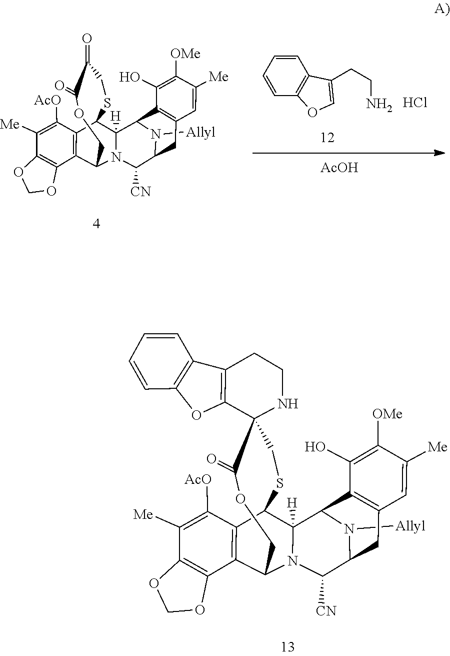

To a solution of 4 (350 mg, 0.54 mmol) in acetic acid (7 mL, 0.08 M) was added 2-benzofuran-3-yl-ethylamine hydrochloride (12) (1.52 g, 7.70 mmol, Sigma Aldrich). The reaction mixture was stirred at 52° C. for 72 h and then acetic acid was evaporated. An aqueous saturated solution of NaHCO₃ was added and the mixture was extracted with CH₂Cl₂. The combined organic layers were dried over anhydrous Na₂SO₄, filtered, and concentrated under vacuum. Flash chromatography (Hexane:EtOAc, 1:1) yields pure 13 (180 mg, 42%).

R_{f}=0.5 (Hexane:EtOAc, 1:1).

¹H NMR (400 MHz, CDCl₃): δ 7.39-7.29 (m, 2H), 7.23-7.07 (m, 2H), 6.64 (s, 1H), 6.19 (d, J=1.3 Hz, 1H), 6.04 (d, J=1.3 Hz, 1H), 5.97-5.80 (m, 1H), 5.78 (s, 1H), 5.19-4.97 (m, 3H), 4.54 (s, 1H), 4.36 (dd, J=4.8, 1.6 Hz, 1H), 4.31 (s, 1H), 4.20 (dd, J=11.4, 1.9 Hz, 2H), 3.80 (s, 3H), 3.59-3.49 (m, 1H), 3.47 (dd, J=7.0, 2.9 Hz, 1H), 3.25 (ddd, J=11.4, 8.1, 5.0 Hz, 1H), 3.04 (d, J=18.0 Hz, 1H), 2.98-2.72 (m, 5H), 2.59-2.49 (m, 2H), 2.37 (s, 3H), 2.27 (s, 3H), 2.23-2.12 (m, 1H), 2.07 (s, 3H).

ESI-MS m/z: Calcd. for C₄₃H₄₂N₄O₉S: 790.9. Found: 791.5 (M+1)⁺.

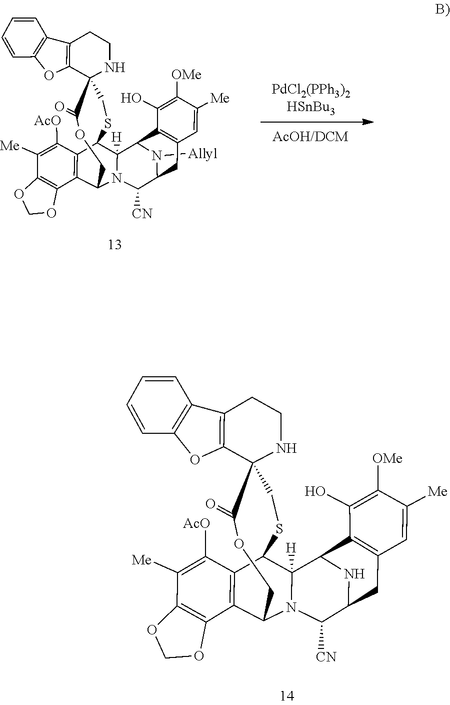

To a solution of 13 (320 mg, 0.40 mmol) in CH₂Cl₂ was added PdCl₂(PPh₃)₂ (170 mg, 0.24 mmol), acetic acid (0.86 mL, 15 mmol) and SnBu₃H (1.78 mL, 6.60 mmol). The reaction mixture was stirred at 23° C. for 1.5 h. The crude was concentrated under vacuum. Flash chromatography (Hexane:EtOAc, from 1:9 to 9:1) affords pure 14 (130 mg, 43%).

R_{f}=0.15 (Hexane:EtOAc, 1:1).

¹H NMR (400 MHz, CDCl₃): δ 7.40-7.31 (m, 2H), 7.19-7.10 (m, 2H), 6.65 (s, 1H), 6.19 (d, J=1.5 Hz, 1H), 6.04 (d, J=1.5 Hz, 1H), 5.05 (d, J=11.5 Hz, 1H), 4.43 (s, 1H), 4.51-4.47 (m, 1H), 4.30 (s, 1H), 4.21 (d, J=2.3 Hz, 2H), 3.86-3.76 (m, 2H), 3.79 (d, J=1.9 Hz, 2H), 3.46 (d, J=4.7 Hz, 1H), 3.29-3.22 (m, 1H), 3.19 (d, J=17.9 Hz, 1H), 2.99 (dd, J=17.9, 9.4 Hz, 1H), 2.83 (s, 1H), 2.53 (dt, J=7.9, 4.8 Hz, 2H), 2.35 (s, 3H), 2.33-2.23 (m, 1H), 2.27 (s, 3H), 2.20-2.14 (m, 1H), 2.07 (m, 3H).

ESI-MS m/z: Calcd. for C₄₀H₃₈N₄O₉S: 750.8. Found: 751.9 (M+1)⁺.

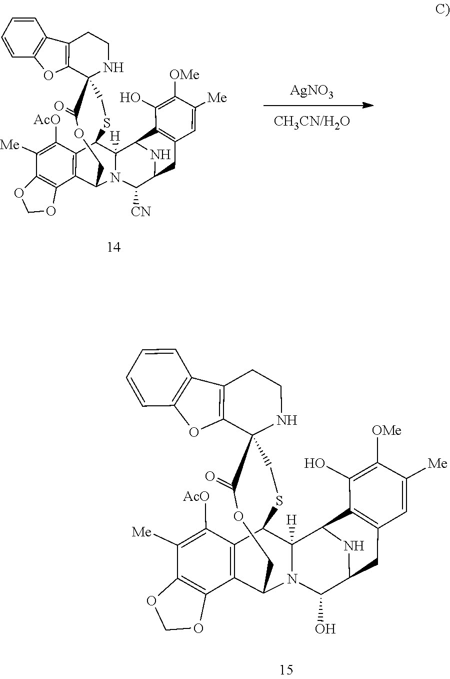 [SMILES: COc1c(C)cc2c(c1O)[C@H]1N[C@@H](C2)[C@H](C#N)N2[C@H]1[C@@H]1SC[C@]3(NCCc4c3oc3ccccc43)C(=O)OC[C@H]2c2c3c(c(C)c(OC(C)=O)c21)OCO3 ; "AgNO3" ; COc1c(C)cc2c(c1O)[C@H]1N[C@@H](C2)[C@H](O)N2[C@H]1[C@@H]1SC[C@]3(NCCc4c3oc3ccccc43)C(=O)OC[C@H]2c2c3c(c(C)c(OC(C)=O)c21)OCO3]

To a solution of 14 (130 mg, 0.17 mmol) in CH₃CN:H₂O (1.39:1, 12 mL, 0.015 M) was added AgNO₃ (578 mg, 3.40 mmol). After 3 h at 23° C., a mixture 1:1 of saturated aqueous solutions of brine and NaHCO₃ was added, stirred for 15 min, diluted with CH₂Cl₂, stirred for 5 min, and extracted with CH₂Cl₂. The combined organic layers were dried over anhydrous Na₂SO₄, filtered, and concentrated under vacuum. The residue obtained was purified by flash chromatography (CH₂Cl₂:CH₃OH, from 99:1 to 85:15) to obtain pure 15 (80 mg, 64%).

R_{f}=0.25 (CH₂Cl₂:CH₃OH, 9:1).

¹H NMR (500 MHz, CD₃OD): δ 7.45-7.34 (m, 2H), 7.26-7.09 (m, 2H), 6.60 (s, 1H), 6.06 (d, J=1.1 Hz, 1H), 6.24 (d, J=1.1 Hz, 1H), 5.24 (d, J=11.5 Hz, 1H), 4.74 (s, 1H), 4.52 (s, 1H), 4.47 (d, J=4.9 Hz, 1H), 4.19-4.09 (m, 2H), 3.74 (s, 3H), 3.64 (d, J=9.2 Hz, 1H), 3.57 (d, J=4.9 Hz, 1H), 3.43-3.37 (m, 1H), 3.20-3.09 (m, 1H), 3.04 (dd, J=17.8, 9.5 Hz, 1H), 2.96-2.90 (m, 1H), 2.83 (d, J=15.4 Hz, 1H), 2.59-2.56 (m, 2H), 2.34 (s, 3H), 2.30 (s, 3H), 2.10-2.02 (m, 1H), 2.05 (s, 3H).

¹³C NMR (126 MHz, CD₃OD): δ 171.9, 170.7, 156.0, 150.5, 148.7, 147.0, 144.8, 142.4, 142.1, 132.6, 131.2, 128.6, 125.5, 124.7, 123.8, 122.3, 121.2, 120.2, 116.8, 114.9, 114.0, 112.3, 103.5, 91.4, 90.7, 63.7, 62.3, 60.4, 58.7, 57.1, 47.2, 43.5, 40.8, 39.3, 28.2, 21.5, 20.6, 16.2, 9.6.

ESI-MS m/z: Calcd. for C₃₉H₃₉N₃O₁₀S: 741.8. Found: 724.9 (M−H₂O+1)⁺.

(+)-HR-ESI-TOF-MS m/z: 741.2416 [M+H]⁺ (calcd. for C₃₉H₃₉N₃O₁₀S: 741.2356).

### Example 1-5

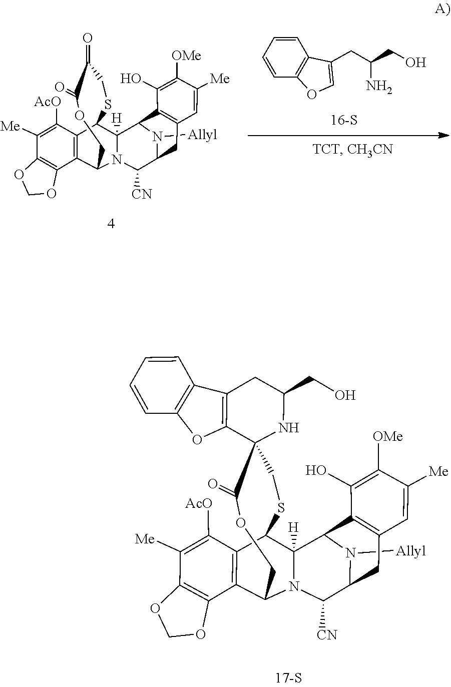

To a solution of 4 (150 mg, 0.24 mmol) in CH₃CN (15 mL, 0.016 M) was added 16-S (230 mg, 1.20 mmol) and Cyanuric Chloride (TCT) (45 mg, 30%). The reaction mixture was stirred for 24 h at 85° C. and then an aqueous saturated solution of NaHCO₃ was added and the mixture was extracted with CH₂Cl₂. The combined organic layers were dried over anhydrous Na₂SO₄, filtered, and concentrated under vacuum. Flash chromatography (Hexane:EtOAc, from 9:1 to 1:9) gives pure 17-S (145 mg, 73% yield).

¹H NMR (400 MHz, CDCl₃): δ 7.35 (dt, J=8.2, 0.9 Hz, 1H), 7.31 (ddd, J=7.6, 1.5, 0.7 Hz, 1H), 7.20 (ddd, J=8.4, 7.2, 1.5 Hz, 1H), 7.13 (td, J=7.4, 1.1 Hz, 1H), 6.62 (s, 1H), 6.20 (d, J=1.5 Hz, 1H), 6.05 (d, J=1.4 Hz, 1H), 5.85 (m, 1H), 5.74 (s, 1H), 5.16-5.08 (m, 3H), 4.58 (s, 1H), 4.40-4.32 (m, 2H), 4.28 (dd, J=11.5, 2.2 Hz, 1H), 4.19 (d, J=2.9 Hz, 1H), 3.80 (s, 3H), 3.58-3.53 (m, 1H), 3.50 (dd, J=11.3, 4.1 Hz, 2H), 3.42-3.30 (m, 1H), 2.96 (s, 1H), 2.90-2.73 (m, 4H), 2.58 (dd, J=15.7, 4.9 Hz, 1H), 2.52 (d, J=15.0 Hz, 1H), 2.37 (s, 3H), 2.36-2.26 (m, 2H), 2.28 (s, 3H), 2.04 (s, 3H).

ESI-MS m/z: 821.3 (M+H)⁺.

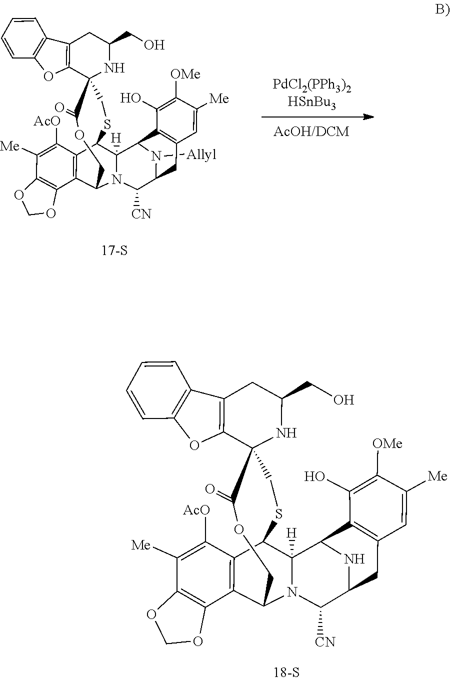

To a solution of 17-S (140 mg, 0.17 mmol) in CH₂Cl₂ was added PdCl₂(PPh₃)₂ (19 mg, 0.027 mmol), acetic acid (0.097 mL, 1.70 mmol) and SnBu₃H (1.65 mL, 6.12 mmol). The reaction mixture was stirred for 3 h at 23° C. The crude was concentrated under vacuum. Flash chromatography (Hexane:EtOAc, from 1:9 to 9:1) affords pure 18-S (94 mg, 71% yield).

¹H NMR (400 MHz, CDCl₃): δ 7.35 (dt, J=8.2, 0.9 Hz, 1H), 7.31 (dt, J=7.6, 1.0 Hz, 1H), 7.20 (ddd, J=8.3, 7.2, 1.5 Hz, 1H), 7.13 (td, J=7.4, 1.1 Hz, 1H), 6.62 (s, 1H), 6.20 (d, J=1.4 Hz, 1H), 6.05 (d, J=1.4 Hz, 1H), 5.09 (dd, J=11.5, 1.1 Hz, 1H), 4.58 (s, 1H), 4.52 (d, J=5.0 Hz, 1H), 4.39-4.35 (m, 1H), 4.27 (dd, J=11.5, 2.1 Hz, 1H), 4.20 (d, J=2.6 Hz, 1H), 3.85 (d, J=18.3 Hz, 1H), 3.79 (s, 3H), 3.54-3.44 (m, 2H), 3.34 (dd, J=11.2, 9.2 Hz, 1H), 3.04-2.97 (m, 2H), 2.92 (tt, J=8.6, 4.3 Hz, 1H), 2.60-2.47 (m, 2H), 2.35 (s, 3H), 2.34-2.28 (m, 2H), 2.28 (s, 3H), 2.05 (s, 3H).

ESI-MS m/z: 781.3 (M+H)⁺.

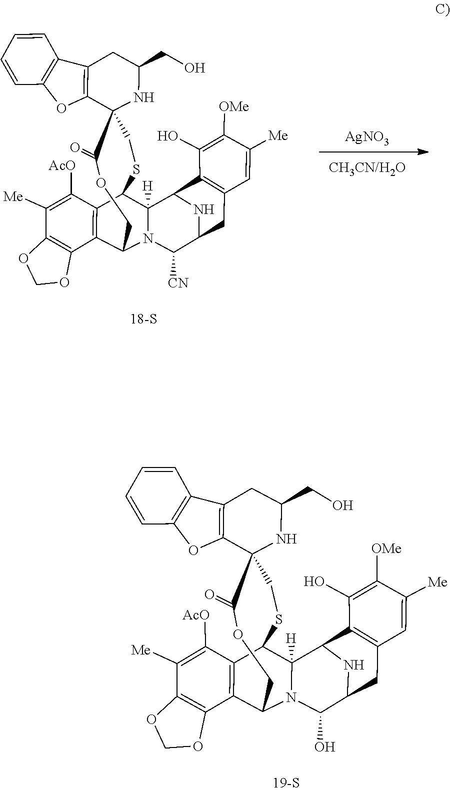 [SMILES: COc1c(C)cc2c(c1O)[C@H]1N[C@@H](C2)[C@H](C#N)N2[C@H]1[C@@H]1SC[C@]3(N[C@H](CO)Cc4c3oc3ccccc43)C(=O)OC[C@H]2c2c3c(c(C)c(OC(C)=O)c21)OCO3 ; "AgNO3" ; COc1c(C)cc2c(c1O)[C@H]1N[C@@H](C2)[C@H](O)N2[C@H]1[C@@H]1SC[C@]3(N[C@H](CO)Cc4c3oc3ccccc43)C(=O)OC[C@H]2c2c3c(c(C)c(OC(C)=O)c21)OCO3]

To a solution of 18-S (90 mg, 0.11 mmol) in CH₃CN:H₂O (1.39:1, 8 mL, 0.015 M) was added AgNO₃ (580 mg, 3.45 mmol). After 18 h at 23° C., a mixture 1:1 of saturated aqueous solutions of brine and NaHCO₃ was added, stirred for 15 min, diluted with CH₂Cl₂, stirred for 5 min, and extracted with CH₂Cl₂. The combined organic layers were dried over anhydrous Na₂SO₄, filtered, and concentrated under vacuum. The residue obtained was purified by flash chromatography (CH₂Cl₂:CH₃OH, from 99:1 to 85:15) to afford pure 19-S (60 mg, 68% yield).

¹H NMR (400 MHz, CDCl₃): δ 7.35 (d, J=8.1 Hz, 1H), 7.32-7.28 (m, 1H), 7.19 (td, J=8.3, 7.8, 1.4 Hz, 1H), 7.16-7.09 (m, 1H), 6.58 (s, 1H), 6.18 (d, J=1.5 Hz, 1H), 6.04 (d, J=4.6 Hz, 1H), 5.18 (d, J=11.3 Hz, 1H), 4.91 (s, 1H), 4.63 (s, 1H), 4.60-4.46 (m, 2H), 4.18 (d, J=10.8 Hz, 2H), 3.83-3.71 (m, 2H), 3.78 (s, 1H), 3.69 (s, 1H), 3.56-3.44 (m, 2H), 3.32 (t, J=10.3 Hz, 1H), 3.08-2.86 (m, 2H), 2.54 (dd, J=15.6, 5.0 Hz, 2H), 2.37-2.23 (m, 2H), 2.32 (s, 3H), 2.27 (s, 3H), 2.04 (s, 3H).

ESI-MS m/z: 754.3 (M−H₂O+H)⁺.

### Example 2. Synthesis of Linkers

### Preparation of LIN 1: MC-Val-Cit-PABC-PNP

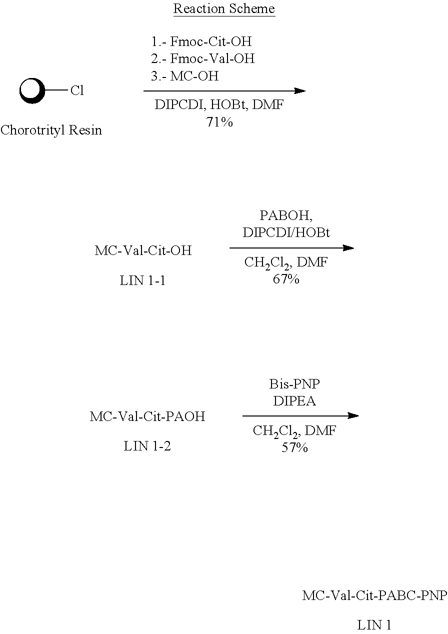

### (a) Preparation of LIN 1-1: MC-Val-Cit-OH

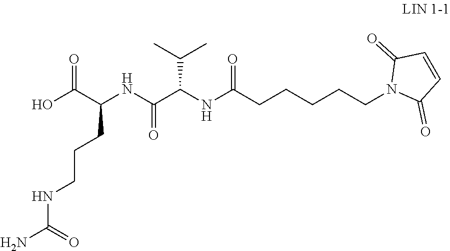 [SMILES: CC(C)[C@H](NC(=O)CCCCCN1C(=O)C=CC1=O)C(=O)N[C@@H](CCCNC(N)=O)C(=O)O]

Cl-TrtCl-resin (20 g, 1.49 mmol/g) (Iris Biotech, Ref.: BR-1065, 2-Chlorotrityl chloride resin (200-400 mesh, 1% DVB, 1.0-1.6 mmol/g), CAS 42074-68-0) was placed in a filter plate. 100 mL of DCM was added to the resin and the mixture was stirred for 1 h. The solvent was eliminated by filtration under vacuum. A solution of Fmoc-Cit-OH (11.83 g, 29.78 mmol) and DIPEA (17.15 mL, 98.45 mmol) in DCM (80 mL) was added and the mixture was stirred for 10 min. After that DIPEA (34.82 mmol, 199.98 mmol) was added and the mixture was stirred for 1 h. The reaction was terminated by addition of MeOH (30 mL) after stirring for 15 minutes. The Fmoc-Cit-O-TrtCl-resin produced as a result was subjected to the following washing/treatments: DCM (5×50 mL×0.5 min), DMF (5×50 mL×0.5 min), piperidine:DMF (1:4, 1×1 min, 2×10 min), DMF (5×50 mL×0.5 min), DCM (5×50 mL×0.5 min). The final piperidine wash gave NH₂—Cit-O-TrtCl-resin. The loading was calculated: 1.15 mmol/g.

The NH₂—Cit-O-TrtCl-resin produced above was washed with DMF (5×50 mL×0.5 min) and a solution of Fmoc-Val-OH (31.22 g, 91.98 mmol), HOBt (11.23 g, 91.98 mmol) in DMF (100 mL) was added to the NH₂—Cit-O-TrtCl-resin, stirred and DIPCDI (14.24 mL, 91.98 mmol) was added and the mixture was stirred for 1.5 h. The reaction was terminated by washing with DMF (5×50 mL×0.5 min). The Fmoc-Val-Cit-O-TrtCl-resin thus produced was treated with piperidine:DMF (1:4, 1×1 min, 2×10 min) and washed with DMF (5×50 mL×0.5 min). The final piperidine wash gave NH₂—Val-Cit-O-TrtCl-resin.

A solution of 6-maleimidocaproic acid (MC-OH) (9.7 g, 45.92 mmol), HOBt (6.21 g, 45.92 mmol) in DMF (100 mL) was added to the NH₂—Val-Cit-O-TrtCl-resin produced above, stirred and DIPCDI (7.12 mL, 45.92 mmol) was added and the mixture was stirred for 1.5 h. The reaction was terminated by washing with DMF (5×50 mL×0.5 min) and DCM (5×50 mL×0.5 min).

The peptide was cleaved from the resin by treatments with TFA:DCM (1:99, 5×100 mL). The resin was washed with DCM (7×50 mL×0.5 min). The combined filtrates were evaporated to dryness under reduced pressure and the solid obtained was triturated with Et₂O and filtrated to obtain LIN 1-1 (7.60 g, 71%) as a white solid.

¹H NMR (500 MHz, DMSO-d₆): δ 12.47 (s, 1H), 8.13 (d, J=7.3 Hz, 1H), 7.74 (d, J=9.0 Hz, 1H), 6.99 (s, 2H), 5.93 (s, 1H), 5.35 (s, 2H), 4.20 (dd, J=9.0, 6.8 Hz, 1H), 4.15-4.07 (m, 1H), 3.36 (t, J=7.0 Hz, 2H), 3.00-2.88 (m, 2H), 2.21-2.12 (m, 1H), 2.11-2.03 (m, 1H), 1.98-1.86 (m, 1H), 1.74-1.62 (m, 1H), 1.61-1.50 (m, 1H), 1.50-1.31 (m, 6H), 1.21-1.11 (m, 2H), 0.84 (d, J=6.8 Hz, 3H), 0.80 (d, J=6.8 Hz, 3H).

ESI-MS m/z: Calcd. for C₂₁H₃₃N₅O₇: 467.2. Found: 468.3 (M+H)⁺.

### (b) Preparation of LIN 1-2: MC-Val-Cit-PABOH

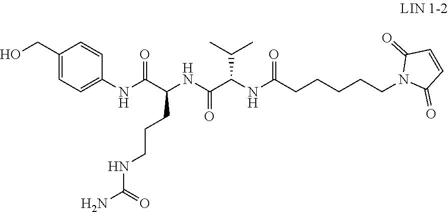 [SMILES: CC(C)[C@H](NC(=O)CCCCCN1C(=O)C=CC1=O)C(=O)N[C@@H](CCCNC(N)=O)C(=O)Nc1ccc(CO)cc1]

To a solution of LIN 1-1 (1.6 g, 3.42 mmol) and 4-aminobenzyl alcohol (PABOH) (0.84 g, 6.84 mmol) in DCM (60 mL) was added a solution of HOBt (0.92 g, 6.84 mmol) in DMF (5 mL). DIPCDI (1.05 mL, 6.84 mmol) was added, the reaction mixture was stirred for 2 h at 23° C., Et₂O (150 mL) was added, and the solid obtained was filtrated in a filter plate under vacuum to obtain LIN 1-2 (1.31 g, 67%).

¹H NMR (500 MHz, DMSO-d₆): δ 9.88 (s, 1H), 8.03 (d, J=7.6 Hz, 1H), 7.77 (dd, J=12.2, 8.5 Hz, 1H), 7.53 (d, J=8.2 Hz, 2H), 7.21 (d, J=8.2 Hz, 2H), 6.99 (s, 3H), 6.01-5.92 (m, 1H), 5.39 (s, 2H), 5.07 (s, 1H), 4.41 (s, 2H), 4.39-4.31 (m, 1H), 4.23-4.12 (m, 1H), 3.36 (t, J=7.0 Hz, 2H), 3.06-2.97 (m, 1H), 2.96-2.90 (m, 1H), 2.22-2.03 (m, 2H), 2.01-1.88 (m, 1H), 1.76-1.62 (m, 1H), 1.63-1.28 (m, 6H), 1.25-1.11 (m, 2H), 0.84 (d, J=6.9 Hz, 3H), 0.81 (d, J=6.8 Hz, 3H).

ESI-MS m/z: Calcd. for C₂₈H₄₀N₆O₇: 572.3. Found: 573.3 (M+H)⁺.

### (c) Preparation of LIN 1: MC-Val-Cit-PAB-PNP

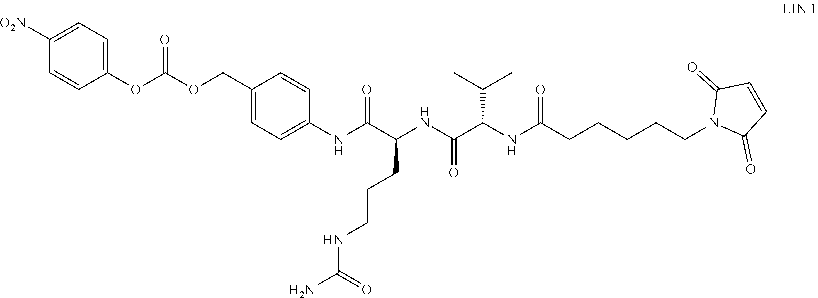 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

To a solution of LIN 1-2 (500 mg, 0.87 mmol) and bis(4-nitrophenyl) carbonate (bis-PNP) (2.64 g, 8.72 mmol) in DCM:DMF (8:2, 25 mL) was added DIPEA (0.45 mL, 2.61 mmol). The reaction mixture was stirred for 20 h at 23° C. and poured onto a silica gel column (DCM:CH₃OH, from 50:1 to 10:1) to afford pure target LIN 1 (364 mg, 57%).

R_{f}=0.40 (CH₂Cl₂:CH₃OH, 9:1).

¹H NMR (400 MHz, CDCl₃/CD₃OD): δ 9.45 (s, 1H), 8.23 (d, J=8.3 Hz, 2H), 7.59 (d, J=8.5 Hz, 2H), 7.35 (d, J=8.3 Hz, 2H), 7.34 (d, J=8.5 Hz, 2H), 6.65 (s, 2H), 5.20 (s, 2H), 4.56 (dt, J=10.5, 5.4 Hz, 1H), 4.15 (d, J=7.2 Hz, 1H), 3.46 (dd, J=8.0, 6.4 Hz, 2H), 3.16-2.89 (m, 2H), 2.21 (dd, J=8.3, 6.6 Hz, 2H), 2.06-1.97 (m, 1H), 1.90-1.83 (m, 1H), 1.73-1.46 (m, 7H), 1.34-1.20 (m, 2H), 0.91 (d, J=6.7 Hz, 3H), 0.90 (d, J=6.7 Hz, 3H).

¹³C NMR (125 MHz, CDCl₃/CD₃OD) δ 174.4, 172.4, 171.1, 170.6, 160.5, 155.5, 152.5, 145.3, 138.7, 134.1, 129.9, 129.5, 125.2, 121.8, 120.0, 70.6, 59.0, 53.2, 37.5, 35.8, 30.6, 29.6, 29.3, 28.1, 26.2, 26.2, 25.1, 19.1, 18.1.

ESI-MS m/z: Calcd. for C₃₅H₄₃N₇O₁₁: 737.3. Found: 738.3 (M+H)⁺.

### Preparation of LIN-2: MC2-PEG4-Val-Cit-PABC-PNP

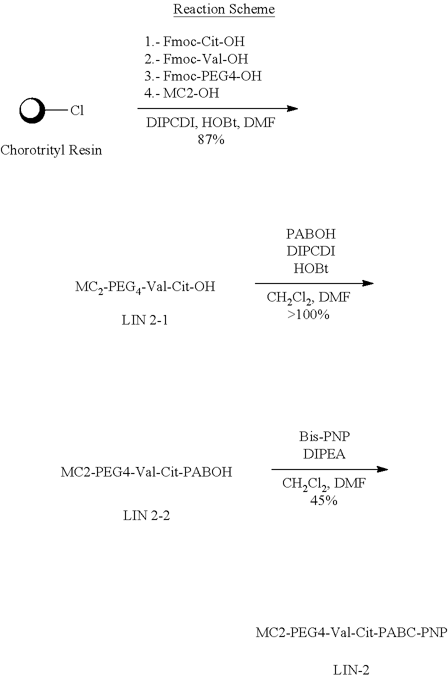

### a) Preparation of LIN 2-1: MC2-PEG4-Val-Cit-OH

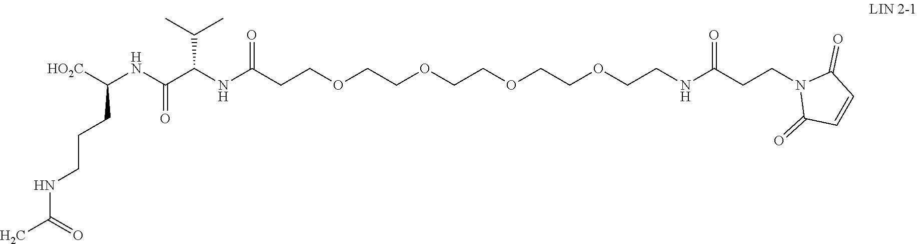

Cl-TrtCl-resin (5 g, 1.49 mmol/g) was placed in a filter plate. To the resin was added CH₂Cl₂ (25 mL) and the mixture was stirred for 1 h at 23° C. The solvent was eliminated by filtration over vacuum. A solution of Fmoc-Cit-OH (2.95 g, 7.44 mmol) and DIPEA (4.29 mL, 24.61 mmol) in CH₂Cl₂ (20 mL) was added and the mixture was stirred for 10 min at 23° C. DIPEA (8.70 mL, 49.99 mmol) was additionally added and the mixture was stirred for 1 h at 23° C. The reaction was stopped by addition of MeOH (10 mL) and stirred 15 min at 23° C. The Fmoc-Cit-O-TrtCl-resin was subjected to the following washing/treatments: CH₂Cl₂ (5×15 mL×0.5 min), DMF (5×15 mL×0.5 min), piperidine:DMF (1:4, 15 mL, 1×1 min, 2×10 min), DMF (5×15 mL×0.5 min), CH₂Cl₂ (5×15 mL×0.5 min). The loading was calculated: 1.17 mmol/g.

The NH₂—Cit-O-TrtCl-resin was washed with DMF (5×15 mL×0.5 min) and a solution of Fmoc-Val-OH (7.80 g, 22.99 mmol) and HOBt (2.80 g, 24.5 mmol) in DMF (25 mL) was added to the NH₂—Cit-O-TrtCl-resin followed by addition of DIPCDI (3.56 mL, 24.5 mmol) at 23° C. The reaction mixture was stirred for 1.5 h at 23° C. The reaction was stopped by washing with DMF (5×15 mL×0.5 min). The Fmoc-Val-Cit-O-TrtCl-resin was treated with piperidine:DMF (1:4, 15 mL, 1×1 min, 2×10 min) and washed with DMF (5×15 mL×0.5 min).

A solution of 15-(9-Fluorenylmethyloxycarbonyl)amino-4,7,10,13-tetraoxa-pentadecanoic acid (Fmoc-NH-PEG4-OH) (4.27 g, 8.75 mmol) and HOBt (1.18 g, 8.72 mmol) in DMF (30 mL) was added to the NH₂—Val-Cit-O-TrtCl-resin followed by addition of DIPCDI (1.35 mL, 8.72 mmol) at 23° C. The reaction mixture was stirred for 24 h at 23° C. The reaction was stopped by washing with DMF (5×15 mL×0.5 min). The Fmoc-NH-PEG4-Val-Cit-O-TrtCl-resin was treated with piperidine:DMF (1:4, 15 mL, 1×1 min, 2×10 min) and washed with DMF (5×15 mL×0.5 min).

A solution of 3-(Maleimido)propionic acid (MC2-OH) (3.95 g, 23.35 mmol) and HOBt (3.16 g, 23.37 mmol) in DMF (30 mL) was added to the NH₂—PEG4-Val-Cit-O-TrtCl-resin followed by addition of DIPCDI (3.62 mL, 23.37 mmol) at 23° C. The reaction mixture was stirred for 2 h at 23° C. The reaction was stopped by washing with DMF (5×15 mL×0.5 min) and CH₂Cl₂ (5×15 mL×0.5 min).

The peptide was cleaved from the resin by treatments with TFA:CH₂Cl₂ (1:99, 5×50 mL). The resin was washed with CH₂Cl₂ (7×50 mL×0.5 min). The combined filtrates were evaporated to dryness under reduced pressure, the solid obtained was triturated with Et₂O and filtrated to obtain LIN 2-1 (4.59 g, 87% yield) as a white solid.

¹H NMR (300 MHz, CDCl₃): δ 7.67-7.57 (m, 1H), 7.44 (d, J=8.3 Hz, 1H), 7.11 (t, J=5.4 Hz, 1H), 6.73 (s, 2H), 4.49 (d, J=7.2 Hz, 1H), 4.35 (t, J=7.7 Hz, 1H), 3.82 (t, J=7.0 Hz, 2H), 3.74 (t, J=6.2 Hz, 2H), 3.68-3.56 (m, 13H), 3.56-3.45 (m, 2H), 3.39 (q, J=5.4 Hz, 2H), 3.17 (s, 2H), 2.55 (q, J=7.0, 6.0 Hz, 4H), 2.16-1.99 (m, 1H), 1.91 (s, 1H), 1.75 (s, 1H), 1.43 (s, 2H), 0.94 (d, =9.7 Hz, 3H), 0.93 (d, =9.7 Hz, 3H).

ESI-MS m/z: 673.3 (M+H)⁺.

### (b) Preparation of LIN 2-2: MC2-PEG4-Val-Cit-PABOH

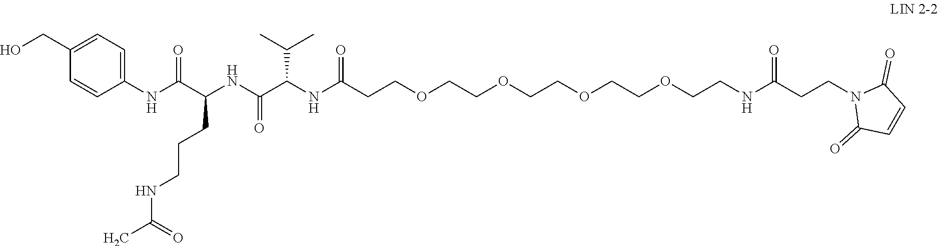

To a solution of LIN 2-1 (1.5 g, 2.22 mmol) and 4-aminobenzyl alcohol (PABOH) (0.55 g, 4.45 mmol) in CH₂Cl₂ (60 mL) was added a solution of HOBt (0.60 g, 4.45 mmol) in DMF (5 mL) followed by addition of DIPCDI (0.69 mL, 4.45 mmol) at 23° C. The reaction mixture was stirred for 5 h at 23° C., Et₂O (150 mL) was added, and the solid obtained was filtrated under vacuum to obtain crude LIN 2-2 (2.37 g, >100% yield) which was used in the next step without further purification.

¹H NMR (500 MHz, DMSO-d₆): δ 7.57 (d, J=8.6 Hz, 2H), 7.30 (d, J=8.6 Hz, 2H), 6.81 (s, 2H), 4.58 (s, 1H), 4.56 (s, 2H), 4.50 (dd, J=9.1, 5.1 Hz, 1H), 4.21 (d, J=7.0 Hz, 1H), 3.80-3.68 (m, 4H), 3.65-3.59 (m, 12H), 3.55-3.47 (m, 1H), 3.20 (dd, J=13.6, 6.9 Hz, 1H), 3.12 (dt, J=13.5, 6.7 Hz, 1H), 2.55 (td, J=6.1, 2.1 Hz, 2H), 2.46 (t, J=6.9 Hz, 2H), 2.15-2.07 (m, 1H), 1.95-1.88 (m, 1H), 1.79-1.70 (m, 1H), 1.67-1.50 (m, 2H), 0.99 (d, J=7.0 Hz, 3H), 0.98 (d, J=7.0 Hz, 3H).

ESI-MS m/z: 778.4 (M+H)⁺.

### (c) Preparation of LIN 2: MC2-PEG4-Val-Cit-PABC-PNP

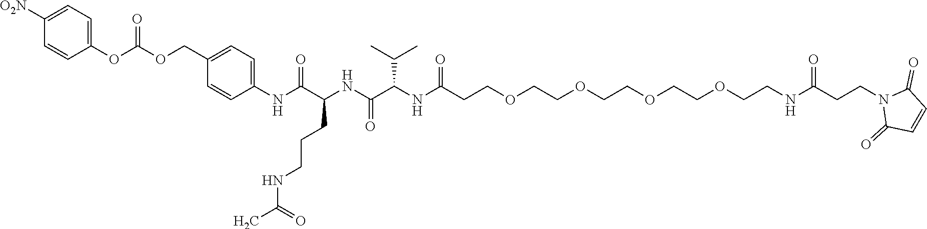

To a solution of LIN 2-2 (1.73 g, 2.22 mmol) and bis(4-nitrophenyl) carbonate (bis-PNP) (3.38 g, 11.12 mmol) in DCM:DMF (8:2, 75 mL) was added DIPEA (1.16 mL, 6.07 mmol) at 23° C. The reaction mixture was stirred for 19 h at 23° C. and poured onto silica gel column (CH₂Cl₂:CH₃OH, from 50:1 to 10:1) to afford pure LIN 2 (945 mg, 45% yield).

¹H NMR (500 MHz, CD₃OD): δ 8.22 (d, J=9.2 Hz, 2H), 7.61 (d, J=8.6 Hz, 2H), 7.34 (d, J=9.2 Hz, 2H), 7.33 (d, J=8.6 Hz, 2H), 6.67 (s, 2H), 4.57-4.47 (m, 1H), 4.23-4.12 (m, 1H), 3.78-3.76 (m, 12H), 3.63-3.50 (m, 16H), 3.49-3.41 (m, 2H), 3.34-3.25 (m, 2H), 3.18-3.03 (m, 2H), 2.51 (t, J=5.9 Hz, 2H), 2.45 (t, J=7.2 Hz, 2H), 2.13-1.99 (m, 1H), 1.92-1.84 (m, 1H), 1.73-1.62 (m, 1H), 1.55-1.45 (m, 2H), 0.92 (d, J=6.8 Hz, 3H), 0.90 (d, J=6.8 Hz, 3H).

¹³C NMR (75 MHz, CDCl₃/CD₃OD): δ 174.4, 172.9, 172.4, 172.4, 171.6, 170.9, 170.8, 170.7, 163.7, 155.8, 155.7, 152.5, 145.4, 138.8, 134.1, 131.3, 130.4, 129.2, 128.7, 125.7, 124.9, 121.8, 119.8 (×2), 115.1, 70.2 (×2), 70.1 (×2), 70.0, 69.9, 69.8, 69.0, 66.9, 59.2, 53.5, 39.0, 36.0, 34.4, 34.1, 30.4, 29.0, 18.5, 17.5.

ESI-MS m/z: 943.4 (M+H)⁺.

R_{f}=0.20 (CH₂Cl₂:CH₃OH, 9:1).

### Preparation of LIN 3: MC2-PEG4-Val-Ala-PABC-PNP

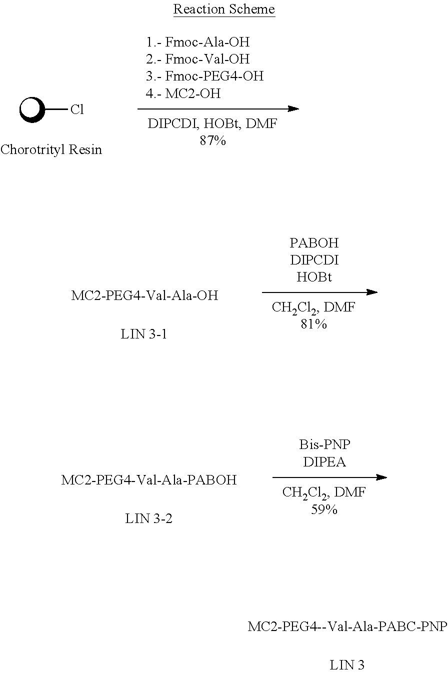

### (a) Preparation of LIN 3-1: MC2-PEG4-Val-Ala-OH

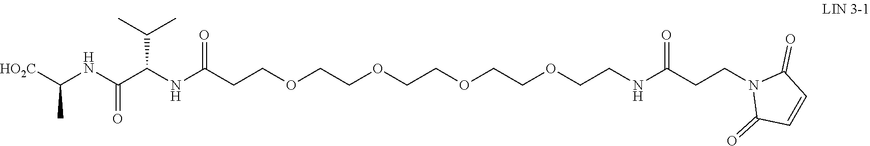

Cl-TrtCl-resin (5 g, 1.49 mmol/g) was placed in a filter plate. To the resin was added CH₂Cl₂ (25 mL) and the mixture was stirred for 1 h at 23° C. The solvent was eliminated by filtration over vacuum. A solution of Fmoc-Ala-OH (2.31 g, 7.41 mmol) and DIPEA (4.28 mL, 24.61 mmol) in CH₂Cl₂ (20 mL) was added and the mixture was stirred for 10 min at 23° C. DIPEA (8.60 mL, 49.37 mmol) was additionally added and the reaction mixture was stirred for 1 h at 23° C. The reaction was stopped by addition of MeOH (10 mL) and stirred 15 min at 23° C. The Fmoc-Ala-O-TrtCl-resin was subjected to the following washing/treatments: CH₂Cl₂ (5×15 mL×0.5 min), DMF (5×15 mL×0.5 min), piperidine:DMF (1:4, 15 mL, 1×1 min, 2×10 min), DMF (5×15 mL×0.5 min), CH₂Cl₂ (5×15 mL×0.5 min). The loading was calculated: 1.34 mmol/g.

The NH₂-Ala-O-TrtCl-resin was washed with DMF (5×15 mL×0.5 min) and a solution of Fmoc-Val-OH (9.09 g, 26.79 mmol) and HOBt (3.62 g, 26.79 mmol) in DMF (25 mL) was added to the NH₂-Ala-O-TrtCl-resin followed by addition DIPCDI (4.14 mL, 26.79 mmol) at 23° C. The mixture was stirred for 1.5 h at 23° C. The reaction was stopped by washing with DMF (5×15 mL×0.5 min). The Fmoc-Val-Ala-O-TrtCl-resin was treated with piperidine:DMF (1:4, 15 mL, 1×1 min, 2×10 min) and washed with DMF (5×15 mL×0.5 min).

A solution of 15-(9-Fluorenylmethyloxycarbonyl)amino-4,7,10,13-tetraoxa-pentadecanoic acid (Fmoc-NH-PEG4-OH) (4.90 g, 8.75 mmol) and HOBt (1.35 g, 9.98 mmol) in DMF (30 mL) was added to the NH₂—Val-Ala-O-TrtCl-resin followed by addition DIPCDI (1.55 mL, 10.0 mmol) at 23° C. The reaction mixture was stirred for 22 h at 23° C. The reaction was stopped by washing with DMF (5×15 mL×0.5 min). The Fmoc-NH-PEG4-Val-Ala-O-TrtCl-resin was treated with piperidine:DMF (1:4, 15 mL, 1×1 min, 2×10 min) and washed with DMF (5×15 mL×0.5 min).

A solution of 3-(Maleimido)propionic acid (MC2-OH) (4.53 g, 26.78 mmol) and HOBt (3.62 g, 26.77 mmol) in DMF (30 mL) was added to the NH₂—PEG4-Val-Ala-O-TrtCl-resin followed by addition of DIPCDI (4.15 mL, 26.80 mmol) at 23° C. The reaction mixture was stirred for 2 h at 23° C. The reaction was stopped by washing with DMF (5×15 mL×0.5 min) and CH₂Cl₂ (5×15 mL×0.5 min).

The peptide was cleaved from the resin by treatments with TFA:CH₂Cl₂ (1:99, 5×50 mL). The resin was washed with CH₂Cl₂ (7×50 mL×0.5 min). The combined filtrates were evaporated to dryness under reduced pressure, the solid obtained was triturated with Et₂O and filtrated to obtain L 3-1 (4.73 g, 87% yield) as a white solid.

¹H NMR (500 MHz, CDCl₃): δ 7.67 (bs, 1H), 7.31 (d, J=8.9 Hz, 1H), 7.17 (d, J=7.0 Hz, 1H), 6.85 (t, J=5.6 Hz, 1H), 6.72 (s, 2H), 4.51 (q, J=7.1 Hz, 1H), 4.38 (dd, J=8.9, 6.9 Hz, 1H), 3.84 (t, J=7.1 Hz, 2H), 3.75 (t, J=5.9 Hz, 2H), 3.69-3.59 (m, 12H), 3.55 (t, J=5.1 Hz, 2H), 3.41 (qd, J=5.0, 1.7 Hz, 2H), 2.62-2.49 (m, 4H), 2.19-2.01 (m, 1H), 1.44 (d, J=7.2 Hz, 3H), 0.95 (d, J=11.9 Hz, 1H), 0.94 (d, J=11.9 Hz, 1H).

### (b) Preparation of LIN 3-2: MC2-PEG4-Val-Ala-PABOH

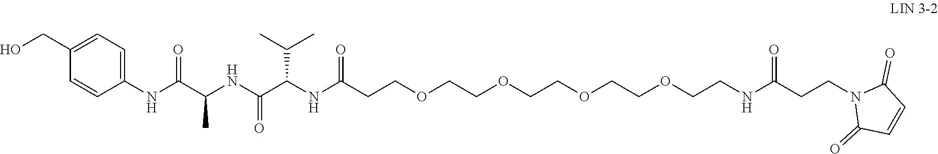

To a solution of LIN 3-1 (1.84 g, 3.13 mmol) and 4-aminobenzyl alcohol (PABOH) (0.77 g, 6.27 mmol) in CH₂Cl₂ (70 mL) was added a solution of HOBt (0.84 g, 6.27 mmol) in DMF (5 mL) followed by addition of DIPCDI (0.97 mL, 6.27 mmol) at 23° C. The reaction mixture was stirred for 5 h at 23° C., Et₂O (150 mL) was added, and the solid obtained was filtrated under vacuum to obtain crude LIN 3-2 (1.74 g, 81% yield) which was used in the next step without further purification.

¹H NMR (500 MHz, DMSO-d₆): δ 7.58 (d, J=8.5 Hz, 2H), 7.30 (d, J=8.5 Hz, 2H), 6.81 (s, 2H), 4.56 (s, 2H), 4.52-4.41 (m, 1H), 4.21 (d, J=6.7 Hz, 1H). 3.91 (p, J=6.5 Hz, 1H), 3.81-3.67 (m, 4H), 3.65-3.54 (m, 12H), 3.49 (t, J=5.5 Hz, 2H), 2.56 (dd, J=6.6, 5.5 Hz, 2H), 2.46 (t, J=6.9 Hz, 2H), 2.12 (h, J=6.8 Hz, 1H), 1.45 (d, J=7.2 Hz, 3H), 1.00 (d, J=12.1 Hz, 3H), 0.98 (d, J=12.1 Hz, 3H).

### (c) Preparation of LIN 3: MC2-PEG4-Val-Ala-PABC-PNP

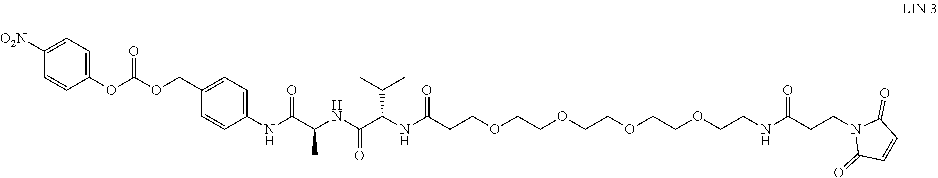

To a solution of LIN 3-2 (1.74 g, 2.51 mmol) and bis(4-nitrophenyl) carbonate (bis-PNP) (3.82 g, 12.57 mmol) in CH₂Cl₂:DMF (8:1, 70 mL) was added DIPEA (1.31 mL, 7.54 mmol) at 23° C. The reaction mixture was stirred for 20 h at 23° C. and poured onto silica gel column (CH₂Cl₂:CH₃OH, from 50:1 to 10:1) to afford pure LIN 3 (1.26 g, 59% yield).

¹H NMR (500 MHz, CDCl₃): δ 8.82 (s, 1H), 8.27 (d, J=9.2 Hz, 2H), 7.73 (d, J=8.6 Hz, 2H), 7.38 (d, J=9.1 Hz, 4H), 7.15 (dd, J=21.8, 7.2 Hz, 2H), 6.69 (s, 2H), 6.62 (t, J=5.7 Hz, 1H), 5.24 (s, 2H), 4.67 (p, J=7.2 Hz, 1H), 4.24 (dd, J=6.8, 5.7 Hz, 1H), 3.91-3.76 (m, 2H), 3.71 (ddd, J=10.1, 6.1, 4.3 Hz, 1H), 3.66-3.54 (m, 14H), 3.53 (t, J=5.1 Hz, 1H), 3.46-3.33 (m, 2H), 2.76-2.57 (m, 1H), 2.57-2.42 (m, 2H), 2.33-2.19 (m, 1H), 1.46 (d, J=7.1 Hz, 3H), 1.01 (d, J=12.1 Hz, 3H), 1.00 (d, J=12.1 Hz, 3H).

¹³C NMR (75 MHz, CD₃OD): δ 173.0, 172.1, 171.6 (×2), 170.7, 163.8, 155.7, 152.5, 145.4, 140.3, 138.9, 134.1, 130.4, 129.1, 125.6, 124.8, 121.9, 119.7, 115.1, 70.2, 70.1 (×3), 70.0, 69.9, 69.8, 69.0, 66.9, 59.1, 53.4, 49.7, 39.0, 36.0, 34.3, 34.1, 30.4, 18.3, 17.3, 16.6.

ESI-MS m/z: 857.3 (M+H)⁺.

R_{f}=0.45 (CH₂Cl₂:CH₃OH, 9:1).

### Example 3: Synthesis of a Compounds of Formula D-X-(AA)_{w}-(T)_{g}-L₁

### Preparation of Compound DL-1

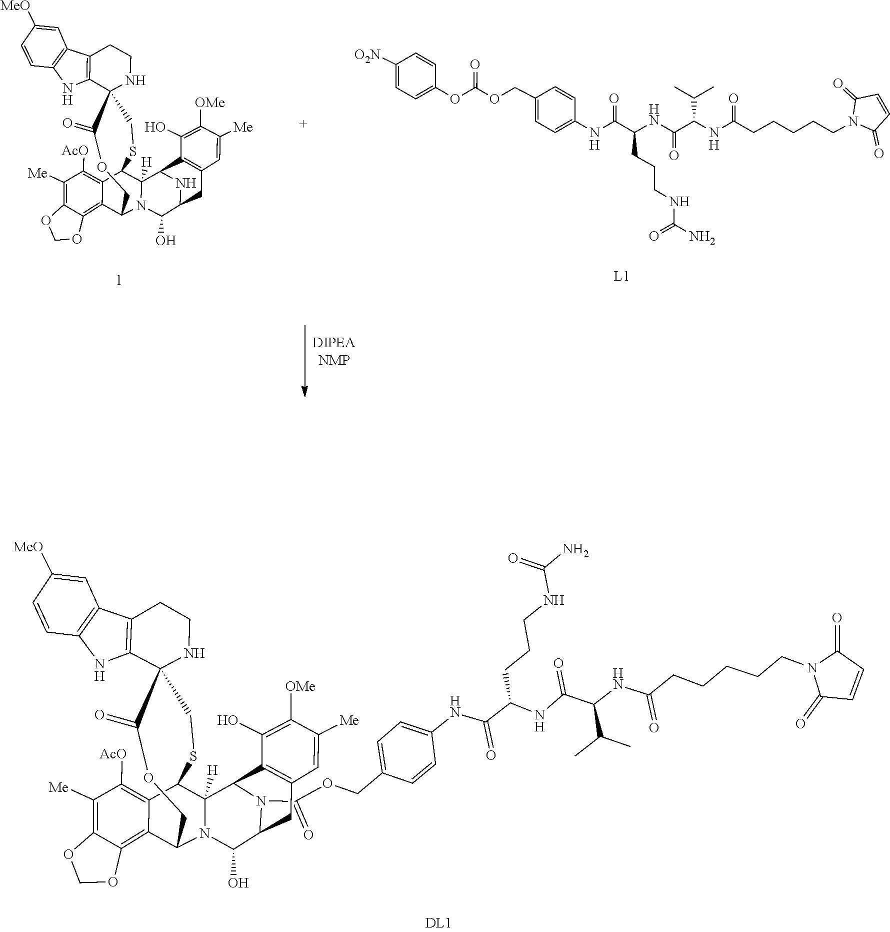 [SMILES: COc1ccc2[nH]c3c(c2c1)CCN[C@]31CS[C@@H]2c3c(OC(C)=O)c(C)c4c(c3[C@H](COC1=O)N1[C@@H]2[C@@H]2N[C@@H](Cc3cc(C)c(OC)c(O)c32)[C@@H]1O)OCO4 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C(=O)OCc1ccc(NC(=O)[C@H](CCCNC(N)=O)NC(=O)[C@@H](NC(=O)CCCCCN2C(=O)C=CC2=O)C(C)C)cc1)OCO4]

To a solution of 1 (15 mg, 0.019 mmol) and L1 (14 mg, 0.019 mmol) in 1-methyl-2-pyrrolidone (NMP) (1 mL, 0.019 M) was added DIPEA (3 μL, 0.019 mmol) at 23° C. After 72 h, EtOAc was added and the reaction mixture was washed with water and the organic layer was dried over anhydrous Na₂SO₄, filtered, and concentrated under vacuum. The residue obtained was purified by HPLC preparative to yield pure DL1 (7.5 mg, 29% yield).

¹H NMR (500 MHz, CD₃OD): δ 7.58 (d, J=8.6 Hz, 1H), 7.50 (d, J=8.6 Hz, 1H), 7.32 (d, J=8.6 Hz, 1H), 7.21 (d, J=8.6 Hz, 1H), 7.11 (dd, J=8.7, 1.8 Hz, 1H), 6.82 (t, J=2.0 Hz, 1H), 6.77 (s, 2H), 6.67 (ddd, J=8.9, 2.5, 1.3 Hz, 1H), 6.58 (s, 1H), 6.23 (dd, J=3.0, 1.3 Hz, 1H), 6.07 (t, J=1.4 Hz, 1H), 5.64 (ddd, J=12.4, 4.9, 1.8 Hz, 1H), 5.21 (dd, J=22.0, 11.1 Hz, 1H), 5.19-5.11 (m, 1H), 5.14-5.04 (m, 1H), 5.04-4.96 (m, 1H), 4.75 (s, 1H), 4.70 (s, 1H), 4.58 (s, 1H), 4.50 (ddd, J=8.7, 5.1, 3.3 Hz, 1H), 4.30 (d, J=3.1 Hz, 1H), 4.22-4.11 (m, 3H), 3.75 (s, 3H), 3.74 (s, 3H), 3.58-3.53 (m, 1H), 3.50-3.44 (m, 2H), 3.35 (s, 3H), 3.24-3.17 (m, 2H), 3.11 (ddd, J=13.7, 10.6, 6.6 Hz, 1H), 3.02 (dd, J=17.5, 9.8 Hz, 1H), 2.90-2.84 (m, 2H), 2.76 (dd, J=15.3, 2.4 Hz, 1H), 2.59 (dd, J=7.0, 4.9 Hz, 2H), 2.36-2.24 (m, 6H), 2.14-2.07 (m, 1H), 2.10-1.97 (m, 4H), 2.04 (s, 3H), 1.93-1.86 (m, 1H), 1.79-1.71 (m, 1H), 1.66-1.60 (m, 2H), 1.59-1.53 (m, 4H), 1.35-1.25 (m, 4H), 0.97 (m, 6H).

ESI-MS m/z: 1352.2 (M−H₂O+H)⁺.

### Preparation of Compound DL-2

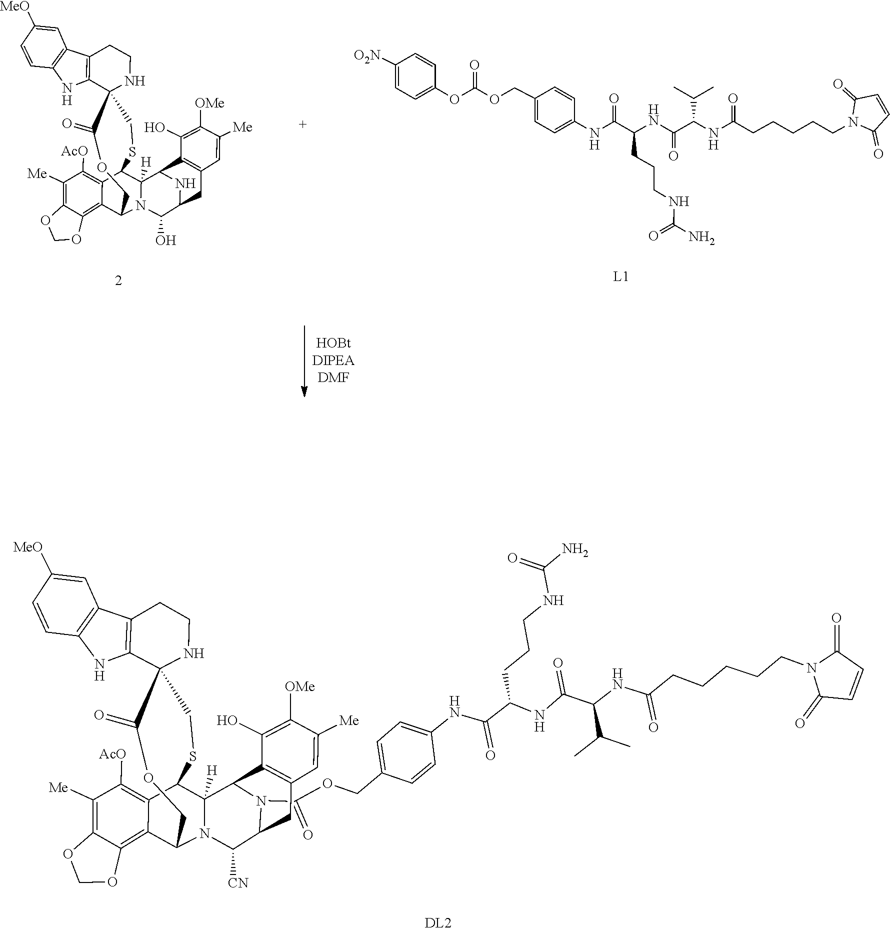 [SMILES: COc1ccc2[nH]c3c(c2c1)CCN[C@]31CS[C@@H]2c3c(OC(C)=O)c(C)c4c(c3[C@H](COC1=O)N1[C@@H]2[C@@H]2N[C@@H](Cc3cc(C)c(OC)c(O)c32)[C@@H]1O)OCO4 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1C#N)N2C(=O)OCc1ccc(NC(=O)[C@H](CCCNC(N)=O)NC(=O)[C@@H](NC(=O)CCCCCN2C(=O)C=CC2=O)C(C)C)cc1)OCO4]

To a solution of 2 (21 mg, 0.027 mmol) in Dimethylformamide (DMF) (2 mL, 0.013 M) was added L1 (22 mg, 0.029 mmol), 1-Hydroxybenzotriazole (HOBt, 3.9 mg, 0.029 mmol) and DIPEA (26 μL, 0.15 mmol) at 23° C. After 72 h, EtOAc was added and the reaction mixture was washed with water and the organic layer was dried over anhydrous Na₂SO₄, filtered, and concentrated under vacuum. The residue obtained was purified by HPLC preparative to yield pure DL2 (3.5 mg, 9% yield).

¹H NMR (400 MHz, CDCl₃): δ 7.74 (d, J=7.8 Hz, 1H), 7.47 (dd, J=21.6, 8.1 Hz, 2H), 7.23 (d, J=7.8 Hz, 1H), 7.12 (d, J=8.2 Hz, 1H), 7.07 (d, J=8.2 Hz, 1H), 6.77 (s, 2H), 6.64 (s, 2H), 6.54 (s, 1H), 6.16 (s, 1H), 5.97 (s, 1H), 5.63 (d, J=17.2 Hz, 1H), 5.11 (d, J=12.5 Hz, 1H), 5.01 (s, 1H), 4.90 (d, J=12.2 Hz, 1H), 4.66 (s, 1H), 4.50 (s, 1H), 4.29-4.19 (m, 2H), 4.13-4.08 (m, 1H), 3.74 (s, 3H), 3.70 (s, 3H), 3.68 (s, 3H), 3.43 (t, J=7.1 Hz, 2H), 3.34 (t, J=1.9 Hz, 1H), 3.33 (s, 2H), 3.08 (s, 2H), 2.98-2.72 (m, 5H), 2.50 (d, J=16.0 Hz, 1H), 2.33 (s, 3H), 2.26 (s, 3H), 2.22-2.14 (m, 3H), 1.99 (s, 3H), 1.81 (s, 1H), 1.63-1.50 (t, J=7.4 Hz, 4H), 1.48-1.39 (m, 4H), 1.28-1.19 (m, 3H), 0.90-0.86 (m, 6H).

ESI-MS m/z: 1379.5 (M+H)⁺.

### Example 4: Preparation of Antibody-Drug Conjugates (ADCs)

In this Example, syntheses of antibody-drug conjugates of the present invention are described. It should be noted that these syntheses are exemplary and that the processes described can be applied to all the compounds and antibodies described herein.

### Example 4a Preparation of Anti-CD13 Monoclonal Antibody

Anti-CD13 monoclonal antibodies were obtained following well known procedures commonly used in the art. Briefly BALB/c mice were immunized with human endothelial cells isolated from umbilical cord. To that end, 1.5E7 of the cells were injected to the mice intraperitoneally on days −45 and −30 and intravenously on day −3. On day 0 spleen from these animals were removed and spleen cells were fused with SP2 mouse myeloma cells at a ratio of 4:1 according to standard techniques to produce the hybridoma and distributed on 96-well tissue culture plates (Costar Corp., Cambridge, MA). After 2 weeks hybridoma culture supernatants were harvested and their reactivity against the cell line used in the immunization step was tested by flow cytometry. Positive supernatants were assayed by immunofluorescence staining the corresponding cells used as antigens. Hybridomas showing a specific staining, immunoprecipitation pattern and cell distribution were selected and cloned and subcloned by limiting dilution.

Once the clones were selected, cells were cultured in RPMI-1640 medium supplemented with 10% (v/v) fetal calf serum, 2 mM glutamine, 100 U/mL penicillin and 100 μg/mL streptomycin at 37° C. during 3-4 days until the medium turned pale yellow. At that point, two thirds of the medium volume were removed, centrifuged at 1,000×g for 10 min to pellet the cells and the supernatant was either centrifuged again for further cleaning at 3,000×g for 10 min or filtered through 22 μm pore size membranes. The clarified supernatant was subjected to precipitation with 55% saturation ammonium sulphate and the resulting pellet was resuspended in 100 mM Tris-HCl pH 7.8 (1 mL per 100 mL of the original clarified supernatant) and dialyzed at 4° C. for 16-24 h against 5 μL of 100 mM Tris-HCl pH 7.8 with 150 mM NaCl, changing the dialyzing solution at least three times. The dialyzed material was finally loaded onto a Protein A-Sepharose column and the corresponding monoclonal antibody was eluted with 100 mM sodium citrate pH 3.0 or alternatively with 1M glycine pH 3.0. Those fractions containing the antibody were neutralized with 2M Tris-HCl pH 9.0 and finally dialyzed against PBS and stored at −80° C. until its use.

### Preparation of Antibody-Drug Conjugate ADC1 with Trastuzumab and DL1

### (a) Preparation of Trastuzumab

Trastuzumab (purchased from Roche as a white lyophilised powder for the preparation of a concentrated solution for infusion) was dissolved in 5 mL of phosphate buffer (50 mM, pH 8.0) and purified by desalting using Sephadex G25 PD-10 columns into phosphate buffer (50 mM, pH 8.0). Concentration of Trastuzumab (17.0 mg/mL) was determined by measuring the absorbance at 280 nm.

### (b) Partial Reduction of Trastuzumab to Give Partially Reduced Trastuzumab

Trastuzumab solution (0.5 mL, 8.5 mg, 56.6 nmol) was diluted to a concentration of 10 mg/mL with phosphate buffer (50 mM, pH 8). Partial reduction of the disulfide bonds in the antibody was performed by the addition of a 5.0 mM tris[2-carboxyethyl]phosphine hydrochloride (TCEP) solution (34 μL, 170 nmol, 3 eq.) The reduction reaction was left to stir for 90 min at 20° C. Immediately after the reduction, an Ellman assay was performed to give a Free Thiol to Antibody ratio (FTAR) of 5.0.

### (c) Preparation of ADC1

To the solution of partially reduced Trastuzumab (0.2 mL, 2 mg, 13.3 nmol), DMA was added (39.4 μL) followed by addition of a freshly prepared solution of DL1 (10 mM in DMA, 10.6 μL, 106 nmol, 8 eq.). The conjugation reaction was stirred for 30 min at 20° C. and the excess of drug was quenched by addition of N-acetylcysteine (NAC) (10 mM, 10.6 μL, 106 nmol) followed by stirring the solution for 20 min. The quenched conjugation reaction was purified by desalting using Sephadex G25 NAP-5 columns into PBS buffer. The final target product ADC1 was concentrated to a final concentration of 3.9 mg/mL as determined by UV and 370 μL (1.44 mg, 9.6 nmol, 72%) ADC solution was obtained. HIC HPLC runs were performed to determine the percentage of conjugation reaction (94%).

### Preparation of Antibody-Drug Conjugate ADC2 with Trastuzumab and Compound DL2

### (a) Preparation of Trastuzumab

Trastuzumab (purchased from Roche as a white lyophilised powder for the preparation of a concentrated solution for infusion) was dissolved in 5 mL of phosphate buffer (50 mM, pH 8.0) and purified by desalting using Sephadex G25 PD-10 columns into phosphate buffer (50 mM, pH 8.0). Concentration of Trastuzumab (17.1 mg/mL) was determined by measuring the absorbance at 280 nm.

### (b) Partial Reduction of Trastuzumab to Give Partially Reduced Trastuzumab

Trastuzumab solution (0.5 mL, 8.55 mg, 57 nmol) was diluted to a concentration of 10 mg/mL with phosphate buffer (50 mM, pH 8). Partial reduction of the disulfide bonds in the antibody was performed by the addition of a 5.0 mM tris[2-carboxyethyl]phosphine hydrochloride (TCEP) solution (34.2 μL, 171 μmol, 3 eq.) The reduction reaction was left to stir for 90 min at 20° C. Immediately after the reduction, an Ellman assay was performed to give a Free Thiol to Antibody ratio (FTAR) of 6.7.

### (c) Preparation of ADC2

To the solution of partially reduced Trastuzumab (171 μL, 1.71 mg, 11.4 nmol), DMA was added (33.6 μL) followed by addition of a freshly prepared solution of DL2 (10 mM in DMA, 9.1 μL, 91 nmol, 8 eq.). The conjugation reaction was stirred for 30 min at 20° C. The excess of drug was quenched by addition of N-acetylcysteine (NAC) (10 mM, 9.1 μL, 91 nmol) followed by stirring the solution for 20 min. The quenched conjugation reaction was purified by desalting using Sephadex G25 NAP-5 columns into PBS buffer. The final target product ADC2 was concentrated to a final concentration of 5.14 mg/mL as determined by UV and 300 μL (1.5 mg, 10 nmol, 87%) ADC solution was obtained. HIC HPLC runs were performed to determine the percentage of conjugation reaction (75%).

### Preparation of Antibody-Drug Conjugate ADC3 with Trastuzumab and Compound DL1

### (a) Preparation of Trastuzumab

Trastuzumab (purchased from Roche as a white lyophilised powder for the preparation of a concentrated solution for infusion) was dissolved in 5 mL of phosphate buffer (50 mM, pH 8.0) and purified by desalting using Sephadex G25 PD-10 columns into phosphate buffer (50 mM, pH 8.0). Concentration of Trastuzumab (16.5 mg/mL) was determined by measuring the absorbance at 280 nm.

### (b) Reaction of Trastuzumab with 2-Iminothiolane (Traut's Reagent) to Give Thiol-Activated Trastuzumab

Trastuzumab solution (0.5 mL, 8.25 mg, 55 nmol) was diluted to a concentration of 10 mg/mL using phosphate buffer (50 mM phosphate, 2 mM EDTA, pH 8). 14 mM solution of Traut's reagent was added (47.1 μL, 660 nmol, 12 eq.), and the reaction stirred for 2 h at 20° C. The mixture was buffer exchanged using two Sephadex G25 NAP-5 columns into PBS buffer, and concentrated to a volume of 0.85 mL (9.7 mg/mL). Immediately after, an Ellman assay was performed to give a Free Thiol to Antibody ratio (FTAR) of 5.5.

### (c) Preparation of ADC3

To the solution of thiol-activated Trastuzumab (200 μL, 1.94 mg, 12.9 nmol), DMA was added (38 μL) followed by addition of a freshly prepared solution of DL1 (10 mM in DMA, 12 μL, 120 nmol, 9.3 eq.). The conjugation reaction was stirred for 2 h at 25° C. and purified by desalting using a Sephadex G25 NAP-5 column into PBS buffer. The final target product ADC3 was concentrated to a final concentration of 1.48 mg/mL as determined by UV and 340 μL (0.5 mg, 3.3 nmol, 25%) ADC solution was obtained.

### Preparation of Antibody-Drug Conjugate ADC4 with Trastuzumab and DL2

### (a) Preparation of Trastuzumab

Trastuzumab (purchased from Roche as a white lyophilised powder for the preparation of a concentrated solution for infusion) was dissolved in 5 mL of phosphate buffer (50 mM, pH 8.0) and purified by desalting using Sephadex G25 PD-10 columns into phosphate buffer (50 mM, pH 8.0). Concentration of Trastuzumab (17.1 mg/mL) was determined by measuring the absorbance at 280 nm.

### (b) Reaction of Trastuzumab with 2-Iminothiolane (Traut's Reagent) to Give Thiol-Activated Trastuzumab

Trastuzumab solution (0.85 mL, 14.5 mg, 96.6 nmol) was diluted to a concentration of 10 mg/mL using phosphate buffer (50 mM phosphate, 2 mM EDTA, pH 8). 14 mM solution of Traut's reagent was added (69 μL, 966 nmol, 10 eq.), and the reaction stirred for 2 h at 20° C. The mixture was buffer exchanged using two Sephadex G25 NAP-5 columns into PBS buffer, and concentrated to a volume of 1.45 mL (10 mg/mL). Immediately after, an Ellman assay was performed to give a Free Thiol to Antibody ratio (FTAR) of 3.7.

### (c) Preparation of ADC4

To the solution of thiol-activated Trastuzumab (290 μL, 2.9 mg, 19.3 nmol), DMA was added (57.1 μL) followed by addition of a freshly prepared solution of DL2 (10 mM in DMA, 15.4 μL, 154 nmol, 8 eq.). The conjugation reaction was stirred for 2 h at 25° C. and purified by desalting using a Sephadex G25 NAP-5 column into PBS buffer. The final target product ADC4 was concentrated to a final concentration of 3.82 mg/mL as determined by UV and 315 μL (1.2 mg, 8.0 nmol, 41%) ADC solution was obtained.

### Example 5. In Vitro Bioassays for the Detection of Antitumor Activity of the Drugs of the Invention

The aim of this assay is to evaluate the in vitro cytostatic (ability to delay or arrest tumor cell growth) or cytotoxic (ability to kill tumor cells) activity of the samples being tested.

### Cell Lines

| Name | No ATCC | Species | Tissue | Characteristics |
|---|---|---|---|---|
| A549 | CCL-185 | human | lung | lung carcinoma |
| | | | | (NSCLC) |
| HT29 | HTB-38 | human | colon | colorectal |
| | | | | adenocarcinoma |
| MDA-MB-231 | HTB-26 | human | breast | breast |
| | | | | adenocarcinoma |
| PSN1 | CRM-CRL- | human | pancreas | pancreas |
| | 3211 | | | adenocarcinoma |

### Evaluation of Cytotoxic Activity Using the SBR Colorimetric Assay

A colorimetric assay, using sulforhodamine B (SRB) reaction has been adapted to provide a quantitative measurement of cell growth and viability (following the technique described by Skehan et al. J. Natl. Cancer Inst. 1990, 82, 1107-1112).

This form of assay employs SBS-standard 96-well cell culture microplates (Faircloth et al. Methods in Cell Science, 1988, 11(4), 201-205; Mosmann et al. Journal of Immunological Methods, 1983, 65 (1-2), 55-63. All the cell lines used in this study were obtained from the American Type Culture Collection (ATCC) and derive from different types of human cancer.

Cells were maintained in Dulbecco's Modified Eagle Medium (DMEM) supplemented with 10% Fetal Bovine Serum (FBS), 2 mM L-glutamine, 100 U/mL penicillin and 100 U/mL streptomycin at 37° C., 5% C₀₂ and 98% humidity. For the experiments, cells were harvested from subconfluent cultures using trypsinization and resuspended in fresh medium before counting and plating.

Cells were seeded in 96 well microtiter plates, at 5×103 cells per well in aliquots of 150 μL, and allowed to attach to the plate surface for 18 hours (overnight) in drug free medium. After that, one control (untreated) plate of each cell line was fixed (as described below) and used for time zero reference value. Culture plates were then treated with test compounds (50 μL aliquots of 4× stock solutions in complete culture medium plus 4% DMSO) using ten serial dilutions (concentrations ranging from 10 to 0.00262 μg/mL) and triplicate cultures (1% final concentration in DMSO). After 72 hours treatment, the antitumor effect was measured by using the SRB methodology: Briefly, cells were washed twice with PBS, fixed for 15 min in 1% glutaraldehyde solution at room temperature, rinsed twice in PBS, and stained in 0.4% SRB solution for 30 min at room temperature. Cells were then rinsed several times with 1% acetic acid solution and air-dried at room temperature. SRB was then extracted in 10 mM trizma base solution and the absorbance measured in an automated spectrophotometric plate reader at 490 nm. Effects on cell growth and survival were estimated by applying the NCI algorithm (Boyd MR and Paull KD. Drug Dev. Res. 1995, 34, 91-104).

Using the mean±SD of triplicate cultures, a dose-response curve was automatically generated using nonlinear regression analysis. Three reference parameters were calculated (NCI algorithm) by automatic interpolation: GI₅₀=compound concentration that produces 50% cell growth inhibition, as compared to control cultures; TGI=total cell growth inhibition (cytostatic effect), as compared to control cultures, and LC₅₀=compound concentration that produces 50% net cell killing cytotoxic effect).

The in vitro cytostatic (ability to delay or arrest tumor cell growth) or cytotoxic (ability to kill tumor cells) of compounds 1, 2, 3 and ET722 and other payloads of this invention, have been disclosed in WO2003066638 (compounds 64, 60, 59 and 63, respectively, at pages 149-151).

Tables 3-6 illustrate data on the biological activity of the drugs of the present invention together with biological activity of the closest prior art compounds.

TABLE 3
Biological activity (Molar)

| | Drug | | | | | Reference compound | | | | |
|---|---|---|---|---|---|---|---|---|---|---|
| | 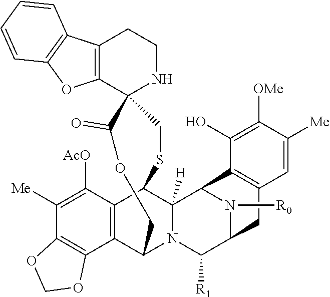 14 R₀ = H, R₁ = CN 15 R₀ = H, R₁ = OH | | | | | 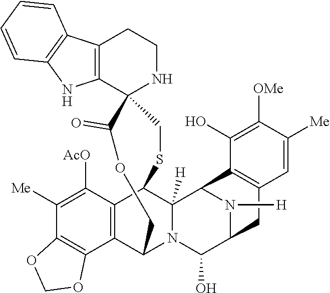 [SMILES: COc1c(C)cc2c(c1O)[C@H]1N[C@@H](C2)[C@H](O)N2[C@H]1[C@@H]1SC[C@]3(NCCc4c3[nH]c3ccccc43)C(=O)OC[C@H]2c2c3c(c(C)c(OC(C)=O)c21)OCO3] ET-722 | | | | |
| | | | | MDA-MB- | | | | | MDA- | |
| | | A549 | HT29 | 231 | PSN1 | | A549 | HT29 | MB-231 | PSN1 |
| GI₅₀ | 14 | 8.79E−09 | 8.52E−09 | 6.66E−09 | 9.72E−09 | | | | | |
| TGI | | 1.73E−08 | 9.19E−09 | 1.13E−08 | 1.33E−08 | | | | | |
| LC₅₀ | | >1.33E−07 | >1.33E−07 | 2.93E−08 | 2.26E−08 | | | | | |
| GI₅₀ | 15 | 1.21E−08 | 8.76E−09 | 7.14E−09 | 9.98E−09 | ET- | 1.35E−09 | 1.35E−09 | 8.91E−10 | 1.48E−09 |
| TGI | | 2.56E−08 | 9.57E−09 | 1.16E−08 | 1.48E−08 | 722 | 1.35E−09 | 1.48E−09 | 1.48E−09 | 2.16E−09 |
| LC₅₀ | | >1.35E−07 | >1.35E−07 | 2.43E−08 | 4.58E−08 | | >1.35E−07 | >1.35E−07 | 2.56E−09 | 3.10E−09 |

TABLE 4
Biological activity (Molar)

| | Drug | | | | | Reference compound | | | | |
|---|---|---|---|---|---|---|---|---|---|---|
| | 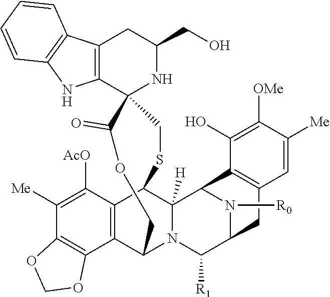 6-S R₀ = H, R₁ = CN 7-S R₀ = H, R₁ = OH | | | | | 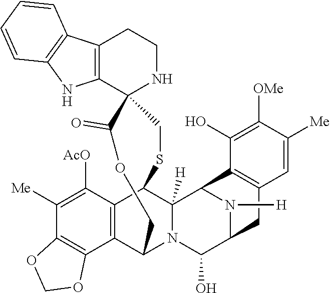 [SMILES: COc1c(C)cc2c(c1O)[C@H]1N[C@@H](C2)[C@H](O)N2[C@H]1[C@@H]1SC[C@]3(NCCc4c3[nH]c3ccccc43)C(=O)OC[C@H]2c2c3c(c(C)c(OC(C)=O)c21)OCO3] ET-722 | | | | |
| | | | | MDA-MB- | | | | | MDA- | |
| | | A549 | HT29 | 231 | PSN1 | | A549 | HT29 | MB-231 | PSN1 |
| GI₅₀ | 6-S | 8.08E−09 | 3.33E−09 | 2.95E−09 | 3.72E−09 | | | | | |
| TGI | | 1.12E−08 | 3.59E−09 | 6.03E−09 | 5.77E−09 | | | | | |
| LC₅₀ | | >1.28E−07 | >1.28E−07 | 2.44E−08 | 1.09E−08 | | | | | |
| GI₅₀ | 7-S | 8.17E−09 | 3.37E−09 | 2.85E−09 | 3.11E−09 | ET- | 1.35E−09 | 1.35E−09 | 8.91E−10 | 1.48E−09 |
| TGI | | 1.28E−08 | 3.63E−09 | 4.28E−09 | 4.15E−09 | 722 | 1.35E−09 | 1.48E−09 | 1.48E−09 | 2.16E−09 |
| LC₅₀ | | >1.30E−07 | >1.30E−07 | 6.88E−09 | 6.62E−09 | | >1.35E−07 | >1.35E−07 | 2.56E−09 | 3.10E−09 |

TABLE 5
Biological activity (Molar)

| | Drug | | | | | Reference compound | | | | |
|---|---|---|---|---|---|---|---|---|---|---|
| | 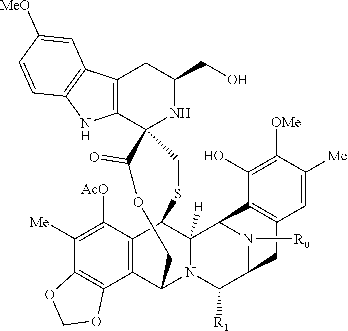 | | | | | | | | | |
| | 10-S R₀ = H, R₁ = CN | | | | | | | | | |
| | 11-S R₀ = H, R₁ = OH | | | | | | | | | |
| | 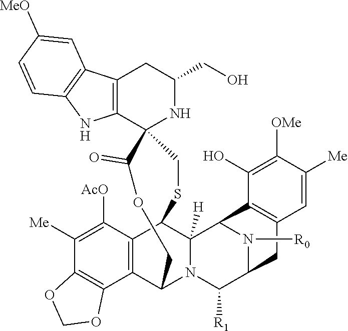 | | | | | 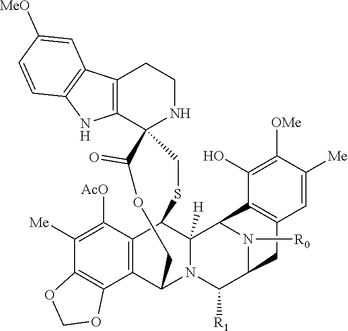 | | | | |
| | 10-R R₀ = H, R₁ = CN | | | | | 2 R₀ = H, R₁ = CN | | | | |
| | 11-R R₀ = H, R₁ = OH | | | | | 1 R₀ = H, R₁ = OH | | | | |
| | | | | MDA- | | | | | MDA- | |
| | | A549 | HT29 | MB-231 | PSN1 | | A549 | HT29 | MB-231 | PSN1 |
| GI₅₀ | 10-S | 4.32E−08 | 1.23E−08 | 1.20E−08 | 8.64E−09 | 2 | 1.28E−08 | 5.13E−09 | 5.00E−09 | 2.05E−09 |
| TGI | | 1.05E−07 | 1.23E−08 | 1.23E−08 | 1.48E−08 | | 1.28E−08 | 5.64E−09 | 5.26E−09 | 3.08E−09 |
| LC₅₀ | | >1.23E−06 | >1.23E−06 | 1.36E−08 | >1.23E−06 | | >1.28E−06 | 1.28E−06 | 5.77E−09 | 5.00E−09 |
| GI₅₀ | 10-R | 4.32E−09 | 8.64E−10 | 6.79E−10 | 7.53E−10 | | | | | |
| TGI | | 1.20E−08 | 1.61E−09 | 1.21E−09 | 1.48E−09 | | | | | |
| LC₅₀ | | >1.23E−07 | >1.23E−07 | 3.09E−09 | >1.23E−07 | | | | | |
| GI₅₀ | 11-S | 6.62E−08 | 1.37E−08 | 1.05E−08 | 1.62E−08 | 1 | 1.82E−09 | 1.28E−09 | 7.52E−10 | 1.22E−09 |
| TGI | | >1.25E−07 | 2.50E−08 | 1.87E−08 | 2.37E−08 | | 3.37E−09 | 1.43E−09 | 1.30E−09 | 1.69E−09 |
| LC₅₀ | | >1.25E−07 | >1.25E−07 | 4.50E−08 | >1.25E−07 | | 7.78E−09 | >1.30E−07 | 2.46E−09 | 2.34E−09 |
| GI₅₀ | 11-R | 1.50E−08 | 2.00E−09 | 1.62E−09 | 2.12E−09 | | | | | |
| TGI | | 4.50E−08 | 3.62E−09 | 2.87E−09 | 3.62E−09 | | | | | |
| LC₅₀ | | >1.25E−07 | >1.25E−07 | 7.24E−09 | 1.50E−08 | | | | | |

TABLE 6
Biological activity (Molar)

| | Drug | | | | | Reference compound | | | | |
|---|---|---|---|---|---|---|---|---|---|---|
| | 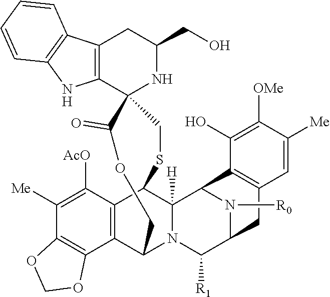 18-S R₀ = H, R₁ = CN 19-S R₀ = H, R₁ = OH | | | | | 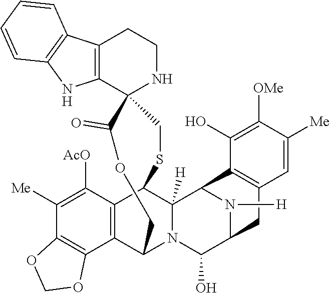 [SMILES: COc1c(C)cc2c(c1O)[C@H]1N[C@@H](C2)[C@H](O)N2[C@H]1[C@@H]1SC[C@]3(NCCc4c3[nH]c3ccccc43)C(=O)OC[C@H]2c2c3c(c(C)c(OC(C)=O)c21)OCO3] ET-722 | | | | |
| | | | | MDA-MB- | | | | | MDA- | |
| | | A549 | HT29 | 231 | PSN1 | | A549 | HT29 | MB-231 | PSN1 |
| GI₅₀ | 18-S | 8.08E−09 | 3.33E−09 | 2.95E−09 | 3.72E−09 | | | | | |
| TGI | | 1.12E−08 | 3.59E−09 | 6.03E−09 | 5.77E−09 | | | | | |
| LC₅₀ | | >1.28E−07 | >1.28E−07 | 2.44E−08 | 1.09E−08 | | | | | |
| GI₅₀ | 19S | 8.17E−09 | 3.37E−09 | 2.85E−09 | 3.11E−09 | ET- | 1.35E−09 | 1.35E−09 | 8.91E−10 | 1.48E−09 |
| TGI | | 1.28E−08 | 3.63E−09 | 4.28E−09 | 4.15E−09 | 722 | 1.35E−09 | 1.48E−09 | 1.48E−09 | 2.16E−09 |
| LC₅₀ | | >1.30E−07 | >1.30E−07 | 6.88E−09 | 6.62E−09 | | >1.35E−07 | >1.35E−07 | 2.56E−09 | 3.10E−09 |

### Example 6: Demonstrating the Cytotoxicity of the Antibody-Drug Conjugates of the Present Invention

### Bioassays for the Detection of Antitumor Activity

The aim of the assay was to evaluate the in vitro cytostatic (ability to delay or arrest tumor cell growth) or cytotoxic (ability to kill tumor cells) activity of the samples being tested.

### Cell Lines and Cell Culture

The following human cell lines were obtained from the American Type Culture Collection (ATCC): SK-BR-3 (ATCC HB-30), HCC-1954 (ATCC CRL-2338) (Breast cancer, HER2+); MDA-MB-231 (ATCC HTB-26) and MCF-7 (ATCC HTB-22) (Breast cancer, HER2−), Cells were maintained at 37° C., 5% CO₂ and 95% humidity in Dulbecco's Modified Eagle's Medium (DMEM) (for SK-BR-3, MDA-MB-231 and MCF-7 cells), or RPMI-1640 (HCC-1954), all media supplemented with 10% Fetal Calf Serum (FCS), 2 mM L-glutamine and 100 units/mL penicillin and streptomycin.

### Cytotoxicity Assay

For SK-BR-3, HCC-1954, MDA-MB-231 and MCF-7 cells, a colorimetric assay using Sulforhodamine B (SRB) was adapted for quantitative measurement of cell growth and cytotoxicity, as described in V. Vichai and K. Kirtikara. Sulforhodamine B colorimetric assay for cytotoxicity screening. Nature Protocols, 2006, 1, 1112-1116. Briefly, cells were seeded in 96-well microtiter plates and allowed to stand for 24 hours in drug-free medium before treatment with vehicle alone or with the tested substances for 72 hours. For quantification, cells were washed twice with phosphate buffered saline (PBS), fixed for 15 min in 1% glutaraldehyde solution, rinsed twice with PBS, stained in 0.4% (w/v) SRB with 1% (v/v) acetic acid solution for 30 min, rinsed several times with 1% acetic acid solution and air-dried. SRB was then extracted in 10 mM Trizma base solution and the optical density measured at 490 nm in a microplate spectrophotometer.

Cell survival was expressed as percentage of control, untreated cell survival. All evaluations were performed in triplicate and the resulting data were fitted by nonlinear regression to a four-parameters logistic curve from which the IC₅₀ value (the concentration of compound causing 50% cell death as compared to the control cell survival) was calculated.

### Bioactivity Example 1—Cytotoxicity of the Conjugate ADC 1 and Related Reagents Against HER2 Positive and Negative Breast Cancer Cells

The in vitro cytotoxicity of the ACD 1 along with the parent cytotoxic compounds 1 and Trastuzumab were evaluated against four different human breast cancer cell lines over-expressing or not the HER2 receptor, including SK-BR-3, HCC-1954 (HER2-positive cells) as well as MDA-MB-231 and MCF-7 (HER2-negative cells). Standard dose-response (DR) curves for 72 hours incubation with the tested substances were performed.

### Cytotoxicity of Trastuzumab

The in vitro cytotoxicity of Trastuzumab was evaluated against the different tumor cell lines by performing triplicate 10-points, 2.5-fold dilution DR curves ranging from 50 to 0.01 g/mL (3.33E-07-8.74E-11). Trastuzumab was completely inactive, not reaching the IC₅₀ in any of the cell lines tested, independently of their HER2 status as shown in Table 7 where results corresponding to the geometric mean of the IC₅₀ values obtained in three independent experiments are presented.

TABLE 7
Summary of the in vitro cytotoxicity of Trastuzumab

| | HER2 positive | | HER2 negative | |
|---|---|---|---|---|
| | SK-BR-3 | HCC-1954 | MDA-MB-231 | MCF-7 |
| IC₅₀, μg/mL | >50 | >50 | >50 | >50 |
| IC₅₀, M | >3.4E−07 | >3.4E−07 | >3.4E−07 | >3.4E−07 |

### Cytotoxicity of 1

The cytotoxicity of payload 1 was evaluated against the different tumor cell lines by performing triplicated 10-points, 2.5-fold dilution DR curves ranging from 100 to 0.03 ng/mL (1.26E-07-3.3E-11 M.

As shown in Table 8, where results corresponding to the geometric mean of the IC₅₀ values obtained in three independent experiments are presented, the cytotoxicity of this compound was similar in all the tumor cell lines regardless of their HER2 expression, with IC₅₀ values in the low nanomolar range, from 8.82E-10 to 1.95E-09 M). The geometric mean IC₅₀ value across the whole cell panel was 1.32E-09 M.

TABLE 8
Summary of the in vitro cytotoxicity of 1

| | HER2 positive | | HER2 negative | |
|---|---|---|---|---|
| | SK-BR-3 | HCC-1954 | MDA-MB-231 | MCF-7 |
| IC₅₀, μg/mL | 8.60E−04 | 1.50E−03 | 6.80E−04 | 1.20E−03 |
| IC₅₀, M | 1.12E−09 | 1.95E−09 | 8.82E−10 | 1.56E−09 |

### Cytotoxicity of ADC1

The cytotoxicity of ADC1 was evaluated against the different tumor cell lines by performing triplicate 10-points, 2.5-fold dilution DR curves ranging from 100 μg/mL to 26 ng/mL (6.67E-07-1.75 E-10 M). The evaluation was performed in three independent experiments, Table 9 summarizes the results corresponding to the geometric mean of the IC₅₀ values obtained in three independent experiments. As observed in Table 9, ADC1 showed a cytotoxicity which is similar to that shown by the parent drug 1 only in HER-2 positive cells. However, in HER2 negative cells such toxicity is significantly lower: nearly 8-fold lower according to the selectivity ratio obtained by dividing the mean IC₅₀ values in HER2 negative cells between that in HER2 positive cells. This selectivity leads us to conclude that the conjugate ADC1 is acting through the interaction of the antibody with the membrane associates HER2 receptor on the tumor cells, followed by intracellular delivery of the cytotoxic drug.

TABLE 9
Summary of in vitro activity of ADC1

| | | | | | IC₅₀ in | IC₅₀ in | |
|---|---|---|---|---|---|---|---|
| | HER2 positive | | HER2 negative | | HER2+ | HER2− | |
| | SK- | HCC- | MDA- | | (geom. | (geom. | Selectivity |
| | BR-3 | 1954 | MB-231 | MCF-7 | Mean) | Mean) | ratio |
| IC₅₀ | 9.00E−01 | 1.00E+00 | 5.70E+00 | 1.00E+01 | 9.49E−01 | 7.55E+00 | 8.0 |
| (μg/mL) | | | | | | | |
| IC₅₀ | 6.00E−09 | 6.67E−09 | 3.80E−08 | 6.67E−08 | 6.33E−09 | 5.03E−08 | |
| (M)) | | | | | | | |

### Bioactivity Example 2—Cytotoxicity of the Conjugate ADC 2 and Related Reagents Against HER2 Positive and Negative Breast Cancer Cells

The in vitro cytotoxicity of the ADC2 along with the parent cytotoxic compound 2 were evaluated against four different human breast cancer cell lines over-expressing or not the HER2 receptor, including XK-BR-3, HCC-1954 (HER2 positive cells) as well as MDA-MB-231 and MCF-7 (HER2 negative cells). Standard dose-response (DR) curves for 72 hours incubation with the tested substances were performed. The results are also compared with the monoclonal antibody Trastuzumab described above.

### Cytotoxicity of 2

The cytotoxicity of the intermediate compound 2 was evaluated against the different tumor cell lines by performing triplicated 10-points, 2.5-fold dilution DR curves ranging from 100 to 0.03 ng/mL (1.26E-07-3.3E-11 M. As shown in Table 10, where results corresponding to the geometric mean of the IC₅₀ values obtained in three independent experiments are presented, the cytotoxicity of this compound was similar in all the tumor cell lines regardless of their HER2 expression, with IC₅₀ values in the low nanomolar range, from 8.85E-10 to 2-31E-09 M). The geometric mean with IC₅₀ value across the whole cell panel was 1.53E-09 M.

TABLE 10
Summary of the in vitro cytotoxicity of 2

| | HER2 positive | | HER2 negative | |
|---|---|---|---|---|
| | SK-BR-3 | HCC-1954 | MDA-MB-231 | MCF-7 |
| IC₅₀, μg/mL | 9.60E−04 | 1.80E−03 | 6.90E−04 | 1.70E−03 |
| IC₅₀, M | 1.23E−09 | 2.31E−09 | 8.85E−10 | 2.18E−09 |

### Cytotoxicity of ADC2

The cytotoxicity of ADC2 was evaluated against the different tumor cell lines by performing triplicate 10-points 2.5-fold dilution DR curves ranging from 100 μg/mL to 26 ng/mL (6.67E-07-1.75E-10 M). The evaluation was performed in three independent experiments, Table 11 summarized the results corresponding to the geometric mean of the IC₅₀ values obtained in the three independent experiments. As observed in Table 11, ADC2 showed a cytotoxicity which is similar to that shown by the parent drug 2 only in HER2-positive cells. However, in HER2-negative cells such toxicity is significantly lower according to the selectivity ratio obtained by dividing the mean IC₅₀ in HER2-negative cells between that in HER2-positive cells. This selectivity leads us to conclude that ADC2 is acting through the interaction of the antibody with the membrane associates HER2 receptor on the tumor cells, followed by intracellular delivery of the cytotoxic drug.

TABLE 11
Summary of the in vitro cytotoxicity of ADC2

| | | | | | IC₅₀ in | IC₅₀ in | |
|---|---|---|---|---|---|---|---|
| | HER2 positive | | HER2 negative | | HER2+ | HER2− | |
| | SK- | HCC- | MDA- | | (geom. | (geom. | Selectivity |
| | BR-3 | 1954 | MB-231 | MCF-7 | Mean) | Mean) | ratio |
| IC₅₀ | 8.50E+00 | 1.80E+01 | >1.0E+02 | >1.0E+02 | 1.24E+01 | >1.0E+02 | >8.09 |
| (μg/mL) | | | | | | | |
| IC₅₀ | 5.67E−08 | 1.20E−07 | >6.67E−07 | >6.67E−07 | 8.25E−08 | >6.67E−07 | |
| (M)) | | | | | | | |

### Bioactivity Example 3—Cytotoxicity of the Conjugate ADC 3 and Related Reagents Against HER2 Positive and Negative Breast Cancer Cells

The in vitro cytotoxicity of ADC3 was evaluated against four different human breast cancer cell lines over-expressing or not the HER2 receptor, including SK-BR-3, HCC-1954 (HER2-positive cells) as well as MDA-MB-231 and MCF-7 (HER2-negative cells. Standard dose-response (DR) curves for 72 hours incubation with the tested substances were performed.

### Cytotoxicity of ADC3

The cytotoxicity of ADC3 was evaluated against the different tumor cell lines by performing triplicate 10-points, 2.5-fold dilution DR curves ranging from 100 μg/mL to 26 ng/mL (6.67E-07-1.75E-10 M). The evaluation was performed in three independent experiments, Table 12 summarizes the results corresponding to the geometric mean of the IC₅₀ values obtained in three independent experiments. As observed in Table 12, ADC3 showed a cytotoxicity which is similar to that shown by the parent drug 1 only in HER2 positive cells. However, in HER2 negative cells such toxicity is significantly lower, nearly 56-fold lower according to the selectivity ratio obtained by dividing the mean IC50 value in HER2-negative cells between that in HER2-positive cells. This selectivity leads us to conclude that the conjugate is acting through the interaction of the antibody with the membrane associated HER2 receptor on the tumor cells, followed by intracellular delivery of the cytotoxic drug.

TABLE 12
In vitro activity of ADC3

| | | | | | IC₅₀ in | IC₅₀ in | |
|---|---|---|---|---|---|---|---|
| | HER2 positive | | HER2 negative | | HER2+ | HER2− | |
| | SK- | HCC- | MDA- | | (geom. | (geom. | Selectivity |
| | BR-3 | 1954 | MB-231 | MCF-7 | Mean) | Mean) | ratio |
| IC₅₀ | 2.50E−01 | 2.70E−01 | 1.40E+01 | 1.70E+01 | 2.60E−01 | 1.94E+00 | 55.7 |
| (μg/mL) | | | | | | | |
| IC₅₀ | 1.67E−09 | 1.80E−09 | 9.33E−08 | 1.00E−07 | 1.73E−09 | 9.66E−08 | |
| (M)) | | | | | | | |

### Bioactivity Example 4: Demonstrating the In Vivo Efficacy of the Antibody-Drug Conjugates of the Present Invention

The in vitro cytotoxicity of the ADC4 along was evaluated against four different human breast cancer cell lines over-expressing or not the HER2 receptor, including SK-BR-3, HCC-1954 (HER-2 positive cells) as well as MDA-MB-231 and MCF-7 (HER2-negative cells). Standard dose-response (DR) curves for 72 hours incubation with the tested substances were performed.

### Cytotoxicity of ADC4

The cytotoxicity of ADC4 was evaluated against the different tumor cell lines by performing triplicate 10-points, 2.5-fold dilution DR curves ranging from 100 μg/mL to 26 ng/mL (6.67E-07-1.75E-10 M). The evaluation was performed in three different experiments, Table 13 summarizes the results corresponding to the geometric mean of the IC₅₀ values obtained in three different experiments. As observed in Table 13, ADC4 showed a cytotoxicity which is similar to that shown by the parent drug 2 only in HER2 positive cells. However, in HER2 negative cells such toxicity in significantly lower: nearly 14-fold lower according to the selectivity ration obtained by dividing the mean IC₅₀ value in HER2-negative cells between that in HER2-positive cells. This selectivity leads us to conclude that the conjugate ADC4 is acting through the interaction of the antibody with the membrane associated HER2 receptor on the tumor cells, followed by intercellular delivery of the cytotoxic drug.

TABLE 13
In vitro activity of ADC4

| | | | | | IC₅₀ in | IC₅₀ in | |
|---|---|---|---|---|---|---|---|
| | HER2 positive | | HER2 negative | | HER2+ | HER2− | |
| | SK- | HCC- | MDA- | | (geom. | (geom. | Selectivity |
| | BR-3 | 1954 | MB-231 | MCF-7 | Mean) | Mean) | ratio |
| IC₅₀ | 3.10E−01 | 6.30E−01 | 7.00E+00 | 5.40E+00 | 4.42E−01 | 6.15E+00 | 13.91 |
| (μg/mL) | | | | | | | |
| IC₅₀ | 2.07E−09 | 4.20E−09 | 4.67E−08 | 3.60E−08 | 2.95E−09 | 4.10E−08 | |
| (M)) | | | | | | | |

1-119. (canceled)

120. A drug conjugate comprising a drug moiety covalently attached to the rest of the drug conjugate, the drug conjugate having formula [D-(X)_{b}-(AA)-(T)_{g}-(L)-]ₙ-Ab wherein:
D is a drug moiety having the following formula (IH) or a pharmaceutically acceptable salt, ester, solvate, tautomer or stereoisomer thereof, 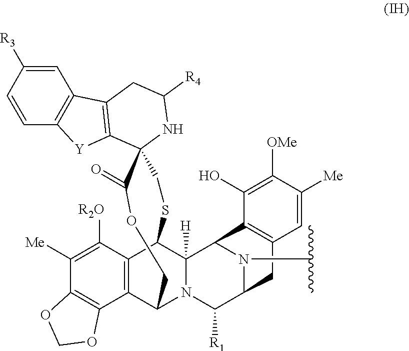
wherein:
the wavy line indicates the point of covalent attachment to (X)_{b} if any, or (AA), if any, or to (T)_{g} if any, or to (L);
Y is —NH— or —O—;
R₁ is —OH or —CN;
R₂ is a —C(═O)Rₐ group;
R₃ is hydrogen or a —OR_{b} group;
R₄ is selected from hydrogen, —CH₂OH, —CH₂OC(═O)R_{c}, —CH₂NH₂, and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂ alkenyl, and substituted or unsubstituted C₂-C₁₂ alkynyl;
R_{c} is selected from substituted or unsubstituted C₁-C₁₂ alkyl, substituted or unsubstituted C₂-C₁₂alkenyl, and substituted or unsubstituted C₂-C₁₂ alkynyl; and
Prot^{NH} is a protecting group for amino;
X and T are extending groups that may be the same or different;
each AA is independently an amino acid unit;
L is a linker group;
w is an integer ranging from 0 to 12;
b is an integer of 0 or 1;
g is an integer of 0 or 1;
Ab is a moiety comprising at least one antigen binding site; and
n is the ratio of the group [D-(X)_{b}-(AA)_{w}-(T)_{g}-(L)-] to the moiety comprising at least one antigen binding site and is in the range from 1 to 20.

121. The drug conjugate according to claim 120, wherein D is a compound of formula: 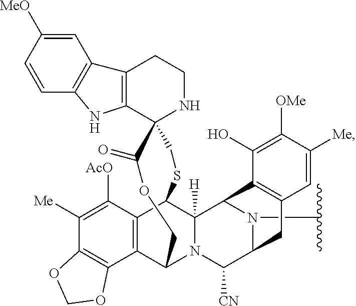 [SMILES: *N1[C@@H]2c3c(cc(C)c(OC)c3O)C[C@H]1[C@H](C#N)N1[C@H]2[C@@H]2SC[C@]3(NCCc4c3[nH]c3ccc(OC)cc43)C(=O)OC[C@H]1c1c3c(c(C)c(OC(C)=O)c12)OCO3]
or a pharmaceutically acceptable salt or ester thereof.

122. The drug conjugate according to claim 120, wherein D is a compound of formula: 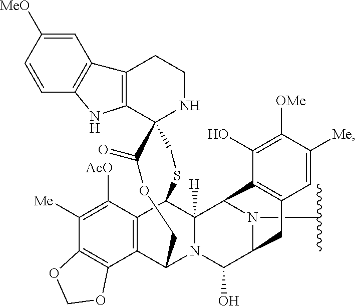 [SMILES: *N1[C@@H]2c3c(cc(C)c(OC)c3O)C[C@H]1[C@H](O)N1[C@H]2[C@@H]2SC[C@]3(NCCc4c3[nH]c3ccc(OC)cc43)C(=O)OC[C@H]1c1c3c(c(C)c(OC(C)=O)c12)OCO3]
or a pharmaceutically acceptable salt or ester thereof.

123. The drug conjugate according to claim 120, wherein the salt is selected from hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate, p-toluenesulfonate, sodium, potassium, calcium, ammonium, ethylenediamine, ethanolamine, N,N-dialkylenethanolamine, triethanolamine and basic aminoacids.

124. The drug conjugate according to claim 120, wherein L is a linker group selected from the group consisting of: 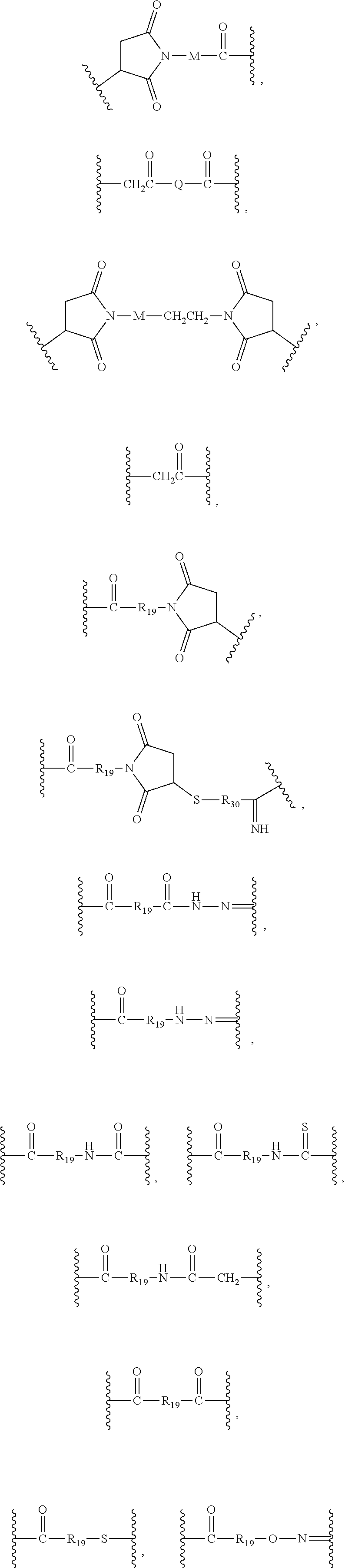
wherein
the wavy lines indicate the point of covalent attachments to an Ab (the wavy line to the right) and to (T)_{g} if any, or (AA), if any, or (X)_{b} if any, or D (the wavy line to the left);
R₁₉ is selected from —C₁-C₁₂ alkylene-, —C₃-C₈ carbocyclo, —O—(C₁-C₁₂ alkylene), —C₆-Cis arylene in one or more rings which may optionally be substituted with one or more substituents Rₓ, —C₁-C₁₂ alkylene-C₆-C₁₈ arylene- wherein the arylene group is in one or more rings which may optionally be substituted with one or more substituents Rₓ, —C₆-Cis arylene-C₁-C₁₂ alkylene- wherein the arylene group is in one or more rings which may optionally be substituted with one or more substituents Rₓ, —C₁-C₁₂ alkylene-(C₃-C₈ carbocyclo)-, —(C₃-C₈ carbocyclo)-C₁-C₁₂ alkylene-, —C₅-C₁₄ heterocyclo- wherein said heterocyclo group may be a saturated or unsaturated group having one or more rings and comprising at least one oxygen, nitrogen or sulphur atom in said ring(s), said group optionally being substituted with one or more substituents Rₓ, —C₁-C₁₂ alkylene-(C₅-C₁₄ heterocyclo)- wherein said heterocyclo group may be a saturated or unsaturated group having one or more rings and comprising at least one oxygen, nitrogen or sulphur atom in said ring(s), said group optionally being substituted with one or more substituents Rₓ, —(C₅-C₁₄ heterocyclo)-C₁-C₁₂ alkylene- wherein said heterocyclo group may be a saturated or unsaturated group having one or more rings and comprising at least one oxygen, nitrogen or sulphur atom in said ring(s), said group optionally being substituted with one or more substituents Rₓ, —(OCH₂CH₂)ᵣ— and —CH₂—(OCH₂CH₂)ᵣ, wherein each of the above alkylene substituents whether alone or attached to another moiety the carbon chain may optionally be substituted by one or more substituents Rₓ;
R₃₀ is a —C₁-C₆ alkylene- group;
M is selected from the group consisting of —C₁-C₆ alkylene-, —C₁-C₆ alkylene-(C₃-C₈ carbocyclo)-, —(CH₂CH₂O)ₛ—, —C₁-C₆ alkylene-(C₃-C₈ carbocyclo)-CON(H or C₁-C₆ alkyl)-C₁-C₆ alkylene-, phenylene which may optionally be substituted with one or more substituents Rₓ, phenylene-C₁-C₆ alkylene- wherein the phenylene moiety may optionally be substituted with one or more substituents Rₓ and —C₁-C₆ alkylene-CON(H or C₁-C₆ alkyl)C₁-C₆ alkylene-;
Q is selected from the group consisting of —N(H or C₁-C₆ alkyl)phenylene- and —N(H or C₁-C₆ alkyl)-(CH₂)ₛ;
r is an integer ranging from 1 to 10; and
s is an integer ranging from 1 to 10;
or
wherein L is a linker group selected from the group consisting of: 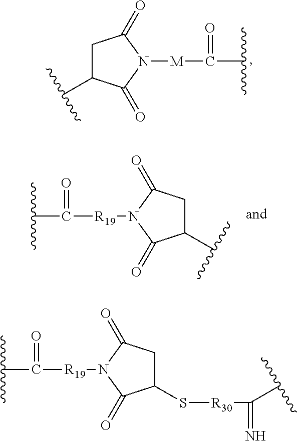
wherein:
the wavy lines indicate the point of covalent attachments to an Ab (the wavy line to the right) and to (T)_{g} if any, or (AA), if any, or to (X)_{b} (the wavy line to the left);
R₁₉ is selected from —C₁-C₁₂ alkylene-, —O—(C₁-C₁₂ alkylene), —C₆-C₁₂ arylene in one or more rings which may optionally be substituted with one or more substituents Rₓ, —C₁-C₁₂ alkylene-C₆-C₁₂ arylene- wherein the arylene group is in one or more rings which may optionally be substituted with one or more substituents Rₓ, —C₆-C₁₂ arylene-C₁-C₁₂ alkylene- wherein the arylene group is in one or more rings which may optionally be substituted with one or more substituents Rₓ, —C₅-C₁₂ heterocyclo- wherein said heterocyclo group may be a saturated or unsaturated group having one or more rings and comprising at least one oxygen, nitrogen or sulphur atom in said ring(s), said group optionally being substituted with one or more substituents Rₓ, —C₁-C₁₂ alkylene-(C₅-C₁₂ heterocyclo)- wherein said heterocyclo group may be a saturated or unsaturated group having one or more rings and comprising at least one oxygen, nitrogen or sulphur atom in said ring(s), said group optionally being substituted with one or more substituents Rₓ, —(C₅-C₁₂ heterocyclo)-C₁-C₁₂ alkylene- wherein said heterocyclo group may be a saturated or unsaturated group having one or more rings and comprising at least one oxygen, nitrogen or sulphur atom in said ring(s), said group optionally being substituted with one or more substituents Rₓ, —(OCH₂CH₂)ᵣ— and —CH₂—(OCH₂CH₂)ᵣ, wherein each of the above alkylene substituents whether alone or attached to another moiety the carbon chain may optionally be substituted by one or more substituents Rₓ;
R₃₀ is a —C₁-C₆ alkylene- group;
M is selected from the group consisting of —C₁-C₆ alkylene-, —C₁-C₆ alkylene-(C₃-C₈ carbocyclo)- and phenylene which may optionally be substituted with one or more substituents Rₓ; and
r is an integer ranging from 1-6.

125. The drug conjugate according to claim 120, selected from the formulas (IV), (V) and (VI): 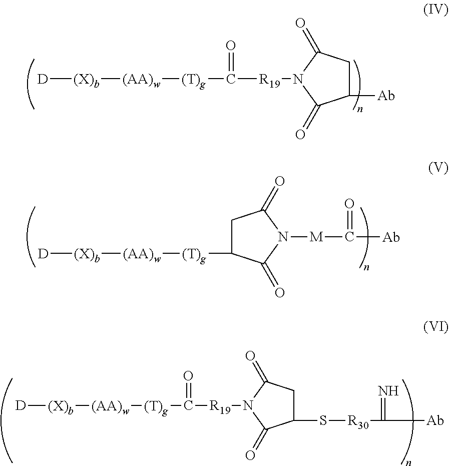
wherein:
X and T are extending groups that may be the same or different;
each AA is independently an amino acid unit;
w is an integer ranging from 0 to 12;
b is an integer of 0 or 1;
g is an integer of 0 or 1;
D is a drug moiety;
Ab is a moiety comprising at least one antigen binding site;
n is the ratio of the group [D-(X)_{b}-(AA)-(T)_{g}-(L)-] wherein L is as defined in formula (IV), (V) or (VI) to the moiety comprising at least one antigen binding site and is in the range from 1 to 20;
R₁₉ is selected from —C₁-C₈ alkylene-, —O—(C₁-C₈ alkylene), —C₁-C₈ alkylene-C₆-C₁₂ arylene- wherein the arylene group is in one or more rings which may optionally be substituted with one or more substituents Rₓ, and —C₆-C₁₂ arylene-C₁-C₈ alkylene- wherein the arylene group is in one or more rings which may optionally be substituted with one or more substituents Rₓ,
wherein each of the above alkylene substituents whether alone or attached to another moiety the carbon chain may optionally be substituted by one or more substituents Rₓ;
R₃₀ is a —C₂-C₄ alkylene- group; and
M is selected from the group consisting of —C₁-C₃ alkylene- and —C₁-C₃ alkylene-(C₅-C₇ carbocyclo)-;
or
selected from the formulas (IV), (V) and (VI): 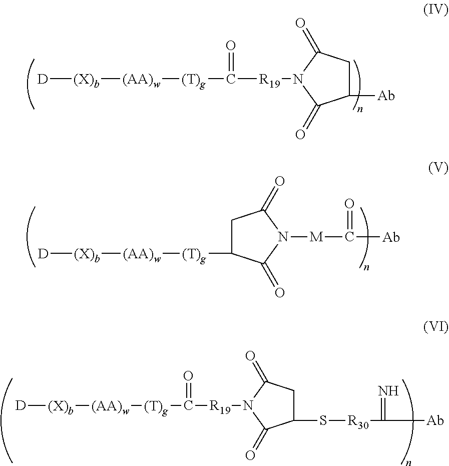
wherein:
X and T are extending groups that may be the same or different;
each AA is independently an amino acid unit;
w is an integer ranging from 0 to 12;
b is an integer of 0 or 1;
g is an integer of 0 or 1;
D is a drug moiety;
Ab is a moiety comprising at least one antigen binding site;
n is the ratio of the group [D-(X)_{b}-(AA)_{w}-(T)_{g}-(L)-] wherein L is as defined in (IV), (V) or (VI) to the moiety comprising at least one antigen binding site and is in the range from 1 to 20;
R₁₉ is selected from —C₁-C₆ alkylene-, phenylene-C₁-C₆ alkylene- wherein the phenylene group may optionally be substituted with one or more substituents Rₓ selected from the group consisting of alkyl groups having from 1 to 6 carbon atoms, alkoxy groups having from 1 to 6 carbon atoms, halogen atoms, nitro groups and cyano groups, wherein each of the above alkylene substituents whether alone or attached to another moiety in the carbon chain may optionally be substituted by one or more substituents Rₓ selected from the group consisting of alkyl groups having from 1 to 6 carbon atoms, alkoxy groups having from 1 to 6 carbon atoms, aryl groups having from 6 to 12 carbon atoms, halogen atoms, nitro groups and cyano groups;
R₃₀ is a —C₂-C₄ alkylene- group; and
M is —C₁-C₃ alkylene-(C₅-C₇ carbocyclo)-.

126. The drug conjugate according to claim 120, wherein (AA), is of formula (II): 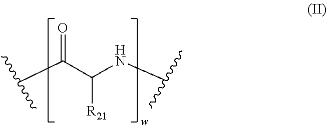
wherein the wavy lines indicate the point of covalent attachments to (X)_{b} if any, or to the drug moiety (the wavy line to the left) and to (T)_{g} if any, or to the linker (the wavy line to the right); and
R₂₁ is, at each occurrence, selected from the group consisting of hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl, 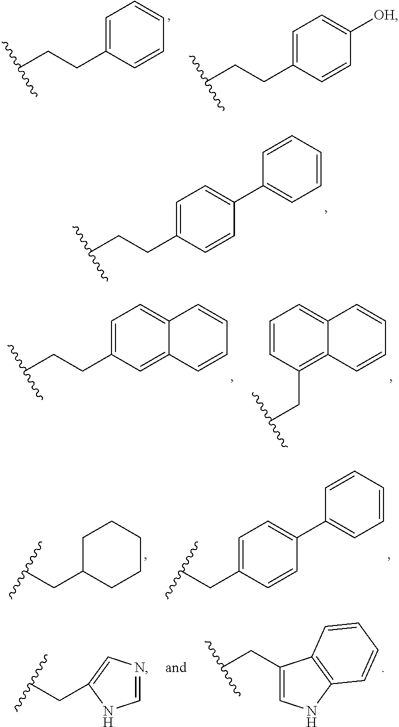
and w is an integer ranging from 0 to 12;
or
wherein (AA), is of formula (II) wherein:
R₂₁ is selected, at each occurrence, from the group consisting of hydrogen, methyl, isopropyl, sec-butyl, benzyl, indolylmethyl, —(CH₂)₃NHCONH₂, —(CH₂)₄NH₂, —(CH₂)₃NHC(═NH)NH₂ and —(CH₂)₄NHC(═NH)NH₂; and
w is an integer ranging from 0 to 6;
or
wherein w is 0 or 2, and where w is 2, then (AA), is of formula (III): 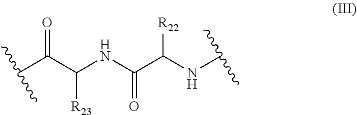
wherein:
the wavy lines indicate the point of covalent attachments to (X)_{b} if any, or to the drug moiety (the wavy line to the left) and to (T)_{g} if any, or to the linker (the wavy line to the right);
R₂₂ is selected from methyl, benzyl, isopropyl, sec-butyl and indolylmethyl; and
R₂₃ is selected from methyl, —(CH₂)₄NH₂, —(CH₂)₃NHCONH₂ and —(CH₂)₃NHC(═NH)NH₂.

127. The drug conjugate according to claim 120, wherein X is an extending group selected from:
where D is covalently attached via an amine group:
—COO—(C₁-C₆ alkylene)NH—;
—COO—CH₂-(phenylene which may optionally be substituted with one or more substituents Rₓ)—NH—;
—COO—(C₁-C₆ alkylene)NH—COO—CH₂-(phenylene which may optionally be substituted with one or more substituents Rₓ)—NH—;
—COCH₂NH—COCH₂—NH—;
—COCH₂NH—;
—COO—(C₁-C₆ alkylene)S—;
—COO—(C₁-C₆ alkylene)NHCO(C₁-C₆ alkylene)S—; and
where D is covalently attached via an hydroxy group:
—CONH—(C₁-C₆ alkylene)NH—;
—COO—CH₂-(phenylene which may optionally be substituted with one or more substituents Rₓ)—NH—;
—CONH—(C₁-C₆ alkylene)NH—COO—CH₂-(phenylene which may optionally be substituted with one or more substituents Rₓ)—NH—;
—COCH₂NH—COCH₂—NH—;
—COCH₂NH—;
—CONH—(C₁-C₆ alkylene)S—;
—CONH—(C₁-C₆ alkylene)NHCO(C₁-C₆ alkylene)S—; and
b is 0 or 1;
or
wherein X is an extending group selected from the group consisting of:
where D is covalently attached via an amine group:
—COO—(C₂-C₄ alkylene)NH—;
—COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups;
—COO—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—;
—COCH₂NH—COCH₂—NH—;
—COO—(C₂-C₄ alkylene)S—;
—COO—(C₂-C₄ alkylene)NHCO(C₁-C₃ alkylene)S—; or
where D is covalently attached via an hydroxy group:
—CONH—(C₂-C₄ alkylene)NH—;
—COO—CH₂-phenylene-NH—, wherein said phenylene group may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups;
—CONH—(C₂-C₄ alkylene)NH—COO—CH₂-(phenylene which may optionally be substituted with from one to four substituents Rₓ selected from the group consisting of alkyl groups having from 1 to 6 carbon atoms, alkoxy groups having from 1 to 6 carbon atoms, halogen atoms, nitro groups and cyano groups)-NH—;
—COCH₂NH—COCH₂—NH—;
—CONH—(C₂-C₄ alkylene)S—;
—CONH—(C₂-C₄ alkylene)NHCO(C₁-C₃ alkylene)S—; and
b is 0 or 1;
or
wherein X is an extending group selected from the group consisting of:
where D is covalently attached via an amine group:
—COO—CH₂-phenylene-NH—
—COO(CH₂)₃NHCOOCH₂-phenylene-NH—;
—COO(CH₂)₃NH—;
—COO(CH₂)₃—S—;
—COO(CH₂)₃NHCO(CH₂)₂S—; or
where D is covalently attached via an hydroxy group:
—COO—CH₂-phenylene-NH—
—CONH(CH₂)₃NHCOOCH₂-phenylene-NH—;
—CONH(CH₂)₃NH—;
—CONH(CH₂)₃—S—;
—CONH(CH₂)₃NHCO(CH₂)₂S—; and
b is 0 or 1.

128. The drug conjugate according to claim 120, wherein T is an extending group selected from the group consisting of —CO—(C₁-C₆ alkylene)-NH—, —CO—(C₁-C₆ alkylene)-[O—(C₂-C₆ alkylene)]ⱼ—NH—, —COO—(C₁-C₆ alkylene)-[O—(C₂-C₆ alkylene)]ⱼ—NH—; where j is an integer from 1 to 25, and g is 0 or 1;
or
wherein T is an extending group selected from the group consisting of —CO—(C₁-C₄ alkylene)NH—, —CO—(C₁-C₄ alkylene)-[O—(C₂-C₄ alkylene)]ⱼ—NH—, —COO—(C₁-C₄ alkylene)-[O—(C₂-C₄ alkylene)]ⱼ—NH—, where j is an integer from 1 to 10; and g is 0 or 1;
or
wherein T is an extending group selected from the group consisting of —CO—(C₁-C₄ alkylene)NH—, —CO—(C₁-C₄ alkylene)-[O—(C₂-C₄ alkylene)]ⱼ—NH—, —COO—(C₁-C₄ alkylene)-[O—(C₂-C₄ alkylene)]ⱼ—NH—; where j is an integer from 1 to 5; and g is 0 or 1.

129. The drug conjugate according to claim 120, wherein D is a drug moiety of formula (IH) or a pharmaceutically acceptable salt, ester, solvate, tautomer or stereoisomer thereof, wherein:
R₁ is CN or OH;
R₂ is C(═O)Rₐ, wherein Rₐ is selected from hydrogen and substituted or unsubstituted C₁-C₆ alkyl, wherein the optional substituents are one or more substituents Rₓ;
R₃ is hydrogen or a —OR_{b} group wherein R_{b} is a substituted or unsubstituted C₁-C₆ alkyl group, wherein the optional substituents are one or more substituents Rₓ,
R₄ is selected from hydrogen, —CH₂OH, and —CH₂NH₂; and
Y is —NH— or —O—;
or
wherein D is a drug moiety of formula (IH) or a pharmaceutically acceptable salt, ester, solvate, tautomer or stereoisomer thereof, wherein:
R₁ is CN or OH;
R₂ is acetyl;
R₃ is hydrogen or methoxy;
R₄ is hydrogen or —CH₂OH; and
Y is —NH— or —O—;
or
wherein D is a drug moiety of formula (IH), or a pharmaceutically acceptable salt, ester, solvate, tautomer or stereoisomer thereof wherein:
R₁ is CN;
R₂ is acetyl:
R₃ is methoxy;
R₄ is hydrogen; and
Y is —NH— or —O—.

130. The drug conjugate according to claim 120, wherein D is selected from: 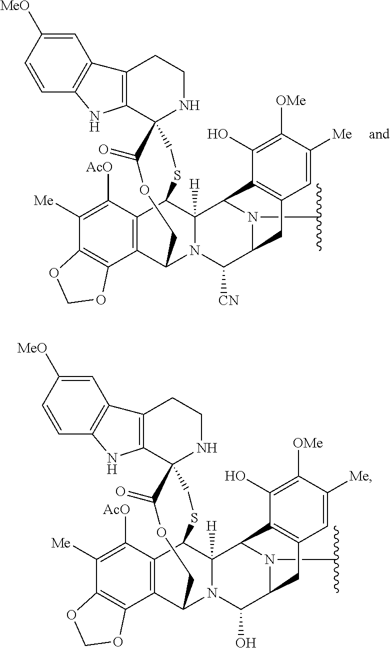 [SMILES: *N1[C@@H]2c3c(cc(C)c(OC)c3O)C[C@H]1[C@H](C#N)N1[C@H]2[C@@H]2SC[C@]3(NCCc4c3[nH]c3ccc(OC)cc43)C(=O)OC[C@H]1c1c3c(c(C)c(OC(C)=O)c12)OCO3 ; "and" ; *N1[C@@H]2c3c(cc(C)c(OC)c3O)C[C@H]1[C@H](O)N1[C@H]2[C@@H]2SC[C@]3(NCCc4c3[nH]c3ccc(OC)cc43)C(=O)OC[C@H]1c1c3c(c(C)c(OC(C)=O)c12)OCO3]
or a pharmaceutically acceptable salt, ester, solvate, tautomer or stereoisomer thereof, wherein the wavy line indicates the point of covalent attachment to (X)_{b} if any, or (AA), if any, or to (T)_{g} if any, or to (L).

131. The drug conjugate according to claim 120, wherein the moiety Ab comprising at least one antigen binding site is an antigen-binding peptide;
including
wherein the moiety Ab comprising at least one antigen binding site is an antibody, a single domain antibody or an antigen-binding fragment thereof;
or
wherein the moiety Ab comprising at least one antigen binding site is a monoclonal antibody, polyclonal antibody or bispecific antibody and/or wherein the antibody or an antigen-binding fragment thereof is derived from a human, mouse or rabbit;
or
wherein the moiety Ab comprising at least one antigen binding site is selected from the group consisting of a human antibody, an antigen-binding fragment of a human antibody, a humanized antibody, an antigen-binding fragment of a humanized antibody, a chimeric antibody, an antigen-binding fragment of a chimeric antibody, a glycosylated antibody and a glycosylated antigen binding fragment;
or
wherein the moiety Ab comprising at least one antigen binding site is an antigen-binding fragment selected from the group consisting of an Fab fragment, an Fab′ fragment, an F(ab′)₂ fragment and an Fv fragment;
or
wherein the moiety Ab comprising at least one antigen binding site is a monoclonal antibody which immunospecifically binds to cancer cell antigens, viral antigens, antigens of cells that produce autoimmune antibodies associated with autoimmune disease, or microbial antigens; or
wherein the moiety Ab comprising at least one antigen binding site is an antibody selected from the group consisting of Alemtuzumab, Anetumab, Atezolizumab, Avelumab, Bevacizumab, Blinatomumab, Brentuximab, Catumaxomab, Cetuximab, Coltuximab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Lifastuzumab, Lorvotuzumab, Milatuzumab, Mirvetuximab, Naratuximab, Necitumumab, Nimotuzumab, Nivolumab, Obinutuzumab, Ofatumumab, Olaratumab, Panitumumab, Pembrolizumab, Pertuzumab, Pinatuzumab, Polatuzumab, Ramucirumab, Rovalpituzumab, Sacituzumab, Siltuximab, Sirtratumab, Sofituzumab, Vadastuximab, Vorsetuzumab, Trastuzumab or other an anti-HER2 antibody, an anti-CD4 antibody, an anti-CD5 antibody, an anti-CD13 antibody and an anti-CD30 antibody, or an antigen-binding fragment or an immunologically active portion thereof;
or
wherein the moiety Ab comprising at least one antigen binding site is an antibody selected from the group consisting of Alemtuzumab, Anetumab, Atezolizumab, Avelumab, Bevacizumab, Blinatomumab, Brentuximab, Catumaxomab, Cetuximab, Daratumumab, Denintuzumab, Denosumab, Depatuxizumab, Dinutuximab, Durvalumab, Elotuzumab, Enfortumab, Glembatumumab, Gemtuzumab, Ibritumomab, Indatuximab, Indusatumab, Inotuzumab, Ipilimumab, Labetuzumab, Ladiratuzumab, Laprituximab, Mirvetuximab, Naratuximab, Necitumumab, Nimotuzumab, Nivolumab, Obinutuzumab, Ofatumumab, Olaratumab, Panitumumab, Pembrolizumab, Pertuzumab, Polatuzumab, Ramucirumab, Rovalpituzumab, Sacituzumab, Siltuximab, Sirtratumab, Vadastuximab, Vorsetuzumab, Trastuzumab or other an anti-HER2 antibody, an anti-CD4 antibody, an anti-CD5 antibody, an anti-CD13 antibody and an anti-CD30 antibody, or an antigen-binding fragment or an immunologically active portion thereof;
or
wherein the moiety Ab comprising at least one antigen binding site is an antibody selected from the group consisting of Alemtuzumab, Atezolizumab, Avelumab, Bevacizumab, Blinatomumab, Brentuximab, Catumaxomab, Cetuximab, Daratumumab, Denosumab, Dinutuximab, Durvalumab, Elotuzumab, Gemtuzumab, Ibritumomab, Inotuzumab, Ipilimumab, Labetuzumab, Necitumumab, Nimotuzumab, Nivolumab, Obinutuzumab, Ofatumumab, Olaratumab, Panitumumab, Pembrolizumab, Pertuzumab, Ramucirumab, Rovalpituzumab, Siltuximab, Trastuzumab or another anti-HER2 antibody, an anti-CD4 antibody, an anti-CD5 antibody, an anti-CD13 antibody and an anti-CD30 antibody, or an antigen-binding fragment or an immunologically active portion thereof;
or
wherein the moiety Ab comprising at least one antigen binding site is an aptamer, including a nucleic acid or a peptide aptamer.

132. The drug conjugate according to claim 120, that is an antibody drug conjugate selected from the group consisting of: 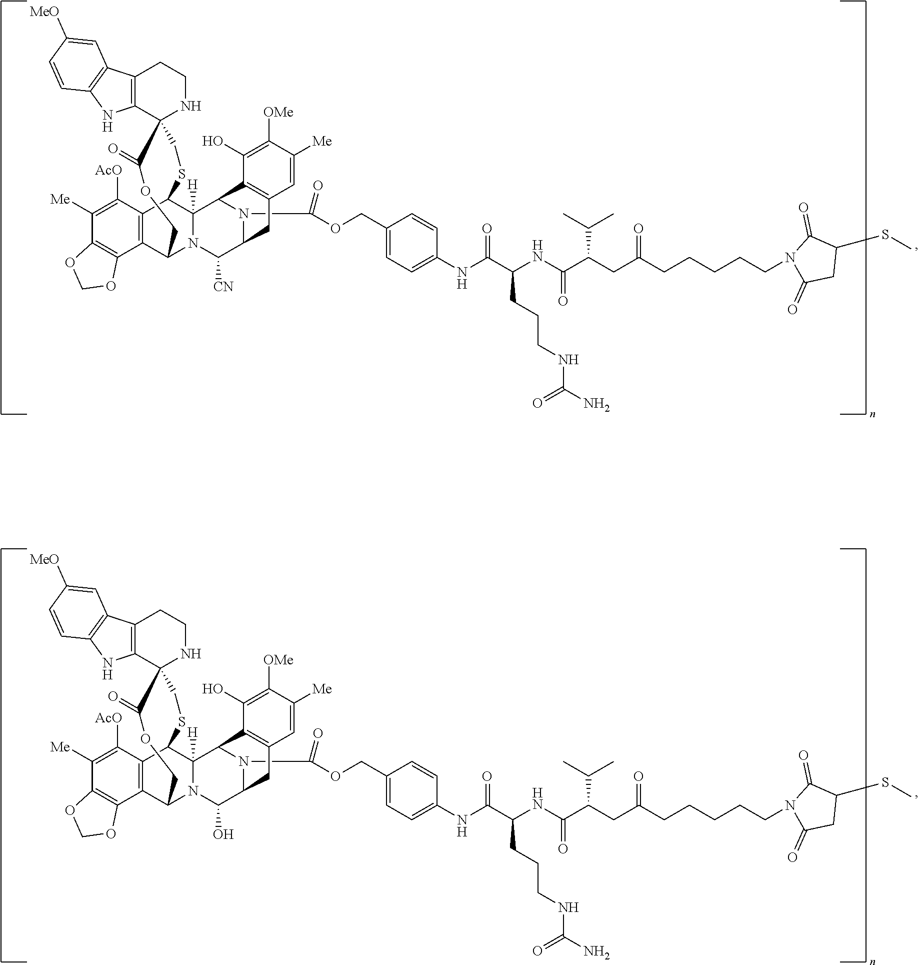 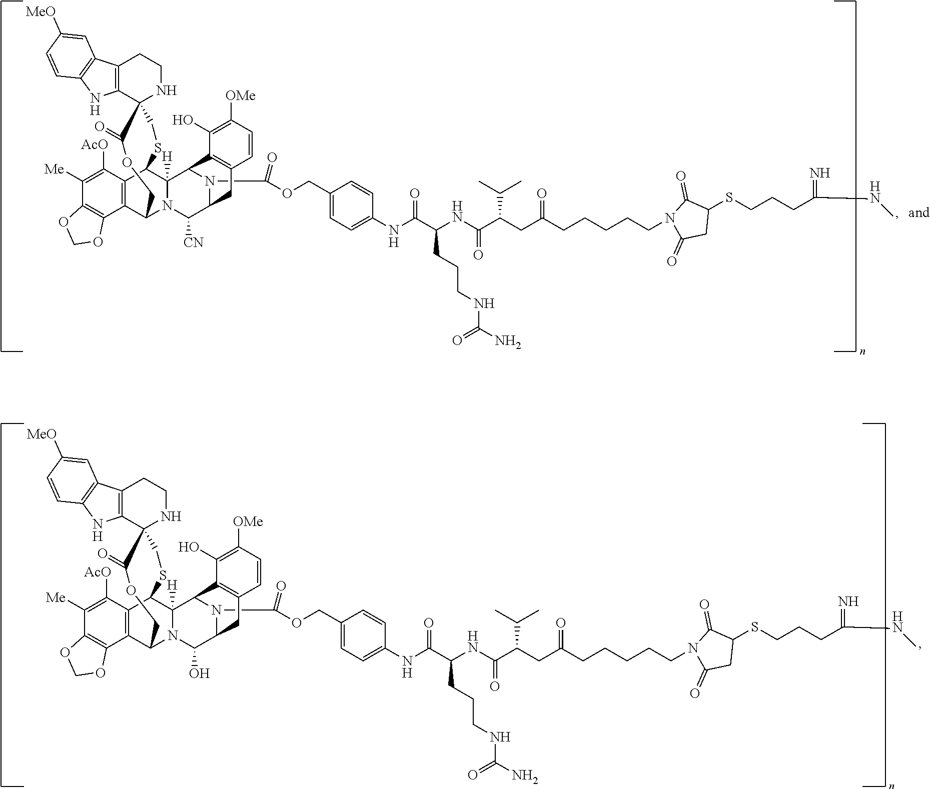
wherein n is from 2 to 6 and each [symbol] and [symbol] is independently selected from Brentuximab, Gemtuzumab, Inozutumab, Rovalpituzumab, Trastuzumab or another an anti-HER2 antibody, an anti-CD4 antibody, an anti-CD5 antibody, an anti-CD13 antibody and an anti-CD30 antibody, or an antigen-binding fragment or an immunologically active portion thereof.

133. The drug conjugate according to claim 120, that is an antibody drug conjugate in isolated or purified form.

134. A compound of formula D-(X)_{b}-(AA)_{w}-(T)_{g}-L₁ or of formula D-(X)_{b}-(AA)_{w}-(T)_{g}-H, wherein:
L₁ is a linker selected from the group of formulas consisting of: 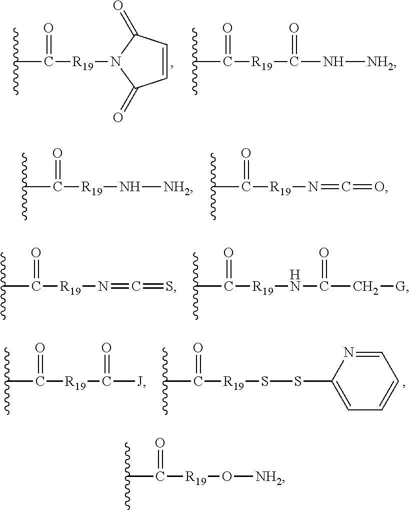
wherein each of the the wavy lines indicates the point of covalent attachment to (T)_{g} if any, or (AA), if any, or to (X)_{b} if any or to D;
G is selected from halo, —O-mesyl and —O-tosyl;
J is selected from halo, hydroxy, —N-succinimidoxy, —O-(4-nitrophenyl), —O— pentafluorophenyl, —O-tetrafluorophenyl and —O—C(O)—OR₂₀;
R₁₉ is selected from —C₁-C₁₂ alkylene-, —C₃-C₈ carbocyclo, —O—(C₁-C₁₂ alkylene), —C₆-Cis arylene in one or more rings which may optionally be substituted with one or more substituents Rₓ, —C₁-C₁₂ alkylene-C₆-C₁₈ arylene- wherein the arylene group is in one or more rings which may optionally be substituted with one or more substituents Rₓ, —C₆-C₁₈ arylene-C₁-C₁₂ alkylene- wherein the arylene group is in one or more rings which may optionally be substituted with one or more substituents Rₓ, —C₁-C₁₂ alkylene-(C₃-C₈ carbocyclo)-, —(C₃-C₈ carbocyclo)-C₁-C₁₂ alkylene-, —C₅-C₁₄ heterocyclo- wherein said heterocyclo group may be a saturated or unsaturated group having one or more rings and comprising at least one oxygen, nitrogen or sulphur atom in said ring(s), said group optionally being substituted with one or more substituents Rₓ, —C₁-C₁₂ alkylene-(C₅-C₁₄ heterocyclo)- wherein said heterocyclo group may be a saturated or unsaturated group having one or more rings and comprising at least one oxygen, nitrogen or sulphur atom in said ring(s), said group optionally being substituted with one or more substituents Rₓ, —(C₅-C₁₄ heterocyclo)-C₁-C₁₂ alkylene-, wherein said heterocyclo group may be a saturated or unsaturated group having one or more rings and comprising at least one oxygen, nitrogen or sulphur atom in said ring(s), said group optionally being substituted with one or more substituents Rₓ, —(OCH₂CH₂)ᵣ— and —CH₂—(OCH₂CH₂)ᵣ, wherein each of the above alkylene substituents whether alone or attached to another moiety the carbon chain may optionally be substituted by one or more substituents Rₓ;
R₂₀ is a C₁-C₁₂ alkyl or an aryl group having from 6 to 18 carbon atoms in one or more aromatic rings, said aryl groups optionally being substituted with one or more substituents Rₓ;
r is an integer ranging from 1-10;
b is an integer of 0 or 1;
g is an integer of 0 or 1;
w is an integer ranging from 0 to 12;
wherein for compounds of formula D-(X-)_{b}(AA)_{w}-(T)_{g}-H, b+w+g≠0;
each of D, Rₓ, X, T, and AA is as defined in claim 120;
including
wherein the compound of formula D-X-(AA)_{w}-(T)_{g}-L₁ is selected from:

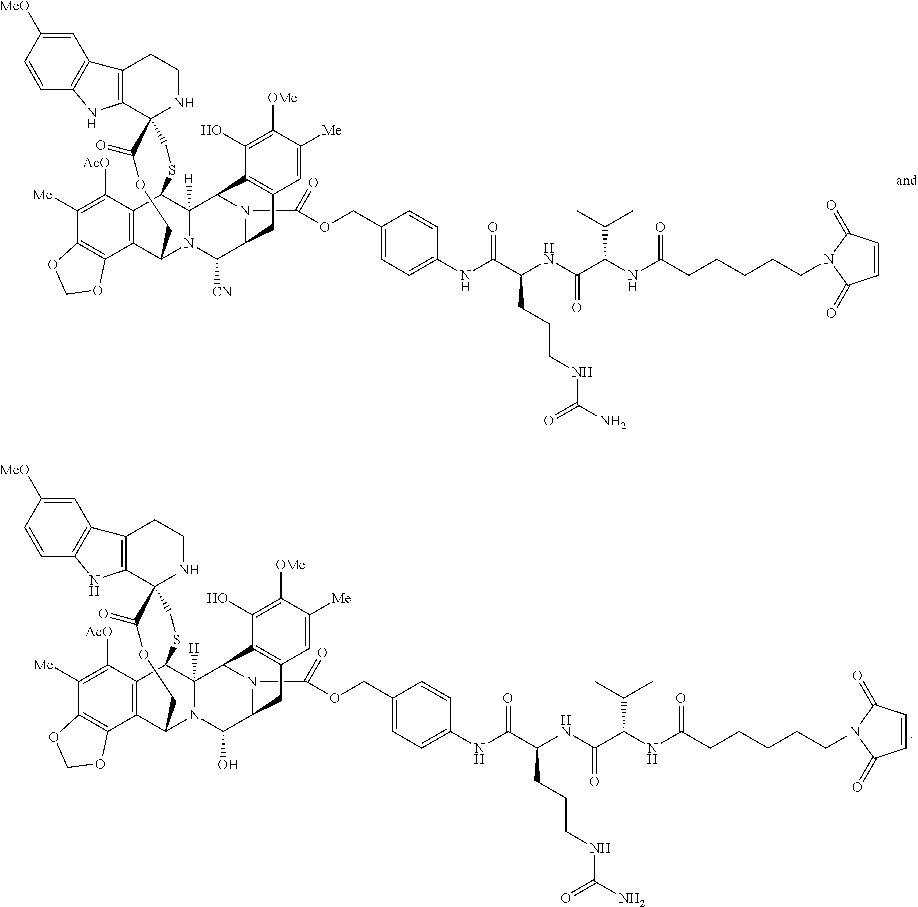 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1C#N)N2C(=O)OCc1ccc(NC(=O)[C@H](CCCNC(N)=O)NC(=O)[C@@H](NC(=O)CCCCCN2C(=O)C=CC2=O)C(C)C)cc1)OCO4 ; "and" ; COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C(=O)OCc1ccc(NC(=O)[C@H](CCCNC(N)=O)NC(=O)[C@@H](NC(=O)CCCCCN2C(=O)C=CC2=O)C(C)C)cc1)OCO4]

135. A compound of formula D-(X)_{b}-(AA)_{w}-(T)_{g}-L₁ or of formula D-(X)_{b}-(AA)_{w}-(T)_{g}-H, wherein each of D, X, AA, T, L₁, b, g and w are as defined in claim 120; but further wherein if the compound is a compound of formula D-(X)_{b}-(AA)_{w}-(T)_{g}-H then b+w+g≠0.

136. The drug conjugate according to claim 120, wherein b+g+w is not 0;
or
wherein b+w is not 0;
or
wherein when w is not 0, then b is 1;
or
wherein when w is 0, then b is 1.

137. The compound according to claim 134, wherein b+g+w is not 0;
or
wherein b+w is not 0;
or
wherein when w is not 0, then b is 1;
or
wherein when w is 0, then b is 1.

138. The compound according to claim 135, wherein b+g+w is not 0;
or
wherein b+w is not 0;
or
wherein when w is not 0, then b is 1;
or
wherein when w is 0, then b is 1.

139. The drug conjugate according to claim 120, wherein, unless otherwise defined, if substituted, substituted groups are substituted with one or more substituents Rₓ that are independently selected from the group consisting of C₁-C₁₂ alkyl groups which may be optionally substituted with at least one group R_{y}, C₂-C₁₂ alkenyl groups which may be optionally substituted with at least one group R_{y}, C₂-C₁₂ alkynyl groups which may be optionally substituted with at least one group R_{y}, halogen atoms, oxo groups, thio groups, cyano groups, nitro groups, OR_{y}, OCOR_{y}, OCOOR_{y}, COR_{y}, COOR_{y}, OCONR_{y}R_{z}, CONR_{y}R_{z}, S(O)R_{y}, SO₂R_{y}, P(O)(R_{y})OR_{z}, NR_{y}R_{z}, NR_{y}COR_{z}, NR_{y}C(═O)NR_{y}R_{z}, NR_{y}C(═NR_{y})NR_{y}R_{z}, aryl groups having from 6 to 18 carbon atoms in one or more rings which may optionally be substituted with one or more substituents which may be the same or different selected from the group consisting of R_{y}, OR_{y}, OCOR_{y}, OCOOR_{y}, NR_{y}R_{z}, NR_{y}COR_{z}, and NR_{y}C(═NR_{y})NR_{y}R_{z}, aralkyl groups comprising an alkyl group having from 1 to 12 carbon atoms substituted with an optionally substituted aryl group as defined above, aralkyloxy groups comprising an alkoxy group having from 1 to 12 carbon atoms substituted with an optionally substituted aryl group as defined above, and a 5- to 14-membered saturated or unsaturated heterocyclic group having one or more rings and comprising at least one oxygen, nitrogen or sulphur atom in said ring(s), said heterocyclic group optionally being substituted with one or more substituents R_{y}, and where there is more than one optional substituents on any given group the optional substituents R_{y} may be the same or different;
each R_{y} and R_{z} is independently selected from the group consisting of hydrogen, C₁-C₁₂ alkyl groups, C₁-C₁₂ alkyl groups that are substituted with at least one halogen atom, aralkyl groups comprising a C₁-C₁₂ alkyl group that is substituted with an aryl group having from 6 to 18 carbon atoms in one or more rings and heterocycloalkyl groups comprising a C₁-C₁₂alkyl group that is substituted with a 5- to 14-membered saturated or unsaturated heterocyclic group having one or more rings and comprising at least one oxygen, nitrogen or sulphur atom in said ring(s).

140. The compound according to claim 134, wherein, unless otherwise defined, if substituted, substituted groups are substituted with one or more substituents Rₓ that are independently selected from the group consisting of C₁-C₁₂ alkyl groups which may be optionally substituted with at least one group R_{y}, C₂-C₁₂ alkenyl groups which may be optionally substituted with at least one group R_{y}, C₂-C₁₂ alkynyl groups which may be optionally substituted with at least one group R_{y}, halogen atoms, oxo groups, thio groups, cyano groups, nitro groups, OR_{y}, OCOR_{y}, OCOOR_{y}, COR_{y}, COOR_{y}, OCONR_{y}R_{z}, CONR_{y}R_{z}, S(O)R_{y}, SO₂R_{y}, P(O)(R_{y})OR_{z}, NR_{y}R_{z}, NR_{y}COR_{z}, NR_{y}C(═O)NR_{y}R_{z}, NR_{y}C(═NR_{y})NR_{y}R_{z}, aryl groups having from 6 to 18 carbon atoms in one or more rings which may optionally be substituted with one or more substituents which may be the same or different selected from the group consisting of R_{y}, OR_{y}, OCOR_{y}, OCOOR_{y}, NR_{y}R_{z}, NR_{y}COR_{z}, and NR_{y}C(═NR_{y})NR_{y}R_{z}, aralkyl groups comprising an alkyl group having from 1 to 12 carbon atoms substituted with an optionally substituted aryl group as defined above, aralkyloxy groups comprising an alkoxy group having from 1 to 12 carbon atoms substituted with an optionally substituted aryl group as defined above, and a 5- to 14-membered saturated or unsaturated heterocyclic group having one or more rings and comprising at least one oxygen, nitrogen or sulphur atom in said ring(s), said heterocyclic group optionally being substituted with one or more substituents R_{y}, and where there is more than one optional substituents on any given group the optional substituents R_{y} may be the same or different;
each R_{y} and R_{z} is independently selected from the group consisting of hydrogen, C₁-C₁₂ alkyl groups, C₁-C₁₂ alkyl groups that are substituted with at least one halogen atom, aralkyl groups comprising a C₁-C₁₂ alkyl group that is substituted with an aryl group having from 6 to 18 carbon atoms in one or more rings and heterocycloalkyl groups comprising a C₁-C₁₂ alkyl group that is substituted with a 5- to 14-membered saturated or unsaturated heterocyclic group having one or more rings and comprising at least one oxygen, nitrogen or sulphur atom in said ring(s).

141. The compound according to claim 135, wherein, unless otherwise defined, if substituted, substituted groups are substituted with one or more substituents Rₓ that are independently selected from the group consisting of C₁-C₁₂ alkyl groups which may be optionally substituted with at least one group R_{y}, C₂-C₁₂ alkenyl groups which may be optionally substituted with at least one group R_{y}, C₂-C₁₂ alkynyl groups which may be optionally substituted with at least one group R_{y}, halogen atoms, oxo groups, thio groups, cyano groups, nitro groups, OR_{y}, OCOR_{y}, OCOOR_{y}, COR_{y}, COOR_{y}, OCONR_{y}R_{z}, CONR_{y}R_{z}, S(O)R_{y}, SO₂R_{y}, P(O)(R_{y})OR_{z}, NR_{y}R_{z}, NR_{y}COR_{z}, NR_{y}C(═O)NR_{y}R_{z}, NR_{y}C(═NR_{y})NR_{y}R_{z}, aryl groups having from 6 to 18 carbon atoms in one or more rings which may optionally be substituted with one or more substituents which may be the same or different selected from the group consisting of R_{y}, OR_{y}, OCOR_{y}, OCOOR_{y}, NR_{y}R_{z}, NR_{y}COR_{z}, and NR_{y}C(═NR_{y})NR_{y}R_{z}, aralkyl groups comprising an alkyl group having from 1 to 12 carbon atoms substituted with an optionally substituted aryl group as defined above, aralkyloxy groups comprising an alkoxy group having from 1 to 12 carbon atoms substituted with an optionally substituted aryl group as defined above, and a 5- to 14-membered saturated or unsaturated heterocyclic group having one or more rings and comprising at least one oxygen, nitrogen or sulphur atom in said ring(s), said heterocyclic group optionally being substituted with one or more substituents R_{y}, and where there is more than one optional substituents on any given group the optional substituents R_{y} may be the same or different;
each R_{y} and R_{z} is independently selected from the group consisting of hydrogen, C₁-C₁₂ alkyl groups, C₁-C₁₂ alkyl groups that are substituted with at least one halogen atom, aralkyl groups comprising a C₁-C₁₂ alkyl group that is substituted with an aryl group having from 6 to 18 carbon atoms in one or more rings and heterocycloalkyl groups comprising a C₁-C₁₂ alkyl group that is substituted with a 5- to 14-membered saturated or unsaturated heterocyclic group having one or more rings and comprising at least one oxygen, nitrogen or sulphur atom in said ring(s).

142. A method of manufacturing an antibody drug conjugate comprising utilizing a drug moiety D as defined in claim 120 or a compound according to claim 134 or 135.

143. A drug conjugate according to claim 120, for use as a medicament.

144. A method of treating cancer comprising administering a therapeutically effective amount of a drug conjugate according to claim 120 to a patient in need thereof; wherein the cancer includes lung cancer including NSCLC, gastric cancer, colorectal cancer, breast cancer, pancreas carcinoma, endometrial cancer, bladder cancer, cervical cancer, esophageal cancer, gallbladder cancer, uterine cancer, salivary duct cancer, ovarian cancer, kidney cancer, leukaemia, multiple myeloma, and lymphoma;
optionally wherein the cancer is a HER2 positive cancer or a HER2 positive lung cancer including HER2 positive NSCLC, HER2 positive gastric cancer, HER2 positive colorectal cancer, HER2 positive breast cancer, HER2 positive pancreas carcinoma, HER2 positive endometrial cancer, HER2 positive bladder cancer, HER2 positive cervical cancer, HER2 positive esophageal cancer, HER2 positive gallbladder cancer, HER2 positive uterine cancer, HER2 positive salivary duct cancer or HER2 positive ovarian cancer.

145. A pharmaceutical composition comprising the drug conjugate according to claim 120 and a pharmaceutically acceptable carrier.

146. The drug conjugate according to claim 120, wherein n is in the range of 1-12, 1-8, 3-8, 3-6, or 3-5 or n is 1, 2, 3, 4, 5 or 6.

147. A process for the preparation of a drug antibody conjugate according to claim 120, comprising conjugating a moiety Ab comprising at least one antigen binding site and a drug D, Ab and D being as defined in claim 120;
including wherein
the preparation of a drug antibody conjugate of formula (G) or (G′): 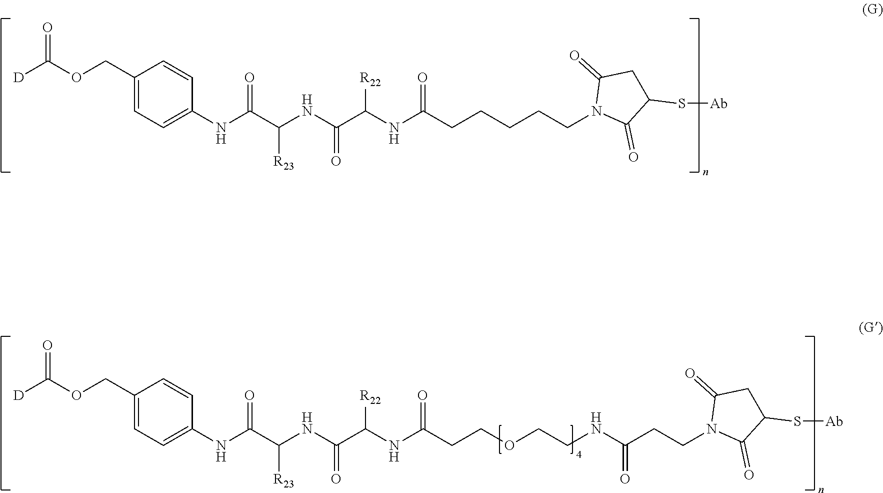
said process comprising the following steps:
(i) reacting a drug D-H of formula (IH)-H: 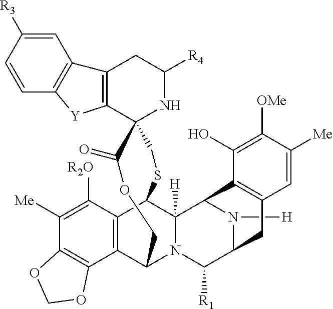
wherein the substituents in the definitions of (IH)-H are as defined in claim 120 with a compound of formula (D′) or (E): 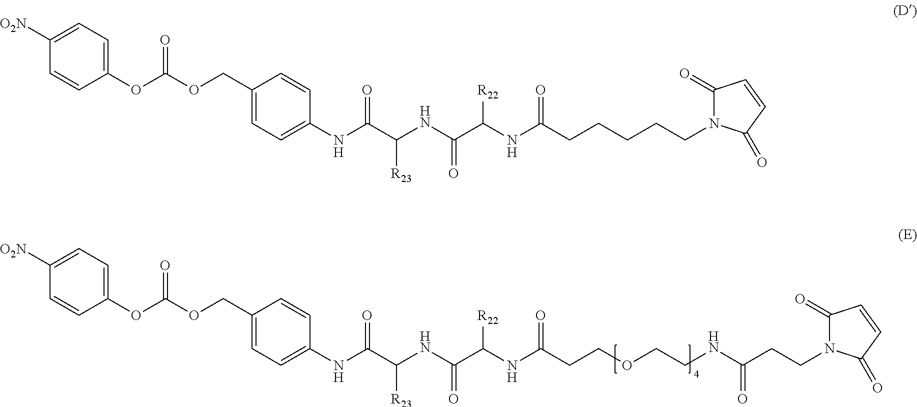
to give a compound of formula (F) or (F′), respectively: 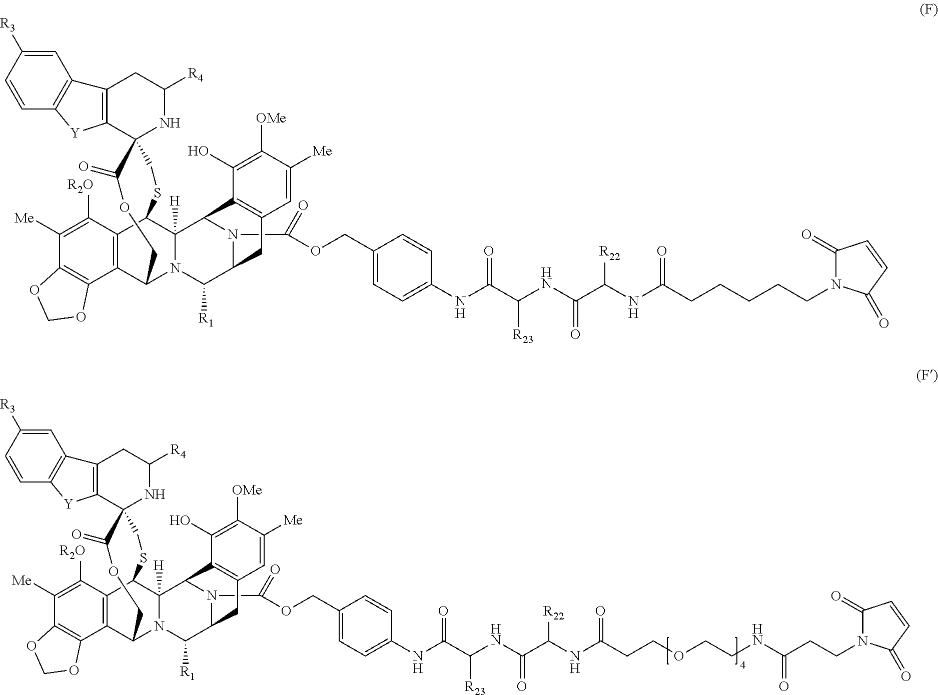
(ii) partial reduction of one or more disulfide bonds in the antibody to be conjugated to give a reduced antibody Ab-SH having free thiol groups: 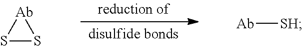
and
(iii) reaction of the partially reduced antibody Ab-SH having free thiol groups with the compound of formula (F) or (F′) produced in step (i) to give the desired drug antibody conjugate of formula (G) or (G′) respectively:

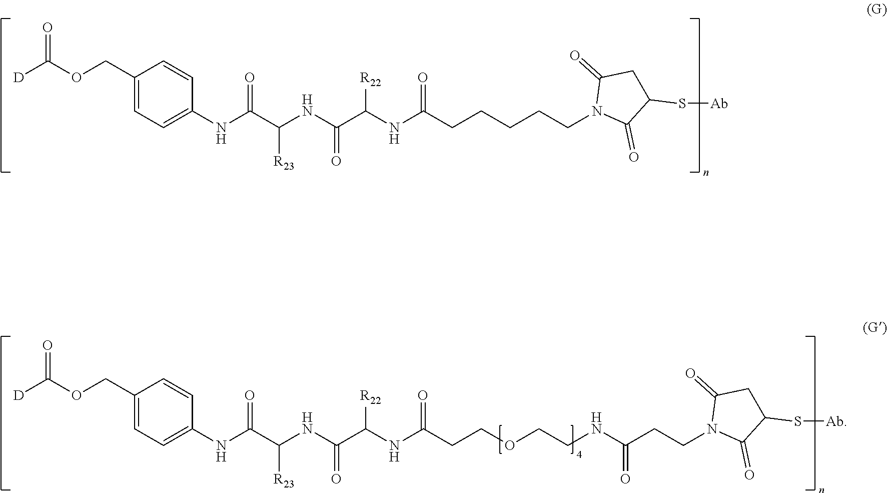

148. A compound of formula (IA) 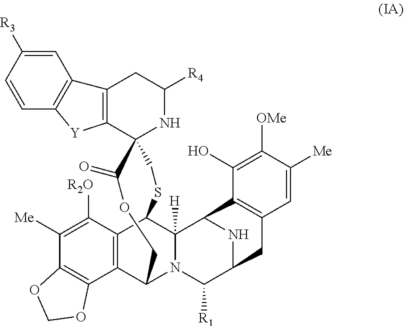
wherein:
Y is —NH— or —O—;
R₁ is —OH or —CN;
R₂ is a —C(═O)Rₐ group;
R₃ is hydrogen or a OR_{b} group;
R₄ is selected from hydrogen, —CH₂OH, —CH₂OC(═O)Re, —CH₂NH₂ and —CH₂NHProt^{NH};
Rₐ is selected from hydrogen, substituted or unsubstituted C₁-C₁₂ alkyl, substituted or unsubstituted C₂-C₁₂ alkenyl, and substituted or unsubstituted C₂-C₁₂ alkynyl;
R_{b} is selected from substituted or unsubstituted C₁-C₁₂ alkyl, substituted or unsubstituted C₂-C₁₂alkenyl, and substituted or unsubstituted C₂-C₁₂ alkynyl;
R_{c} is selected from substituted or unsubstituted C₁-C₁₂ alkyl, substituted or unsubstituted C₂-C₁₂ alkenyl, and substituted or unsubstituted C₂-C₁₂ alkynyl; and
Prot^{NH} is a protecting group for amino;
with the proviso that when R₄ is hydrogen, then Y is —O—.
